(19) United States
(12) Patent Application Publication
(10) Pub. No.: US 2026/0015582 A1
(43) Pub. Date: Jan. 15, 2026

(54) METHODS OF T CELL DIFFERENTIATION AND COMPOSITIONS THEREOF

(71) Applicant: THE CHILDREN'S MEDICAL CENTER CORPORATION, Boston, MA (US)

(72) Inventors: George Q. DALEY, Cambridge, MA (US); Ran JING, Wayland, MA (US)

(73) Assignee: THE CHILDREN'S MEDICAL CENTER CORPORATION, Boston, MA (US)

(21) Appl. No.: 18/994,861

(22) PCT Filed: Jul. 18, 2023
(86) PCT No.: PCT/US2023/070376

§ 371 Date: Jan. 15, 2025

Related U.S. Application Data
(60) Provisional application No. 63/390,050, filed on Jul. 18, 2022.

(51) Int. Cl. C12N 5/0783; A61K 40/11; A61K 40/31; A61K 40/42; A61P 35/00

(52) U.S. Cl. CPC C12N 5/0636; A61K 40/11; A61K 40/31; A61K 40/42; A61P 35/00; C12N 2501/105; C12N 2501/115; C12N 2501/125; C12N 2501/14; C12N 2501/145; C12N 2501/155; C12N 2501/16; C12N 2501/165; C12N 2501/2303; C12N 2501/2306; C12N 2501/2307; C12N 2501/2311; C12N 2501/26; C12N 2501/42; C12N 2501/727; C12N 2506/45; C12N 2510/00

(57) ABSTRACT

The technology described herein is directed to improved methods of T cell differentiation. Also described herein are immune cells differentiated using such methods and compositions comprising such immune cells. In some embodiments, the immune cells can be genetically modified. In some embodiments, the immune cells or compositions comprising said immune cells can be administered to a patient as a cellular replacement therapy to treat a condition.

### CROSS-REFERENCE TO RELATED APPLICATION

This application claims benefit under 35 U.S.C. § 119(e) of the U.S. Provisional Application No. 63/390,050 filed Jul. 18, 2022, the contents of each of which are incorporated herein by reference in their entireties.

### SEQUENCE LISTING

The instant application contains a Sequence Listing that has been submitted in XML format via patent Center and is hereby incorporated by reference in its entirety. Said XML copy, created on Jul. 6, 2023, is named “701039-192140WOPT_SL.xml” and is 94,065 bytes in size.

### TECHNICAL FIELD

The technology described herein relates to immune cell differentiation methods.

### BACKGROUND

There is a lack of supply of functional immune cells for the in vivo cellular replacement therapy, therapy for a host of diseases, disorders and conditions, and for the in vitro studies of disease modeling, drug screening, and hematological diseases. T cells are key components of human adaptive immune system and have great therapeutic potential. However, current T cell-mediated therapy relies on autologous T cells, which prevents its broad application. Human induced pluripotent stem cells (iPSCs) represent an ideal source for scalable manufacture of off-the-shelf products for cell therapy. However, the generation of mature and functional T cells from iPSCs has proven to be difficult. Additionally, the differentiation of iPSC requires co-culture with mouse stromal cells, which limits the translational potential of iPSC-derived T cells. As such there is a need for high-yield, clinically applicable T cell differentiation methods.

### SUMMARY

The method and compositions are based, in part, on the observation that contacting CD34+ hemogenic endothelium with interleukin-3 can increase yields of CD3+ T cells and can also increase the production of mature CD3+ T cells having an αβ phenotype.

Accordingly, provided herein in one aspect are methods for generating CD3+ T cells, the method comprising: (a) contacting a CD34+ hemogenic endothelium (HE) with a CD5+CD7+ differentiation medium comprising interleukin-3 (IL-3) under conditions and for a sufficient time to generate CD5+CD7+ T cell progenitor cells, and (b) contacting the CD5+CD7+ T cell progenitor cells with a CD3+ T cell differentiation medium under conditions and for a sufficient time to generate CD3+ T cells.

In one embodiment of this aspect and all other aspects provided herein, the concentration of IL-3 is 1-10 ng/mL. In another embodiment of this aspect and all other aspects provided herein, the concentration of IL-3 is about 5 ng/mL. In another embodiment of this aspect and all other aspects provided herein, the concentration of IL-3 is 5 ng/mL.

In another embodiment of this aspect and all other aspects provided herein, the CD34+ hemogenic endothelium is contacted with IL-3 for about one week.

In another embodiment of this aspect and all other aspects provided herein, the CD5+CD7+ differentiation medium further comprises at least one of: stem cell factor (SCF), FLT-3, IL-7 and thrombopoietin (TPO).

In another embodiment of this aspect and all other aspects provided herein, the CD5+CD7+ differentiation medium further comprises each of stem cell factor (SCF), FLT-3, IL-7 and thrombopoietin (TPO).

In another embodiment of this aspect and all other aspects provided herein, the concentration of SCF is 5-50 ng/mL, and/or the concentration of FLT-3 is 5-30 ng/mL, and/or the concentration of IL-7 is 10-50 ng/mL, and/or the concentration of TPO is 1-10 ng/mL.

In another embodiment of this aspect and all other aspects provided herein, the CD3+ T cell differentiation medium comprises Fms related receptor tyrosine kinase-3 (FLT3), and interleukin-7 (IL-7).

In another embodiment of this aspect and all other aspects provided herein, the concentration of IL-7 is 1-30 ng/mL, and/or the concentration of FLT-3 is 5-30 ng/mL.

In another embodiment of this aspect and all other aspects provided herein, the yield of CD3+ T cells is higher than a substantially similar method lacking IL-3 in step (a).

In another embodiment of this aspect and all other aspects provided herein, the CD34+ HE undergoes endothelial-to-hematopoietic transition (EHT).

In another embodiment of this aspect and all other aspects provided herein, step (a) is performed for at least 1 week.

In another embodiment of this aspect and all other aspects provided herein, step (a) is performed for 2 weeks.

In another embodiment of this aspect and all other aspects provided herein, step (b) is performed for at least 1 week.

In another embodiment of this aspect and all other aspects provided herein, the CD34+ HE is cultured in the presence of a Notch ligand. In another embodiment of this aspect and all other aspects provided herein, the CD34+ HE is cultured in the presence of a Notch ligand and in the presence of at least one additional adhesion molecule (e.g., vitronectin).

In another embodiment of this aspect and all other aspects provided herein, the Notch ligand is attached to a solid surface. In another embodiment of this aspect and all other aspects provided herein, the Notch ligand and the at least one additional adhesion molecule is attached to a solid surface.

In another embodiment of this aspect and all other aspects provided herein, the Notch ligand is attached to a cell culture dish. In another embodiment of this aspect and all other aspects provided herein, the Notch ligand and the at least one additional adhesion molecule is attached to a cell culture dish.

In another embodiment of this aspect and all other aspects provided herein, the Notch ligand is not derived from a stromal cell. In another embodiment of this aspect and all other aspects provided herein, the Notch ligand and the at least one additional adhesion molecule are not derived from a stromal cell.

In another embodiment of this aspect and all other aspects provided herein, differentiating the CD34+ hemogenic endothelium in the presence of a Notch ligand (with or without at least one additional adhesion molecule) does not comprise co-culturing with a stromal cell expressing a Notch ligand.

In another embodiment of this aspect and all other aspects provided herein, differentiating the hemogenic endothelium in the presence of a Notch ligand (with or without an additional adhesion molecule, such as vitronectin) does not comprise co-culturing with OP9-DL1 cells or OP9-DL4 cells.

In another embodiment of this aspect and all other aspects provided herein, the Notch ligand is selected from the group consisting of Delta-like-1 (DLL1), Delta-like-4 (DLL4), immobilized Delta1^{ext-IgG}, and immobilized Delta4^{ext-IgG}. In one embodiment, the concentration of DLL1, DLL4, immobilized Delta1^{ext-IgG}, or immobilized Delta4^{ext-IgG} is in the range of 1 μg/mL to 40 μg/mL; in other embodiments the concentration is in the range of 5 μg/mL to 30 μg/mL, 10 μg/mL to 30 μg/mL, 20 μg/mL to 30 μg/mL, 5 μg/mL to 20 μg/mL, 5 μg/mL to 10 μg/mL, 5 μg/mL to 25 μg/mL, 10 μg/mL to 20 μg/mL, or any integer therebetween. In other embodiments, the concentration of DLL1, DLL4, immobilized Delta1^{ext-IgG}, or immobilized Delta4^{ext-IgG} is about 10 μg/mL, about 15 μg/mL, about 20 μg/mL or about 25 μg/mL.

In another embodiment of this aspect and all other aspects provided herein, the method of generating CD3+ T cells further comprises culturing the CD34+ HE in the presence of vitronectin.

In another embodiment of this aspect and all other aspects provided herein, the concentration of vitronectin is in the range of 1-20 μg/mL (e.g., 1-15 μg/mL, 1-10 μg/mL, 5-20 μg/mL, 5-15 μg/mL, 5-10 μg/mL, 10-15 μg/mL, or 10-20 μg/mL).

In another embodiment of this aspect and all other aspects provided herein, the concentration of vitronectin is 10 μg/mL. In another embodiment of this aspect and all other aspects provided herein the CD34+ HE is cultured in the presence of both DLL4 and vitronectin, wherein the concentration of DLL4 is 10 μg/mL or 20 μg/mL and the concentration of vitronectin is 10 μg/mL.

In another embodiment of this aspect and all other aspects provided herein, immobilized Delta1^{ext-IgG} consists of an extracellular domain of human Delta-like-1 fused to the Fc domain of human IgG1.

In another embodiment of this aspect and all other aspects provided herein, the CD5+CD7+ T cell differentiation medium and/or the CD3⁺-T-cell-differentiation media are serum-free.

In another embodiment of this aspect and all other aspects provided herein, the method further comprises a step of generating CD34+ hemogenic endothelium from a population of pluripotent stem cells, optionally induced pluripotent stem cells (iPSCs).

In another embodiment of this aspect and all other aspects provided herein, the population of pluripotent stem cells is contacted with an aggregation medium for a sufficient time to generate the CD34+ hemogenic endothelium.

In another embodiment of this aspect and all other aspects provided herein, the population of pluripotent stem cells is differentiated into a population of CD34⁺ hemogenic endothelium by way of embryoid bodies or 2D adherent cultures.

In another embodiment of this aspect and all other aspects provided herein, the sufficient time to promote differentiation into a population of CD34⁺ hemogenic endothelium is at least 8 days.

In another embodiment of this aspect and all other aspects provided herein, the aggregation media comprises BMP4, SB-431542, CHIR99021, bFGF, VEGF, IL-6, IL-11, IGF-1, SCF, and EPO.

In another embodiment of this aspect and all other aspects provided herein, the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/mL IL-11, 25 ng/mL IGF-1, 50 ng/mL SCF, and 2 U/ml EPO.

In another embodiment of this aspect and all other aspects provided herein, the method further comprises selecting or isolating the resultant population of CD34⁺ hemogenic endothelium using expression of surface markers on the population of CD34⁺ hemogenic endothelium.

In another embodiment of this aspect and all other aspects provided herein, the population of CD34⁺ hemogenic endothelium is CD45 negative/low and/or CD38 negative/low.

In another embodiment of this aspect and all other aspects provided herein, the method further comprises the step of genetically modifying the resultant population of CD34+ hemogenic endothelium or the resultant population of CD3+ T cells.

In another embodiment of this aspect and all other aspects provided herein, the genetic modification is editing an endogenous HLA, removing an endogenous TCR, and/or expressing a chimeric antigen receptor (CAR).

In another embodiment of this aspect and all other aspects provided herein, the CD3+ T cells are CD3⁺TCRαβ⁺ T cells.

In another embodiment of this aspect and all other aspects provided herein, the CD3+ T cells comprise a diverse T cell receptor (TCR) repertoire.

In another embodiment of this aspect and all other aspects provided herein, the method further comprises inhibition of EZH1 activity and/or expression in the CD34+ HE cells.

In another embodiment of this aspect and all other aspects provided herein, EZH1 activity and/or expression are inhibited using an RNA guided nuclease system.

In another embodiment of this aspect and all other aspects provided herein, EZH1 activity and expression are inhibited using a doxycycline-inducible CRISPR interference (CRISPRi) system.

In another embodiment of this aspect and all other aspects provided herein, the inhibition of EZH1 activity and/or expression comprises contacting the cells with an inhibitor of EZH1 expression.

In another embodiment of this aspect and all other aspects provided herein, the inhibitor of EZH1 expression comprises an RNA interference molecule.

Another aspect provided herein relates to a cell or population of cells made by any method described herein.

Another aspect provided herein relates to a method of treating cancer, the method comprising administering a cell or population of cells as described herein.

Another aspect provided herein relates to a method for generating mature αβ T cells, the method comprising: (a) contacting a CD34+ hemogenic endothelium (HE) with a CD5+CD7+ differentiation medium comprising interleukin-3 (IL-3) under conditions and for a sufficient time to generate CD5+CD7+ T cell progenitor cells, (b) contacting the CD5+CD7+ T cell progenitor cells with a CD3+ T cell differentiation medium under conditions and for a sufficient time to generate mature αβ T cells.

Another aspect provided herein relates to a method for generating CD3+ T cells, the method comprising: (a) contacting a CD34+ hemogenic endothelium (HE) lacking EZH1 activity and/or expression with a CD5+CD7+ differentiation medium comprising interleukin-3 (IL-3) under conditions and for a sufficient time to generate CD5+CD7+ T cell progenitor cells, (b) contacting the CD5+CD7+ T cell progenitor cells with a CD3+ T cell differentiation medium under conditions and for a sufficient time to generate CD3+ T cells.

In one embodiment of this aspect and all other aspects provided herein, the CD34+ hemogenic endothelium lacking EZH1 activity and/or expression is generated using an RNA guided nuclease system.

In another embodiment of this aspect and all other aspects provided herein, the EZH1 activity and/or expression is inhibited using an EZH1-specific RNA interference agent or a vector encoding an EZH1-specific RNA interference agent.

Another aspect provided herein relates to a method for generating mature αβ T cells, the method comprising: (a) contacting a CD34+ hemogenic endothelium (HE) lacking EZH1 activity and/or expression with a CD5+CD7+ differentiation medium comprising interleukin-3 (IL-3) under conditions and for a sufficient time to generate CD5+CD7+ T cell progenitor cells, (b) contacting the CD5+CD7+ T cell progenitor cells with a CD3+ T cell differentiation medium under conditions and for a sufficient time to generate mature αβ T cells.

In one embodiment of this aspect and all other aspects provided herein, the CD34+ hemogenic endothelium lacking EZH1 activity and/or expression is generated using an RNA guided nuclease system.

In another embodiment of this aspect and all other aspects provided herein, the EZH1 activity and/or expression is inhibited using an EZH1-specific RNA interference agent or a vector encoding an EZH1-specific RNA interference agent.

### BRIEF DESCRIPTION OF THE DRAWINGS

FIGS. 1A-1G show stroma-free differentiation of human iPSCs into T cells. FIG. 1A, Schematic illustration of T cell differentiation from iPSCs via the Stroma-free system. CD34⁺ HE generated from embryoid bodies were seeded on immobilized DL4 in serum-free media supplemented with cytokines to induce T cell differentiation. FIG. 1B, Representative images showing day 0 CD34⁺ HE cells, day 14 T cell progenitor cells, and day 35 T cells. Scale bar: 200 μm. FIG. 1C, Representative flow cytometry plots showing expression of T cell lineage-specific markers during the stroma-free T cell differentiation of iPSCs (gated on CD45⁺ cells). FIG. 1D, Bar graph showing frequencies of CD4 SP, CD8 SP, and DP T cells in CD3⁺ cells after 6 weeks of differentiation (n=3, mean±SEM). FIG. 1E, Bar graph showing the numbers of week 6 CD3⁺ T cells generated via OP9-DL1 or stroma-free differentiation, normalized to numbers of CD34⁺ HE cells seeded on day 0 (n=3, mean±SEM, *P≤0.05). FIG. 1F, Bar graph showing frequencies of B cells (CD19⁺), NK cells (CD56⁺), Myeloid cells (CD33⁺), T cell precursors (CD5⁺CD3⁻), and T cells (CD3⁺) in CD45⁺ hematopoietic cells after 6 weeks of T cell differentiation using the OP9-DL1 or stroma-free method (n=3, mean±SEM, *P≤0.05, **P≤0.01). FIG. 1G, Bar graph showing the frequencies of Vβ family genes in stroma-free iPSC-T cells determined by sequencing of the TCRβ CDR3 region (n=2)

FIGS. 2A-2I show EZH1 knockdown facilitates in vitro T cell differentiation from iPSCs. FIG. 2A, Schematic illustration of EZ-T cell generation. EB-derived CD34⁺ HE cells were transduced with a viral vector expressing an EZH1 shRNA before stroma-free T cell differentiation. FIG. 2B, Absolute numbers of live cells during T cell differentiation using control or EZH1 knockdown (KD) iPSC-derived HE cells (n=3, mean±SEM, *P≤0.05). FIG. 2C, Bar graph showing frequencies of CD3⁺ T cells in CD45⁺ cells generated from control or EZH1 KD cells after stroma-free T cell differentiation (n=3, mean±SEM, ***P<0.001). FIG. 2D, Bar graph showing the numbers of CD3⁺ T cells generated from control or EZH1 KD cells, normalized to numbers of CD34⁺ HE cells seeded on day 0 (n=3, mean±SEM, **P<0.01). FIG. 2E, Bar graph showing frequencies of CD4 SP, CD8 SP, and DP T cells generated from control or EZH1 KD cells after 6 weeks of stroma-free T cell differentiation (n=3, mean±SEM, ***P<0.001). FIGS. 2F-2H, Representative flow cytometry plots showing expression levels of CD3 and TCRαβ/TCRγδ/CD1a in control and EZH1 KD cells after stroma-free T cell differentiation, gated on CD45⁺ cells. FIG. 2I, Representative flow cytometry plots showing expression levels of CD8α and CD8β in control and EZH1 KD cells after stroma-free T cell differentiation, gated on CD8 T cells.

FIGS. 3A-3E show EZ-T cells display molecular features of mature TCRαβ T cells. FIG. 3A, Heatmap showing CellNet analysis of RNA-seq data from iPSC-derived CD34+ HSPCs and iPSC-derived T cells generated via stroma-free protocol, either with EZH1 knockdown (iPSC-EZ-T), or without (iPSC-SF-T). FIG. 3B, Dendrogram representing hierarchical cluster analysis based on expression of TCR pathway genes (BIOCARTA_TCR_PATHWAY, M19784) (n=3). αβT, peripheral blood TCRαβ T cells; γδT, peripheral blood TCRγδ T cells; NK, peripheral blood NK cells; CB-T, T cells differentiated from cord blood CD34⁺ HSPCs; iPSC-OP9-T, iPSC-T cells generated via OP9-DL1 co-culture; iPSC-SF-T, iPSC-T cells generated via stroma-free system without EZH1 knockdown; iPSC-EZ-T, EZ-T cells. FIG. 3C, Heatmap showing unsupervised clustering analysis based on TCRαβ signature genes (n=3). Color key is corresponding to the distance calculated by 1-Pearson correlation of gene expression profiles of each sample. FIG. 3D, Graph showing top GO terms of biological process enriched in iPSC-EZ-T cells vs. iPSC-SF-T cells by GSEA analysis (n=3). FIG. 3E, GSEA enrichment plots showing over-representation of gene sets related to T cell development and functions.

FIGS. 4A-4C show TCR repertoire analysis of EZ-T cells. FIG. 4A, Bar graph showing the frequencies of Vβ family genes in EZ-T cells determined by sequencing of the TCRβ CDR3 region (n=2). FIG. 4B, Bar graph showing TCRβ CDR3 length of iPSC-EZ-T cells compared to control iPSC-SF-T cells without EZH1 knockdown (n=2). FIG. 4C, Bar graph showing relative expression levels of TdT/DNTT in undifferentiated iPSCs or iPSC-SF-T cells with or without EZH1 KD (n=3, mean±SEM, *P≤0.05).

FIGS. 5A-5G show single cell RNA-seq analysis identifies memory-like T cell subsets in EZ-T cells after activation. FIG. 5A, Uniform Manifold Approximation and Projection (UMAP) visualization of all the CD45⁺ cells generated from EZ-T cell differentiation with and without activation. Colors indicate cell type or state. FIG. 5B, UMAP visualization of the expression of hematopoietic and T cell markers. FIG. 5C, Heatmap showing expression levels of T/NK cell signature genes across all clusters. FIG. 5D, GSEA analysis of the memory-like CD8 cluster showing over-representation of genes enriched in memory T cells but not naive or effector T cells. FIG. 5E, UMAP analysis comparing cell types in CD45⁺ cells generated from EZ-T cell differentiation before and after activation. FIG. 5F, Proportion of cells in unactivated and activated CD45⁺ cells generated via EZ-T cell differentiation. FIG. 5G, CellRouter analysis showing transcriptional regulators enriched in the memory-like CD8 T cell cluster.

FIGS. 6A-6G show EZ-T cells display enhanced effector functions. FIG. 6A, Bar graph showing CD69 expression and FIG. 6B, CD107a degranulation of iPSC-derived T cells and peripheral blood T cells after 6 hours of PMA/ionomycin stimulation, determined by flow cytometry analysis (n=3, mean±SEM). iPSC-OP9-T, iPSC-derived T cell via OP9-DL1 co-culture; iPSC-SF-T, control iPSC-SF-T cells generated via stroma-free system without EZH1 knockdown; iPSC-EZ-T, iPSC-EZ-T cells; PBMC-T, peripheral blood T cells. FIG. 6C, CAR-T cells generated from iPSC-derived T cells or peripheral blood T cells were co-cultured with JeKo1 and FIG. 6D, OCI-Ly1 tumor cell lines at indicated effector to target (E:T) ratios. Bar graph showing percentages of specific cytolysis of target tumor cells (n=3, mean±SEM). UTD PBMC-T, untransduced peripheral blood T cells; CD19 iPSC-OP9-T, CD19 CAR-transduced iPSC-derived T cell via OP9-DL1 co-culture; CD19 iPSC SF-T, CD19 CAR-transduced iPSC-T cells generated via stroma-free system without EZH1 knockdown; CD19 EZ-T, CD19 CAR-transduced iPSC-EZ-T cells; CD19 PBMC-T, CD19 CAR-transduced peripheral blood T cells. FIG. 6E, Bar graph showing production of IL-2, FIG. 6F, INFγ, and FIG. 6G, TNFα by CD19 CAR T cells generated from iPSC-SF-T, iPSC-EZ-T, and PBMC-T cells cultured in the absence (Unstim) and presence of OCI-Ly1 target tumor cells (n=3, mean±SEM, ****P≤0.0001).

FIGS. 7A-7D show CD19 CAR EZ-T cells mediate more robust in vivo tumor clearance FIG. 7A, Schematic illustration of in vivo CAR T cell functional studies using a DLBCL mouse model. NSG mice were intravenously injected with 1×10⁶ Luciferase-expressing OCI-Ly1 tumor cells. After two weeks, animals with engrafted tumor cells detected by bioluminescence were intravenously injected with 2×10⁶ CAR T cells generated from control iPSC-SF-T, iPSC-EZ-T, or PBMC-T cells. Tumor burden was assessed weekly by bioluminescent imaging (BLI) (n=9 animals from 3 experiments). FIG. 7B, Representative bioluminescent images of tumor xenografts over time and quantification of the tumor burden over time in each animal, represented by mean total flux (photons/sec). FIG. 7C, Plot showing absolute numbers of CAR T cells per 100 μl peripheral blood from each animal, determined by flow cytometry 3 weeks after CAR T cell injection (n=8, *** P≤0.001). FIG. 7D, Kaplan-Meier curve showing percentage survival of untreated animals and animal groups treated with CD19 CAR T cells generated from control iPSC-SF-T, iPSC-EZ-T, or PBMC-T cells (n=9, **P≤0.01 by log-rank test).

FIGS. 8A-8E show in vitro differentiation of T cells from iPSCs. FIG. 8A, Representative flow cytometry plots showing CD19+, CD56+, and CD33+ expression in stroma-free T cell differentiation (gated on CD45+ cells). FIG. 8B, Bar graph showing fold change of EZH1 mRNA levels in iPSC-derived T cell progenitor cells treated with control or EZH1 shRNA (n=5, **P≤0.01). FIG. 8C, Western blot result showing the steady-state levels of EZH1 in iPSC-derived T cell progenitor cells treated with control or EZH1 shRNA. TBP was used as a loading control. FIG. 8D, Bar graph showing frequencies of B cells (CD19+), NK cells (CD56+), Myeloid cells (CD33+), and T cell precursors (CD5+, CD3−) in CD45+ hematopoietic cells generated from stroma-free T cell differentiation with and without EZH1 knockdown (n=3, mean±SEM). FIG. 8E, Bar graph showing percentage of CD3+ T cells in CD45+ hematopoietic cells derived from two iPS cell lines (273 and 1381.3) after 5 weeks of T cell differentiation. CD34+ HE cells derived from both cell lines were treated with control or EZH1 shRNA at day 0 of T cell differentiation (n=5, **P<0.01).

FIGS. 9A-9H show EZH1 knockdown facilitates T cell differentiation from iPSCs.

FIG. 9A, Schematic of dCas9-KRAB and EZH1 sgRNA vectors. FIG. 9B, Bar chart showing fold change of CD3+ T cells generated from Dox-inducible EZH1-CRISPRi iPSC line after 5 weeks of stroma-free T cell differentiation. Cells were untreated or treated with Dox at different time points during differentiation (n=3, mean±SEM, **P≤0.01). FIGS. 9C-9D, Relative expression levels of EZH1 and EZH2 in iPSC-derived HE, T cell progenitor, and T cells, with and without EZH1 knockdown (n=3, mean±SEM). FIGS. 9E-9H, Bar charts showing frequencies of TCRαβCD3+/TCRγδ+CD3+/CD−1a+/CD8αβ+ cells in control and EZH1 KD cells after stroma-free T cell differentiation (n=3, mean±SEM, **P≤0.01, ***P≤0.001).

FIGS. 10A-10B show molecular Characterizations of iPSC-derived T cells. FIG. 10A, Bar chart showing T cell GRN scores of iPSC-derived CD34+ HSPCs and stroma-free iPSC T cells with or without EZH1 knockdown. FIG. 10B, Plot showing differentially expressed genes between TCRαβ T cells in control iPSC-SF-T and TCRαβ T cells in iPSC-EZ-T cells via scRNA-seq analysis.

FIGS. 11A-11D show scRNA-seq analysis of EZ-T cells. FIG. 11A, Representative flow cytometry plots showing expression levels of CD45RA, CD45RO, and CCR7 in activated iPSC-EZ-T cells, gated on CD3+ cells. FIG. 11B, UMAP visualization of the expression of PDCD1 and FOXP3 in EZ-T cells before and after activation. FIG. 11C, Binary regulon activity matrix by SCENIC analysis showing regulons that are active across all cell subsets after activation. FIG. 11D, UMAP visualization of enrichment of BATF, and IRF9 regulons in the memory-like cells.

FIGS. 12A-12C show compare EZ-T cells with in vivo hematopoietic cells. FIG. 12A, UMAP analysis color-coded by cell types identified by Granja et al. FIG. 12B, Mapping and classification of EZ-T cells using Symphony based on the reference atlas presented in FIG. 12A. FIG. 12C, kNN probability scores calculated by Symphony averaged based on scores calculated for each EZ-T cell mapping to an in vivo cell type from the reference atlas.

FIGS. 13A-13B show antitumor activities of TCRαβ EZ-T cells. FIG. 13A, Presorted TCRαβ CAR-T cells generated from iPSC-derived T cells were co-cultured with OCI-Ly1 tumor cell lines at indicated effector to target (E:T) ratios. Bar graph showing percentages of specific cytolysis of target tumor cells (n=3, mean±SEM, *P≤0.05, **P≤0.01). CD19 iPSC-SF-T, CD19 CAR-transduced iPSC-T cells generated via stroma-free system without EZH1 knockdown; CD19 iPSC-EZ-T, CD19 CAR-transduced iPSC-EZ-T cells. FIG. 13B, Bar chart showing absolute numbers of CAR T cells per 100 μl peripheral blood from each animal, determined by flow cytometry 3 weeks after CAR T cell injection (n=3, *P≤0.05).

FIGS. 14A-14B show in vivo characterizations of CAR EZ-T cells. FIG. 14A, Representative bioluminescent images of tumor xenografts over time and quantification of tumor burden represented by mean total flux (photons/sec) 11 weeks post tumor engraftment (n=3). FIG. 14B, Bar graph showing numbers of CAR T cells in peripheral blood samples collected on week 5 and week 20 post CAR T cell injection (n=3).

FIG. 15 shows flow cytometry plots showing frequencies of CD7+ lymphoid progenitor cells after 14 days of stroma-free T cell differentiation. Human iPSC-derived CD34+ HE cells were seeded on plates coated with 1) 10 ug/ml DLL4, 2) 20 ug/ml DLL4, or 3) 10 ug/ml DLL4+10 ug/ml Vitronectin on day 0.

FIG. 16 shows representative flow cytometry plots showing expression levels of CD5 and CD7 (top) or FSC and CD33 (bottom) in cells at Day 14 that were differentiated without IL-3 present (left) or with IL-3 present (right) in the first differentiation medium that promotes differentiation from CD34+ HE cells to CD5+CD7+ T cell progenitors for 1 week with quantification of the CD5+CD7+ T cell progenitors and CD33+ cells with and without the addition of IL-3 in the bar graph. The data demonstrate that addition of IL-3 during the first week of differentiation from CD34+ HE cells to CD5+CD7+ T cell progenitors promoted cell proliferation and that the effect was not restricted to lymphoid cells.

FIG. 17 shows the frequency of CD3+ cells after 35 days of T cell differentiation. (Top) Representative flow cytometry plots showing expression levels of FSC and CD3 in cells that were differentiated without IL-3 present in the first differentiation medium (left), or with IL-3 present in the first differentiation medium (right). (Bottom) Yield of CD3+ cells per 1000 CD34+ HE cells. The data demonstrate that addition of IL-3 during the first week of differentiation from CD34+ HE cells to CD5+CD7+ T cell progenitors resulted in a modest increase in the yield of CD3+ cells despite a slight reduction of frequency.

### DETAILED DESCRIPTION

Embodiments of the technology described herein include methods of differentiating T cells. The method and compositions described herein are based, in part, on the observation that contacting a CD34+ hemogenic endothelium with interleukin-3 (IL-3) during a T cell differentiation protocol can increase the yield of CD3+ T cells and can also produce cells having a phenotype similar to mature αβ T cells. Such mature cells are fully functional and can be used in a broad range of iPSC-based adoptive T cell therapies.

In one aspect, the method described herein is a stroma-free T cell differentiation method, i.e., a method that does not comprise co-culturing with stromal cells or any other type of supporting cell. Co-culture with stromal cells such as mouse stromal cells limits the translational potential of iPSC-derived T cells; for example, there can be fears of transplantation rejection due to the presence of stromal cells. Furthermore, T cells differentiated using stromal cells exhibit an innate-like phenotype (e.g., as measured by TCRγδ expression, which is a marker for gamma delta T cells). It is preferred that T cells exhibit an adaptive phenotype, for example characterized by expression of TCR α and β.

Additionally, as described herein, stroma-free T cell differentiation methods in combination with IL-3 treatment results in increased numbers of CD3+ T cells (e.g., CD4+CD8+ cells) compared to differentiation methods comprising stromal co-culture. Accordingly, T cells differentiated without stromal cell methods, and in one embodiment, in combination with inhibition of an epigenetic regulator (e.g., an HMT; e.g., EZH1, G9a/GLP), exhibit at least the following unexpected benefits compared to stromal co-culture methods: (1) increased potential for transplantation in humans; (2) decreased number of innate-like T cells; (3) increased number and/or percentage of resultant T cells (e.g., CD5+CD7+ Pro-T cells; CD3+ T cells; CD4+CD8+ T cells; CD4+ T cells; CD8+ T cells; alpha-beta T cells); (4) gene expression profiles most similar to alpha beta T cells; (5) a more diverse TCR repertoire; and/or (6) increased TCR CDR length.

### Pluripotent Stem Cells

In some embodiments, the stroma-free T cell differentiation method comprises differentiating a population of pluripotent stem cells. Pluripotent stem cells (PSCs) have the potential to give rise to all the somatic tissues. In one embodiment of any method, cells, or composition described herein, the population of pluripotent stem cells is induced pluripotent stem cells (iPSCs) or embryonic stem cells (ESC). IPSC and ESC can be produced by any method known in the art. In some embodiments, the population of pluripotent stem cells comprises embryonic stem cells (ESC). Embryonic stem cells (ESCs) are stem cells derived from the undifferentiated inner mass cells of a human embryo. In some embodiments, the pluripotent stem cells are not produced from an embryo.

Directed differentiation of PSCs aims to recapitulate embryonic development to generate patient-matched tissues by specifying the three germ layers. A common theme in directed differentiation across all germ layers is the propensity of PSCs to give rise to embryonic- and fetal-like cell types, which poses a problem for integration and function in an adult recipient. This distinction is particularly striking in the hematopoietic system, which emerges in temporally and spatially separated waves at during ontogeny. The earliest “primitive” progenitors emerge in the yolk sac at 8.5 dpc and give rise to a limited repertoire of macrophages, megakaryocytes and nucleated erythrocytes. These early embryonic-like progenitors are generally myeloid-based and cannot functionally repopulate the bone marrow of adult recipients. By contrast, “definitive” cells with hematopoietic stem cell (HSC) potential emerge later in arterial endothelium within the aorta-gonad-mesonephros (AGM) and other anatomical sites. Directed differentiation of PSCs gives rise to hematopoietic progenitors, which resemble those found in the yolk sac of the early embryo. These lack functional reconstitution potential, are biased to myeloid lineages, and express embryonic globins. Thus, understanding key fate determining mechanisms that promote development of either primitive or definitive lineages is critical for specifying HSCs, and other adult-like cell types (e.g., red blood cells) from PSCs.

In some embodiments, the population of pluripotent stem cells (PSCs) comprises induced pluripotent stem cells (iPS cells). In some embodiments, the induced pluripotent stem cells are produced by introducing reprogramming factors OCT4, SOX2, KLF4 and optionally c-MYC or nanog and LIN28 into mature cells. In some embodiments, the induced pluripotent stem cells are produced by introducing the reprogramming factors two or more times into the mature cells.

In some embodiments, the pluripotent stem cells (PSCs) described herein are induced pluripotent stem cells (iPSCs). An advantage of using iPSCs is that the cells can be derived from the same subject to which the eventual immune cells would be reintroduced. That is, a somatic cell can be obtained from a subject, reprogrammed to an induced pluripotent stem cell, and then transfected and differentiated into a modified immune cell to be administered to the subject (e.g., autologous cells). Since the progenitors are essentially derived from an autologous source, the risk of engrafiment rejection or allergic responses is reduced compared to the use of cells from another subject or group of subjects. In some embodiments, the cells for generating iPSCs are derived from non-autologous sources. In addition, the use of iPSCs negates the need for cells obtained from an embryonic source. Thus, in one embodiment, the PSCs used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a common myeloid stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not necessarily critical to the methods described. Thus, any method that re-programs a somatic cell to the pluripotent phenotype would be appropriate for use in the methods described herein.

Reprogramming methodologies for generating pluripotent cells using defined combinations of transcription factors have been described to induce pluripotent stem cells from somatic cells. Yamanaka and Takahashi converted mouse somatic cells to ES cell-like cells with expanded developmental potential by the direct transduction of Oct4, Sox2, Klf4, and optionally c-Myc. See U.S. Pat. Nos. 8,058,065 and 9,045,738 to Yamanaka and Takahashi. iPSCs resemble ES cells as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission, and tetraploid complementation.

Subsequent studies have shown that human iPS cells can be obtained using similar transduction methods, and the transcription factor trio, OCT4, SOX2, and NANOG, has been established as the core set of transcription factors that govern pluripotency. The production of iPS cells can be achieved by the introduction of nucleic acid sequences encoding stem cell-associated genes into an adult, somatic cell, using viral vectors.

OCT4, SOX2, KLF4 and c-MYC are the original four transcription factors identified to reprogram mouse fibroblasts into iPSCs. These same four factors were also sufficient to generate human iPSCs. OCT3/4 and SOX2 function as core transcription factors of the pluripotency network by regulating the expression of pluripotency-associated genes. Krüppel-like factor 4 (KLF4) is a downstream target of LIF-STAT3 signaling in mouse ES cells and regulates self-renewal. Human iPSCs can also be generated using four alternative factors; OCT4 and SOX2 are required but KLF4 and c-MYC could be replaced with NANOG, a homeobox protein important for the maintenance of pluripotency in both ES cells and early embryos, and LIN28, an RNA binding protein. The combination of OCT4, SOX2, NANOG and LIN28 reprogramming factors have been reported to be also sufficient to generate human iPSCs.

In one embodiment of any method, cells, or composition described herein, the iPSCs are produced, for example, by introducing exogenous copies of only three reprogramming factors OCT4, SOX2, and KLF4 into mature or somatic cells. In one embodiment of any method, cells, or composition described herein, c-MYC, or nanog and/or LIN28 are further introduced to iPSCs having exogenous gene coding copies of OCT4, SOX2, and KLF4 to differentiate into mature or somatic cells. In one embodiment of any method, cells, or composition described herein, the iPSCs are produced by introducing exogenous copies of reprogramming factors OCT4, SOX2, and KLF4, and optionally with c-MYC or nanog and/or LIN28 to differentiate into mature or somatic cells.

In one embodiment of any method, cells, or composition described herein, the iPSCs are produced by contacting mature cells with at least one vector, wherein the at least one vector carries an exogenous gene coding copy of reprogramming factors OCT4, SOX2, and KLF4, and optionally with c-MYC, or nanog and/or LIN28 to differentiate into mature or somatic cells, and wherein the reprogramming factors are expressed in vivo in the contacted mature or somatic cells. The contacting is in vitro or ex vivo. The reprogramming factors needed for differentiation can all be expressed by one vector (e.g., a vector that carries an exogenous gene coding copy of OCT4, SOX2, KLF4, and c-MYC). Alternatively, the reprogramming factors can be expressed in more than one vector that is each used to contact the iPSCs. For example, an iPSCs can be contacted by a first vector that carries an exogenous gene coding copy of OCT4, SOX2, and a second vector that carries an exogenous gene coding copy KLF4 and c-MYC.

In one embodiment of any disclosed methods, the iPS cell comprises at least an exogenous copy of a nucleic acid sequence encoding a reprogramming factor selected from the group consisting of genes Oct4 (Pou5fl), Sox2, cMyc, Klf4, Nanog, Lin 28 and Glis1. In some embodiments, combinations of reprogramming factors are used. For example, a combination of four reprogramming factors consisting of Oct4, Sox2, cMyc, and Klf4, or a combination of four reprogramming factors consisting of Oct4, Sox2, Nanog, and Lin 28.

In one embodiment of any method, cells, or composition described herein, the iPSCs are produced by introducing the disclosed reprogramming factors, or any combination of the reprograming factors two or more times into the mature or somatic cells. In one embodiment, the combination of reprograming factors is different when a combination is introduced to the iPSC more than once, for example, the combination of Oct4 (Pou5fl), Sox2, cMyc, Klf4, Nanog is first introduced to the iPSCs, and the combination of Oct4 (Pou5fl), Sox2, cMyc is subsequently introduced to the iPSCs. In one embodiment of any method, cells, or composition described herein, the iPSCs are produced by contacting mature cells with the disclosed vector(s) factors two or more times into the mature/somatic cells.

In some embodiments, the population of pluripotent stem cells (e.g., iPSCs) are not differentiated in the presence of a Notch ligand. In some embodiments, the aggregation media used to promote the differentiation of the population of pluripotent stem cells (e.g., iPSCs) into a population of CD34+ hemogenic endothelium does not comprise a Notch ligand. In some embodiments, the cell culture vessel used during the differentiation of the population of pluripotent stem cells (e.g., iPSCs) into the population of CD34+ hemogenic endothelium does not comprise a Notch ligand.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5; 7(5):618-30, this reference is incorporated herein by reference in its entirety). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135, the contents of each of which are incorporated herein by reference in its entirety. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Many US patents and patent Application Publications teach and describe methods of generating iPSCs and related subject matter. For examples, U.S. Pat. Nos. 8,058,065, 9,347,044, 9,347,042, 9,347,045, 9,340,775, 9,341,625, 9,340,772, 9,250,230, 9,132,152, 9,045,738, 9,005,975, 9,005,976, 8,927,277, 8,993,329, 8,900,871, 8,852,941, 8,802,438, 8,691,574, 8,735,150, 8,765,470, 8,058,065, 8,048,675, and US Patent Publication Nos: 20090227032, 20100210014, 20110250692, 20110201110, 20110200568, 20110223669, 20110306516, 20100021437, 20110256626, 20110044961, 20120276070, 20120214243, 20120263689, 20120128655, 20120100568, 20130295064, 20130029866, 20130059386, 20130183759, 20130189786, 20130295579, 20130130387, 20130157365, 20140234973, 20140227736, 20140093486, 20140301988, 20140170746, 20140178989, 20140349401, 20140065227, and 20150140662, all of which are incorporated herein by reference in their entireties.

In some embodiments, the iPSCs can be derived from somatic cells. Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells. In one embodiment of any method, cells, or composition described herein, the mature cells from which iPS cells are made include any somatic cells such as B lymphocytes (B-cells), T lymphocytes, (T-cells), and fibroblasts and keratinocytes.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, skin, immune cells, hepatic, splenic, lung, peripheral circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; beta-III-tubulin; alpha-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sa114; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived. In one embodiment, the iPSCs are derived from mature, differentiated, somatic cells.

In some embodiments, the population of pluripotent stem cells used in the differentiation methods described herein does not comprise CD34+ HSPCs or multipotent lymphoid progenitors (MLPs) purified from a patient sample. In some embodiments, the population of pluripotent stem cells does not comprise stem cells purified or isolated from cord blood or bone marrow samples. In some embodiments, the population of pluripotent stem cells is not derived from stem cells isolated from a patient sample (e.g., cord blood or bone marrow). In a preferred embodiment, the population of pluripotent stem cells comprise iPSCs, such as those derived from a somatic cell sample from a patient. See e.g., Tabatabaei-Zavareh et al., J Immunol May 1, 2017, 198 (1 Supplement) 202.9.

### Hemogenic Endothelium

In some embodiments, the methods described herein comprise differentiating a population of pluripotent stem cells (e.g., iPSCs) into a population of cells with hematopoietic potential. In some embodiments, the population of cells with hematopoietic potential comprises hemogenic endothelium (e.g., CD34+ hemogenic endothelium) and/or hematopoietic stem cells (HSCs). The cells with hematopoietic potential (e.g., hemogenic endothelium, HSCs) can be produced using any method known in the art.

One exemplary approach to generate HSCs from hPSCs is to specify HSCs from its ontogenetic precursors. It is now widely accepted that HSCs originate from hemogenic endothelium (HE) in the aorta-gonad-mesonephros (AGM) and arterial endothelium in other anatomical sites.

As used herein, the term “hemogenic endothelium” refers to a unique subset of endothelial cells scattered within blood vessels that can differentiate into hematopoietic cells. In the developing mouse, HSCs arise beginning embryonic day 10.5 from a small population of endothelial cells with hemogenic potential (hemogenic endothelium) located within the aorta-gonad-mesonephros region. In a process known as endothelial to hematopoietic transition (EHT), endothelial cells in the floor of the aorta round up and bud into the extravascular space followed by reentry into the circulation via the underlying vein. In some embodiments, a population of cells comprising the properties of hemogenic endothelium is differentiated in vitro from a population of pluripotent stem cells (e.g., iPSCs). Said “cells comprising the properties of hemogenic endothelium” can also be referred to herein as hemogenic endothelium.

Efforts to derive HSCs from pluripotent stem cells (PSCs) are complicated by the fact that embryonic hematopoiesis consists of two programs, primitive and definitive, but only definitive hematopoiesis generates HSCs and thus the lymphoid lineage. Definitive hematopoiesis, as measured by T-lymphoid potential, emerges after the establishment of the primitive hematopoietic program and develops from a progenitor population that displays characteristics of hemogenic endothelium.

In some embodiments, the T cell differentiation method comprises differentiating a population of pluripotent stem cells in aggregation media for a sufficient time to promote differentiation into a population of CD34+ hemogenic endothelium. In some embodiments, the resultant CD34+ hemogenic endothelium can undergo definitive hematopoiesis and/or exhibits lymphoid potential. In some embodiments, the hemogenic endothelium differentiates or is differentiated into hematopoietic stem cells (HSCs).

In some embodiments, the population of pluripotent stem cells (e.g., iPSCs) is differentiated into a population of CD34+ hemogenic endothelium using embryoid bodies (EBs) or 2D adherent cultures; see e.g., Pineda et al., Differentiation patterns of embryonic stem cells in two versus three dimensional culture, Cells Tissues Organs. 2013; 197(5): 399-410, which is incorporated herein by reference. EBs are three-dimensional aggregates of pluripotent stem cells produced and cultured in vitro in the presence of serum. The EBs can generate a mixture of primitive and definitive hematopoietic progenitor cell types. Primitive progenitors equate to those that arise in vivo naturally in the earliest stages of embryonic development, whereas at later stages of maturation the embryonic populations give rise to definitive progenitor cells, which behave similarly to the cells typical of adult hematopoiesis.

In some embodiments, the sufficient time to promote differentiation into a population of CD34+ hemogenic endothelium is at least 8 days (e.g., at least 7, at least 8, at least 9, at least 10 days, or more). In some embodiments, the sufficient time to promote differentiation into a population of CD34+ hemogenic endothelium is at most 8 days, at most 9 days, at most 10 days or more.

In some embodiments, the aggregation media comprises BMP4, SB-431542, CHIR99021, bFGF, VEGF, IL-6, IL-11, IGF-1, SCF, and EPO or any combination of the same. In some embodiments, the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/ml IL-11, 25 ng/ml IGF-1, 50 ng/ml SCF, and 2 U/ml EPO; see e.g., Example 2 presented herein.

In some embodiments, the components of the aggregation media are varied during the differentiation of pluripotent stem cells into hemogenic endothelium. As a non-limiting example, embryoid bodies are differentiated in the presence of BMP4, followed by stage-specific addition of bFGF, VEGF, and hematopoietic cytokines (e.g., IL-6, IL-11, IGF-1, SCF, and EPO). Activin-nodal signaling can be manipulated (e.g., using SB-431542 and CHIR99021) between days 2 and 3. See e.g., WO 2021/150919; and Sturgeon et al., Wnt signaling controls the specification of definitive and primitive hematopoiesis from human pluripotent stem cells, Nat Biotechnol. 2014 June; 32(6): 554-561, which are each incorporated herein by reference in their entirety.

In some embodiments, the aggregation media comprises BMP (e.g., 10 ng/mL BMP) during days 0, 1, and/or 2 of differentiation. In some embodiments, the aggregation media does not comprise BMP during days 3, 4, 5, 6, 7, or 8 of differentiation. In some embodiments, the concentration of BMP is within the range of 1-20 ng/mL (e.g., 1-15 ng/mL, 1-10 ng/mL, 1-5 ng/mL, 5-20 ng/mL, 10-20 ng/mL, 15-20 ng/mL, 8-10 ng/mL, 10-12 ng/mL, or any integer therebetween).

In some embodiments, the aggregation media comprises SB-431542 (e.g., 6 mM SB-431542) and/or CHIR99021 (e.g., 3 mM CHIR99021) during day 2 of differentiation. SB-431542 is a small-molecule antagonist of activin-nodal signaling. CHIR99021 is a GSK-3 inhibitor and a Wnt agonist. Inhibition of activin-nodal signaling and activation of Wnt signaling has been shown to drive PSC differentiation into definitive progenitors (KDR⁺CD235a⁻) with lymphoid potential (see e.g., Sturgeon 2014, supra, which is incorporated herein by reference). In some embodiments, the aggregation media comprises does not SB-431542 and/or CHIR99021 during days 0, 1, 3, 4, 5, 6, 7, and/or 8 of differentiation. In some embodiments, the concentration of SB-431542 in the aggregation media is in the range of 1-15 mM, 1-10 mM, 1-9 mM, 1-8 mM, 1-7 mM, 1-6 mM, 1-5 mM, 1-4 mM, 1-3 mM, 1-2 mM, 2-15 mM, 3-15 mM, 4-15 mM, 5-15 mM, 6-15 mM, 7-15 mM, 8-15 mM, 9-15 mM, 10-15 mM, 2-8 mM, 4-8 mM, 4-10 mM, 5-7 mM, 5-8 mM, or any range therebetween and/or the concentration of CHIR99021 in the aggregation media is in the range of 1-10 mM, 1-9 mM, 1-8 mM, 1-7 mM, 1-6 mM, 1-5 mM, 1-4 mM, 1-3 mM, 1-2 mM, 2-10 mM, 3-10 mM, 4-10 mM, 5-10 mM, 6-10 mM, 7-10 mM, 8-10 mM, 9-10 mM, 2-4 mM, 2-5 mM, 3-4 mM, 2-3 mM, 3-5 mM, or any range therebetween.

In some embodiments, the aggregation media comprises bFGF (e.g., 5 ng/ml bFGF) during days 1, 2, 3, 4, 5, 6, 7, and/or 8 of differentiation. In some embodiments, the aggregation media does not comprise bFGF during day 0 of differentiation. In some embodiments, the concentration of bFGF in the aggregation medium is in the range of 1-10 ng/mL, 1-9 ng/mL, 1-8 ng/mL, 1-7 ng/mL, 1-6 ng/mL, 1-5 ng/mL, 1-4 ng/mL, 1-3 ng/mL, 1-2 ng/mL, 2-10 ng/mL, 3-10 ng/mL, 4-10 ng/mL, 5-10 ng/mL, 6-10 ng/mL, 7-10 ng/mL, 8-10 ng/mL, 9-10 ng/mL, 2-8 ng/mL, 2-6 ng/mL, 3-8 ng/mL, 3-7 ng/mL, 4-6 ng/mL, 4-5 ng/mL, 5-6 ng/mL or any range therebetween.

In some embodiments, the aggregation media comprises VEGF (e.g., 15 ng/ml VEGF) during days 3, 4, 5, 6, 7, and/or 8 of differentiation. In some embodiments, the aggregation media does not comprise VEGF during days 0, 1, or 2 of differentiation. In some embodiments, the concentration of VEGF in the aggregation medium is in the range of 1-30 ng/mL, 1-25 ng/mL, 1-20 ng/mL, 1-15 ng/mL, 1-10 ng/mL, 1-5 ng/mL, 5-20 ng/mL, 10-20 ng/mL, 15-20 ng/mL, 12-16 ng/mL, 14-16 ng/mL, 14-15 ng/mL, 15-16 ng/mL or any range or integer therebetween.

In some embodiments, the aggregation media comprises hematopoietic cytokine(s) during days 6, 7, and/or 8 of differentiation. In some embodiments, the aggregation media does not comprise hematopoietic cytokine(s) during days 0, 1, 2, 3, 4, or 5 of differentiation. In some embodiments, the hematopoietic cytokines are selected from the group consisting of: IL-6 (e.g., 5, 10, 15, or 20 ng/ml IL-6), IL-11 (e.g., 1, 2, 5, 10, or 15 ng/ml IL-11), IGF-1 (e.g., 5, 10, 15, 20, 25, 30, 35 or 40 ng/ml IGF-1), SCF (e.g., 10, 20, 30, 40, 50, 60, 70, or 80 ng/ml SCF), and EPO (e.g., 1, 2, 3, 4 or 5 U/ml EPO). In one embodiment, the hematopoietic cytokines in the aggregation medium comprise or consist essentially of: IL6 (e.g., 10 ng/mL), IL-11 (e.g., 5 ng/mL), IGF-1 (e.g., 20 ng/mL), SCF (e.g., 50 ng/mL) and EPO (e.g., 2 U/mL).

In some embodiments, the differentiation method further comprises selecting or isolating the resultant population of CD34+ hemogenic endothelium using expression of surface markers on the population of CD34+ hemogenic endothelium. Non-limiting examples of methods for selecting or isolating hemogenic endothelium include magnetic-activated cell sorting (MACS) and fluorescence-activated cell sorting (FACS). In some embodiments, the surface marker for hemogenic endothelium is CD34 (e.g., high CD34 surface expression).

In some embodiments, additional positive or negative markers for hemogenic endothelium can include, but are not limited to, CD45, CD38, KDR, CD235, and CD43. In some embodiments, the population of CD34+ hemogenic endothelium is CD45 negative/low. In some embodiments, the population of CD34+ hemogenic endothelium is CD38 negative/low. In some embodiments, the population of CD34+ hemogenic endothelium is KDR+. In some embodiments, the population of CD34+ hemogenic endothelium is CD235 negative/low. In some embodiments, the population of CD34+ hemogenic endothelium is CD43 negative/low.

In some embodiments, the hemogenic endothelium and/or HSCs are produced using any method known in the art. As a non-limiting example, the method of differentiating PSCs into hemogenic endothelium can comprise the introduction of transcription factors such as ERG, HOXA5, HOXA9, HOXA10, LCOR, RUNX1, and/or SPI1; see e.g., International Application No. WO 2018/048828, US Patent Application No. 2019/0225940, Doulatov et al., Cell Stem Cell. 2013 Oct. 3, 13(4); Vo et al., Nature 2018, 553(7689): 506-510; the contents of each of which are incorporated herein by reference in their entireties.

In some embodiments, the hemogenic endothelium is not derived from PSCs but is rather derived directly from endothelial cells. For example, endothelial cells (e.g., from lung, brain, and other tissues) can be directly reprogrammed into hemogenic endothelium by transduction of with transcription factors (e.g., Fosb, Gfi1, Runx1, and Spi1) and co-culture with an immortalized endothelial cell line; the endothelial cells can be further exposed to cell-extrinsic factors (e.g., serum, SB-431542, and/or endothelial mitogen). See, e.g., Lis et al., Nature. 2017 May 25, 545(7655):439-445; Blaser and Zon, Blood. 2018 Sep. 27; 132(13): 1372-1378, which are incorporated herein by reference.

### Inhibition of an Epigenetic Regulator

In some aspects described herein is a T-cell differentiation method comprising a step of inhibiting at least one epigenetic regulator. As used herein, the term “epigenetic regulator” refers to a factor, e.g., a polypeptide, e.g., an enzyme, that influences DNA methylation and/or histone modifications (e.g., histone acetylation, histone methylation), and as such affect the transcription levels of genes without an alteration (e.g., substitution or deletion) to the nucleotide sequence of the genome. Non-limiting examples of epigenetic regulators include: DNA-methyltransferase (DNMT; e.g., DNMT1; DNMT3a; DNMT3b); methyl-CpG-binding domain (MBD) protein (e.g., MeCP2; MBD1; MBD2; MCD4; KAISO; ZBTB4; ZBTB38; UHRHRF2); DNA demethylase (e.g., 5′-methylcytokine hydroxylase; TET1; TET2; TET3); histone methyl transferase (HMT; e.g., SUV39s; SETIs; EZH1; EZH2; Set2s; PRDMs; SMYDs; DOT1L; PRMTs; G9a; GLP); methyl-histone binding protein (e.g., HP1; Chd1; BPTF; L3MBTL1; ING2; BHC80; JMJD2A); histone demethylase (e.g., KDMs; e.g., LSDs; JHDMs; JMJDs; JARID; Uts; PHFs); histone acetyl transferase (HAT; e.g., HAT1; GCN5; PCAF; MYSTs; p300; CBP; SRC/p160); acetyl-binding proteins (e.g., BROMO-domain, DPF-domain, or YEATS-domain-containing proteins); histone deacetylase (HDAC; e.g., HDAC1; HDAC2; HDAC3; HDAC4; HDAC5; HDAC6; HDAC7; HDAC8; HDAC9; HDAC10; HDAC11; Sirt1; Sirt2; Sirt3; Sirt4; Sirt5; Sirt6; Sirt7). See e.g., Cheng et al., Signal Transduction and Targeted Therapy volume 4, Article number: 62 (2019); the content of which is incorporated herein by reference in its entirety.

In some embodiments, the method comprises the step of, after the step of differentiating a population of pluripotent stem cells in aggregation media for a sufficient time to promote differentiation into a population of CD34+ hemogenic endothelium, inhibiting an epigenetic regulator in the resultant population of CD34+ hemogenic endothelium. In some embodiments, the method comprises the step of, prior to the step of differentiating a population of CD34+ hemogenic endothelium in a CD3+-T-cell differentiation media in the presence of a Notch ligand for a sufficient time to promote differentiation into a population of CD3+ T cells, inhibiting an epigenetic regulator in the population of CD34+ hemogenic endothelium.

Accordingly, in one aspect, described herein is a method comprising: (a) differentiating a population of pluripotent stem cells in aggregation media for a sufficient time to promote differentiation into a population of CD34+ hemogenic endothelium; (b) inhibiting an epigenetic regulator in the resultant population of CD34+ hemogenic endothelium; and (c) differentiating the resultant population of CD34+ hemogenic endothelium in a CD3+-T-cell differentiation media with interleukin-3 (IL-3) in the presence of a Notch ligand for a sufficient time to promote differentiation into a population of CD3+ T cells.

In some embodiments, CD34+ hemogenic endothelium is treated with an inhibitor of an epigenetic regulator. Exemplary inhibitors of an epigenetic regulator include an inhibitor of at least one of the following: DNMT; MBD; DNA demethylase; HMT; methyl-histone binding protein; histone demethylase; HAT; acetyl-binding protein; or HDAC. In some embodiments, the epigenetic regulator is an H3K9 methyltransferase. Methylation of H3K9 in humans relies mostly on members of the Suv39 family, namely EHMT1/GLP, EHMT2/G9a, SUV39H1, SUV39H2, SETDB1 and SETDB2, as well as then non-Suv39 enzymes PRDM2 and ASH1L.

Non-limiting examples of DNMT inhibitors include azacitidine; decitabine; guadecitabine; hydralazine. Non-limiting examples of HMT inhibitors include pinometostat; tazemetostat; GSK2816126; CPI-1205; TCP; ORY-2001; GSK2879552; 4SC-202. Non-limiting examples of HDAC inhibitors include valproic acid, phenylbutyrate; vorinostat; trichostatin A; belinostat; entinostat; panobinostat; mocetinostat; CI-994; romidepsin; nicotinamide; suramin; PRI-724; GSK525762; CPI-0610; R06870810; MK-8628.

In some embodiments, the inhibitor of an epigenetic regulator is selected from Table 1. In some embodiments, the inhibitor of an epigenetic regulator is selected from the group consisting of: SB939 (Pracinostat); 4-iodo-SAHA; Scriptaid; Oxaflatin (i.e., Oxamflatin); s-HDAC-42; UNC0224; Pyroxamide; MC1568; CAY10398; CAY10591; SAHA (Vorinostat) (SIH-359); SGI-1027; and Rucaparib (Rubraca™). In some embodiments, the inhibitor of an epigenetic regulator is selected from the group consisting of: SB939 (Pracinostat); 4-iodo-SAHA; Scriptaid; Oxaflatin (i.e., Oxamflatin); s-HDAC-42; UNC0224; Pyroxamide; MC1568; CAY10398; CAY10591; and SAHA (Vorinostat) (SIH-359); see e.g., Table 1.

TABLE 1
Small molecule inhibitors that can promote T cell differentiation (e.g., at 500
nM; small molecules with a Z score greater than 3 are shown bolded).

| | CD5⁺ | | | | |
|---|---|---|---|---|---|
| Well | CD7⁺ | | Small | Function of | |
| location | % | Z score | molecule | small molecule | Structure of small molecule |
| 1A4 | 39.4% | 5.042400516 | SB939 (Pracinostat) | Pan-HDAC inhibitor (e.g., with IC50 of 40-140 nM with exception for HDAC6). It has no activity against the class III isoenzyme SIRT1. | 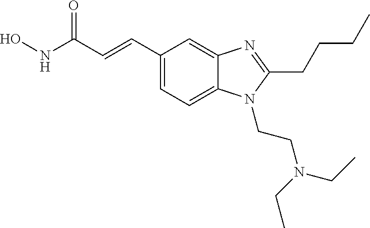 [SMILES: CCCCc1nc2cc(/C=C/C(=O)NO)ccc2n1CCN(CC)CC] |
| 1A6 | 25.3% | 5.894930088 | 4-iodo- SAHA | 4-iodo-SAHA is a hydrophobic derivative of the class I and class II HDAC (e.g., HDAC1 or HDAC6) inhibitor SAHA. | 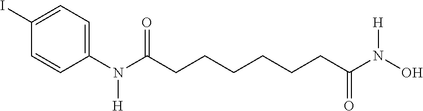 [SMILES: O=C(CCCCCCC(=O)Nc1ccc(I)cc1)NO] |
| 1B3 | 19.7% | 3.857176477 | Scriptaid | HDAC inhibitor (see e.g., US Patent 6,544,957). | 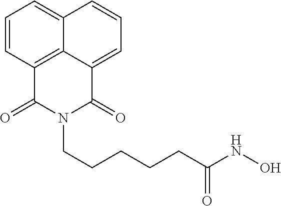 [SMILES: O=C(CCCCCN1C(=O)c2cccc3cccc(c23)C1=O)NO] |
| 1H7 | 12.4% | 6.539525618 | Oxaflatin (i.e., Oxamflatin) | HDAC inhibitor | 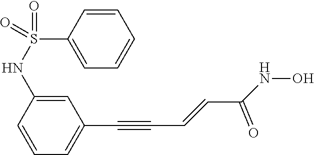 [SMILES: O=C(/C=C/C#Cc1cccc(NS(=O)(=O)c2ccccc2)c1)NO] |
| 1H11 | 43.2% | 4.262647859 | s-HDAC-42 | HDAC inhibitor | 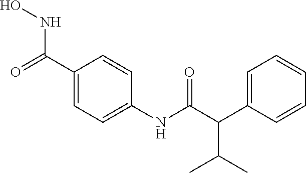 [SMILES: CC(C)C(C(=O)Nc1ccc(C(=O)NO)cc1)c1ccccc1] |
| 2A8 | 15.0% | 3.475075737 | UNC0224 | G9a and GLP inhibitor (H3K9 methyltransferase) | 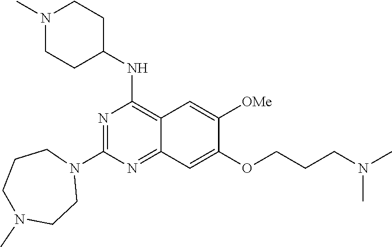 |
| 2B2 | 11.6% | 3.121963203 | Pyroxamide | HDAC (e.g., HDAC1) inhibitor | 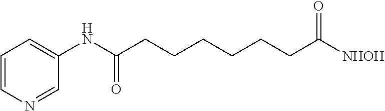 |
| 2E5 | 14.0% | 5.829159302 | MC1568 | Class II HDAC (e.g., HDAC4 and HDAC5) inhibitor. Displays no inhibition of class I HDAC activity (e.g., HDAC1, HDAC2, HDAC3). | 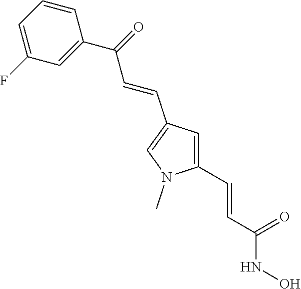 [SMILES: Cn1cc(/C=C/C(=O)c2cccc(F)c2)cc1/C=C/C(=O)NO] |
| 2F10 | 36.9% | 4.181300807 | CAY10398 | HDAC (e.g., HDAC1) inhibitor | 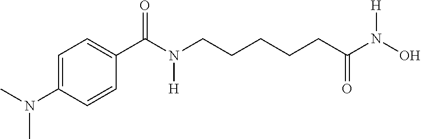 [SMILES: CN(C)c1ccc(C(=O)NCCCCCC(=O)NO)cc1] |
| 2G4 | 25.9% | 8.575590126 | CAY10591 | SIRT1 activator. Sirtuins (SIRTs) represent a distinct class of trichostatin A- insensitive lysyl- deacetylases (class III HDACs). | 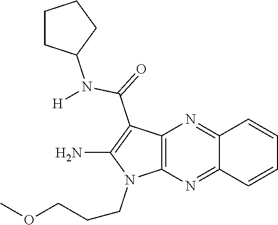 [SMILES: COCCCn1c(N)c(C(=O)NC2CCCC2)c2nc3ccccc3nc21] |
| 2G6 | 34.7% | 7.987069235 | SAHA (Vorinostat) (SIH-359) | Potent reversible panhistone deacetylase HDAC inhibitor, including both class I and class II HDACs. | 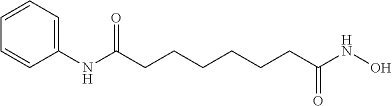 [SMILES: O=C(CCCCCCC(=O)Nc1ccccc1)NO] |
| 1D11 | 19.9% | 0.093570319 | SGI-1027 | DNA Methyltransferase (DNMT; e.g., DNMT1, DNMT3A, or DNMT3B) inhibitor | 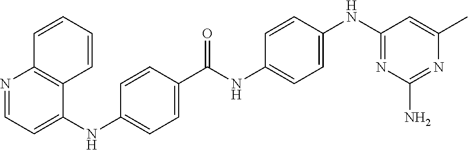 [SMILES: Cc1cc(Nc2ccc(NC(=O)c3ccc(Nc4ccnc5ccccc45)cc3)cc2)nc(N)n1] |
| 1E8 | 37.2% | 0.467851594 | Rucaparib (Rubraca ™) | poly(ADP- ribose) polymerase (PARP; e.g., PARP1, PARP2, PARP3) inhibitor | 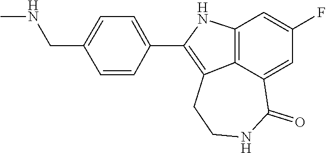 [SMILES: CNCc1ccc(-c2[nH]c3cc(F)cc4c3c2CCNC4=O)cc1] |
| Control | 11.60% | | | | |
| cells | | | | | |

In some embodiments, the inhibitor of an epigenetic regulator is selected from the group consisting of: UNC0224; MC1568; and CAY10591. In some embodiments, the inhibitor of an epigenetic regulator is UJNC0224. In some embodiments, the inhibitor of an epigenetic regulator is MC1568. In some embodiments, the inhibitor of an epigenetic regulator is CAY10591.

In some embodiments, the inhibitor of an epigenetic regulator is UJNC0224 or 5-Methyl-2′-deoxycytidine (see e.g., structure in Formula I below). In some embodiments, the inhibitor of an epigenetic regulator is 5-Methyl-2′-deoxycytidine. 5-Methyl-2′-deoxycytidine is a pyrimidine nucleoside that when incorporated into single-stranded DNA can act in cis to signal de novo DNA methylation; see e.g., Christman et al. Proceedings of the National Academy of Sciences of the United States of America 92(16), 7347-7351 (1995).

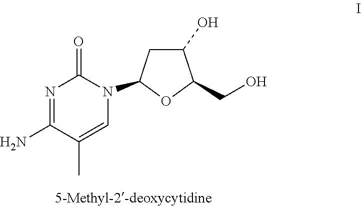 [SMILES: Cc1cn([C@H]2C[C@H](O)[C@@H](CO)O2)c(=O)nc1N]

In some embodiments, the inhibitor of an epigenetic regulator is provided at a concentration of at least 500 nM. In some embodiments, the inhibitor of an epigenetic regulator is provided at a concentration of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 300 nM, at least 400 nM, at least 500 nM, at least 600 nM, at least 700 nM, at least 800 nM, at least 900 nM, at least 1.0 uM, at least 1.25 uM, at least 1.5 uM, at least 1.75 uM, at least 2.0 uM, at least 2.5 uM, at least 3 uM, at least 4 uM, at least 5 uM, at least 6 uM, at least 7 uM, at least 8 uM, at least 9 uM, or at least 10 uM. In some embodiments, the inhibitor of an epigenetic regulator is provided at a concentration of 1 nM-10 nM, 10 nM-50 nM, 50 nM-100 nM, 100 nM-500 nM, 500 nM-1 uM, 1 uM-5 uM, or 5 uM-10 uM.

In some embodiments, the cells (e.g., CD34+ hemogenic endothelium) in culture are exposed to an inhibitor of an epigenetic regulator until the development of CD5+CD7+ T cell progenitor cells. In some embodiments, the cells (e.g., CD34+ hemogenic endothelium) in culture are exposed to an inhibitor of an epigenetic regulator for about 14 days. In some embodiments, the cells (e.g., CD34+ hemogenic endothelium) are cultured and exposed to an inhibitor of an epigenetic regulator for at least 1 day, at least 2 days, at least 3 days, at least 4 days, at least 5 days, at least 6 days, at least 7 days, at least 8 days, at least 9 days, at least 10 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, or more.

Inhibition of G9a and/or GLP

In some aspects described herein is a T-cell differentiation method comprising a step of inhibiting G9a and/or GLP. In some aspects described herein is a T-cell differentiation method comprising a step of inhibiting G9a. G9a can also be referred to interchangeably as Euchromatic Histone Lysine Methyltransferase 2 (EHMT2); Histone H3-K9 Methyltransferase 3; KMT1C; Lysine N-Methyltransferase 1C; BAT8; or NG36. G9a is a methyltransferase that methylates lysine residues of histone H3 (see e.g., NCBI Gene ID: 10919; SEQ ID NOs: 45-46 or a sequence that is at least 95% identical and maintains the same function, or a functional fragment thereof). In some aspects described herein is a T-cell differentiation method comprising a step of inhibiting G9a-like protein (GLP). GLP is also referred to interchangeably as Euchromatic Histone Lysine Methyltransferase 1 (EHMT1); KMT1D; Eu-HMTase1; or Histone-Lysine N-Methyltransferase, H3 Lysine-9 Specific 5 (see e.g., NCBI Gene ID: 79813; SEQ ID NOs: 47-48 or a sequence that is at least 95% identical and maintains the same function, or a functional fragment thereof).

G9a and GLP exist predominantly as a G9a-GLP heteromeric complex. G9a and GLP are the primary enzymes for mono- and dimethylation at Lys 9 of histone H3 (H3K9me1 and H3K9me2) in euchromatin. H3K9me represents a specific tag for epigenetic transcriptional repression by recruiting HP1 proteins to methylated histones. G9a/GLP also weakly methylates ‘Lys-27’ of histone H3 (H3K27me). G9a/GLP is also required for DNA methylation; the histone methyltransferase activity of G9a/GLP is not required for DNA methylation, suggesting that these two activities function independently. G9a/GLP is probably targeted to histone H3 by different DNA-binding proteins, e.g., E2F6, MGA, MAX and/or DP1. In addition to the histone methyltransferase activity, G9a/GLP also methylates non-histone proteins, e.g., dimethylation of ‘Lys-373’ of p53/TP53.

G9a also mediates monomethylation of ‘Lys-56’ of histone H3 (H3K56me1) in G1 phase, leading to promote interaction between histone H3 and PCNA and regulating DNA replication. G9a is also though to methylate histone H1. G9a also methylates CDYL, WIZ, ACIN1, DNMT1, HDAC1, ERCC6, KLF12, and itself. During G0 phase, GLP may contribute to silencing of MYC- and E2F-responsive genes, suggesting a role in G0/G1 transition in cell cycle. In addition to the histone methyltransferase activity, GLP also methylates non-histone proteins: mediates dimethylation of ‘Lys-373’ of p53/TP53.

Homo sapiens euchromatic histone lysine methyltransferase 2
(EHMT2), transcript variant 1, mRNA, NCBI Reference Sequence:
NM_001289413.1 (region 5-3706), 3702 bp
SEQ ID NO: 45
ATGCGGGGTCTACCGAGAGGGAGGGGGTTGATGCGGGCCCGGGGGAGGGGTCGTGCGG
CCCCTCCGGGCAGCCGAGGCCGCGGAAGGGGGGGGCCCCACAGAGGAAGAGGTAGGCC
CCGGAGCCTACTCTCTCTTCCCAGGGCCCAGGCATCCTGGACCCCCCAACTCTCTACTGG
GCTGACCAGCCCTCCTGTCCCTTGTCTCCCCTCCCAGGGGGAGGCCCCCGCTGAGATGGG
GGCGCTGCTGCTGGAGAAGGAAACCAGAGGAGCCACCGAGAGAGTTCATGGCTCTTTGG
GGGACACCCCTCGTAGTGAAGAAACCCTGCCCAAGGCCACCCCCGACTCCCTGGAGCCT
GCTGGCCCCTCATCTCCAGCCTCTGTCACTGTCACTGTTGGTGATGAGGGGGCTGACACC
CCTGTAGGGGCTACACCACTCATTGGGGATGAATCTGAGAATCTTGAGGGAGATGGGGA
CCTCCGTGGGGGCCGGATCCTGCTGGGCCATGCCACAAAGTCATTCCCCTCTTCCCCCAG
CAAGGGGGGTTCCTGTCCTAGCCGGGCCAAGATGTCAATGACAGGGGGGGGAAAATCAC
CTCCATCTGTCCAGAGTTTGGCTATGAGGCTACTGAGTATGCCAGGAGCCCAGGGAGCTG
CAGCAGCAGGGTCTGAACCCCCTCCAGCCACCACGAGCCCAGAGGGACAGCCCAAGGTC
CACCGAGCCCGCAAAACCATGTCCAAACCAGGAAATGGACAGCCCCCGGTCCCTGAGAA
GCGGCCCCCTGAAATACAGCATTTCCGCATGAGTGATGATGTCCACTCACTGGGAAAGGT
GACCTCAGATCTGGCCAAAAGGAGGAAGCTGAACTCAGGAGGTGGCCTGTCAGAGGAGT
TAGGTTCTGCCCGGCGTTCAGGAGAAGTGACCCTGACGAAAGGGGACCCCGGGTCCCTG
GAGGAGTGGGAGACGGTGGTGGGTGATGACTTCAGTCTCTACTATGATTCCTACTCTGTG
GATGAGCGCGTGGACTCCGACAGCAAGTCTGAAGTTGAAGCTCTAACTGAACAACTAAG
TGAAGAGGAGGAGGAGGAAGAGGAGGAAGAAGAAGAAGAGGAAGAGGAGGAGGAAG
AGGAAGAAGAAGAGGAAGATGAGGAGTCAGGGAATCAGTCAGATAGGAGTGGTTCCAG
TGGCCGGCGCAAGGCCAAGAAGAAATGGCGAAAAGACAGCCCATGGGTGAAGCCGTCT
CGGAAACGGCGCAAGCGGGAGCCTCCGCGGGCCAAGGAGCCACGAGGGGTGTCCAATG
ACACATCTTCGCTGGAGACAGAGCGAGGGTTTGAGGAGTTGCCCCTGTGCAGCTGCCGC
ATGGAGGCACCCAAGATTGACCGCATCAGCGAGAGGGCGGGGCACAAGTGCATGGCCA
CTGAGAGTGTGGACGGAGAGCTGTCAGGCTGCAATGCCGCCATCCTCAAGCGGGAGACC
ATGAGGCCATCCAGCCGTGTGGCCCTGATGGTGCTCTGTGAGACCCACCGCGCCCGCATG
GTCAAACACCACTGCTGCCCGGGCTGCGGCTACTTCTGCACGGCGGGCACCTTCCTGGAG
TGCCACCCTGACTTCCGTGTGGCCCACCGCTTCCACAAGGCCTGTGTGTCTCAGCTGAAT
GGGATGGTCTTCTGTCCCCACTGTGGGGAGGATGCTTCTGAAGCTCAAGAGGTGACCATC
CCCCGGGGTGACGGGGTGACCCCACCGGCCGGCACTGCAGCTCCTGCACCCCCACCCCT
GTCCCAGGATGTCCCCGGGAGAGCAGACACTTCTCAGCCCAGTGCCCGGATGCGAGGGC
ATGGGGAACCCCGGCGCCCGCCCTGCGATCCCCTGGCTGACACCATTGACAGCTCAGGG
CCCTCCCTGACCCTGCCCAATGGGGGCTGCCTTTCAGCCGTGGGGCTGCCACTGGGGCCA
GGCCGGGAGGCCCTGGAAAAGGCCCTGGTCATCCAGGAGTCAGAGAGGCGGAAGAAGC
TCCGTTTCCACCCTCGGCAGTTGTACCTGTCCGTGAAGCAGGGCGAGCTGCAGAAGGTGA
TCCTGATGCTGTTGGACAACCTGGACCCCAACTTCCAGAGCGACCAGCAGAGCAAGCGC
ACGCCCCTGCATGCAGCCGCCCAGAAGGGCTCCGTGGAGATCTGCCATGTGCTGCTGCA
GGCTGGAGCCAACATAAATGCAGTGGACAAACAGCAGCGGACGCCACTGATGGAGGCC
GTGGTGAACAACCACCTGGAGGTAGCCCGTTACATGGTGCAGCGTGGTGGCTGTGTCTAT
AGCAAGGAGGAGGACGGTTCCACCTGCCTCCACCACGCAGCCAAAATCGGGAACTTGGA
GATGGTCAGCCTGCTGCTGAGCACAGGACAGGTGGACGTCAACGCCCAGGACAGTGGGG
GGTGGACGCCCATCATCTGGGCTGCAGAGCACAAGCACATCGAGGTGATCCGCATGCTA
CTGACGCGGGGCGCCGACGTCACCCTCACTGACAACGAGGAGAACATCTGCCTGCACTG
GGCCTCCTTCACGGGCAGCGCCGCCATCGCCGAAGTCCTTCTGAATGCGCGCTGTGACCT
CCATGCTGTCAACTACCATGGGGACACCCCCCTGCACATCGCAGCTCGGGAGAGCTACC
ATGACTGCGTGCTGTTATTCCTGTCACGTGGGGCCAACCCTGAGCTGCGGAACAAAGAG
GGGGACACAGCATGGGACCTGACTCCCGAGCGCTCCGACGTGTGGTTTGCGCTTCAACTC
AACCGCAAGCTCCGACTTGGGGTGGGAAATCGGGCCATCCGCACAGAGAAGATCATCTG
CCGGGACGTGGCTCGGGGCTATGAGAACGTGCCCATTCCCTGTGTCAACGGTGTGGATG
GGGAGCCCTGCCCTGAGGATTACAAGTACATCTCAGAGAACTGCGAGACGTCCACCATG
AACATCGATCGCAACATCACCCACCTGCAGCACTGCACGTGTGTGGACGACTGCTCTAGC
TCCAACTGCCTGTGCGGCCAGCTCAGCATCCGGTGCTGGTATGACAAGGATGGGCGATTG
CTCCAGGAATTTAACAAGATTGAGCCTCCGCTGATTTTCGAGTGTAACCAGGCGTGCTCA
TGCTGGAGAAACTGCAAGAACCGGGTCGTACAGAGTGGCATCAAGGTGCGGCTACAGCT
CTACCGAACAGCCAAGATGGGCTGGGGGGTCCGCGCCCTGCAGACCATCCCACAGGGGA
CCTTCATCTGCGAGTATGTCGGGGAGCTGATCTCTGATGCTGAGGCTGATGTGAGAGAGG
ATGATTCTTACCTCTTCGACTTAGACAACAAGGATGGAGAGGTGTACTGCATAGATGCCC
GTTACTATGGCAACATCAGCCGCTTCATCAACCACCTGTGTGACCCCAACATCATTCCCG
TCCGGGTCTTCATGCTGCACCAAGACCTGCGATTTCCACGCATCGCCTTCTTCAGTTCCCG
AGACATCCGGACTGGGGAGGAGCTAGGGTTTGACTATGGCGACCGCTTCTGGGACATCA
AAAGCAAATATTTCACCTGCCAATGTGGCTCTGAGAAGTGCAAGCACTCAGCCGAAGCC
ATTGCCCTGGAGCAGAGCCGTCTGGCCCGCCTGGACCCACACCCTGAGCTGCTGCCCGAG
CTCGGCTCCCTGCCCCCTGTCAACACATGA,
histone-lysine N-methyltransferase EHMT2 isoform c (Homo sapiens),
NCBI Reference Sequence: NP_001276342.1, 1233 aa
SEQ ID NO: 46
MRGLPRGRGLMRARGRGRAAPPGSRGRGRGGPHRGRGRPRSLLSLPRAQASWTPQLSTGLT
SPPVPCLPSQGEAPAEMGALLLEKETRGATERVHGSLGDTPRSEETLPKATPDSLEPAGPSSPA
SVTVTVGDEGADTPVGATPLIGDESENLEGDGDLRGGRILLGHATKSFPSSPSKGGSCPSRAK
MSMTGAGKSPPSVQSLAMRLLSMPGAQGAAAAGSEPPPATTSPEGQPKVHRARKTMSKPGN
GQPPVPEKRPPEIQHFRMSDDVHSLGKVTSDLAKRRKLNSGGGLSEELGSARRSGEVTLTKG
DPGSLEEWETVVGDDFSLYYDSYSVDERVDSDSKSEVEALTEQLSEEEEEEEEEEEEEEEEEE
EEEEEEDEESGNQSDRSGSSGRRKAKKKWRKDSPWVKPSRKRRKREPPRAKEPRGVSNDTSS
LETERGFEELPLCSCRMEAPKIDRISERAGHKCMATESVDGELSGCNAAILKRETMRPSSRVA
LMVLCETHRARMVKHHCCPGCGYFCTAGTFLECHPDFRVAHRFHKACVSQLNGMVFCPHC
GEDASEAQEVTIPRGDGVTPPAGTAAPAPPPLSQDVPGRADTSQPSARMRGHGEPRRPPCDPL
ADTIDSSGPSLTLPNGGCLSAVGLPLGPGREALEKALVIQESERRKKLRFHPRQLYLSVKQGE
LQKVILMLLDNLDPNFQSDQQSKRTPLHAAAQKGSVEICHVLLQAGANINAVDKQQRTPLM
EAVVNNHLEVARYMVQRGGCVYSKEEDGSTCLHHAAKIGNLEMVSLLLSTGQVDVNAQDS
GGWTPIIWAAEHKHIEVIRMLLTRGADVTLTDNEENICLHWASFTGSAAIAEVLLNARCDLH
AVNYHGDTPLHIAARESYHDCVLLFLSRGANPELRNKEGDTAWDLTPERSDVWFALQLNRK
LRLGVGNRAIRTEKIICRDVARGYENVPIPCVNGVDGEPCPEDYKYISENCETSTMNIDRNITH
LQHCTCVDDCSSSNCLCGQLSIRCWYDKDGRLLQEFNKIEPPLIFECNQACSCWRNCKNRVV
QSGIKVRLQLYRTAKMGWGVRALQTIPQGTFICEYVGELISDAEADVREDDSYLFDLDNKDG
EVYCIDARYYGNISRFINHLCDPNIIPVRVFMLHQDLRFPRIAFFSSRDIRTGEELGFDYGDRFW
DIKSKYFTCQCGSEKCKHSAEAIALEQSRLARLDPHPELLPELGSLPPVNT,
Homo sapiens euchromatic histone lysine methyltransferase 1 (EHMT1),
transcript variant 2, mRNA, NCBI Reference Sequence: NM_001145527.2
(region 25-2451), 2427 bp
SEQ ID NO: 47
ATGGCCGCCGCCGATGCCGAGGCAGTTCCGGCGAGGGGGGAGCCTCAGCAGGATTGCTG
TGTGAAAACCGAGCTGCTGGGAGAAGAGACACCTATGGCTGCCGATGAAGGCTCAGCAG
AGAAACAGGCAGGAGAGGCCCACATGGCTGCGGACGGTGAGACCAATGGGTCTTGTGA
AAACAGCGATGCCAGCAGTCATGCAAATGCTGCAAAGCACACTCAGGACAGCGCAAGG
GTCAACCCCCAGGATGGCACCAACACACTAACTCGGATAGCGGAAAATGGGGTTTCAGA
AAGAGACTCAGAAGCGGCGAAGCAAAACCACGTCACTGCCGACGACTTTGTGCAGACTT
CTGTCATCGGCAGCAACGGATACATCTTAAATAAGCCGGCCCTACAGGCACAGCCCTTG
AGGACTACCAGCACTCTGGCCTCTTCGCTGCCTGGCCATGCTGCAAAAACCCTTCCTGGA
GGGGCTGGCAAAGGCAGGACTCCAAGCGCTTTTCCCCAGACGCCAGCCGCCCCACCAGC
CACCCTTGGGGAGGGGAGTGCTGACACAGAGGACAGGAAGCTCCCGGCCCCTGGCGCCG
ACGTCAAGGTCCACAGGGCACGCAAGACCATGCCGAAGTCCGTCGTGGGCCTGCATGCA
GCCAGTAAAGATCCCAGAGAAGTTCGAGAAGCTAGAGATCATAAGGAACCAAAAGAGG
AGATCAACAAAAACATTTCTGACTTTGGACGACAGCAGCTTTTACCCCCCTTCCCATCCC
TTCATCAGTCGCTACCTCAGAACCAGTGCTACATGGCCACCACAAAATCACAGACAGCTT
GCTTGCCTTTTGTTTTAGCAGCTGCAGTATCTCGGAAGAAAAAACGAAGAATGGGAACCT
ATAGCCTGGTTCCTAAGAAAAAGACCAAAGTATTAAAACAGAGGACGGTGATTGAGATG
TTTAAGAGCATAACTCATTCCACTGTGGGTTCCAAGGGGGAGAAGGACCTGGGCGCCAG
CAGCCTGCACGTGAATGGGGAGAGCCTGGAGATGGACTCGGATGAGGACGACTCAGAG
GAGCTCGAGGAGGACGACGGCCATGGTGCAGAGCAGGCGGCCGCGTTCCCCACAGAGG
ACAGCAGGACTTCCAAGGAGAGCATGTCGGAGGCTGATCGCGCCCAGAAGATGGACGG
GGAGTCCGAGGAGGAGCAGGAGTCCGTGGACACCGGGGAGGAGGAGGAAGGCGGTGAC
GAGTCTGACCTGAGTTCGGAATCCAGCATTAAGAAGAAATTTCTCAAGAGGAAAGGAAA
GACCGACAGTCCCTGGATCAAGCCAGCCAGGAAAAGGAGGCGGAGAAGTAGAAAGAAG
CCCAGCGGTGCCCTCGGTTCTGAGTCGTATAAGTCATCTGCAGGAAGCGCTGAGCAGAC
GGCACCAGGAGACAGCACAGGGTACATGGAAGTTTCTCTGGACTCCCTGGATCTCCGAG
TCAAAGGAATTCTGTCTTCACAAGCAGAAGGGTTGGCCAACGGTCCAGATGTGCTGGAG
ACAGACGGCCTCCAGGAAGTGCCTCTCTGCAGCTGCCGGATGGAAACACCGAAGAGTCG
AGAGATCACCACACTGGCCAACAACCAGTGCATGGCTACAGAGAGCGTGGACCATGAAT
TGGGCCGGTGCACAAACAGCGTGGTCAAGTATGAGCTGATGCGCCCCTCCAACAAGGCC
CCGCTCCTCGTGCTGTGTGAAGACCACCGGGGCCGCATGGTGAAGCACCAGTGCTGTCCT
GGCTGTGGCTACTTCTGCACAGCGGGTAATTTTATGGAGTGTCAGCCCGAGAGCAGCATC
TCTCACCGTTTCCACAAAGACTGTGCCTCTCGAGTCAATAACGCCAGCTATTGTCCCCAC
TGTGGGGAGGAGAGCTCCAAGGCCAAAGAGGTGACGATAGCTAAAGCAGACACCACCT
CGACCGTGACACCAGTCCCCGGGCAGGAGAAGGGCTCGGCCCTGGAGGGCAGGGCCGA
CACCACAACGGGCAGTGCTGCCGGGCCACCACTCTCGGAGGACGACAAGCTGCAGGGTG
CAGCCTCCCACGTGCCCGAGGGCTTTGATCCAACGGGACCTGCTGGGCTTGGGAGGCCA
ACTCCCGGCCTTTCCCAGGGACCAGGGAAGGAAACCTTGGAGAGCGCTCTCATCGCCCTC
GACTCGGAAAAACCCAAGAAGCTTCGCTTCCACCCAAAGCAGCTGTACTTCTCCGCCAG
GCAAGGGGAGCTTCAGAAGGTGCTCCTCATGCTGGTGGACGGAATTGACCCCAACTTCA
AAATGGAGCACCAGAATAAGCGCTCTCCACTGCACGCCGCGGCAGAGGCTGGACACGTG
GACATCTGCCACATGCTGGTTCAGTTCTGCAGGCTGGGAAGCCCAAGGTCGAGGGGCTG
CCTTTGGTGA,
histone-lysine N-methyltransferase EHMT1 isoform 2 (Homo sapiens),
NCBI Reference Sequence: NP_001138999.1, 808 aa
SEQ ID NO: 48
MAAADAEAVPARGEPQQDCCVKTELLGEETPMAADEGSAEKQAGEAHMAADGETNGSCE
NSDASSHANAAKHTQDSARVNPQDGTNTLTRIAENGVSERDSEAAKQNHVTADDFVQTSVI
GSNGYILNKPALQAQPLRTTSTLASSLPGHAAKTLPGGAGKGRTPSAFPQTPAAPPATLGEGS
ADTEDRKLPAPGADVKVHRARKTMPKSVVGLHAASKDPREVREARDHKEPKEEINKNISDF
GRQQLLPPFPSLHQSLPQNQCYMATTKSQTACLPFVLAAAVSRKKKRRMGTYSLVPKKKTK
VLKQRTVIEMFKSITHSTVGSKGEKDLGASSLHVNGESLEMDSDEDDSEELEEDDGHGAEQA
AAFPTEDSRTSKESMSEADRAQKMDGESEEEQESVDTGEEEEGGDESDLSSESSIKKKFLKRK
GKTDSPWIKPARKRRRRSRKKPSGALGSESYKSSAGSAEQTAPGDSTGYMEVSLDSLDLRVK
GILSSQAEGLANGPDVLETDGLQEVPLCSCRMETPKSREITTLANNQCMATESVDHELGRCT
NSVVKYELMRPSNKAPLLVLCEDHRGRMVKHQCCPGCGYFCTAGNFMECQPESSISHRFHK
DCASRVNNASYCPHCGEESSKAKEVTIAKADTTSTVTPVPGQEKGSALEGRADTTTGSAAGP
PLSEDDKLQGAASHVPEGFDPTGPAGLGRPTPGLSQGPGKETLESALIALDSEKPKKLRFHPK
QLYFSARQGELQKVLLMLVDGIDPNFKMEHQNKRSPLHAAAEAGHVDICHMLVQFCRLGSP
RSRGCLW,

In some embodiments, the method comprises the step of, after the step of differentiating a population of pluripotent stem cells in aggregation media for a sufficient time to promote differentiation into a population of CD34+ hemogenic endothelium, inhibiting G9a and/or GLP in the resultant population of CD34+ hemogenic endothelium. In some embodiments, the method comprises the step of, before the step of differentiating a population of CD34+ hemogenic endothelium in a CD3+-T-cell differentiation media in the presence of a Notch ligand for a sufficient time to promote differentiation into a population of CD3+ T cells, inhibiting G9a and/or GLP in the population of CD34+ hemogenic endothelium.

Accordingly, in one aspect described herein is a method comprising: (a) differentiating a population of pluripotent stem cells in aggregation media for a sufficient time to promote differentiation into a population of CD34⁺ hemogenic endothelium; (b) inhibiting G9a and/or GLP in the resultant population of CD34⁺ hemogenic endothelium; and (c) differentiating the resultant population of CD34⁺ hemogenic endothelium in a CD3⁺-T-cell differentiation media in the presence of a Notch ligand for a sufficient time to promote differentiation into a population of CD3+ T cells.

In one embodiment, the inhibitor is a G9a/GLP inhibitor. In one embodiment, the G9a/GLP inhibitor is selected from a compound listed in Table 2, or a derivative or analog thereof. In one embodiment, the G9a/GLP inhibitor is selected from the group consisting of: UNC0224; UNC0638; A366; BRD4770; BIX01294; UNC0642; UNC0631; UNC0646; UNC0321; E72; BIX-01338; BRD9539; Chaetocin; and DCG066. In one embodiment, the G9a/GLP inhibitor is selected from the group consisting of: UNC0224; UNC0638; A366; BRD4770; BIX01294; and UNC0642. In some embodiments, the G9a/GLP inhibitor is selected from the group consisting of: UNC0224; UNC0638; BRD4770; BIX01294; and UNC0642.

In some embodiments, the G9a/GLP inhibitor is a Type I G9a/GLP inhibitor (e.g., a BIX-01294 derivative) selected from the group consisting of: UNC0224; UNC0638; A366; BIX01294; UNC0642; UNC0631; UNC0646; UNC0321; and E72. In some embodiments, the G9a/GLP inhibitor is a Type II G9a/GLP inhibitor (e.g., a BIX-01338 derivative) selected from the group consisting of: BRD4770; BIX-01338; and BRD9539. In some embodiments, the G9a/GLP inhibitor is a Type III G9a/GLP inhibitor such as Chaetocin. In some embodiments, the G9a/GLP inhibitor is a Type IV G9a/GLP inhibitor selected from the group consisting of: DCG066.

TABLE 2
G9a/GLP inhibitors that can promote T cell differentiation. All references
cited in Table 2 are specifically incorporated herein by reference in their entireties.

| | | Exemplary | |
|---|---|---|---|
| G9a/GLP | | effective | |
| inhibitor | Exemplary results | dose(s) | Small molecule structure |
| UNC0224 (e.g., Type I G9a inhibitor, BIX-01294 derivative) | See e.g., WO 2021/150919 | 312 nM, 625 nM, 1.25 uM, 2.5 uM, 5 uM | 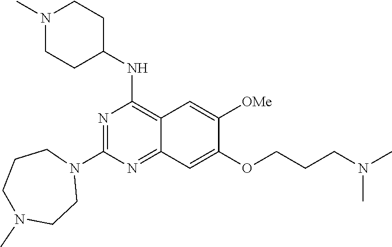 |
| UNC0638 (e.g., Type I G9a inhibitor, BIX-01294 derivative) | See e.g., WO 2021/150919 | 8 nM | 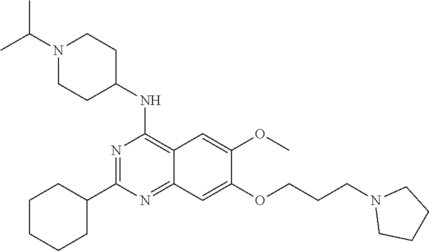 [SMILES: COc1cc2c(NC3CCN(C(C)C)CC3)nc(C3CCCCC3)nc2cc1OCCCN1CCCC1] |
| A366 (e.g., Type I G9a inhibitor, BIX-01294 derivative) | See e.g., WO 2021/150919 | N/A | 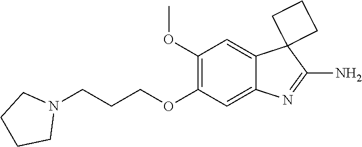 [SMILES: COc1cc2c(cc1OCCCN1CCCC1)N=C(N)C21CCC1] |
| BRD4770 (e.g., Type II G9a inhibitor, BIX-01338 derivative) | See e.g., WO 2021/150919; see e.g., Yuan et al., ACS Chem Biol. 2012 Jul 20; 7(7): 1152-1157. | 200 nM | 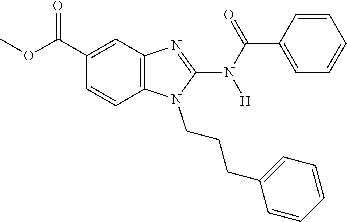 [SMILES: COC(=O)c1ccc2c(c1)nc(NC(=O)c1ccccc1)n2CCCc1ccccc1] |
| BIX01294 (e.g., Type I G9a inhibitor) | See e.g., WO 2021/150919 | 200 nM | 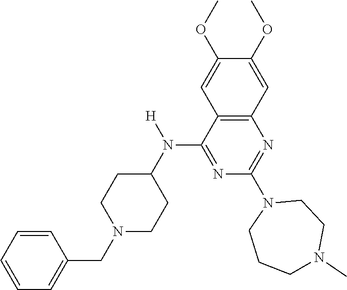 [SMILES: COc1cc2nc(N3CCCN(C)CC3)nc(NC3CCN(Cc4ccccc4)CC3)c2cc1OC] |
| UNC0642 (e.g., Type I G9a inhibitor, BIX-01294 derivative) | See e.g., WO 2021/150919 | 40 nM | 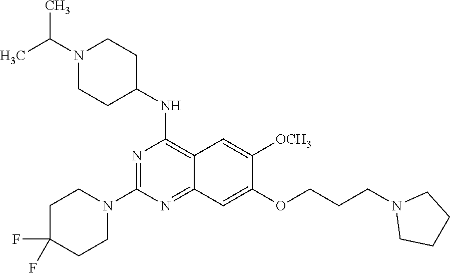 [SMILES: COc1cc2c(NC3CCN(C(C)C)CC3)nc(N3CCC(F)(F)CC3)nc2cc1OCCCN1CCCC1] |
| UNC0631 (e.g., Type I G9a inhibitor, BIX-01294 derivative) | See e.g., Liu et al. Journal of Medicinal Chemistry 54(17), 6139-6150 (2011). | E.g., in MDA-MB- 231 cells, IC₅₀ = 25 nM | 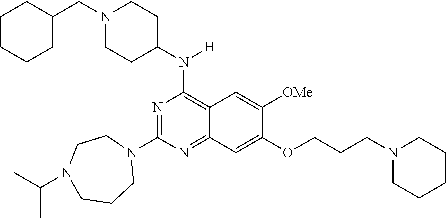 [SMILES: COc1cc2c(NC3CCN(CC4CCCCC4)CC3)nc(N3CCCN(C(C)C)CC3)nc2cc1OCCCN1CCCCC1] |
| UNC0646 (e.g., Type I G9a inhibitor, BIX-01294 derivative) | See e.g., Liu et al. Journal of Medicinal Chemistry 54(17), 6139-6150 (2011). | E.g., in MDA-MB- 231 cells, IC₅₀ = 26 nM | 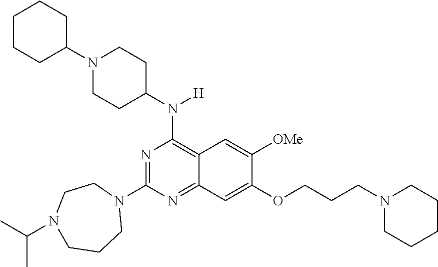 [SMILES: COc1cc2c(NC3CCN(C4CCCCC4)CC3)nc(N3CCCN(C(C)C)CC3)nc2cc1OCCCN1CCCCC1] |
| UNC0321 (e.g., Type I G9a inhibitor, BIX-01294 derivative) | See e.g., Liu et al. J Med Chem. 2010 Aug 12; 53(15): 5844-5857; Liu et al. J Med Chem. 2009 Dec 24; 52(24): 7950-7953. A cell- permeable, quinazoline analog that potently and selectively inhibits PKMT G9a (IC₅₀ = 6 nM and 9 nM in two biochemical assays for CLOT and ECSD, respectively, and Morrison Ki = 63 pM, which is approximately 250-fold more potent than a closely- related analog, BIX01294. It inhibits GLP with reduced potency (e.g., 15 nM) and is found to be inactive (IC50> 40 μM) toward other protein lysine and arginine methyltransferases, such as SET7/9 (aH3K4 PKMT), SET8/PreSET7 (aH4K20 PKMT), and PRMT3, as well as JMJD2E (>1000- fold selectivity) in ECSD enzymatic assays. | E.g., G9a (IC₅₀ = 6 nM and 9 nM, and Morrison Ki = 63 pM; GLP (e.g., 15 nM) | 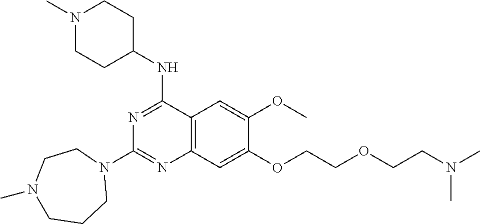 |
| E72 (e.g., Type I G9a inhibitor, BIX-01294 derivative) | See e.g., Chang et al. J Mol Biol. 2010 Jul 2; 400(1): 1-7 | E.g., G9a EC₅₀ 100 nM | 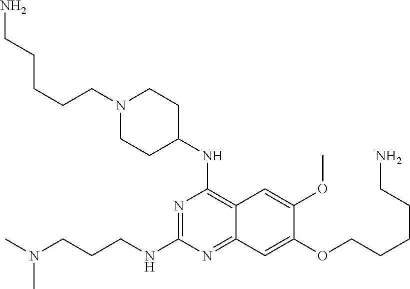 [SMILES: COc1cc2c(NC3CCN(CCCCCN)CC3)nc(NCCCN(C)C)nc2cc1OCCCCCN] |
| BIX-01338 (e.g., Type II G9a inhibitor) | See e.g., Greiner et al. Nature Chemical Biology 1(3), 143-145 (2005). A cell- permeable amino- benzimidazolo compound that is shown to inhibit a broad-spectrum of histone methyltransferases, including the PRMT1 H4R3 me2 activity, SET7/9 H3K4 me activity, G9a H3K9 me2 activity, as well as the H3K9 me3 activity of SUV39H1 (wild-type and H320R hyperactive mutant) and GLP (effective conc. = 15 μM) in a SAM-competitive manner. | G9a/GLP effective conc. = 15 μM | 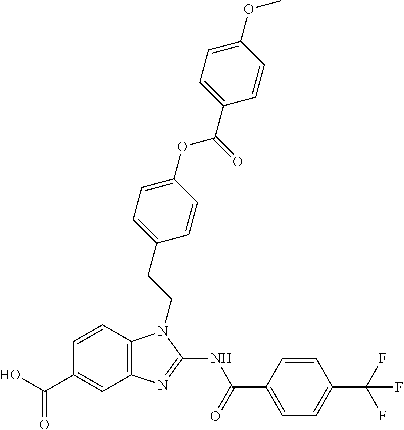 [SMILES: COc1ccc(C(=O)Oc2ccc(CCn3c(NC(=O)c4ccc(C(F)(F)F)cc4)nc4cc(C(=O)O)ccc43)cc2)cc1] |
| BRD9539 (e.g., Type II G9a inhibitor, BIX-01338 derivative) | See e.g., Yuan et al., ACS Chem Biol. 2012 Jul 20; 7(7): 1152-1157. BRD9539 is an inhibitor of G9a, with an IC50 value of 6.3 μM. It inhibits polycomb repressive complex 2 (PRC2) to a similar extent with 54 and 43% activity remaining for G9a and PRC2, respectively, when used at a concentration of 10 μM. It is selective for G9a and PRC2 over SU39H1 and NDMT1 up to a concentration of 40 μM. It is more potent than BRD4770 in enzyme assays, but may require a higher concentration for cell-based assays. | E.g., G9a, IC50 value of 6.3 μM | 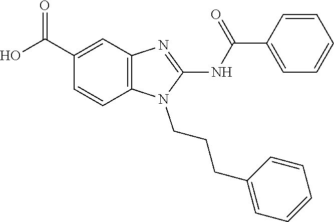 [SMILES: O=C(O)c1ccc2c(c1)nc(NC(=O)c1ccccc1)n2CCCc1ccccc1] |
| Chaetocin (e.g., (+)- Chaetocin; Type III G9a inhibitor) | See e.g., Greiner et al. Nature Chemical Biology 1(3), 143-145 (2005). Chaetocin is a fungal mycotoxin that inhibits the HMT SU(VAR)3-9 (IC₅₀ = 0.8 μM).1 It inhibits other Lys9-specific HMTs, including G9a (IC₅₀ = 2.5 μM) and DIM5 (IC₅₀ = 3 μM).1 Selectivity for Lys9- HMTs is indicated by higher IC50 values (>90 μM) for Lys20-specific PRSET7, Lys27-specific EZH2, and Lys4-specific SET7/9. | E.g., G9a IC₅₀ = 2.5 μM | 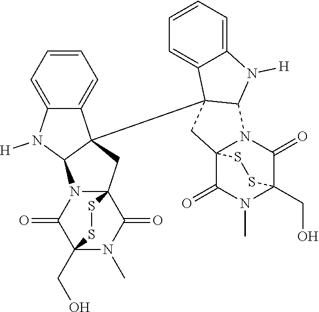 |
| DCG066 (e.g., Type IV G9a inhibitor) | See e.g., Kondengaden et al., Eur J Med Chem. 2016 Oct 21;122:382-393. | E.g., G9a EC₅₀ 6.5 uM | 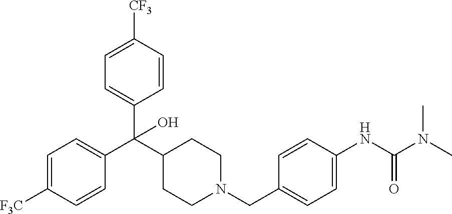 [SMILES: CN(C)C(=O)Nc1ccc(CN2CCC(C(O)(c3ccc(C(F)(F)F)cc3)c3ccc(C(F)(F)F)cc3)CC2)cc1] |

In some embodiments, the G9a/GLP inhibitor is provided at a concentration of at least 500 nM. In some embodiments, the G9a/GLP inhibitor is provided at a concentration of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 300 nM, at least 400 nM, at least 500 nM, at least 600 nM, at least 700 nM, at least 800 nM, at least 900 nM, at least 1.0 uM, at least 1.25 uM, at least 1.5 uM, at least 1.75 uM, at least 2.0 uM, at least 2.5 uM, at least 3 uM, at least 4 uM, at least 5 uM, at least 6 uM, at least 7 uM, at least 8 uM, at least 9 uM, or at least 10 uM. In some embodiments, the G9a/GLP inhibitor is provided at a concentration of 1 nM-10 nM, 10 nM-50 nM, 50 nM-100 nM, 100 nM-500 nM, 500 nM-1 uM, 1 uM-5 uM, or 5 uM-10 uM.

In some embodiments, the G9a/GLP inhibitor (e.g., UNC0224) is provided at a concentration of at least 312 nM, at least 625 nM, at least 1.25 uM, at least 2.5 uM, or at least 5 uM. In some embodiments, the G9a/GLP inhibitor (e.g., UNC0638) is provided at a concentration of at least 8 nM. In some embodiments, the G9a/GLP inhibitor (e.g., BRD4770) is provided at a concentration of at least 200 nM. In some embodiments, the G9a/GLP inhibitor (e.g., BIX01294) is provided at a concentration of at least 200 nM. In some embodiments, the G9a/GLP inhibitor (e.g., UNC0642) is provided at a concentration of at least 40 nM.

In some embodiments, the cells (e.g., CD34+ hemogenic endothelium) are cultured exposed to a G9a/GLP inhibitor until the development of CD5+CD7+ T cell progenitor cells. In some embodiments, the cells (e.g., CD34+ hemogenic endothelium) are cultured exposed to a G9a/GLP inhibitor for about 14 days. In some embodiments, the cells (e.g., CD34+ hemogenic endothelium) are cultured exposed to a G9a/GLP inhibitor for at least 1 day, at least 2 days, at least 3 days, at least 4 days, at least 5 days, at least 6 days, at least 7 days, at least 8 days, at least 9 days, at least 10 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, or more.

In some embodiments, culturing cells (e.g., CD34+ hemogenic endothelium) in the presence of a G9a/GLP inhibitor increases the number of resultant cells (e.g., CD5+CD7+ Pro-T cells; CD3+ T cells; CD4+CD8+ T cells; CD4+ T cells; CD8+ T cells; alpha-beta T cells) by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to cells not cultured in the presence of a G9a/GLP inhibitor.

In some embodiments, culturing cells (e.g., CD34+ hemogenic endothelium) in the presence of a G9a/GLP inhibitor decreases the number of erythroid or myeloid lineage cells (e.g., erythroid cell; macrophage; granulocyte; megakaryocyte) by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to cells not cultured in the presence of a G9a/GLP inhibitor.

In some embodiments, culturing cells (e.g., CD34+ hemogenic endothelium) in the presence of a G9a/GLP inhibitor decreases the total number of differentiated cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to cells not cultured in the presence of a G9a/GLP inhibitor.

In some embodiments, culturing cells (e.g., CD34+ hemogenic endothelium) in the presence of a G9a/GLP inhibitor increases the percentage of resultant cells of interest (e.g., CD5+CD7+ Pro-T cells; CD3+ T cells; CD4+CD8+ T cells; CD4+ T cells; CD8+ T cells; alpha-beta T cells) amongst the total number of differentiated cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to cells not cultured in the presence of a G9a/GLP inhibitor.

In some embodiments, a method for differentiating T cells as described herein (e.g., G9a/GLP inhibition and stromal-free T cell differentiation) produces a population that comprises at least 1%, at least 5%, at least 10%, at least 15%, at least 20%, at least 30%, at least 40%, at least 50%, at least 60%, at least 70%, at least 80%, at least 90%, at least 95%, or at least 99% of the cells of interest (e.g., CD5+CD7+ Pro-T cells; CD3+ T cells; CD4+CD8+ T cells; CD4+ T cells; CD8+ T cells; alpha-beta T cells). In some embodiments, a method for differentiating T cells as described herein (e.g., G9a/GLP inhibition and stromal-free T cell differentiation) produces a population that comprises at least 15% CD5+CD7+ T cell progenitor cells. See e.g., Greiner et al. Nature Chemical Biology 1(3), 143-145 (2005); Liu et al. Journal of Medicinal Chemistry 54(17), 6139-6150 (2011); Liu et al. J Med Chem. 2010 Aug. 12; 53(15): 5844-5857; Liu et al., J Med Chem. 2009 Dec. 24; 52(24): 7950-7953; Kondengaden et al., Eur J Med Chem. 2016 Oct. 21, 122:382-393; Yuan et al. ACS Chem Biol. 2012 Jul. 20; 7(7): 1152-1157; Chang et al. J Mol Biol. 2010 Jul. 2; 400(1): 1-7; Christman et al. Proceedings of the National Academy of Sciences of the United States of America 92(16), 7347-7351 (1995); Cheng et al., Signal Transduction and Targeted Therapy volume 4, Article number: 62 (2019); the contents of each of which are incorporated herein by reference in their entireties.

### Inhibition of a Histone Methyltransferase

In some embodiments, the differentiation method can comprise inhibiting a histone methyltransferase. The step of inhibiting a histone methyltransferase (e.g., EZH1 knockdown) can increase differentiation efficiency (e.g., of the T cells). Accordingly, in some embodiments, the differentiation method comprises inhibiting a histone methyltransferase, e.g., in the resultant population of CD34+ hemogenic endothelium. Methods of inhibiting a histone methyltransferase are known in the art; see e.g., International Application No. WO 2018/048828, US Application No. 2019/0225940, Doulatov et al., Cell Stem Cell. 2013 Oct. 3, 13(4); Vo et al., Nature 2018, 553(7689): 506-510; the contents of each of which are incorporated herein by reference in their entireties.

However, the step of inhibiting a histone methyltransferase (e.g., EZH1 knockdown) is not required. Thus, in some embodiments, the differentiation method does not comprise inhibiting a histone methyltransferase, e.g., in the resultant population of CD34+ hemogenic endothelium.

In the course of these experiments, the inventors discovered that inhibition of specific histone modifying enzymes targeting H3K9 and H3K27 promotes lymphoid potential of hematopoietic progenitors derived from pluripotent stem cells. The histone modifying enzymes are histone lysine methyltransferases. Post-translational modifications of histone proteins regulate chromatin compaction, mediate epigenetic regulation of transcription, and control cellular differentiation in health and disease. Methylation of histone tails is one of the fundamental events of epigenetic signaling. Tri-methylation of lysine 9 of histone H3 (H3K9) mediates chromatin recruitment of HP1, heterochromatin condensation and gene silencing. Similarly, methylation of H3K27 and H4K20 are associated with a repressed state of chromatin, whereas expressed genes are methylated at H3K4, H3K36 and H3K79. Methylation of H3K9 in humans relies mostly on members of the Suv39 family, namely EHMT1/GLP, EHMT2/G9a, SUV39H1, SUV39H2, SETDB1 and SETDB2, as well as then non-Suv39 enzymes PRDM2 and ASH1L (see e.g., Hong Wu et al., Structural Biology of Human H3K9 Methyltransferases, 2010, PLoS ONE, 5(2): e8570, which is incorporated herein by reference). In contrast, the methylation of H3K27 is carry out by the polycomb repressive complex 2 (PRC2).

Di/trimethylation of H3K9 is mainly catalyzed by the conserved SUV39H1/2 histone methyltransferases, while the polycomb repressive complex 2 (PRC2) ensures di/trimethylation of H3K27 (see e.g., Rea S, 2000. Nature 406:593-599; Margueron R, and Reinberg D. 2011. Nature 469:343-349). PRC2 comprises the EZH1/2 catalytic subunit, SUZ12, EED, and RBBP7/4 (see e.g., Margueron R, and Reinberg D, 2011).

It is specifically contemplated herein that inhibiting the histone lysine methyltransferases that target H3K9 and H3K27 relieves transcriptional repression that results from methylation of histone H3, and thereby promotes gene expression which facilitates cell differentiation, specifically T cell specification.

In one embodiment, the histone methyltransferase catalyzes the addition of methyl group to the histone H3 lysine residue 9 (H3K9) and/or histone H3 lysine residue 27 (H3K27).

In one embodiment, the histone methyltransferase inhibitor inhibits the G9a/GLP heteromeric complex.

G9a (EC 2.1.1.43) (UniProtKB: Q96KQ7) is also known as EHMT2, (Euchromatic Histone-Lysine N-Methyltransferase 2), G9A Histone Methyltransferase and protein G9a.

GLP (EC 2.1.1.43) (UniProtKB: Q9H9B1) is also known as EHMT1 (Euchromatic Histone-Lysine N-Methyltransferase 1), G9a-Like Protein 1 and GLP1.

In one embodiment, the histone methyltransferase inhibitor inhibits EZH1 (Enhancer of Zeste 1 Polycomb Repressive Complex 2 Subunit).

In one embodiment, the H3K27 histone methyltransferase is EZH1 (EC:2.1.1.43) (UniproKB Q92800-1).

In one embodiment, the H3K27 histone methyltransferase is not EZH2 (EC:2.1.1.43) (Unipro Q15910-1).

In one embodiment, the inhibitor of histone methyltransferase inhibits the gene expression or protein catalytic activity of the histone methyltransferase.

In one embodiment, the histone methyltransferase H3K9 and/or H3K27 is inhibited by a small molecule or a nucleic acid or a CRISPR-mediated target genetic interference.

In some embodiments, the histone methyltransferase H3K9 and/or H3K27 is inhibited by a small molecule inhibitor or a nucleic acid inhibitor. In one embodiment of any method, cells, or composition described, the histone methyltransferase small molecule inhibitor is a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. In some embodiments, the small molecule is a heterorganic compound or an organometallic compound.

In one embodiment, the histone methyltransferase small molecule inhibitor include but are not limited to AMI-1, A-366, BIX-01294, BIX01338, BRD4770, chaetocin, E72, UNC0224, UNC0631, UNC0638, UNC0642, UNC0646, EPZ5676, EPZ005687, GSK343, EPZ-6438 (E7438), 3-deazaneplanocin A (DZNeP) HCl, UNC1999, MM-102, SGC 0946, Entacapone, EPZ015666, UNC0379, Eli, MI-2 (Menin-MLL Inhibitor), MI-3 (Menin-MLL Inhibitor), PFI-2, GSK126, or EPZ004777.

In one embodiment, the histone methyltransferase small molecule inhibitor is selected from the group consisting of UNC0631, BRD4770, UNC1999, CPI-360, and BIX 01294.

In one embodiment, the nucleic acid inhibitor is a nucleic acid targeting the expression of histone methyltransferase. For example, targeting the mRNA or primary transcript of the histone methyltransferase, EZH1, thereby inhibiting protein expression of the enzyme. Histone-lysine N-methyltransferase aka Enhancer of Zeste 1 Polycomb Repressive Complex 2 Subunit (EZH1) or EC 2.1.1.43, is a component of a noncanonical Polycomb repressive complex-2 (PRC2) that mediates methylation of histone H3 (see MIM 602812) lys27 (H3K27) and functions in the maintenance of embryonic stem cell pluripotency and plasticity. The external identification for the human EZH1 gene are as follows: HGNC: 3526; Entrez Gene: 2145; Ensembl: ENSG00000108799; OMIM: 601674; UniProtKB: Q92800; EMBL: AB002386 mRNA and the corresponding mRNA translation: BAA20842.2; GENBANK: BT009782 mRNA and the corresponding mRNA translation: AAP88784.1.

In one embodiment, the nucleic acid inhibitor targets the human EZH1 mRNA.

In one embodiment, the nucleic acid inhibitor is a RNA interference inhibitor or CRISPR-mediated genetic interference inhibitor. The RNA interference inhibitor can be designed using the predictor RNAi softwares found at the Whitehead Institute, MIT, siRNA website, BLOCK-iT™ RNAi Designer at Invitrogen/ThermoFisher, and other online siRNA design tools at The RNAi Web using the mRNA of EZH1 as the target.

Similarly, Crisper guide RNA can be designed using the Broad Institute (MIT) CRISPR software (available on the world-wide web at, for example, portals.broadinstitute.org/gpp/public/analysis-tools/sgrna-design), dna20, Clontech, AddGene, e-crisp, and Innovative Genomic using the mRNA or genomic gene of EZH1 as the target.

CRISPR (Clustered Regularly Interspaced Short Palindromic Repeats) Cas9-mediated gene disruption has been widely used in generating loss-of-function mutations in diverse organisms including mammals (Cong et al., 2013, Science, 339(6121):819-23; reviewed in Hsu et al., 2014, Cell, 157(6):1262-78)). Cas9-based knockout screens have been applied in identifying essential genes and genes involved in drug resistance in various cell lines. With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830, US 2014-0287938, US 2014-0273234, US2014-0273232, US 2014-0273231, US 2014-0256046, US 2014-0248702, US 2014-0242700, US 2014-0242699, US 2014-0242664, US 2014-0234972, US 2014-0227787, US 2014-0189896, US 2014-0186958, US 2014-0186919, US 2014-0186843, US 2014-0179770 and US 2014-0179006, US 2014-0170753; European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and International Application No. WO 2014/093661, all of which are incorporated herein by reference in their entirety.

The CRISPR/Cas system envisaged for use in the context of the invention can make use of any suitable CRISPR enzyme. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes, or S. thermophilus Cas9, and may include mutated Cas9 derived from these organisms. The enzyme may be a Cas9 homolog or ortholog. In some embodiments, the CRISPR enzyme is codon-optimized for expression in a eukaryotic cell.

As described herein, the CRISPR/Cas system is used to specifically target a multitude of sequences within the continuous genomic region of interest. The targeting typically comprises introducing into each cell of a population of cells a vector system of one or more vectors comprising an engineered, non-naturally occurring CRISPR-Cas system comprising: at least one Cas protein, and one or more guide RNAs of the guide RNA library described herein.

In these methods, the Cas protein and the one or more guide RNAs may be on the same or on different vectors of the system and are integrated into each cell, whereby each guide sequence targets a sequence within the continuous genomic region in each cell in the population of cells. The Cas protein is operably linked to a regulatory element to ensure expression in said cell, more particularly a promoter suitable for expression in the cell of the cell population. In particular embodiments, the promoter is an inducible promoter, such as a doxycycline inducible promoter. When transcribed within the cells of the cell population, the guide RNA comprising the guide sequence directs sequence-specific binding of a CRISPR-Cas system to a target sequence in the continuous genomic region. Typically binding of the CRISPR-Cas system induces cleavage of the continuous genomic region by the Cas protein.

RNA interference (RNAi) mediated by short interfering RNAs (siRNA) or microRNAs (miRNA) is a powerful method for post-transcriptional regulation of gene expression. RNAi has been extensively used for the study of biological processes in mammalian cells and could constitute a therapeutic approach to human diseases in which selective modulation of gene expression would be desirable. Depending on the degree of complementarity between miRNA and target mRNA sequences, loss of gene expression occurs by inducing degradation of the cognate mRNA or by translational attenuation. Endogenous miRNAs are transcribed as primary transcripts and subsequently processed by the RNAse III enzyme Drosha to create a stem loop structure. Nuclear export and cleavage by Dicer generates a mature short double stranded molecule (siRNA) that is separated into guide and passenger strands. The guide strand is loaded into the RNA induced silencing complex (RISC), the effector complex mediating cleavage of target mRNAs with the functional guide strand binding to RISC proteins while the passenger strand is degraded. The loading of guide versus passenger strands into RISC largely depends on the 5′ end stability of the siRNA, with the less stable strand preferentially incorporated into RISC, although the exact regulation in mammalian cells is incompletely understood. The 5′ end of the guide strand contains the “seed region,” which is critical for target identification. Precise cleavage by Drosha and Dicer is critical for the generation of guide RNAs with defined seed regions that mediate efficient binding to the appropriate target mRNAs. Inaccurate processing results in binding to off-target molecules but a shift in cleavage sites also alters the nucleotide composition of duplex ends, which may have a profound effect on strand loading into RISC.

The inhibiting the expression of selected target polypeptides is through the use of RNA interference agents. RNA interference (RNAi) uses small interfering RNA (siRNA) duplexes that target the messenger RNA encoding the target polypeptide for selective degradation. siRNA-dependent post-transcriptional silencing of gene expression involves cleaving the target messenger RNA molecule at a site guided by the siRNA. RNAi is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see e.g., Coburn, G. and Cullen, B. (2002) J. Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be of at least 10%, at least 20%, at least 30%, at least 40%, at least 50%, at least 60%, at least 70%, at least 80%, at least 90%, at least 95%, at least 99%, or more as compared to the expression of a target gene or the activity or level of the protein encoded by a target gene which has not been targeted by an RNA interfering agent.

The terms “RNA interference agent” and “RNA interference” as they are used herein are intended to encompass those forms of gene silencing mediated by double-stranded RNA, regardless of whether the RNA interfering agent comprises an siRNA, miRNA, shRNA or other double-stranded RNA molecule. siRNA is defined as an RNA agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). The target gene or sequence of the RNA interfering agent may be a cellular gene or genomic sequence, e.g., the G9a/GLP or EZH1 sequence. An siRNA may be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target. The siRNA preferably targets only one sequence. Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST. siRNA sequences are chosen to maximize the uptake of the antisense (guide) strand of the siRNA into RISC and thereby maximize the ability of RISC to target G9a/GLP or EZH1 mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the human G9a/GLP or EZH1 mRNA. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. Preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA. The Examples herein provide specific examples of RNA interfering agents, such as shRNA molecules that effectively target mRNA.

In one embodiment, the nucleic acid is a G9a/GLP or EZH1 specific RNA interference agent or a vector encoding the RNA interference agent. In one embodiment, the RNA interference agent comprises one or more of the nucleotide sequences selected from the group consisting of

(SEQ ID NO: 11)
CTATCTGGCAGTGCGAGAATG,
(SEQ ID NO: 12)
AGACGTGCAAGCAGGTCTTTC,
(SEQ ID NO: 13)
TGGATGACTTATGCGTGATTT,
(SEQ ID NO: 14)
CAACAGAACTTTATGGTAGAA,
(SEQ ID NO: 15)
CCGCCGTGGTTTGTATTCATT,
(SEQ ID NO: 16)
GCTTCCTCTTCAACCTCAATA,
(SEQ ID NO: 17)
CCGCCGTGGTTTGTATTCATT,
(SEQ ID NO: 18)
GCTCTTCTTTGATTACAGGTA,
and
(SEQ ID NO: 19)
GCTACTCGGAAAGGAAACAAA.

In some embodiments, the nucleic acid inhibitor is a EZH1 specific nucleic acid that is selected from the group consisting of an aptamer that binds EZH1, a EZH1 specific RNA interference agent, and a vector encoding a EZH1 specific RNA interference agent, wherein the RNA interference agent comprises one or more of the nucleotide sequences selected from SEQ ID NO: 11-19.

In one embodiment, the multilineage hematopoietic progenitor cells are contacted with the viral vector or vector carrying a nucleic acid molecule comprising a nucleic acid sequence selected from a group consisting of SEQ ID NO: 11-19.

In one embodiment, the contacting with the histone methyltransferase inhibitor occurs more than once. For example, after the initial first contacting of the multilineage hematopoietic progenitor cell with the virus or vector carrying a nucleic acid molecule comprising a nucleic acid sequence selected from a group consisting of SEQ ID NO: 11-19, or contacting with a small molecule inhibitor described herein, the contacted cell is washed to remove that virus or vector, and the washed cell is then contacted for a second time with the same virus or vector used in the first contact.

It is contemplated herein that the Cas9/CRISPR system of genome editing be employed with the methods, cells and compositions described herein. Clustered regularly interspaced short palindromic repeats (CRISPR)/CRISPR-associated (Cas) systems is useful for RNA-programmable genome editing (see e.g., Jinek, M. et al. Science (2012) 337(6096):816-821).

Trans-activating crRNA (tracrRNA) is a small trans-encoded RNA. It was first discovered in the human pathogen Streptococcus pyogenes. (See Deltcheva E, et al. (2011). Nature 471 (7340): 602-7). In bacteria and archaea, CRISPR/Cas (clustered, regularly interspaced short palindromic repeats/CRISPR-associated proteins) constitute an RNA-mediated defense system which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. (See e.g., Terns M P and Terns R M (2011). Curr Opin Microbiol 14 (3): 321-7). There are several pathways of CRISPR activation, one of which requires a tracrRNA which plays a role in the maturation of crRNA. TracrRNA is complementary to and base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid. (see e.g., Deltcheva E, et al. supra; Jinek M, et al. (2012), Science 337 (6096): 816-21; and Brouns S J (2012), Science 337 (6096): 808-9).

In some embodiments, Cas9/CRISPR system guide RNAs are designed to target the exon 3 of EZH1 gene, which is present in all transcripts of EZH1 known. Exon 3 sequence is

(SEQ ID NO: 20)
ATTACAGCAAGATGGAAATACCAAATCCCCCTACCTCCAAATGTATCACT
TACTGGAAAAGAAAAGTGAAATCTGAATACATGCGACTTCGACAACTTAA
ACGGCTTCAGGCAAATATGGGTGCAAAG.
Non-limiting exemplary gRNAs that target exon 3
are
(SEQ ID NO: 21)
TCGACAACTTAAACGGCTTC,
(SEQ ID NO: 22)
TGCGACTTCGACAACTTAAA,
(SEQ ID NO: 23)
CCTCCAAATGTATCACTTAC,
(SEQ ID NO: 24)
TAAACGGCTTCAGGCAAATA
(SEQ ID NO: 25)
AAACGGCTTCAGGCAAATAT,
(SEQ ID NO: 26)
CATTTGGAGGTAGGGGGATT,
(SEQ ID NO: 27)
CCAGTAAGTGATACATTTGG,
(SEQ ID NO: 28)
GTGATACATTTGGAGGTAGG,
(SEQ ID NO: 29)
AAGTGATACATTTGGAGGTA,
(SEQ ID NO: 30)
AGTGATACATTTGGAGGTAG,
(SEQ ID NO: 31)
TTTCCAGTAAGTGATACATT,
and
(SEQ ID NO: 32)
TAAGTGATACATTTGGAGGT

In other embodiments, Cas9/CRISPR system guide RNAs are designed to target the exon 4 of EZH1 gene, which is also present in all transcripts of EZH1 known. Exon 4 sequence is

(SEQ ID NO: 33)
GCTTTGTATGTGGCAAATTTTGCAAAGGTTCAAGAAAAAACCCAGATCCT
CAATGAAGAATGGAAGAAGCTTCGTGTCCAACCTGTTCAGTCAATGAAGC
CTGTGAGTGGACACCCTTTTCTCAAAAAG.
Non-limiting exemplary gRNAs that target exon 4
are
(SEQ ID NO: 34)
GCTTCATTGACTGAACAGGT,
(SEQ ID NO: 35)
ACAGGCTTCATTGACTGAAC,
(SEQ ID NO: 36)
AGAAAAGGGTGTCCACTCAC),
(SEQ ID NO: 37)
TCCATTCTTCATTGAGGATC,
(SEQ ID NO: 38)
CCATTCTTCATTGAGGATCT,
(SEQ ID NO: 39)
CCCAGATCCTCAATGAAGAA,
(SEQ ID NO: 40)
GTATGTGGCAAATTTTGCAA,
and
(SEQ ID NO: 41)
CAGTCAATGAAGCCTGTGAG

In one embodiment, a vector is used as a transport vehicle to introduce any of the herein described nucleic acid inhibitors of a histone methyltransferase into the target cells selected from the cell populations as described herein (e.g., ESCs; PSCs; iPSCs; hemogenic endothelium; HSCs). In one embodiment, a vector is used as a transport vehicle to introduce any of the herein described nucleic acid comprising the described nucleic acid inhibitors of a histone methyltransferase into the target cells selected from the cell populations as described herein (e.g., ESCs; PSCs; iPSCs; hemogenic endothelium; HSCs). The in vivo expression of the nucleic acid inhibitor is for degrading the mRNA of the targeted histone methyltransferase such as G9a/GLP or EZH1 so as to reduce and inhibit the expression of the respective histone methyltransferase, with the goal being to reduce methylation of the histone H3 in the transfected cells and relief repression of gene expression therein.

In one embodiment, the host cell is an embryonic stem cell, a somatic stem cell, a progenitor cell, a bone marrow cell, a hematopoietic stem cell, a hematopoietic progenitor cell, an immune cell such as a T cell or B cell, an erythrocyte, a fibroblast, a keratinocyte, or a myeloid progenitor cell. In one embodiment, the host cell is isolated from a subject. In one embodiment, the host cell is isolated from a subject who has been diagnosed with a hematological disease.

In one embodiment, the vector further comprises a spleen focus-forming virus promoter, a tetracycline-inducible promoter, a Doxycycline (Dox)-inducible, or a s3-globin locus control region and a p3-globin promoter. In one embodiment, the promoter provides for targeted expression of the nucleic acid molecule therein. Other examples of promoters include but are not limited to the CMV promoter and EF1-alpha promoters for the various transgenes, and U6 promoter for shRNAs targeting EZH1.

In one embodiment, the vector is a virus or a non-viral vector. Non-limiting examples of viral vectors for gene delivery and expressions in cells are retrovirus, adenovirus (types 2 and 5), adeno-associated virus (AAV), Helper-dependent adenoviral vector (HdAd), hybrid adenoviral vectors, herpes virus, pox virus, human foamy virus (HFV), and lentivirus. Exemplary vectors useful in the invention described herein include episomal vectors, integrating vectors, non-integrating vectors, and excisable vectors.

### Stroma-Free T Cell Differentiation

In some embodiments, the present disclosure provides a method of generating CD3+ T cells comprising: (a) contacting CD34+ hemogenic endothelial (HE) cells with a first differentiation medium comprising interleukin-3 (IL-3) under conditions and for a sufficient time to generate CD5+ CD7+ T cell progenitors, and (b) contacting the CD5+CD7+ T cell progenitors with a second differentiation medium under conditions for a sufficient time to generate CD3+ T cells.

In some embodiments, the method comprises co-culture of the CD34+ HE cells in the presence of a Notch ligand, wherein the Notch ligand is not expressed by a stromal cell (e.g., a stroma-free differentiation method). Compared to differentiation with stromal cells expressing a Notch ligand, stroma-free differentiation can result in an increased number of differentiated T cells (see e.g., WO 2021/150919, the contents of which are incorporated herein by reference in its entirety), with a smaller portion of these T cells being innate-like cells. In some embodiments, the stroma-free protocol begins with culturing of hemogenic endothelium (HE), not iPSC or HE-derived progenitors (e.g., lymphoid progenitor).

### Notch Ligands

In nature, the hematopoietic stem cells (HSCs) in the bone marrow give rise to multipotent progenitors (MPPs) before differentiating into common myeloid progenitors (CMPs) and common lymphoid progenitors (CLPs). CLPs migrate from the bone marrow to the thymus, where thymic epithelial cells that express Delta-like ligand 4 (DLL4) trigger canonical Notch 1 signaling in early thymic progenitors (ETPs). This Notch 1 signal is essential for T cell lineage commitment and is further required during early phases of thymocyte differentiation up to the double-negative 3 (DN3) stage. Active Notch signaling during these early stages of T cell development inhibits other lineage potentials, such as B cell and myeloid cell (including dendritic cell (DC)) potential. During β-selection, Notch signaling is turned off as a consequence of pre-T cell receptor signaling. Thus subsequent stages of T cell development exhibit very low levels of Notch signaling. Notch was also suggested to influence the development of regulatory T (T_{Reg}) cells (specifically, thymic T_{Reg} cells). Notch signaling is mediated by the Notch 2 receptor. Notch signaling pathway is highly conserved in both vertebrate and invertebrate species and it regulates many different cell fate decisions. It is important for pattern formation during development such as neurogenesis, angiogenesis or myogenesis and regulates T cell development and stem cell maintenance. Notch signaling is also involved in cellular processes throughout adulthood. Signaling via Notch occurs between neighboring cells and both the receptor and its ligands are transmembrane proteins. See, e.g., Schmitt T. M., Zúñiga-Pflücker J. C. (2002) Induction of T cell development from hematopoietic progenitor cells by delta-like-1 in vitro. Immunity 17:749-756; Mohtashami M. (2010) Direct Comparison of DII1- and DII4-Mediated Notch Activation Levels Shows Differential Lymphomyeloid Lineage Commitment Outcomes. J Immunol. 185(2):867-76; Ohishi K et al, which are incorporated herein by reference. Delta-1 enhances marrow and thymus repopulating ability of human CD34(⁺) CD38(⁻) cord blood cells. J Clin Invest. 2002 October; 110(8):1165-74; and Dallas M H et al. Density of the Notch ligand Delta1 determines generation of B and T cell precursors from hematopoietic stem cells J Exp Med. 2005 May 2; 201(9): 1361-1366, which are incorporated herein by reference.

Accordingly, to initiate differentiation in the lymphoid lineage and T cell lineage commitment, the hemogenic endothelium is exposed to a Notch ligand to activate the Notch signaling pathway therein. In some embodiments, the stroma-free protocol described herein includes a step of contacting cells with a Notch ligand starting with hemogenic endothelium (HE), not iPSC or HE-derived progenitors (e.g., lymphoid progenitor). Accordingly, in some embodiments, iPSC or HE-derived progenitors are not the initial population that is differentiated into T cells in the presence of a Notch ligand.

Notch ligands are single-pass transmembrane proteins with a DSL (Delta, Serrate, LAG-2)-domain and varying numbers of EGF-like repeats. There are two classes of canonical Notch ligands, the Delta/Delta-like and the Serrate/Jagged class. The later has an additional domain of cysteine rich repeats close to the transmembrane domain. There are 5 canonical Notch ligands in mammals: Jagged-1, Jagged-2, DLL1, DLL3 and DLL4. These can bind to the four Notch receptors Notch 1-4. DLL1, also known as Notch Delta ligand, Delta-like 1, is a protein which interacts with a NOTCH2 receptor. See e.g., Shimizu K, et al., 2001, J. Biol. Chem. 276 (28): 25753-8; Blaumueller C M, et al., 1997, Cell 90 (2): 281-91; Shimizu K, et al., 2000, Mol. Cell. Biol. 20 (18): 6913-22. DLL1 is a protein that in humans is encoded by the DLL1 gene. DLL1 is a human homolog of the Notch Delta ligand.

In some embodiments, the Notch ligand is selected from the group consisting of Delta-like-1 (DLL1, also referred to as DL1), Delta-like-4 (DLL4, also referred to as DL4), immobilized Delta1ext-IgG, and immobilized Delta4ext-IgG. In some embodiments, immobilized Delta1ext-IgG consists of an extracellular domain of human Delta-like-1 fused to the Fc domain of human IgG1. “Immobilized Delta1ext-IgG” refers to recombinant Notch ligand made by fusing the extracellular domain of Delta-like 1 to the Fc domain of human IgG1 (see e.g., SEQ ID NO: 42). This is a synthetic way of providing a titratable dose of NOTCH ligand. See e.g., Varnum-Finney et al., J Cell Sci. 2000 December; 113 Pt 23:4313-8, which is incorporated herein by reference in its entirety. Recombinant Notch ligands and Fc-fusions are commercially available at AdipoGen™. “Immobilized Delta4ext-IgG” refers to recombinant Notch ligand made by fusing the extracellular domain of Delta-like 4 to the Fc domain of human IgG1 (see, e.g., SEQ ID NO: 43).

In some embodiments, the IgG domain of Delta1ext-IgG or Delta4ext-IgG can comprise any known IgG domain in the art. In some embodiments, Delta1ext-IgG or Delta4ext-IgG can be immobilized to a solid substrate (e.g., tissue culture plate) by coating the solid substrate with a composition that binds IgG Fc, including but not limited to anti-human IgG antibody, Protein G, or Protein A.

In some embodiments, the nucleic acid sequence of the Notch ligand (e.g., DLL1) comprises SEQ ID NO: 1-3 or a sequence that is at least 85%, at least 87%, at least 90%, at least 91%, at least 92%, at least 93%, at least 94%, at least 95%, at least 96%, at least 97%, at least 98%, or at least 99%) identical to the sequence of SEQ ID NO: 1-3 that maintains the same functions as SEQ ID NO: 1-3 (e.g., binding and/or activating a Notch receptor).

In some embodiments, the present methods comprise culture of the CD34+ HE cells in the presence of a Notch ligand, wherein the Notch ligand is not expressed on a stromal cell. In some embodiments, the Notch ligand is selected from DLL1, DLL4, immobilized Delta1^{ext-IgG}, or immobilized Delta4^{ext-IgG}. In one embodiment, the concentration of DLL1, DLL4, immobilized Delta1^{ext-IgG}, or immobilized Delta4^{ext-IgG} is in the range of 1 μg/mL to 40 μg/mL; in other embodiments the concentration is in the range of 5 μg/mL to 30 μg/mL, 10 μg/mL to 30 μg/mL, 20 μg/mL to 30 μg/mL, 5 μg/mL to 20 μg/mL, 5 μg/mL to 10 μg/mL, 5 μg/mL to 25 μg/mL, 10 μg/mL to 20 μg/mL, or any integer therebetween. In other embodiments, the concentration of DLL1, DLL4, immobilized Delta1^{ext-IgG}, or immobilized Delta4^{ext-IgG} is about 10 μg/mL, about 15 μg/mL, about 20 μg/mL or about 25 μg/mL.

DLL1 delta like canonical Notch ligand 1 [Homo sapiens (human)], Gene
ID: 28514, NCBI Reference Sequence: NG_027940.1, 8873 bp
SEQ ID NO: 1
actgaccatttggcgatccattgagaggagggtttggaaaagtggctcctttgtgacagctctcgccagattggggggctgctgatttgcatctcatta
gccatgcgggcggccggctgaatataagggcggcaggcgccggegagagccagatcctctgcgcgcaccegcggagacccgacccggccg
agggcagagcgcaggggaacccgggcagccgeggegcagagcctcctcccacggcccggcccctccggtcctgcgcgtgtgtactggatgg
cattggctggattcatcggaaagacgcggatctttgctgtgacaccggagatcggagcccggagtgctcccggaacgaccgccgccgccgagtg
acaccgggccgcgatccgcaggggccgccgegcacacccgccgccgccgaccgtcccctcagegegegecgctggccccggattatcgcctt
gcccgtgggatttccagaccgcggctttctaatcggctcgggaggaagctctgcagctctcttgggaattaagctcaatctctggactctctctctttct
ctttctccccctccctctcctgcgaagaagctcaagacaaaaccaggaagccggcgaccctcacctcctcgggggctgggaggaaggaggaaaa
cgaaagtcgccgccgccgcgctgtcccccgagagctgcctttcctcgggcatccctggggctgccgcgggacctcgcagggcggatataaaga
accgcggccttgggaagaggcggagaccggcttttaaagaaagaagtcctgggtcctgcggtctggggcgaggcaagggcgcttttctgcccac
gctccccgtggcccategatcccccgegcgtccgccgctgttctaaggagagaagtgggggccccccaggctcgcgcgtggagcgaagcagc
atgggcagtcggtgcgcgctggccctggcggtgctctcggccttgctgtgtcaggtaggcgggcaggtgggggcgccgcggccccgcggggt
ctcacgggtagccggggcgcggggcaggagcgcgcggggaggggcggacagcggcacgggccgcgccagccacggcccggaagatgaa
tcccgggggcgacgaccccagcgccggccgtgcagcgagegcgctcggcccctgagcccttccaggctctccgcacaccccccacccaggcc
tcacgccccctagctcggggggacccgcgtcctcacgcccccgccctcccccgtgcaggtctggagctctggggtgttcgaactgaagctgca
ggagttcgtcaacaagaaggggctgctggggaaccgcaactgctgccgcgggggcgcggggccaccgccgtgcgcctgccggaccttettcc
gcgtgtgcctcaagcactaccaggccagcgtgtcccccgagccgccctgcacctacggcagegccgtcacccccgtgctgggegtcgactcctt
cagtctgcccgacggegggggcgccgactccgcgttcagcaaccccatccgcttccccttcggcttcacctggccggtgagtgccgcacctgcg
cgcgccgggccggccctgaagctggggggctgcaggacgcgctgggatcccgccttgggcgctcggtgggggacctcggggaccccgc
gaggcgcaggtgggcgctgcgatctgcctagcggcggccccaggactccagcccagcagegeggacacctcgccccggggccccgcggcct
gcaggaggggaccgcgctggggcgaggaggagaggccgagcgcgcccgggagatttccgtatccggcctctgtgccaggtctccagtcaga
ggcgccccttcacgtgggaaggttctggtttcccgactcctagacgcgttggtggcgcgattaccegcgcagegcgaccgctaccacccggagc
gtgcccatcccccaagaaaaatgacaagggccctcgggcctcttccaccccatcctgcctgcattctctctctctctctaattaaaaaaacaacgtaat
atcctgtagtacaggctgaaaaaacacgtcaggaaaccactctttaaaaagttcttccatttccttagggaaggtgagagcaggcaggaggtgcgtg
gagaccctctccagacacgctgccccagacctgcagccttcaggcctctgttgctgacctggctgttaggaatgactgctttttgccgttttcttttcgtt
acctttctgggttgtctaacgtcttctcccctctctcccagggcaccttctctctgattattgaagctctccacacagattctcctgatgacctcgcaacag
gtaaaaacaaaacccaaaccccaaaactgctttccccagttaatagcattggactttgcccacccatcccccagccaaacccggacagctttcattct
gcacgtgccccagaaagttcagggtggagcagcttgggcctccttcccgtgctgaatgtctcggcccacccccgctctgtcccgagtcacagggtt
ctcgttcagaaccaaccaggagcatcttctccccgtagaaaacccagaaagactcatcagccgcctggccacccagaggcacctgacggtgggc
gaggagtggtcccaggacctgcacagcagcggccgcacggacctcaagtactcctaccgcttcgtgtgtgacgaacactactacggagagggct
gctccgttttctgccgtccccgggacgatgccttcggccacttcacctgtggggagcgtggggagaaagtgtgcaaccctggctggaaagggccc
tactgcacagagcgtgagtctctgggaaggcaccgctggctcactcgtccacgaacacggaccgcgcgcagggacggggcttcctgagccacg
gggggcttgggactgtagagatgttctggtggggaaactgaggcccagaggacagaagtggattgctataagtcacagctcgtcagtggggggg
ttggggtcaacgcagacattttaacatcccaggctgtgtttatccactatcggaactgcctttcttaatcagggaggattttagagacagggccagggg
tcaggaagtaaagccagtgctacccccagggtgtgtgtattagagagggagaggaggaaggaagggaggaacacagagagagcttgtgtgtca
ggggcaccatttcaacccgagttcccagtgctggaacagcatcacactgggaaacgttccattttctctctggagctggtgtgcttgacctctctgga
gcaaacgcctttccggatactccctgtgacacgcactgtctatgctggccagagagcaggctttcactcctgtgggctgctgaggccaggtctccaa
ggcctgtgtgggcgaggggtgcacagccccgtctggcttgaatgctcaggcagcaccttgtctggagaagcaatgtcttcccaatagtgacagag
gctctacctgcctcttattaggtattgatgtgtcaatgtcatggcaggcaggtgactagggcagggttggggccgtgctggctcctggttctggctcat
ggggacctcaggagccctctctccagctgactgaggcctcgcctgcacgcctggccgtcccagcccattggtaccggatttctctacagctgggg
attgggtaggtcctggagctgcccagaaactccagggaactgtcattctccttccttggaactggacaaccttggagaggggctctgggaggccca
gaacctctggcaggagctgggtagtgcctggggttgagggtgggtcttcccattcactgagtgccttgatgtccttgctccttagcttcccaaattccc
tccggaacttactgagctccttctaagctttgccttggcctgaactggttctggggaaaaacaaaaaaacaaaaaacaacttgtggagctgcttgttaa
tgagtttcataaccaggcagcaagagccagctccaagcctcaagcccactgtctactccctgccctgegggagcctctggccagtctgctgcctcc
cacccttcctccctgcctctcttcaccacagggtagccagaaacttaaacttttttcttcaaacactgaagtctctccccgcccccagctegegegtgcc
atagattagatctctccggggataggcgcagggacacccgccggctcccattggcggaaggggtgcgtgtgcgtgtgtgtgtgtgtgtgtgtgtgt
acacgcgaggggtgtgtgtgaggaggtggggccgggggcgcgggggaggccggcattgttgcgctggggcagctgccgtggaggacagac
aatggagcagctgtcctgccctggcaccctgcataccagctgtccactcttatctgcacacacactttctgggatattaagaggtggagctttgtgcac
agaattgggaagtgggggaggaggagggggaagacttctgaccctctcttagaagaaaaggggatagggtgggggtgggggcttccgagagc
ccttttgtccttgagcccctgtgttaagaagaatgctcatccccagggctgagtcaagtcccaggctactaggcaggggggtcagtcctccacaacc
tgggaagattaactcagctgggatttgctgactgaagccggcgagttgtgtcctggccccaagggcggcagccctgttgggacgtacttggcgtgg
ggcttgaccctgtttttcctttgcttgtagcgatctgcctgcctggatgtgatgagcagcatggattttgtgacaaaccaggggaatgcaagtaagtctg
cacaaggtggtgttttgttttgttgccttttcttgttatcttttcacagctggtgtatttgtaaaaacagccctaggtgatcattcgaaaaactccagtaagatt
gattgaacagggggccgttttctcatgtttctacttaatcaatgtttggcagcatgtaaggtcatggagttgtcattcgtctaagccccttaacggctatg
agaatttacagatagtagtttaaaaagagttggcacaggaaatgatagtatagttcaatggttctcaaatgttgcctcatcctagaatcactcagggagt
gatttttgagatgctgacactggtgctgccctaacacccaagaagccagaacctctggtggggcccaggcccaggctgcagctcccaaggtgacc
cagtgttctgctaatctggagaaccagaggctcactggtgctgcgggaagatggtttctagggtgagaatgtccactgcaaagccagcaacagtca
acgtccatctgagtcttctgcttttctccaaggtgcagagtgggctggcagggccggtactgtgacgagtgtatccgctatccaggctgtctccatgg
cacctgccagcagccctggcagtgcaactgccaggaaggctgggggggccttttctgcaaccagggtaagccttctctccctgaggcagcctgct
ccctccagagcagccctggacttccctggctgtttgatcactggaaaaataaagtcttcctgcatttgatgtcgagcttcctatctcctacttttcctgtcc
ccacccttcacagacctgaactactgcacacaccataagccctgcaagaatggagccacctgcaccaacacgggccaggggagctacacttgct
cttgccggcctgggtacacaggtgccacctgcgagctggggattgacgagtgtgaccccagcccttgtaagaacggagggagctgcacggtga
gtcggaggctccatggcatctcacccggaagctggggtgccctggtgttgaatggagtgtgtgggctccttggagcaactttggaaagccttttctg
acctctccategtgtaggatctcgagaacagctactcctgtacctgcccacccggcttctacggcaaaatctgtgaattgagtgccatgacctgtgcg
gacggcccttgctttaacgggggtcggtgctcagacagccccgatggagggtacagctgccgctgccccgtgggctactccggcttcaactgtga
gaagaaaattgactactgcagctcttcaccctgttctaatggtaagggggcagctggtgattgctcagagactcgggcgagcggtcaatactgaggt
ggcattaaaaacaagcatttgtgagtgacctcgagtttatgaatcacttttatccagaccgccaggaattctcgatggaaactctatctttgagtctgga
aaggcctggggaatgagagaggccagggcatttgttatgaagttctctgtggaaacctagaccaagcagtgaatgacttgctcagggccacaagg
tgcttcgggcacctgcggccgcctgaggttcagtaagtgatgcccacaggtgccggccactccagcttgggaggatggcccagctgtgtggcca
cccagcacagtagttgggggtgtccctgagtgaggacagagagcctcctgctagcagcgaggggctggctgcccaaaggagacacacagcaa
ggagagctgggccccagatgtgccggagcattccggaatggtcatccttcccctccctccctcccctgttgtcagtgcctgctcctctcacttgctgt
gtaactgtgggcaaggacaccctcgttaagcctcagtttccccatctgaaacctgggtcgagtggcacatgctcttgcccggctgttgtggcgacta
atgcagccaccagagtgttctgcacagcgcctgtccagatgctggccgtgtggtttctgacttgtagagctagacctggacacctctcgtatttgagg
tcctaaaccatgtcaccttgcgctgtggactcattcaggccacagactgtctttggtttgtctggtttctacagtgtcagacagatagatgcttcagagtg
actttttggtgaacaaacctacgaggagacacgtgatgttcatgtccctgtgttccaggtgccaagtgtgtggacctcggtgatgcctacctgtgccg
ctgccaggccggcttctcggggaggcactgtgacgacaacgtggacgactgcgcctcctccccgtgcgccaacgggggcacctgccgggatg
gcgtgaacgacttctcctgcacctgcccgcctggctacacgggcaggaactgcagtgcccccgtcagcaggtgcgagcacgcaccctgccacaa
tggggccacctgccacgagaggggccaccgctatgtgtgcgagtgtgcccgaggctacgggggtcccaactgccagttcctgctccccgagctg
cccccgggcccagcggtggtggacctcactgagaagctagagggccaggggggccattcccctgggtggccgtgtgcgccggggtcatcctt
gtcctcatgctgctgctgggctgtgccgctgtggtggtctgcgtccggctgaggctgcagaagcaccggcccccagccgacccctgccgggggg
agacggagaccatgaacaacctggccaactgccagcgtgagaaggacatctcagtcagcatcatcggggccacgcagatcaagaacaccaaca
agaaggcggacttccacggggaccacagcgccgacaagaatggcttcaaggcccgctacccagcggtggactataacctcgtgcaggacctca
agggtgacgacaccgccgtcagggacgcgcacagcaagcgtgacaccaagtgccagccccagggctcctcaggggaggagaaggggaccc
cgaccacactcagggggtgcgtgctgcgggccgggcatcaggagggggtacctggggggtgtcttcctggaaccactgctccgtttctcttccca
aatgttctcatgcattcattgtggattttctctattttccttttagtggagaagcatctgaaagaaaaaggccggactcgggctgttcaacttcaaaagaca
ccaagtaccagtcggtgtacgtcatatccgaggagaaggatgagtgcgtcatagcaactgaggtcagtgcaggcagcagccgctccctcctcctc
ggcatgggagcacctgaagctggagcacgggaatcggtctcaggctaacttcccatttgtcttgtggccccccaggtgtaaaatggaagtgagatg
gcaagactcccgtttctcttaaaataagtaaaattccaaggatatatgccccaacgaatgctgctgaagaggagggaggcctcgtggactgctgctg
agaaaccgagttcagaccgagcaggttctcctcctgaggtcctcgacgcctgccgacagcctgtcgeggcccggccgcctgcggcactgccttcc
gtgacgtcgccgttgcactatggacagttgctcttaagagaatatatatttaaatgggtgaactgaattacgcataagaagcatgcactgcctgagtgt
atattttggattcttatgagccagtcttttcttgaattagaaacacaaacactgcctttattgtcctttttgatacgaagatgtgctttttctagatggaaaaga
tgtgtgttattttttggatttgtaaaaatatttttcatgatatctgtaaagcttgagtattttgtgatgttcgttttttataatttaaattttggtaaatatgta
caaaggcacttcgggtctatgtgactatatttttttgtatataaatgtatttatggaatattgtgcaaatgttatttgagttttttactgttttgttaatgaaga
aattcctttttaaaatatttttccaaaataaattttatgaatgacaa,
Homo sapiens delta like canonical Notch ligand 1 (DLL1), mRNA, NCBI
Reference Sequence: NM_005618.4, 3779 bp
SEQ ID NO: 2
actgaccatttggcgatccattgagaggagggtttggaaaagtggctcctttgtgacagctctcgccagattggggggctgctgatttgcatctcatta
gccatgcgggcggccggctgaatataagggcggcaggcgccggcgagagccagatcctctgegcgcacccgeggagacccgacccggccg
agggcagagcgcaggggaacccgggcagccgcggcgcagagcctcctcccacggcccggcccctccggtcctgcgcgtgtgtactggatgg
cattggctggattcatcggaaagacgcggatctttgctgtgacaccggagatcggagcccggagtgctcccggaacgaccgccgccgccgagtg
acaccgggccgcgatccgcaggggccgccgegcacacccgccgccgccgaccgtcccctcagegegegecgctggccccggattatcgcctt
gcccgtgggatttccagaccgcggctttctaatcggctcgggaggaagctctgcagctctcttgggaattaagctcaatctctggactctctctctttct
ctttctccccctccctctcctgcgaagaagctcaagacaaaaccaggaagccggcgaccctcacctcctcgggggctgggaggaaggaggaaaa
cgaaagtcgccgccgccgcgctgtcccccgagagctgcctttcctcgggcatccctggggctgccgcgggacctcgcagggcggatataaaga
accgcggccttgggaagaggcggagaccggcttttaaagaaagaagtcctgggtcctgcggtctggggcgaggcaagggcgcttttctgcccac
gctccccgtggcccatcgatccccegcgcgtccgccgctgttctaaggagagaagtgggggccccccaggctegcgcgtggagcgaagcagc
atgggcagtcggtgcgcgctggccctggcggtgctctcggccttgctgtgtcaggtctggagctctggggtgttcgaactgaagctgcaggagttc
gtcaacaagaaggggctgctggggaaccgcaactgctgccgcgggggcgcggggccaccgccgtgcgcctgccggaccttcttccgcgtgtg
cctcaagcactaccaggccagcgtgtcccccgagccgccctgcacctacggcagegccgtcacccccgtgctgggcgtcgactccttcagtctgc
ccgacggcgggggcgccgactccgcgttcagcaaccccatccgcttccccttcggcttcacctggccgggcaccttctctctgattattgaagctct
ccacacagattctcctgatgacctcgcaacagaaaacccagaaagactcatcagccgcctggccacccagaggcacctgacggtgggcgagga
gtggtcccaggacctgcacagcagcggccgcacggacctcaagtactcctaccgcttcgtgtgtgacgaacactactacggagagggctgctcc
gttttctgccgtccccgggacgatgccttcggccacttcacctgtggggagcgtggggagaaagtgtgcaaccctggctggaaagggccctactg
cacagagccgatctgcctgcctggatgtgatgagcagcatggattttgtgacaaaccaggggaatgcaagtgcagagtgggctggcagggccgg
tactgtgacgagtgtatccgctatccaggctgtctccatggcacctgccagcagccctggcagtgcaactgccaggaaggctgggggggccttttc
tgcaaccaggacctgaactactgcacacaccataagccctgcaagaatggagccacctgcaccaacacgggccaggggagctacacttgctctt
gccggcctgggtacacaggtgccacctgcgagctggggattgacgagtgtgaccccagcccttgtaagaacggagggagctgcacggatctcg
agaacagctactcctgtacctgcccacccggcttctacggcaaaatctgtgaattgagtgccatgacctgtgcggacggcccttgctttaacggggg
tcggtgctcagacagccccgatggagggtacagctgccgctgccccgtgggctactccggcttcaactgtgagaagaaaattgactactgcagct
cttcaccctgttctaatggtgccaagtgtgtggacctcggtgatgcctacctgtgccgctgccaggccggcttctcggggaggcactgtgacgacaa
cgtggacgactgcgcctcctccccgtgcgccaacgggggcacctgccgggatggcgtgaacgacttctcctgcacctgcccgcctggctacacg
ggcaggaactgcagtgcccccgtcagcaggtgcgagcacgcaccctgccacaatggggccacctgccacgagaggggccaccgctatgtgtg
cgagtgtgcccgaggctacgggggtcccaactgccagttcctgctccccgagctgcccccgggcccagcggtggtggacctcactgagaagcta
gagggccaggggggccattcccctgggtggccgtgtgcgccggggtcatccttgtcctcatgctgctgctgggctgtgccgctgtggtggtctgc
gtccggctgaggctgcagaagcaccggcccccagccgacccctgccggggggagacggagaccatgaacaacctggccaactgccagcgtg
agaaggacatctcagtcagcatcatcggggccacgcagatcaagaacaccaacaagaaggcggacttccacggggaccacagcgccgacaag
aatggcttcaaggcccgctacccageggtggactataacctcgtgcaggacctcaagggtgacgacaccgccgtcagggacgcgcacagcaag
cgtgacaccaagtgccagccccagggctcctcaggggaggagaaggggaccccgaccacactcaggggtggagaagcatctgaaagaaaaa
ggccggactcgggctgttcaacttcaaaagacaccaagtaccagtcggtgtacgtcatatccgaggagaaggatgagtgcgtcatagcaactgag
gtgtaaaatggaagtgagatggcaagactcccgtttctcttaaaataagtaaaattccaaggatatatgccccaacgaatgctgctgaagaggaggg
aggcctcgtggactgctgctgagaaaccgagttcagaccgagcaggttctcctcctgaggtcctcgacgcctgccgacagcctgtcgcggcccg
gccgcctgcggcactgccttccgtgacgtcgccgttgcactatggacagttgctcttaagagaatatatatttaaatgggtgaactgaattacgcataa
gaagcatgcactgcctgagtgtatattttggattcttatgagccagtcttttcttgaattagaaacacaaacactgcctttattgtcctttttgatacgaagat
gtgctttttctagatggaaaagatgtgtgttattttttggatttgtaaaaatatttttcatgatatctgtaaagcttgagtattttgtgatgttcgttttttat
aatttaaattttggtaaatatgtacaaaggcacttcgggtctatgtgactatatttttttgtatataaatgtatttatggaatattgtgcaaatgttatttgag
ttttttactgttttgttaatgaagaaattcctttttaaaatatttttccaaaataaattttatgaatgacaa
Homo sapiens delta like canonical Notch ligand 1 (DLL1), CDS mRNA,
NCBI Reference Sequence: NM_005618.4, 2172 bp
SEQ ID NO: 3
atgggcagtcggtgcgcgctggccctggcggtgctctcggccttgctgtgtcaggtctggagctctggggtgttcgaactgaagctgcaggagttc
gtcaacaagaaggggctgctggggaaccgcaactgctgccgcgggggcgcggggccaccgccgtgcgcctgccggaccttcttccgcgtgtg
cctcaagcactaccaggccagegtgtcccccgagccgccctgcacctacggcagegccgtcacccccgtgctgggcgtcgactccttcagtctgc
ccgacggcgggggcgccgactccgcgttcagcaaccccatccgcttccccttcggcttcacctggccgggcaccttctctctgattattgaagctct
ccacacagattctcctgatgacctcgcaacagaaaacccagaaagactcatcagccgcctggccacccagaggcacctgacggtgggcgagga
gtggtcccaggacctgcacagcagcggccgcacggacctcaagtactcctaccgcttcgtgtgtgacgaacactactacggagagggctgctcc
gttttctgccgtccccgggacgatgccttcggccacttcacctgtggggagcgtggggagaaagtgtgcaaccctggctggaaagggccctactg
cacagagccgatctgcctgcctggatgtgatgagcagcatggattttgtgacaaaccaggggaatgcaagtgcagagtgggctggcagggccgg
tactgtgacgagtgtatccgctatccaggctgtctccatggcacctgccagcagccctggcagtgcaactgccaggaaggctgggggggccttttc
tgcaaccaggacctgaactactgcacacaccataagccctgcaagaatggagccacctgcaccaacacgggccaggggagctacacttgctctt
gccggcctgggtacacaggtgccacctgcgagctggggattgacgagtgtgaccccagcccttgtaagaacggagggagctgcacggatctcg
agaacagctactcctgtacctgcccacccggcttctacggcaaaatctgtgaattgagtgccatgacctgtgcggacggcccttgctttaacggggg
tcggtgctcagacagccccgatggagggtacagctgccgctgccccgtgggctactccggcttcaactgtgagaagaaaattgactactgcagct
cttcaccctgttctaatggtgccaagtgtgtggacctcggtgatgcctacctgtgccgctgccaggccggcttctcggggaggcactgtgacgacaa
cgtggacgactgcgcctcctccccgtgegccaacgggggcacctgccgggatggcgtgaacgacttctcctgcacctgcccgcctggctacacg
ggcaggaactgcagtgcccccgtcagcaggtgcgagcacgcaccctgccacaatggggccacctgccacgagaggggccaccgctatgtgtg
cgagtgtgcccgaggctacgggggtcccaactgccagttcctgctccccgagctgcccccgggcccagcggtggtggacctcactgagaagcta
gagggccagggcgggccattcccctgggtggccgtgtgcgccggggtcatccttgtcctcatgctgctgctgggctgtgccgctgtggtggtctgc
gtccggctgaggctgcagaagcaccggcccccagccgacccctgccggggggagacggagaccatgaacaacctggccaactgccagcgtg
agaaggacatctcagtcagcatcatcggggccacgcagatcaagaacaccaacaagaaggcggacttccacggggaccacagcgccgacaag
aatggcttcaaggcccgctacccageggtggactataacctcgtgcaggacctcaagggtgacgacaccgccgtcagggacgcgcacagcaag
cgtgacaccaagtgccagccccagggctcctcaggggaggagaaggggaccccgaccacactcaggggtggagaagcatctgaaagaaaaa
ggccggactcgggctgttcaacttcaaaagacaccaagtaccagtcggtgtacgtcatatccgaggagaaggatgagtgcgtcatagcaactgag
gtgtaa

In some embodiments, the amino acid sequence of the Notch ligand (e.g., DLL1) comprises SEQ ID NO: 4 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 4 that maintains the same functions as SEQ ID NO: 4 (e.g. binding and/or activating a Notch receptor).

delta-like protein 1 precursor [Homo sapiens],
NCBI Reference Sequence: NP_005609.3, 723 aa
SEQ ID NO: 4
MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMTCADGPCFNGGRCSDSPDGGYSCRCPVGYSGF
NCEKKIDYCSSSPCSNGAKCVDLGDAYLCRCQAGFSGRHCDDNVDDCASS
PCANGGTCRDGVNDFSCTCPPGYTGRNCSAPVSRCEHAPCHNGATCHERG
HRYVCECARGYGGPNCQFLLPELPPGPAVVDLTEKLEGQGGPFPWVAVCA
GVILVLMLLLGCAAVVVCVRLRLQKHRPPADPCRGETETMNNLANCQREK
DISVSIIGATQIKNTNKKADFHGDHSADKNGFKARYPAVDYNLVQDLKGD
DTAVRDAHSKRDTKCQPQGSSGEEKGTPTTLRGGEASERKRPDSGCSTSK
DTKYQSVYVISEEKDECVIATEV

In some embodiments, the Notch ligand (e.g., Delta1ext-IgG) comprises the extracellular domain of human DLL1, which corresponds to approximately amino acids 1-536, or amino acids 22-544, or amino acids 22-537 of DLL1 (see, e.g., SEQ ID NO: 4 for full-length sequence of DLL1). In some embodiments, the extracellular domain of human DLL1 comprises SEQ ID NO: 5, or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 5, and that maintains the same functions as SEQ ID NO: 5 (e.g., binding and/or activating a Notch receptor).

human DLL1 extracellular domain, 536 amino acids
SEQ ID NO: 5
MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMTCADGPCFNGGRCSDSPDGGYSCRCPVGYSGF
NCEKKIDYCSSSPCSNGAKCVDLGDAYLCRCQAGFSGRHCDDNVDDCASS
PCANGGTCRDGVNDFSCTCPPGYTGRNCSAPVSRCEHAPCHNGATCHERG
HRYVCECARGYGGPNCQFLLPELPPGPAVVDLTEKL,

In some embodiments, the nucleic acid sequence of the Notch ligand (e.g., DLL4) comprises SEQ ID NO: 6-9 or a sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 6-9, and that maintains the same functions as SEQ ID NO: 6-9 (e.g., binding and/or activating a Notch receptor).

DLL4 delta like canonical Notch ligand 4 [Homo sapiens (human)], Gene
ID: 54567, NCBI Reference Sequence: NG_046974.1, 9734 bp
SEQ ID NO: 6
agtagcggcgctgcgcgcaggccgggaacacgaggccaagagccgcagccccagccgccttggtgcagegtacaccggcactagcccgctt
gcagccccaggattagacagaagacgcgtcctcggegcggtcgccgcccagccgtagtcacctggattacctacagcggcagctgcagcggag
ccagcgagaaggccaaaggggagcagcgtcccgagaggagcgcctcttttcagggaccccgccggctggcggacgcgcgggaaagcggcg
tcgcgaacagagccagattgagggcccgcgggtggagagagcgacgcccgaggggatggcggcagcgtcccggagcgcctctggctgggc
gctactgctgctggtggcactttggcagcaggtaacacgtcccgcgccctctccgtcccctctgccgcgctctgggcctcagccccgggcaccag
ctgagctgaccggtcccctccctccttccctcggtccctgtgcaatagegcgeggccggctccggcgtcttccagctgcagctgcaggagttcatca
acgagcgcggcgtactggccagtgggcggccttgcgagcccggctgccggactttcttccgcgtctgccttaagcacttccaggcggtcgtctcg
cccggaccctgcaccttcgggaccgtctccacgccggtattgggcaccaactccttcgctgtccgggacgacagtagcggcggggggcgcaac
cctctccaactgcccttcaatttcacctggccggtgagcacagcctgggcgcactgggaggtcgcagaagccgagagaggaggcgccctggga
ccaaagccccctccccagatttccttgtacacacacccccacccccaaaaagcccaggatgcattctttcctggctcttcccgactctctcctgagact
gatcccagaaaaggctctcaccagtctccgtcttcccagtttatgtcctcccgtccccagctcttgggacacgattttcattacctaccactctggggcg
gtaccctaccaccccctcctccagtggctctcccttacactctcccgtctctcaaccctccctctaccgggggttctcctctcgccttccctgctcaagc
gctacactgtgcacagccccgttatgttgacccgggcgcagtaactgaatcctgcaattagattaattaaacaggctgccgcaaggcacccccacct
ctccccgcttgctcatctcgccatctctccgtccccccaccccctttcccagggtaccttctcgctcatcatcgaagcttggcacgcgccaggagacg
acctgcggccaggtgagtagctcgctccgccaccacaggggggcgacacggcgcagcgccgaaagagttaatctgttctaggcgggggaagt
gcgggcttggggggggaggcaggacgcttagcttggcctggagctgcgccccgcgctggacgctcggattccgctcgctgcctggactcaga
gcacaattgcgtttcctgcgggttatttttggcgtgggaacgcggggagtacggcggtgagaaaggctgaagctgccagcgccgctgacgggcc
ccttcctgtattttacacctttcgcgaattccgctcctttggaaagggaataatggctttgggatgttgttctgacacagaggaaaaggatatttcagcag
cacaacaattctcactttgaaaaggaaaaaagaaaaccattacccacctctggaggcagaacccctgaatgggcaccaaaggaccccctgctcccaggg
tcctctctagcctggggagcttttctttctttttctcttttttccattttgacctcttttcctctttcccctccctatctgcctccaagaccctgggatatctt
aacatccttctattgtcccctttttgaatactatcaggccccctgcacatgcacacacgtagggcagctacgtagcggggctttgggtccctctggcct
gttcttgctggcaggcgggggtcatctggataactgggctgattggttggctgatcaccatcatcacagccaagaaggacattggccagccgtcact
ggcacccttggggactggcgacccttccctgacccgaccctctgccccctcagaggccttgccaccagatgcactcatcagcaagategccatcc
agggctccctagctgtgggtcagaactggttattggatgagcaaaccagcaccctcacaaggctgcgctactcttaccgggtcatctgcagtgacaa
ctactatggagacaactgctcccgcctgtgcaagaagcgcaatgaccacttcggccactatgtgtgccagccagatggcaacttgtcctgcctgccc
ggttggactggggaatattgccaacagcgtaagcagtcaagctcccacctgtgtggaaggggagggtcccctgaggaaacacagtggagcttctt
ggtcacagcttgcctcccttgaagagtgggtctgggcctcctactagctgggcctcagggatgctgagggtgggcttgacctcagacctcctgtctc
ttcccagtgctcctcccatcatgccaaagcccacaagaaccccatcatgacattccatccagtttggcttctccttccctgtgccattatttcactttaaga
cactcggggctcctctgggaggccaggagtaggaagagggcccaggagagctaggggatccccagggccagcaggtgagaatggggcttaa
gagtccttggtatcccagcctcacccagctctgtgttcttcccttagctatctgtctttcgggctgtcatgaacagaatggctactgcagcaagccagca
gagtgcctgtgagtaggggacaggaagtggtgagtgggagccctcccttggccaaggcctctcacctcactctgcctctctcttgttccccagctgc
cgcccaggctggcagggccggctgtgtaacgaatgcatcccccacaatggctgtcgccacggcacctgcagcactccctggcaatgtacttgtga
tgagggctggggaggcctgttttgtgaccaaggtgagtcagggtgaagagagggtgcagagggtgcaagagatatggggctggggggtggaa
atccgattcgtcacctggatccttcttacttggtgactgcagacttggctttcccatgatcttccaaggatcttgggtcttttaaggatctttacaactggcc
cagaatgaggcggtgggtccttctccaggtgcggcggcagggggtggtggagccagggtggctgaaaaacccaggggggtgacaaggtcgg
cagcctggaggttgcactcataaatcctagcaaagccaaagagagagggatggcaggctcagttcctctttcaaccccgtagttacctattaacccc
ctgagtgtttgcttaccttccagggctgtttgagcagctctcccctaaacagctgtccggtggggtgtgcccaccggccacctgaggctgtgggtga
gctgggcctctgggcggagtggcatctaaccgacttttcggtgtgggcacaaacggcctcccctgctcttacctagttaccacctgcctgaacccat
gcggtctctacctggtgtttaggggtagtcactctctggctatacaggggcctttcagccccaaccttgggggaggaggaagccttttttcttgcatcc
tgctagccagctgcagccagctgcagctcccattttcaggatcaaatgggtgcacctgctgcccagagacaccggcgcaggcctgggtagggtg
ggcagagagcttgccagggtggaaagaaattgcctaggccctgacttgctgtcaacaaggggcttgggattcagtccctgtgttgtgtgtgtgtgtgt
gtgtgtgtgtgtgtgtctgtccctttactaccatccccaccccaacactcacacacctggttcctgctcattctcttccctctccaccatatttgctcccag
gtgacacagtcatatactcatcatatgcaaacacagcacttgcaggccatatatttactctgtctggttctccctccctgtccttcccaaataaaaaaaca
aatacttatatttcaaaatacccttgtaacacctcttcctttaaaaaatgcccgattactgcctatggtggctctcatctctcctctaccatttctacctgttga
aattttatccctccttccaggcttatctcagctgcccctcctccatgaagccttttctgacttcctccccgacatgtggccttgccctctgctcttcttccttat
cttcatcctacttgggttggcagtttgtgagtttccctggcaggacgtcttccagttccagttgtgttgtttcacttttggttgactgcactggtcatatgtga
ttcaaggtgctttaagaaacatgattttcatcctggctaacacagtgaaaccctgtctgtattaaaaatacaaaagttagccaggtgtggtggcaggca
cctgtagccccagctgctgggaaggctgaggcaggagaatggcgaagtagagcttgcagtgagccgaggtcgtgccactgcactccagcctga
gtgacagagcaagactccgtctcaaaaaaaaaaaaaaaaaaaaaaaaaagaaacatgattttaggctgggtgcgatggcctgtaatcccagcactt
tgggaggccgaggtaggtggatcacttgaagtcaggagttcgagaccatcctggccatcctggtgaaacccctgtaaaaatacaaatattaatcgg
gcacagtggcgcatgcctgtaatcccagctacttagaaggttgaggtatgagaatcgcttgaacccggaaggcgaaggttgtagtgagcctatatc
acatcactgcactccagcctgggcgacagagtgagactctgttaaaaaaaaaaaaaaaagaaggaaagaaagagaaagagagagaaagaaag
aaagaaagagaaagaaaaaagattttattggtggtggaggaaggatgtttgggcctgggagactttgagttgaggtgtctttgagccaaacatggg
ggcaaacatggactgcaaggagcctggaggtgagtgcattccctggccctgctcagctgcttggttcctgtttctgcagatctcaactactgcaccca
ccactccccatgcaagaatggggcaacgtgctccaacagtgggcagegaagctacacctgcacctgtcgcccaggctacactggtgtggactgtg
agctggagctcagcgagtgtgacagcaacccctgtcgcaatggaggcagctgtaaggtgaggcccagaccagcgcaggaagacagaggtgtc
aggtggtgtctgggcatccctaacctaggcagttagtggatgtacagccatggacaggcattgtgggcaggtggagcccagccttcagtcacacat
ccctgccccccagggtctgactttggcccctttatggtctctctccaggaccaggaggatggctaccactgcctgtgtcctccgggctactatggcct
gcattgtgaacacagcaccttgagctgcgccgactccccctgcttcaatgggggctcctgccgggagcgcaaccagggggccaactatgcttgtg
aatgtccccccaacttcaccggctccaactgcgagaagaaagtggacaggtgcaccagcaacccctgtgccaacggtgcgtgctgctgccctgct
aacctggtggactggccctggggctgagagagacttctggtgagggagggtcaggagaggagcgaggcattgtctgccactctggccccccatc
tgctctggagggcgaagagcttgcttgatcagctggggggctgtggaagcggagctggttagttgcacgcaggccttaggagcaggggtggtat
gcaccctgcatagcttccattcctattcccatgtcagaaccccgtcctggctggggggcctctgaccctccccaggaagtcctgagctggagagag
ggatgttggaggcttcatgtttctcctcaaaggaggcagtgattcagtcagagccctgctcctggaggcctcatcttgccccgtgcccaggtagagc
atgaggtagcatgaggcatcttgaatgtttgcaccttttgaggcacaaagcctgttggtaatccttgtctatctggctcccaggtgaccctctgtgaggc
aggcaggcaggcagcgctcaggagctggagagggggggaagggctgagagggagtctgctctctcactgaagcctctggcactgccatttctt
catcactgaatgggaaactataatacctgtcctctgtccttcatgtggttgtgaagatgaagtaaaacagtcatgattgtacttatccgagcattaactata
taccaaacatgggctcttgccttcatgtaccttcccggctatcctatgaaggggctagcattctactccagtctaacaaatggggaaactgaggcttag
agacacggttaagcagcaagtgccagatctcaggccacagagtgacagctgaggtcccaactcaagcctatctgtctgattctacgttaaagttctgt
aagatgctagtcatttttatacatgagcccactgaggccgagagaatcaaggtcatgctaaactccaggtctcctgactctgtgcagttctctttgtagt
gggctctgcaggtggaggtagaagggcccgaacgtgttcctggaatggggctcccaccccctgccccagggagctcccaggctatcactgactt
gtgtctcatgcgtcctcacagggggacagtgcctgaaccgaggtccaagccgcatgtgccgctgccgtcctggattcacgggcacctactgtgaa
ctccacgtcagcgactgtgcccgtaacccttgcgcccacggtggcacttgccatgacctggagaatgggctcatgtgcacctgccctgccggcttct
ctggccgacgctgtgaggtgcggacatccatcgatgcctgtgcctcgagtccctgcttcaacagggccacctgctacaccgacctctccacagaca
cctttgtgtgcaactgcccttatggctttgtgggcagccgctgcgagttccccgtgggcttgccgcccagcttcccctgggtggccgtctcgctgggt
gtggggctggcagtgctgctggtactgctgggcatggtggcagtggctgtgcggcagctgcggcttcgacggccggacgacggcagcaggga
agccatgaacaacttgtcggacttccagaaggacaacctgattcctgccgcccagcttaaaaacacaaaccagaagaaggagctggaagtggact
gtggcctggacaagtccaactgtggcaaacagcaaaaccacacattggactataatctggccccagggcccctggggcgggggaccatgccag
gaaagtttccccacagtgacaagagcttaggagagaaggcgccactgcggttacacaggtgagtggcacccagaagcccagggcctggccacc
ggccccgacatggttctgcctaggctcctcttaggccaggcgggaagcagttaagcagctgaggttttgttactgacaggaagatcctccagtaggattt
ctgtcaggggtcctttgtccttccctcccattcattcatttgttcattcacacatgtcaagtgtccctagggtgtctcttgtgacttccgtctttccacag
tgtggcttgcctctagtggcagcactggctttatgcagggctcagacccttctggtgaggttgggaggcctgtgactctcttaggggccttttcctaag
tgcccccctgcagcagcccagcactgggcacgtccagcccctgtgtcttccccaagaaccaccctgcagatgccctttggctctccagggtcctcc
ctccccccaagcctctccccgtccctcccttacacgcctgtcttgtgttccctcagtgaaaagccagagtgtcggatatcagegatatgctcccccag
ggactccatgtaccagtctgtgtgtttgatatcagaggagaggaatgaatgtgtcattgccacggaggtgagtgctgggctcgcctttccttctgccttt
tgtgggagggaaagtggcctggtcactcttgacccatgggccattcctgaagggtaggtcagaaccctgccttggcaggccaagttcagtggactc
ttgggtccctgctggcctcattgccactaagggtgtgaaacaggaaccatggcggcaagcctggtctggtcctttcctgctgtattggtgctgggttg
ggcagccacggcactgctggccagcctctgatgggtgagggggcccctcaccccttgtgcccttcctgccccttcccactggettcctccattgacc
tcatgagcgcaagctcccaggcccgtgtgtgtgttgggccgaagactggggaggactgccccacctgcccttagcccctgcctgccccategcct
tctcccagggaggcccagggagggcctggagggagtgcgcatgcccagggtaacctgtttccctgccttccgcttgctcccaggtataaggcag
gagcctacctggacatccctgctcagccccgcggctggaccttccttctgcattgtttacattgcatcctggatgggacgtttttcatatgcaacgtgct
gctctcaggaggaggagggaatggcaggaaccggacagactgtgaacttgccaagagatgcaatacccttccacacctttgggtgtctgtctggc
atcagattggcagctgcaccaaccagaggaacagaagagaagagagatgccactgggcactgccctgccagtagtggccttcagggggctcctt
ccggggctccggcctgttttccagagagagtggcagtagccccatggggcccggagctgctgtggcctccactggcatccgtgtttccaaaagtg
cctttggcccaggctccacggcgacagttgggcccaaatcagaaaggagagagggggccaatgagggcagggcctcctgtgggctggaaaac
cactgggtgcgtctcttgctggggtttgccctggaggtgaggtgagtgctcgagggaggggagtgctttctgccccatgcctccaactactgtatgc
aggcctggctctctggtctaggccctttgggcaagaatgtccgtctacccggcttccaccaccctctggccctgggcttctgtaagcagacaggcag
agggcctgcccctcccaccagccaagggtgccaggcctaactggggcactcagggcagtgtgttggaaattccactgagggggaaatcaggtg
ctgcggccgcctgggccctttcctccctcaagcccatctccacaacctcgagcctgggctctggtccactactgccccagaccaccctcaaagctg
gtcttcagaaatcaataatatgagtttttattttgtttttttttttttttttgtagtttattttggagtctagtatttcaataatttaagaatcaga
agcactgacctttctacattttataacattattttgtatataatgtgtatttataatatgaaacagatgtgtacagga,
Homo sapiens delta like canonical Notch ligand 4 (DLLA), mRNA,
NCBI Reference Sequence: NM_019074.4, 3426 bp
SEQ ID NO: 7
agtagcggcgctgcgcgcaggccgggaacacgaggccaagagccgcagccccagccgccttggtgcagcgtacaccggcactagcccgctt
gcagccccaggattagacagaagacgcgtcctcggcgcggtcgccgcccagccgtagtcacctggattacctacagcggcagctgcagcggag
ccagcgagaaggccaaaggggagcagcgtcccgagaggagcgcctcttttcagggaccccgccggctggcggacgcgcgggaaagcggcg
tcgcgaacagagccagattgagggcccgcgggtggagagagcgacgcccgaggggatggcggcagcgtcccggagcgcctctggctgggc
gctactgctgctggtggcactttggcagcagcgcgcggccggctccggcgtcttccagctgcagctgcaggagttcatcaacgagcgcggcgta
ctggccagtgggcggccttgcgagcccggctgccggactttcttccgcgtctgccttaagcacttccaggeggtcgtctegcceggaccctgcacc
ttcgggaccgtctccacgccggtattgggcaccaactccttcgctgtccgggacgacagtagcggcggggggcgcaaccctctccaactgccctt
caatttcacctggccgggtaccttctcgctcatcatcgaagcttggcacgcgccaggagacgacctgcggccagaggccttgccaccagatgcact
catcagcaagatcgccatccagggctccctagctgtgggtcagaactggttattggatgagcaaaccagcaccctcacaaggctgcgctactcttac
cgggtcatctgcagtgacaactactatggagacaactgctcccgcctgtgcaagaagcgcaatgaccacttcggccactatgtgtgccagccagat
ggcaacttgtcctgcctgcccggttggactggggaatattgccaacagcctatctgtctttcgggctgtcatgaacagaatggctactgcagcaagc
cagcagagtgcctctgccgcccaggctggcagggccggctgtgtaacgaatgcatcccccacaatggctgtcgccacggcacctgcagcactcc
ctggcaatgtacttgtgatgagggctggggaggcctgttttgtgaccaagatctcaactactgcacccaccactccccatgcaagaatggggcaac
gtgctccaacagtgggcagcgaagctacacctgcacctgtcgcccaggctacactggtgtggactgtgagctggagctcagcgagtgtgacagc
aacccctgtcgcaatggaggcagctgtaaggaccaggaggatggctaccactgcctgtgtcctccgggctactatggcctgcattgtgaacacag
caccttgagctgcgccgactccccctgcttcaatgggggctcctgccgggagcgcaaccagggggccaactatgcttgtgaatgtccccccaactt
caccggctccaactgcgagaagaaagtggacaggtgcaccagcaacccctgtgccaacgggggacagtgcctgaaccgaggtccaagccgca
tgtgccgctgccgtcctggattcacgggcacctactgtgaactccacgtcagcgactgtgcccgtaacccttgcgcccacggtggcacttgccatga
cctggagaatgggctcatgtgcacctgccctgccggcttctctggccgacgctgtgaggtgcggacatccategatgcctgtgcctcgagtccctg
cttcaacagggccacctgctacaccgacctctccacagacacctttgtgtgcaactgcccttatggctttgtgggcagccgctgcgagttccccgtgg
gcttgccgcccagcttcccctgggtggccgtctcgctgggtgtggggctggcagtgctgctggtactgctgggcatggtggcagtggctgtgcgg
cagctgcggcttcgacggccggacgacggcagcagggaagccatgaacaacttgtcggacttccagaaggacaacctgattcctgccgcccag
cttaaaaacacaaaccagaagaaggagctggaagtggactgtggcctggacaagtccaactgtggcaaacagcaaaaccacacattggactata
atctggccccagggcccctgggggggggaccatgccaggaaagtttccccacagtgacaagagcttaggagagaaggcgccactgcggttac
acagtgaaaagccagagtgtcggatatcagcgatatgctcccccagggactccatgtaccagtctgtgtgtttgatatcagaggagaggaatgaatg
tgtcattgccacggaggtataaggcaggagcctacctggacatccctgctcagccccgcggctggaccttccttctgcattgtttacattgcatcctg
gatgggacgtttttcatatgcaacgtgctgctctcaggaggaggagggaatggcaggaaccggacagactgtgaacttgccaagagatgcaatac
ccttccacacctttgggtgtctgtctggcatcagattggcagctgcaccaaccagaggaacagaagagaagagagatgccactgggcactgccct
gccagtagtggccttcagggggctccttccggggctccggcctgttttccagagagagtggcagtagccccatggggcccggagctgctgtggc
ctccactggcatccgtgtttccaaaagtgcctttggcccaggctccacggcgacagttgggcccaaatcagaaaggagagagggggccaatgag
ggcagggcctcctgtgggctggaaaaccactgggtgcgtctcttgctggggtttgccctggaggtgaggtgagtgctcgagggaggggagtgctt
tctgccccatgcctccaactactgtatgcaggcctggctctctggtctaggccctttgggcaagaatgtccgtctacccggcttccaccaccctctggc
cctgggcttctgtaagcagacaggcagagggcctgcccctcccaccagccaagggtgccaggcctaactggggcactcagggcagtgtgttgg
aaattccactgagggggaaatcaggtgctgcggccgcctgggccctttcctccctcaagcccatctccacaacctcgagcctgggctctggtccactact
gccccagaccaccctcaaagctggtcttcagaaatcaataatatgagtttttattttgtttttttttttttttttgtagtttattttggagtctagtattt
caataatttaagaatcagaagcactgacctttctacattttataacattattttgtatataatgtgtatttataatatgaaacagatgtgtacagga,
Homo sapiens delta like canonical Notch ligand 4 (DLL4), CDS mRNA,
NCBI Reference Sequence: NM_019074.4, 2058 bp
SEQ ID NO: 8
atggcggcagcgtcccggagcgcctctggctgggcgctactgctgctggtggcactttggcagcagcgegcggccggctccggcgtcttccagc
tgcagctgcaggagttcatcaacgagcgcggcgtactggccagtgggcggccttgcgagcccggctgccggactttcttccgcgtctgccttaag
cacttccaggcggtcgtctcgcccggaccctgcaccttcgggaccgtctccacgccggtattgggcaccaactccttcgctgtccgggacgacagt
agcggcggggggcgcaaccctctccaactgcccttcaatttcacctggccgggtaccttctcgctcatcatcgaagcttggcacgcgccaggaga
cgacctgcggccagaggccttgccaccagatgcactcatcagcaagatcgccatccagggctccctagctgtgggtcagaactggttattggatga
gcaaaccagcaccctcacaaggctgcgctactcttaccgggtcatctgcagtgacaactactatggagacaactgctcccgcctgtgcaagaagcg
caatgaccacttcggccactatgtgtgccagccagatggcaacttgtcctgcctgcccggttggactggggaatattgccaacagcctatctgtctttc
gggctgtcatgaacagaatggctactgcagcaagccagcagagtgcctctgccgcccaggctggcagggccggctgtgtaacgaatgcatcccc
cacaatggctgtcgccacggcacctgcagcactccctggcaatgtacttgtgatgagggctggggaggcctgttttgtgaccaagatctcaactact
gcacccaccactccccatgcaagaatggggcaacgtgctccaacagtgggcagegaagctacacctgcacctgtcgcccaggctacactggtgt
ggactgtgagctggagctcagcgagtgtgacagcaacccctgtcgcaatggaggcagctgtaaggaccaggaggatggctaccactgcctgtgt
cctccgggctactatggcctgcattgtgaacacagcaccttgagctgcgccgactccccctgcttcaatgggggctcctgccgggagcgcaacca
gggggccaactatgcttgtgaatgtccccccaacttcaccggctccaactgcgagaagaaagtggacaggtgcaccagcaacccctgtgccaac
gggggacagtgcctgaaccgaggtccaagccgcatgtgccgctgccgtcctggattcacgggcacctactgtgaactccacgtcagcgactgtg
cccgtaacccttgcgcccacggtggcacttgccatgacctggagaatgggctcatgtgcacctgccctgccggcttctctggccgacgctgtgagg
tgcggacatccategatgcctgtgcctcgagtccctgcttcaacagggccacctgctacaccgacctctccacagacacctttgtgtgcaactgccct
tatggctttgtgggcagccgctgcgagttccccgtgggcttgccgcccagcttcccctgggtggccgtctcgctgggtgtggggctggcagtgctg
ctggtactgctgggcatggtggcagtggctgtgcggcagctgcggcttcgacggccggacgacggcagcagggaagccatgaacaacttgtcg
gacttccagaaggacaacctgattcctgccgcccagcttaaaaacacaaaccagaagaaggagctggaagtggactgtggcctggacaagtcca
actgtggcaaacagcaaaaccacacattggactataatctggccccagggcccctgggggggggaccatgccaggaaagtttccccacagtga
caagagcttaggagagaaggcgccactgcggttacacagtgaaaagccagagtgtcggatatcagegatatgctcccccagggactccatgtacc
agtctgtgtgtttgatatcagaggagaggaatgaatgtgtcattgccacggaggtataa,

In some embodiments, the amino acid sequence of the Notch ligand (e.g., DLL4) comprises SEQ ID NO: 4 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 4, and that maintains the same functions as SEQ ID NO: 4 (e.g. binding and/or activating a Notch receptor).

delta-like protein 4 precursor [Homo sapiens]
NCBI Reference Sequence: NP_061947.1,
685 amino acids
SEQ ID NO: 9
MAAASRSASGWALLLLVALWQQRAAGSGVFQLQLQEFINERGVLASGRPC
EPGCRTFFRVCLKHFQAVVSPGPCTFGTVSTPVLGTNSFAVRDDSSGGGR
NPLQLPFNFTWPGTFSLIIEAWHAPGDDLRPEALPPDALISKIAIQGSLA
VGQNWLLDEQTSTLTRLRYSYRVICSDNYYGDNCSRLCKKRNDHFGHYVC
QPDGNLSCLPGWTGEYCQQPICLSGCHEQNGYCSKPAECLCRPGWQGRLC
NECIPHNGCRHGTCSTPWQCTCDEGWGGLFCDQDLNYCTHHSPCKNGATC
SNSGQRSYTCTCRPGYTGVDCELELSECDSNPCRNGGSCKDQEDGYHCLC
PPGYYGLHCEHSTLSCADSPCFNGGSCRERNQGANYACECPPNFTGSNCE
KKVDRCTSNPCANGGQCLNRGPSRMCRCRPGFTGTYCELHVSDCARNPCA
HGGTCHDLENGLMCTCPAGFSGRRCEVRTSIDACASSPCFNRATCYTDLS
TDTFVCNCPYGFVGSRCEFPVGLPPSFPWVAVSLGVGLAVLLVLLGMVAV
AVRQLRLRRPDDGSREAMNNLSDFQKDNLIPAAQLKNTNQKKELEVDCGL
DKSNCGKQQNHTLDYNLAPGPLGRGTMPGKFPHSDKSLGEKAPLRLHSEK
PECRISAICSPRDSMYQSVCLISEERNECVIATEV,

In some embodiments, the Notch ligand comprises the extracellular domain of human DLL4, which corresponds to amino acids 1-526 of DLL4, or amino acids 1-524 of DLL4, or amino acids 27-524 of DLL4, (see e.g., SEQ ID NO: 9 for full-length sequence of DLL4). In some embodiments, the extracellular domain of human DLL4 comprises SEQ ID NO: 10 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 5, and that maintains the same functions as SEQ ID NO: 10 (e.g., binding and/or activating a Notch receptor).

human DLL4 extracellular domain, 526 amino acids
SEQ ID NO: 10
MAAASRSASGWALLLLVALWQQRAAGSGVFQLQLQEFINERGVLASGRPC
EPGCRTFFRVCLKHFQAVVSPGPCTFGTVSTPVLGTNSFAVRDDSSGGGR
NPLQLPFNFTWPGTFSLIIEAWHAPGDDLRPEALPPDALISKIAIQGSLA
VGQNWLLDEQTSTLTRLRYSYRVICSDNYYGDNCSRLCKKRNDHFGHYVC
QPDGNLSCLPGWTGEYCQQPICLSGCHEQNGYCSKPAECLCRPGWQGRLC
NECIPHNGCRHGTCSTPWQCTCDEGWGGLFCDQDLNYCTHHSPCKNGATC
SNSGQRSYTCTCRPGYTGVDCELELSECDSNPCRNGGSCKDQEDGYHCLC
PPGYYGLHCEHSTLSCADSPCFNGGSCRERNQGANYACECPPNFTGSNCE
KKVDRCTSNPCANGGQCLNRGPSRMCRCRPGFTGTYCELHVSDCARNPCA
HGGTCHDLENGLMCTCPAGFSGRRCEVRTSIDACASSPCFNRATCYTDLS
TDTFVCNCPYGFVGSRCEFPVGLPPS,

In some embodiments, the Notch ligand (e.g., Delta1ext-IgG) comprises SEQ ID NO: 42 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 42, and that maintains the same functions as SEQ ID NO: 42 (e.g., binding and/or activating a Notch receptor).

Recombinant Human DLL1 Fc Chimera Protein, R&D
SYSTEMS 10184-DL: Human DLL1 (Ser22-Glu537)
Accession #000548 + IEGRMDP (SEQ ID NO: 58) +
Human IgG1 Fc (Pro100-Lys330)
SEQ ID NO: 42
SGVFELKLQEFVNKKGLLGNRNCCRGGAGPPPCACRTFFRVCLKHYQASV
SPEPPCTYGSAVTPVLGVDSFSLPDGGGADSAFSNPIRFPFGFTWPGTFS
LIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYC
TEPICLPGCDEQHGFCDKPGECKCRVGWQGRYCDECIRYPGCLHGTCQQP
WQCNCQEGWGGLFCNQDLNYCTHHKPCKNGATCTNTGQGSYTCSCRPGYT
GATCELGIDECDPSPCKNGGSCTDLENSYSCTCPPGFYGKICELSAMTCA
DGPCFNGGRCSDSPDGGYSCRCPVGYSGFNCEKKIDYCSSSPCSNGAKCV
DLGDAYLCRCQAGFSGRHCDDNVDDCASSPCANGGTCRDGVNDFSCTCPP
GYTGRNCSAPVSRCEHAPCHNGATCHERGHRYVCECARGYGGPNCQFLLP
ELPPGPAVVDLTEKLEIEGRMDPPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSL
SPGK,

In some embodiments, the Notch ligand (e.g., Delta4ext-IgG) comprises SEQ ID NO: 43 or an amino acid sequence that is at least 85%, at least 87%, at least 90%, at least 91%, at least 92%, at least 93%, at least 94%, at least 95%, at least 96%, at least 97%, at least 98%, or at least 99%) identical to the sequence of SEQ ID NO: 43, and that maintains the same functions as SEQ ID NO: 43 (e.g., binding and/or activating a Notch receptor).

Human DLL4 Protein Fc Tag, ACRO BIOSYSTEMS
DLA-H5259: Human DLL4 (Ser27-Pro524) + Human
IgG1 Fc (Pro100-Lys330)
SEQ ID NO: 43
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYCQQPICLSGC
HEQNGYCSKPAECLCRPGWQGRLCNECIPHNGCRHGTCSTPWQCTCDEGW
GGLFCDQDLNYCTHHSPCKNGATCSNSGQRSYTCTCRPGYTGVDCELELS
ECDSNPCRNGGSCKDQEDGYHCLCPPGYYGLHCEHSTLSCADSPCFNGGS
CRERNQGANYACECPPNFTGSNCEKKVDRCTSNPCANGGQCLNRGPSRMC
RCRPGFTGTYCELHVSDCARNPCAHGGTCHDLENGLMCTCPAGFSGRRCE
VRTSIDACASSPCFNRATCYTDLSTDTFVCNCPYGFVGSRCEFPVGLPPK
SCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK,

In some embodiments, the Notch ligand comprises an extracellular domain of a Notch ligand as described herein linked (e.g., through an optional linker sequence) to the Fc domain of human IgG1. In some embodiments, the human IgG1 Fc domain comprises SEQ ID NO: 44 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 44, and that maintains the same functions as SEQ ID NO: 44.

Pro100-Lys330 of P01857 (IGHG1_HUMAN)
SEQ ID NO: 44
PKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK,

In one embodiment, the concentration of DLL1, DLL4, immobilized Delta1^{ext-IgG}, or immobilized Delta4^{ext-IgG} is in the range of 1 μg/mL to 40 μg/mL; in other embodiments the concentration is in the range of 5 μg/mL to 30 μg/mL, 10 μg/mL to 30 μg/mL, 20 μg/mL to 30 μg/mL, 5 μg/mL to 20 μg/mL, 5 μg/mL to 10 μg/mL, 5 μg/mL to 25 μg/mL, 10 μg/mL to 20 μg/mL, or any integer therebetween. In other embodiments, the concentration of DLL1, DLL4, immobilized Delta1^{ext-IgG}, or immobilized Delta4^{ext-IgG} is about 10 μg/mL, about 15 μg/mL, about 20 μg/mL or about 25 μg/mL.

There are several ways to provide a Notch ligand, for example by providing a purified recombinant form of a Notch ligand or a Notch receptor-binding fragment, the receptor-binding fragment being sufficient to elicit cell signaling events in vivo upon contact and binding with the extracellular Notch receptors on these cells. In some embodiments, the Notch ligand is attached to a solid substrate, for example using a covalent or non-covalent bond or linkage. In some embodiments, the Notch ligand is attached to a cell culture dish.

In some embodiments, the Notch ligand further comprises a domain to immobilize the Notch ligand to a solid substrate. As a non-limiting example, the Notch ligand comprises a first member of an affinity pair, and the solid substrate comprises a second member of an affinity pair. In some embodiments, the first and second members of the affinity pair are selected from the group consisting of: a haptenic or antigenic compound in combination with a corresponding antibody or binding portion or fragment thereof (e.g., FLAG and anti-FLAG monoclonal antibody, the sequence of which are known in the art); digoxigenin and anti-digoxigenin; mouse immunoglobulin and goat anti-mouse immunoglobulin; a non-immunological binding pair; biotin and avidin; biotin and streptavidin; a hormone and a hormone-binding protein; thyroxine and cortisol-hormone binding protein; a receptor and a receptor agonist; a receptor and a receptor antagonist; acetylcholine receptor and acetylcholine or an analog thereof; IgG and protein A; lectin and carbohydrate; an enzyme and an enzyme cofactor; an enzyme and an enzyme inhibitor; complementary oligonucleotide pairs capable of forming nucleic acid duplexes; and a first molecule that is negatively charged and a second molecule that is positively charged.

In some embodiments, the population of hemogenic endothelium is differentiated into a population of CD3+ T cells by culturing in a non-tissue culture treated culture vessel; said another way, the culture vessel is not exposed to a plasma gas in order to modify the hydrophobic plastic surface to make it more hydrophilic. As used herein, the term “culture vessel” includes dishes, flasks, plates, multi-well plates, and the like. In some embodiments, the culture vessel is coated with recombinant human DL1-Fc protein (e.g., commercially available via R&D SYSTEMS, item number 10184-DL), recombinant human DL4-Fc protein (e.g., commercially available via ACRO BIOSYSTEMS, item number DL4-H5259), or a mixture of both Notch ligands, or any Notch ligand as described herein. In some embodiments, the culture vessel is coated with Notch ligand for at least 0.5 hour, at least 1.0 hour, at least 1.5 hours, at least 2.0 hours, at least 2.5 hours, at least 3.0 hours, at least 3.5 hours, at least 4.0 hours, at least 4.5 hours, or at least 5.0 hours. In some embodiments, the culture vessel is coated with Notch ligand at room temperature.

As described in the Examples, the present disclosure provides methods of cell differentiation that do not comprise co-culture of CD34+ HE cells or T cell progenitors with stromal cells. As such, in some embodiments, the Notch ligands described herein are immobilized on a solid support (e.g., a tissue culture plate). In some embodiments, the non-stromal Notch ligand (e.g., the Notch ligand immobilized on a tissue culture plate) is provided at a concentration of 1 μg/mL to 100 μg/mL or a concentration of 5 μg/mL to 15 μg/mL. In some embodiments, the non-stromal-derived Notch ligand is provided at a concentration of at least 1 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, at least 10 μg/mL, at least 11 μg/mL, at least 12 μg/mL, at least 13 μg/mL, at least 14 μg/mL, at least 15 μg/mL, at least 16 μg/mL, at least 17 μg/mL, at least 18 μg/mL, at least 19 μg/mL, at least 20 μg/mL, at least 25 μg/mL, at least 30 μg/mL, at least 35 μg/mL, at least 40 μg/mL, at least 45 μg/mL, at least 50 μg/mL, at least 55 μg/mL, at least 60 μg/mL, at least 65 μg/mL, at least 70 μg/mL, at least 75 μg/mL, at least 80 μg/mL, at least 85 μg/mL, at least 90 μg/mL, at least 95 μg/mL, or at least 100 μg/mL. In a preferred embodiment, the non-stromal Notch ligand is provided at a concentration of 10 μg/mL.

In some embodiments, the cells are cultured in the presence of a non-stromal Notch ligand (e.g., a Notch ligand immobilized on a tissue culture plate) for at least 1 day, at least 2 days, at least 3 days, at least 4 days, at least 5 days, at least 6 days, at least 7 days, at least 8 days, at least 9 days, at least 10 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, or more.

### Adhesion Proteins

In some embodiments, the cells are further cultured in the presence of at least one additional adhesion molecule. Any adhesion molecule (or combination thereof) can be used in combination with a Notch ligand in the methods described herein. Exemplary adhesion molecules include, but are not limited to, an integrin (e.g., vitronectin, fibronectin, fibrinogen, laminin, or collagen), a selectin (e.g., Platelet-selectin (P-selectin), Leukocyte-selectin (L-selectin), or Endothelial-selectin (E-selectin)), or a cadherin (e.g., epithelial (E-cadherins), placental (P-cadherins), neural (N-cadherins), retinal (R-cadherins), brain (B-cadherins and T-cadherins), and muscle (M-cadherins)), gelatin, or polylysine. In one embodiment, the adhesion molecule comprises an integrin selected from the group consisting of vitronectin, fibronectin, fibrinogen, laminin, or collagen). In another embodiment, the adhesion molecule comprises vitronectin.

In some embodiments, the adhesion proteins are used to coat a cell culture dish, or are attached to a solid substrate or solid support.

In certain embodiments, the method comprises culturing the CD34+ hemogenic endothelial cells in the presence of vitronectin or a combination of a Notch ligand and vitronectin. In certain embodiments, the method comprises culturing the CD34+ hemogenic endothelial cells in the presence of a combination of DLL4 and vitronectin. In some embodiments, the concentration of vitronectin is in the range of 1-20 μg/mL (e.g., 1-15 μg/mL, 1-10 μg/mL, 5-20 μg/mL, 5-15 μg/mL, 5-10 μg/mL, 10-15 μg/mL, or 10-20 μg/mL). In another embodiment, the concentration of vitronectin is 10 μg/mL. In another embodiment, the CD34+ HE is cultured in the presence of DLL4 and vitronectin, wherein the concentration of DLL4 is 10 μg/mL or 20 μg/mL and the concentration of vitronectin is 10 μg/mL.

### Stroma-Free Differentiation

The methods and compositions described herein including a step of culturing in the presence of IL-3, can be performed using either conventional stromal cell co-culture or using stroma-free methods. That is, while it is specifically contemplated herein that CD3+ T cells can be differentiated using conventional stromal cells co-culture in the presence of IL-3, the methods and compositions do not require the use of stromal cells. In some embodiments, the methods described herein comprise a stroma-free T cell differentiation method, i.e., a method that does not comprise co-culturing with stromal cells or any other type of supporting cell. Co-culture with stromal cells such as mouse stromal cells limits the translational potential of iPSC-derived T cells; for example, there can be fears of transplantation rejection due to the presence of stromal cells. Furthermore, T cells differentiated using stromal cells exhibit an innate-like phenotype (e.g., as measured by TCRgd expression, which is a marker for gamma delta T cells). It is preferred that T cells exhibit an adaptive phenotype, for example characterized by expression of TCR α and β. Additionally, as described herein, stroma-free T cell differentiation methods result in increased numbers of CD3+ T cells (e.g., CD4+CD8+ cells) compared to differentiation methods comprising stromal co-culture.

Accordingly, T cells differentiated using stromal-free methods, and in one embodiment, in combination with inhibition of an epigenetic regulator (e.g., an HMT; e.g., EZH1, G9a/GLP), exhibit at least the following unexpected benefits compared to stromal co-culture methods: (1) increased potential for transplantation in humans; (2) decreased number of innate-like T cells; (3) increased number and/or percentage of resultant T cells (e.g., CD5+CD7+ Pro-T cells; CD3+ T cells; CD4+CD8+ T cells; CD4+ T cells; CD8+ T cells; alpha-beta T cells); (4) gene expression profiles most similar to alpha beta T cells; (5) a more diverse TCR repertoire; and/or (6) increased TCR CDR length.

As used herein, the term “supporting cell or stromal cell” when used in the context of cell differentiation refers to any cells that are capable of creating, promoting, or supporting a microenvironment for the growth, proliferation, differentiation, or expansion of multipotent hematopoietic progenitor cells or T cells or B cells. Non-limiting examples of supporting cells that are not comprised by the differentiation methods described herein include, but are not limited to, stromal cells and fibroblast cells.

Supporting cells used previously in co-cultures for cell differentiation purposes are typically stromal cells. However, the methods described herein do not comprise co-cultures comprising stromal cells. Examples of stromal cell lines that are not comprised by the differentiation methods described herein include, but are not limited to, murine MS5 stromal cell line; murine bone marrow-derived stromal cell lines, such as S10, S17, OP9 (e.g., OP9-DL1 cells or OP9-DL4 cells) and BMS2 cell lines; human marrow stromal cell lines such as those described in U.S. Pat. No. 5,879,940, which is incorporated herein by reference in its entirety; or any other similar cells that express and display extracellular or secretes a Notch ligand. OP9-DL1 cells are a bone-marrow-derived stromal cell line that ectopically expresses the Notch ligand, Delta-like 1 (DLL1). Method of differentiating pluripotent stem cells to T-cells using OP9-Notch ligand expressing cells are known in the art. See, e.g., U.S. Pat. Nos. 7,575,925, 8,772,028, 8,871,510, and 9,206,394 and US Patent Publication Nos: 20090217403, 20110123502, 20110052554 20110027881, 20110236363, 20120149100, 20130281304, 20140322808, 20140248248, and 20140037599. These references are incorporated herein by reference in their entirety.

Described herein are methods of differentiating T cells from pluripotent stem cells, wherein the methods do not comprise a step of co-culturing the cells with supporting cells or stromal cells. In some embodiments, the Notch ligand used herein is not derived from a stromal cell. In some embodiments, differentiating the hemogenic endothelium in the presence of a Notch ligand does not comprise co-culturing with a stromal cell expressing a Notch ligand. In some embodiments, differentiating the hemogenic endothelium in the presence of a Notch ligand does not comprise co-culturing with OP9-DL1 cells or OP9-DL4 cells.

### Differentiation Medias

In some embodiments, the method comprises contacting a population of CD34+ hemogenic endothelial cells in a first differentiation medium comprising IL-3 for a sufficient time to generate CD5+CD7+ T cell progenitors. In some embodiments, the method further comprises contacting the CD5+CD7+ T cell progenitors with a second differentiation medium for a sufficient time to generate CD3+ T cell.

In some embodiments, the first differentiation medium is a CD5+CD7+ differentiation medium comprising IL-3 and one or more additional factors that promote the differentiation of the CD34+ hemogenic endothelial cells into CD5+CD7+ T cell progenitors. In some embodiments, the first differentiation medium comprises IL-3 and one or more additional factors selected from IL-7, SCF, Flt3, and TPO. In some embodiments, the first differentiation medium comprises IL-3, IL-7, SCF, Flt3, and TPO. In some embodiments, the first differentiation medium comprises 30 ng/ml SCF, 20 ng/ml Flt3, 30 ng/ml IL-7, 5 ng/mL IL-3, and 5 ng/mL TPO.

Interleukin-7 (IL-7) is a hematopoietic growth factor secreted by stromal cells in the bone marrow and thymus, and it is involved in B and T cell development. Stem cell factor (also known as SCF, KIT-ligand, KL, or steel factor) is a cytokine that binds to the c-KIT receptor (CD117) and is involved in T cell differentiation. FLT3 (also referred to as Flit3 or Fms-Like Tyrosine Kinase 3) is a class III receptor tyrosine kinase that regulates hematopoiesis. Thrombopoietin (TPO or THPO) is a cytokine that is chiefly responsible for megakaryocyte production but also has a role in maintaining hematopoietic stem cells (HSCs). See, e.g., Wang et al., Distinct roles of IL-7 and stem cell factor in the OP9-DL1 T cell differentiation culture system. Exp Hematol. 2006 December; 34(12):1730-40.

In some embodiments, the second differentiation medium is a CD3+ T cell differentiation medium comprising one or more factors that promote differentiation of CD5+CD7+ T cell progenitors into CD3+ T cells. In some embodiments, the one or more additional factors are selected from SCF, FLT3, and IL-7. In some embodiments, the second differentiation medium comprises FLT3 and IL-7. In some embodiments, the second differentiation medium comprises at least one of SCF, FLT3, and/or IL-7. In some embodiments, the second differentiation medium comprises SCF, FLT3, and IL-7. In some embodiments, the second differentiation medium comprises 30 ng/ml SCF, 15 ng/ml FLT3, and 25 ng/ml IL-7. In some embodiments, the second differentiation medium comprises 100 ng/ml SCF, 100 ng/ml FLT3, and 50 ng/ml IL-7. In some embodiments, the second differentiation medium comprises FLT3 and IL-7. In some embodiments, the second differentiation medium comprises 15 ng/ml FLT3 and 25 ng/ml IL-7. In some embodiments, the second differentiation medium comprises 100 ng/ml FLT3 and 50 ng/ml IL-7.

In some embodiments, the time sufficient to generate CD5+CD7+ T cell progenitors from CD34+ HE cells is about 1 week. In some embodiments, the time sufficient to generate CD5+CD7+ T cell progenitors from CD34+ HE cells is 5, 6, 7, 8, or 9 days. In some embodiments, the time sufficient to generate CD5+CD7+ T cell progenitors from CD34+ HE cells is about 2 weeks. In some embodiments, the time sufficient to generate CD5+CD7+ T cell progenitors from CD34+ HE cells is 10, 11, 12, 13, 14, 15, 16, or 17 days.

In some embodiments, the time sufficient to generate CD3+ T cells from CD5+CD7+ T cell progenitors is about 1 week. In some embodiments, the time sufficient to generate CD3+ T cells from CD5+CD7+ T cell progenitors is 5, 6, 7, 8, or 9 days. In some embodiments, the time sufficient to generate CD3+ T cells from CD5+CD7+ T cell progenitors is about 2 weeks, about 3 weeks, about 3.5 weeks, about 4 weeks, about 4.5 weeks, about 5 weeks, about 5.5 weeks, or about 6 weeks. In some embodiments, the time sufficient to generate CD3+ T cells from CD5+CD7+ T cell progenitors is at least 1 week, at least 2 weeks, at least 3 weeks, at least 3.5 weeks, at least 4 weeks, at least 4.5 weeks, at least 5 weeks, at least 5.5 weeks, at least 6 weeks, or more. In some embodiments, the sufficient time to promote differentiation into a population of CD3+ T cells is at most 6 weeks.

In some embodiments, the first and/or second differentiation media is serum-free.

The concentrations of SCF, FLT3, and/or IL7 should be used such that they promote the differentiation of hemogenic endothelium into a population of CD3+ T cells. The concentration of SCF can range from 1 ng/mL to 200 ng/mL. In some embodiments, the concentration of SCF (e.g., in the CD3+-T-cell-differentiation media) is 30 ng/mL. In some embodiments, the concentration of SCF (e.g., in the CD3+-T-cell-differentiation media) is 100 ng/ml. The concentration of FLT3 can range from 1 ng/mL to 200 ng/mL. In some embodiments, the concentration of FLT3 (e.g., in the CD3+-T-cell-differentiation media) is 15 ng/ml. In some embodiments, the concentration of FLT3 (e.g., in the CD3+-T-cell-differentiation media) is 100 ng/ml. The concentration of IL7 can range from 1 ng/mL to 200 ng/mL. In some embodiments, the concentration of IL7 (e.g., in the CD3+-T-cell-differentiation media) is 25 ng/ml. In some embodiments, the concentration of IL7 (e.g., in the CD3+-T-cell-differentiation media) is 50 ng/ml.

In some embodiments, the CD3+-T-cell-differentiation media further comprises thrombopoietin (TPO) for at least the first 2 weeks of differentiating in the CD3+-T-cell-differentiation media. As a non-limiting example, the CD3+-T-cell-differentiation media further comprises thrombopoietin (TPO) for at least the first 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of differentiating in the CD3+-T-cell-differentiation media. In some embodiments, CD3+-T-cell-differentiation media comprising TPO promotes differentiation into a population of CD5+CD7+ T cell progenitor cells. Such CD5+CD7+ T cell progenitor cells can be detected after at least 1 day, 2 days, 3 days, 4 days, 5 days, 6 days, 7 days, 8 days, 9 days, 10 days, 11 days, 12 days, 13 days, or 14 days of differentiating in the CD3+-T-cell-differentiation media. In some embodiments, CD5+CD7+ T cell progenitor cells can be detected after at least 2 weeks of differentiating in the CD3+-T-cell-differentiation media.

In some embodiments, the concentration of TPO should be used such that it promotes the differentiation of hemogenic endothelium into a population of CD3+ T cells. In some embodiments, the concentration of TPO can range from 1 ng/mL to 200 ng/mL. In some embodiments, the concentration of TPO (e.g., in the CD3+-T-cell-differentiation media) is 5 ng/mL. In some embodiments, the concentration of TPO (e.g., in the CD3+-T-cell-differentiation media) is 50 ng/ml.

In some embodiments, the CD3+-T-cell-differentiation media (e.g., comprising IL-7 and/or FLT3) further comprises SCF for at least the first 2 weeks of differentiating in the CD3+-T-cell-differentiation media. As a non-limiting example, the CD3+-T-cell-differentiation media further comprises SCF for at least the first 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of differentiating in the CD3+-T-cell-differentiation media. In some embodiments, CD3+-T-cell-differentiation media comprising SCF promotes differentiation into a population of CD5+CD7+ T cell progenitor cells.

In some embodiments, SCF, FLT3, IL7, and/or TPO are provided in the CD3+-T-cell-differentiation media at a concentration of at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, at least 10 ng/mL, at least 11 ng/mL, at least 12 ng/mL, at least 13 ng/mL, at least 14 ng/mL, at least 15 ng/mL, at least 16 ng/mL, at least 17 ng/mL, at least 18 ng/mL, at least 19 ng/mL, at least 20 ng/mL, at least 25 ng/mL, at least 30 ng/mL, at least 35 ng/mL, at least 40 ng/mL, at least 45 ng/mL, at least 50 ng/mL, at least 55 ng/mL, at least 60 ng/mL, at least 65 ng/mL, at least 70 ng/mL, at least 75 ng/mL, at least 80 ng/mL, at least 85 ng/mL, at least 90 ng/mL, at least 95 ng/mL, at least 100 ng/mL, at least 105 ng/mL, at least 110 ng/mL, at least 115 ng/mL, at least 120 ng/mL, at least 125 ng/mL, at least 130 ng/mL, at least 135 ng/mL, at least 140 ng/mL, at least 145 ng/mL, at least 150 ng/mL, at least 155 ng/mL, at least 160 ng/mL, at least 165 ng/mL, at least 170 ng/mL, at least 175 ng/mL, at least 180 ng/mL, at least 185 ng/mL, at least 190 ng/mL, at least 195 ng/mL, or at least 200 ng/mL. The concentration of SCF, FLT3, IL7, and/or TPO can be the same or different.

In some embodiments, CD3+ T cells can be detected after at least 5.0 weeks of differentiating in the CD3+-T-cell-differentiation media. In some embodiments, CD3+ T cells can be detected after at least 1.5 weeks, 2 weeks, 2.5 weeks, 3.0 weeks, 3.5 weeks, 4.0 weeks, 4.5 weeks, or 5.0 weeks of differentiating in the CD3+-T-cell-differentiation media. In some embodiments, the population of CD3+ T cells comprises a population of CD4+CD8+ T cells, also referred to herein as double-positive or DP T cells. Such CD4+CD8+CD3+ T cells can be detected after at least 1.5 weeks, 2 weeks, 2.5 weeks, 3.0 weeks, 3.5 weeks, 4.0 weeks, 4.5 weeks, or 5.0 weeks of differentiating in the CD3+-T-cell-differentiation media.

In some embodiments, the method further comprises differentiating the population of CD4+CD8+ T cells in a single-positive-T-cell-differentiation media for a sufficient time to promote differentiation into a population of CD4+ cells and a population of CD8+ cells. In some embodiments, the sufficient time to promote differentiation from the population of CD4+CD8+ T cells into a population of CD4+ T cells and a population of CD8+ cells is at least 1 day, at least 2 days, at least 3 days, at least 4 days, at least 5 days, at least 6 days, at least 7 days, at least 8 days, at least 9 days, or at least 10 days. In some embodiments, the sufficient time to promote differentiation from the population of CD34+ hemogenic endothelium into a population of CD4+ T cells and a population of CD8+ cells is at least 4.0 weeks, 4.5 weeks, 5.0 weeks, 5.5. weeks, or 6.0 weeks.

In some embodiments, the single-positive-T-cell-differentiation media comprises 10 ng/ml IL-15 and a T cell activator. Interleukin-15 (IL-15), like IL-7, is a member of the interleukin 2 (IL-2) superfamily, and shares many activities with IL-2, including the ability to stimulate lymphocytes. In some embodiments, a variety of concentrations of IL-15 can be used as long as it still promotes the differentiation of CD4+CD8+ T cells into single positive CD4+ cells and CD8+ cells. In some embodiments, the concentration of IL5 can range from 1 ng/mL to 200 ng/mL, with a preferred concentration of 10 ng/ml.

In some embodiments, the T cell activator comprises components (e.g., soluble tetrameric antibody complexes) that bind CD3 and CD28 (and optionally CD2) cell surface ligands. Binding of the T cell activator results in the cross-linking of CD3 and CD28 (and optionally CD2) cell surface ligands, thereby providing the required primary and co-stimulatory signals for T cell activation.

In some embodiments, the T cell activator comprises a CD3/CD28 T cell activator (e.g., at a concentration of 10 ul/ml). Such a CD3/CD28 T cell activator is available commercially (e.g., via StemCell Technology™, item #10970). In some embodiments, the concentration of the CD3/CD28 T cell activator should be used such that it promotes the differentiation of CD4+CD8+ T cells into single positive CD4+ cells and CD8+ cells. In some embodiments, the concentration can range from 1 ul/mL to 200 ul/mL, with a preferred concentration of 10 ul/ml.

In some embodiments, the T cell activator comprises CD3/CD28 T cell activator Dynabeads (e.g., used at one bead per cell). Such CD3/CD28 T cell activator Dynabeads are available commercially (e.g., via ThermoFisher™ #11132D). In some embodiments, the concentrations of CD3/CD28 T cell activator Dynabeads should be used such that it promotes the differentiation of CD4+CD8+ T cells into single positive CD4+ cells and CD8+ cells. In some embodiments, the concentration can range from 1 bead/cell to 20 beads/cell, with a preferred concentration of 1 bead/cell.

In some embodiments, the method further comprises, after at least 1 week (e.g., in the single-positive-T-cell-differentiation media), a step of CD4+ cell enrichment and/or CD8+ cell enrichment. In some embodiments, a step of CD4+ cell enrichment and/or CD8+ cell enrichment can occur at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days of culturing in the single-positive-T-cell-differentiation media.

Methods of enriching for CD4+ or CD8+ cells are known in the art. As non-limiting examples, the CD4+ or CD8+ cells can be enriched using magnetic-activated cell sorting (MACS) and fluorescence-activated cell sorting (FACS) with anti-CD4 or anti-CD8 antibodies accordingly.

In some embodiments, the entire T cell differentiation protocol described herein occurs in a stromal-free environment, e.g., the cells are cultured exposed to a non-stromal-derived Notch ligand (e.g., Notch ligand immobilized on a tissue culture plate). In some embodiments, at least a portion of the T cell differentiation protocol (e.g., comprising culturing in the CD3+-T-cell-differentiation media and in the single-positive-T-cell-differentiation media) occurs in a stromal-free environment, e.g., the cells are cultured exposed to a non-stromal-derived Notch ligand (e.g., Notch ligand immobilized on a tissue culture plate).

### Derived T Cell Population

As described herein, the population of T cells derived using stromal-free methods as described herein, and in one embodiment, in combination with inhibition of an epigenetic regulator (e.g., an HMT; e.g., EZH1, G9a/GLP), exhibits at least the following unexpected benefits compared to stromal co-culture methods: (1) increased potential for transplantation in humans; (2) decreased number of innate-like T cells; (3) increased number and/or percentage of resultant T cells (e.g., CD5+CD7+ Pro-T cells; CD3+ T cells; CD4+CD8+ T cells; CD4+ T cells; CD8+ T cells; alpha-beta T cells); (4) gene expression profiles most similar to alpha beta T cells; (5) a more diverse TCR repertoire; and/or (6) increased TCR CDR length.

In some embodiments, the population of T cells (e.g., CD3+ T cells; CD4+CD8+ T cells; CD4+ T cells; CD8+ T cells) derived using stromal-free methods and/or inhibition of an epigenetic regulator (e.g., an HMT; e.g., EZH1, G9a/GLP) as described herein exhibits at least a 10% higher transplantation or engraftment rate than a population of T cells derived using a stromal method. In some embodiments, the population of T cells derived using stromal-free methods and/or inhibition of an epigenetic regulator as described herein exhibits at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, or at least 500% or more, or at least 10×, 20×, 30×, 40×, 50×, 60×, 70×, 80×, 90×, 100×, 500×, 1,000×, or more higher transplantation or engrafiment rate than a population of T cells derived using a stromal method or without inhibition of an epigenetic regulator.

In some embodiments, a minority of the population of T cells (e.g., CD3+ T cells; CD4+CD8+ T cells; CD4+ T cells; CD8+ T cells) derived using stromal-free methods and/or inhibition of an epigenetic regulator as described herein are TCRgd⁺ (i.e., innate-like gamma delta T cells). Gamma delta T cells (γδ T cells) are T cells that have a distinctive T-cell receptor (TCR) on their surface. Most T cells are αβ (alpha beta) T cells with a TCR composed of two glycoprotein chains called α (alpha) and β (beta) TCR chains. In contrast, gamma delta (γδ) T cells have a TCR that is made up of one γ (gamma) chain and one δ (delta) chain. Like other ‘unconventional’ T cell subsets bearing invariant TCRs, such as CDId-restricted Natural Killer T cells, gamma delta T cells exhibit several characteristics that place them at the border between the more evolutionarily primitive innate immune system that permits a rapid beneficial response to a variety of foreign agents and the adaptive immune system, where B and T cells coordinate a slower but highly antigen-specific immune response leading to long-lasting memory against subsequent challenges by the same antigen. Gamma delta T cells may be considered a component of adaptive immunity in that they rearrange TCR genes to produce junctional diversity and can develop a memory phenotype. However, the various subsets may also be considered part of the innate immunity in which a specific TCR can function as a pattern recognition receptor. See, e.g., Born W K, Reardon C L, O'Brien R L (February 2006). “The function of gammadelta T cells in innate immunity”. Current Opinion in Immunology. 18 (1): 31-8.

In some embodiments, at most 10% of the population of T cells (e.g., CD3+ T cells; CD4+CD8+ T cells; CD4+ T cells; CD8+ T cells) derived using stromal-free methods and/or inhibition of an epigenetic regulator as described herein are TCRgd⁺. In some embodiments, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 21%, at most 22%, at most 23%, at most 24%, at most 25%, at most 26%, at most 27%, at most 28%, at most 29%, at most 30%, at most 31%, at most 32%, at most 33%, at most 34%, at most 35%, at most 36%, at most 37%, at most 38%, at most 39%, at most 40%, at most 41%, at most 42%, at most 43%, at most 44%, at most 45%, at most 46%, at most 47%, at most 48%, or at most 49% of the population of T cells (e.g., CD3+ T cells; CD4+CD8+ T cells; CD4+ T cells; CD8+ T cells) are TCRgd⁺.

In some embodiments, the population of T cells (e.g., CD3+ T cells; CD4+CD8+ T cells; CD4+ T cells; CD8+ T cells) derived using stromal-free methods and/or inhibition of an epigenetic regulator as described herein comprises at least 10% more T cells than a population of T cells derived using a stromal method or without inhibition of an epigenetic regulator. In some embodiments, the population of T cells derived using stromal-free methods and/or inhibition of an epigenetic regulator as described herein comprises at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, or at least 500% or more, or at least 10×, 20×, 30×, 40×, 50×, 60×, 70×, 80×, 90×, 100×, 500×, 1,000×, or more T cells than a population of T cells derived using a stromal method or without inhibition of an epigenetic regulator.

In some embodiments, the population of T cells (e.g., CD3+ T cells; CD4+CD8+ T cells; CD4+ T cells; CD8+ T cells) derived using stromal-free methods and/or inhibition of an epigenetic regulator as described herein exhibits a gene expression profile that is more similar to αβ T cells, than to other cells (e.g., γδ T cells; NK cells; iPSCs derived T cells using a OP9-DL4 co-culture system; T cells differentiated from cord blood CD34+ HSPCs), e.g., the gene profile of the derived T cells is at least 0.5% more similar to a αβ T cells as compared to another cell type. In one embodiment, the population of T cells derived using stromal-free methods and/or inhibition of an epigenetic regulator as described herein exhibits a gene expression profile of T cell signature genes and/or αβ T cell signature genes that is at most 10% divergent from the gene expression profile of αβ T cells. In one embodiment, the population of T cells derived using stromal-free methods and/or inhibition of an epigenetic regulator as described herein exhibits a gene expression profile of T cell signature genes and/or αβ T signature cell genes that is at most 20% (e.g., at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, or more) divergent from the gene expression profile of αβ T cells. In one embodiment, the population of T cells derived using stromal-free methods and/or inhibition of an epigenetic regulator as described herein exhibits a gene expression profile of T cell signature genes and/or αβ T cell signature genes that is 1%-5%, 2%-6%, 3%-7%, 4%-8%, 5%-9%, 5%-10%, 5%-15%, 10%-15%, or 15%-20% divergent from the gene expression profile of αβ T cells.

In one embodiment, the population of T cells derived using stromal-free methods and/or inhibition of an epigenetic regulator as described herein exhibits a gene expression profile that is at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more similar to the gene expression profile of αβ T cells compared to a population of T cells derived using a stromal method or without inhibition of an epigenetic regulator. In one embodiment, the derived T cell has a greater percentage of similarity to the gene expression profile of an αβ T cell than the gene profile of another cell type. One skilled in the art can determine the similarity of gene expression in a T cell derived from stromal-free methods described herein and an αβ T cell using standard methods, e.g., transcriptome sequencing of specific cell types (FACS-sorted cells).

In one embodiment, the population of T cells derived using stromal-free methods and/or inhibition of an epigenetic regulator as described herein exhibits a gene expression profile with a Pearson's correlation coefficient compared to peripheral blood alpha beta T cells that is at least 0.75, 0.755, 0.76, 0.765, 0.77, 0.775, 0.78, 0.785, 0.79, 0.795, 0.8, 0.805, 0.81, 0.815, 0.82, 0.825, 0.83, 0.835, 0.84, 0.845, 0.85, 0.855, 0.86, 0.865, 0.87, 0.875, 0.88, 0.885, 0.89, 0.895, 0.9, 0.905, 0.91, 0.915, 0.92, 0.925, 0.93, 0.935, 0.94, 0.945, 0.95, 0.955, 0.96, 0.965, 0.97, 0.975, 0.98, 0.985, 0.99, 0.995, or 1.0.

In some embodiments, the population of CD3+ T cells exhibits a gene expression profile that is most similar to alpha beta T cells. In some embodiments, the population of CD3+ T cells exhibits a gene expression profile that is similar or substantially similar to alpha beta T cells. In some embodiments, the population of CD3⁺ T cells exhibits a gene expression profile that is at least 10%, 20%, 30%, 40% or more similar to alpha beta T cells. In some embodiments, the population of CD3+T cells exhibits a gene expression profile with a Pearson's correlation coefficient compared to peripheral blood alpha beta T cells that is at least 0.85.

In some embodiments, the immune cell, e.g., derived using stromal-free and/or inhibition of an epigenetic regulator as described herein, exhibits a gene expression profile that is most similar to alpha beta T cells. In some embodiments, the immune cell exhibits a gene expression profile that is similar or substantially similar to alpha beta T cells. In some embodiments, the immune cell exhibits a gene expression profile that is at least 10%, 20%, 30%, 40% or more similar to alpha beta T cells. In some embodiments, the immune cell exhibits a gene expression profile with a Pearson's correlation coefficient compared to peripheral blood alpha beta T cells that is at least 0.85.

In some embodiments, the population of T cells derived using stromal-free methods and/or inhibition of an epigenetic regulator as described herein expresses at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 125, at least 150 or more signature genes from an αβ T cell. In one embodiment, the derived T cell expresses a greater number of signature genes from an αβ T cell than signature genes from another cell type. As used herein, the term “signature gene” refers to a gene that exhibits a characteristic expression pattern in a specific cell type (e.g., T cell, αβ T cell); a signature gene can be required for the function of a specific cell type. Non-limiting examples of T cell signature genes and αβ T cell signature genes are described further herein. A specific cell type (e.g., T cell, αβ T cell) exhibits a gene signature or gene expression signature, which comprises a single or combined group of genes in a cell with a uniquely characteristic pattern of gene expression (i.e., signature genes).

In some embodiments, the population of T cells derived using stromal-free methods and/or inhibition of an epigenetic regulator as described herein expresses at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 125, at least 150 or more genes from an αβ T cell. In one embodiment, the derived T cell expresses a greater number of genes from a αβ T cells than signature genes from another cell type.

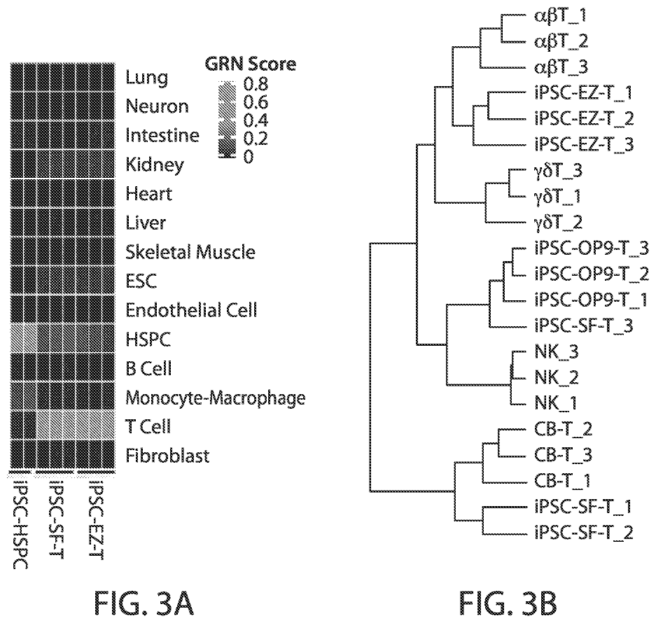

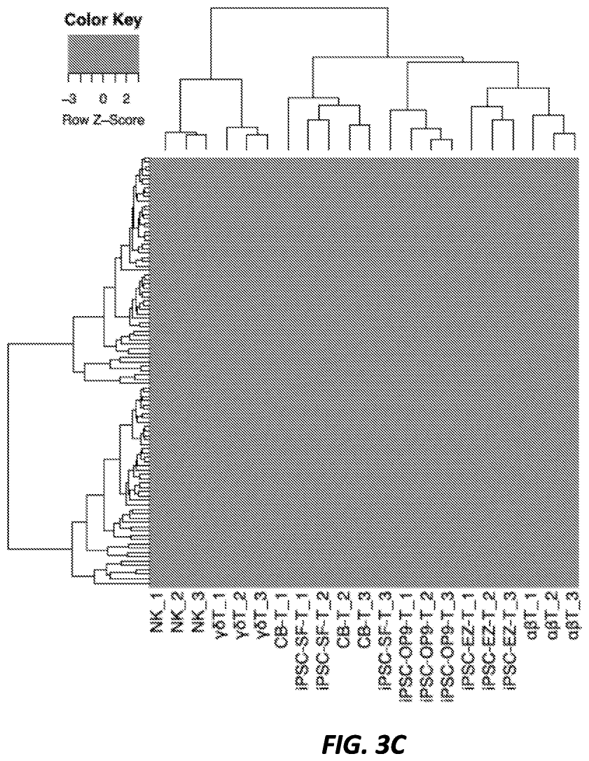

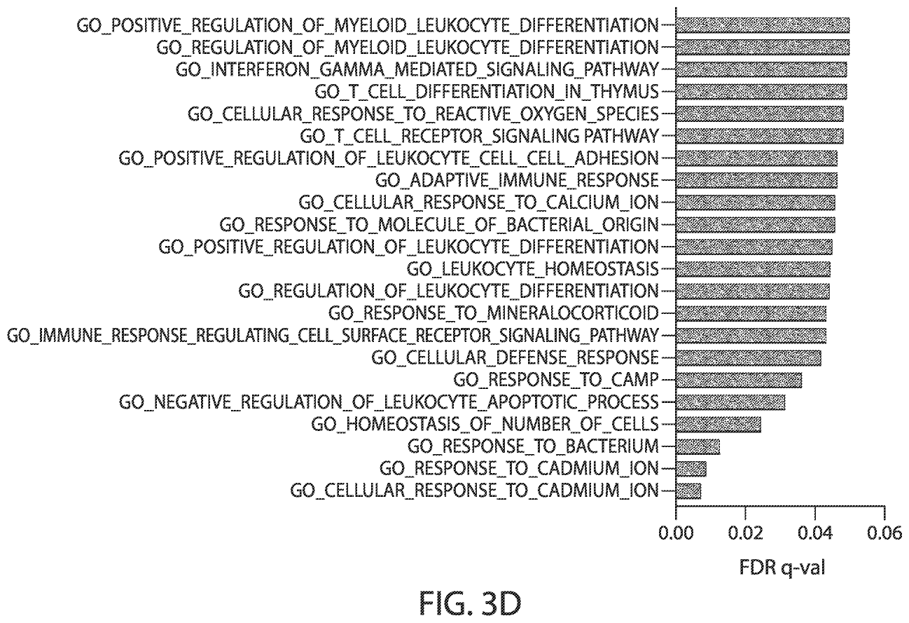

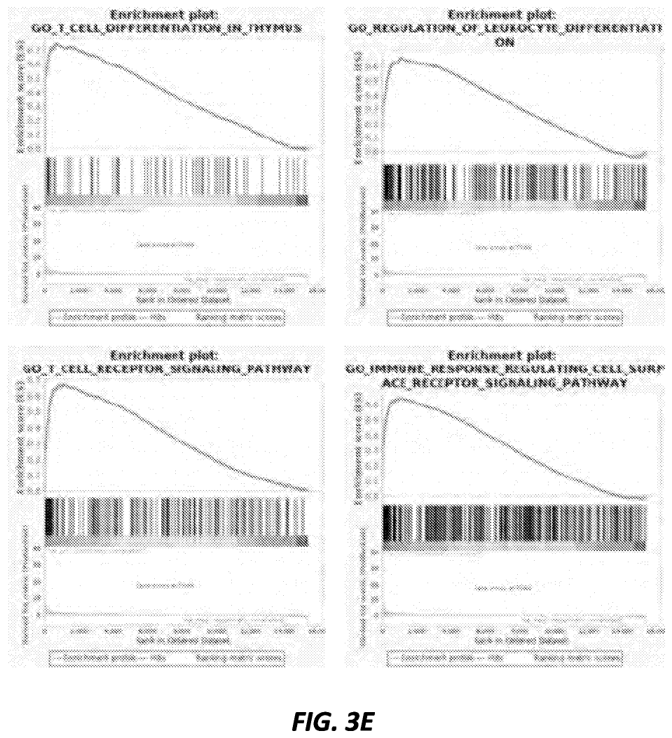

Non-limiting examples of T cell signature genes include GRB2 (Growth Factor Receptor Bound Protein 2); NFATC3 (Nuclear Factor Of Activated T Cells 3); ZAP70 (Zeta Chain Of T Cell Receptor Associated Protein Kinase 70); RAF1 (Raf-1 Proto-Oncogene, Serine/Threonine Kinase); PIK3CG (Phosphatidylinositol-4,5-Bisphosphate 3-Kinase Catalytic Subunit Gamma); PIK3R1 (Phosphoinositide-3-Kinase Regulatory Subunit 1); CALM3 (Calmodulin 3); PTPN7 (Protein Tyrosine Phosphatase Non-Receptor Type 7); LAT (Linker For Activation Of T Cells); NFKBIA (NFKB Inhibitor Alpha); VAV1 (Vav Guanine Nucleotide Exchange Factor 1); SHC1 (SHC (Src Homology 2 Domain Containing) Adaptor Protein 1); PRKCB (Protein Kinase C Beta); MAP2K4 (Mitogen-Activated Protein Kinase Kinase 4); MAP2K1 (Mitogen-Activated Protein Kinase Kinase 1); RAC1 (Rac Family Small GTPase 1); FYN (Fyn Proto-Oncogene, Src Family Tyrosine Kinase); RELA (RELA Proto-Oncogene, NF-KB Subunit, v-rel avian reticuloendotheliosis viral oncogene homolog A); LCK (Lck Proto-Oncogene, Src Family Tyrosine Kinase); CALM2 (Calmodulin 2); CD3D (CD3 Antigen, Delta Subunit); CALM1 (Calmodulin 1); CD247 (T-Cell Surface Glycoprotein CD3 Zeta Chain); CD3E (T-Cell Surface Glycoprotein CD3 Epsilon Chain); CD3G (T-Cell Surface Glycoprotein CD3 Gamma Chain); FOS (Fos Proto-Oncogene, AP-1 Transcription Factor Subunit); PIK3CA (Phosphatidylinositol-4,5-Bisphosphate 3-Kinase Catalytic Subunit Alpha); PLCG1 (Phospholipase C Gamma 1); SOS1 (Son Of Sevenless Homolog 1, SOS Ras/Rac Guanine Nucleotide Exchange Factor 1); ELK1 (ETS Transcription Factor ELK1); PPP3CC (Protein Phosphatase 3 Catalytic Subunit Gamma); MAP3K1 (Mitogen-Activated Protein Kinase Kinase Kinase 1); PPP3CA (Protein Phosphatase 3 Catalytic Subunit Alpha); NFKB1 (Nuclear Factor Kappa B Subunit 1); NFATC2 (Nuclear Factor Of Activated T Cells 2); NFATC1 (Nuclear Factor Of Activated T Cells 1, AP-1 Transcription Factor Subunit); JUN (Jun Proto-Oncogene; MAPK8 (Mitogen-Activated Protein Kinase 8); RASA1 (RAS P21 Protein Activator 1); PPP3CB (Protein Phosphatase 3 Catalytic Subunit Beta); PRKCA (Protein Kinase C Alpha); MAPK3 (Mitogen-Activated Protein Kinase 3); and NFATC4 (Nuclear Factor Of Activated T Cells 4) (see e.g., FIG. 3A).

Non-limiting examples of αβ T cell signature genes include ATP11B (ATPase Phospholipid Transporting 11B); PPP4R3A (Protein Phosphatase 4 Regulatory Subunit 3A); CAB39 (Calcium Binding Protein 39); GLS (Glutaminase); UBE2Z (Ubiquitin Conjugating Enzyme E2 Z); INPP4A (Inositol Polyphosphate-4-Phosphatase Type I A); RAB22A (Ras-Related Protein Rab-22A, Member Ras Oncogene Family); SMARCD2 (SWI/SNF (SWItch/Sucrose Non-Fermentable) Related, Matrix Associated, Actin Dependent Regulator Of Chromatin, Subfamily D, Member 2); VPS26B (VPS26, Retromer Complex Component B, Vacuolar Protein Sorting-Associated Protein 26B); CERK (Ceramide Kinase); ESYT2 (Extended Synaptotagmin 2); RAC1 (Rac Family Small GTPase 1); EIF3B (Eukaryotic Translation Initiation Factor 3 Subunit B); NEK7 (NIMA (Never In Mitosis Gene A)-Related Kinase 7); MDFIC (MyoD (myoblast determination protein 1) Family Inhibitor Domain Containing); YWHAH (Tyrosine 3-Monooxygenase/Tryptophan 5-Monooxygenase Activation Protein Eta); MCMBP (Minichromosome Maintenance Complex Binding Protein); GOLPH3 (Golgi Phosphoprotein 3); PTGER4 (Prostaglandin E Receptor 4); B3GNT2 (UDP-GlcNAc:BetaGal Beta-1,3-N-Acetylglucosaminyltransferase 2, Galactosyltransferase 7); PITPNC1 (Phosphatidylinositol Transfer Protein Cytoplasmic 1); ARAP2 (ArfGAP With RhoGAP Domain, Ankyrin Repeat And PH Domain 2; Arf And Rho GAP Adapter Protein 2); ZFP36L2 (Zinc Finger Protein 36, C3H1 Type-Like 2); EFHD2 (EF-Hand Domain Family Member D2, Swiprosin-1); CPD (Carboxypeptidase D); KLRB1 (Killer Cell Lectin Like Receptor B1); DUSP1 (Dual Specificity Phosphatase 1); CMPK1 (Cytidine/Uridine Monophosphate Kinase 1); RASGRP1 (Ras Guanyl Releasing Protein 1); TM9SF3 (Transmembrane 9 Superfamily Member 3); MAPK1 (Mitogen-Activated Protein Kinase 1); GSPT1 (G1 To S Phase Transition 1); PNRC1 (Proline Rich Nuclear Receptor Coactivator 1); TMEM248 (Transmembrane Protein 248); STT3B (STT3 (STaurosporine and Temperature sensitive) Oligosaccharyltransferase Complex Catalytic Subunit B); KHDRBS1 (KH (K Homology) RNA Binding Domain Containing, Signal Transduction Associated 1); GNPTAB (N-Acetylglucosamine-1-Phosphate Transferase Subunits Alpha And Beta); GRSF1 (G-Rich RNA Sequence Binding Factor 1); TARP (TCR Gamma Alternate Reading Frame Protein, T-Cell Receptor Gamma-Chain); ZBTB16 (Zinc Finger And BTB (for BR-C, ttk and bab) Domain Containing 16, Zinc Finger Protein 145 (Kruppel-Like, Expressed In Promyelocytic Leukemia)); TGFBR1 (Transforming Growth Factor Beta Receptor 1); LGALS3BP (Galectin 3 Binding Protein); CD5 (T-Cell Surface Glycoprotein CD5); CD4 (T-Cell Surface Glycoprotein CD4); LRRN3 (Leucine Rich Repeat Neuronal 3); SLC40A1 (Solute Carrier Family 40 Member 1); CYSLTR1 (Cysteinyl Leukotriene Receptor 1); H4C3 (H4 Clustered Histone 3); CISH (Cytokine Inducible SH2 (Src Homology 2) Containing Protein); CD8B (T-Cell Surface Glycoprotein CD8 Beta Chain); MAL (Mal, T Cell Differentiation Protein, Myelin And Lymphocyte Protein); SUN2 (Sad1 And Unc84 Domain Containing 2, Rab5-Interacting Protein); CCR7 (C-C Motif Chemokine Receptor 7); GNLY (Granulysin); ANKLE2 (Ankyrin Repeat And LEM (LAP2, emerin, MAN1) Domain Containing 2); PSIP1 (PC4 (Positive Cofactor 4) And SFRS1 (Serine And Arginine Rich Splicing Factor 1) Interacting Protein 1, Lens Epithelium-Derived Growth Factor); PITPNA (Phosphatidylinositol Transfer Protein Alpha); RBM15B (RNA Binding Motif Protein 15B); PTPRA (Protein Tyrosine Phosphatase Receptor Type A); MARK2 (Microtubule Affinity Regulating Kinase 2); BLOC1S4 (Biogenesis Of Lysosomal Organelles Complex 1 Subunit 4); SIAH2 (Siah E3 Ubiquitin Protein Ligase 2); MXD4 (Max Dimerization Protein 4); SRM (Spermidine Synthase); SESN1 (Sestrin 1); SSBP4 (Single Stranded DNA Binding Protein 4); TAF10 (TATA-Box Binding Protein Associated Factor 10); DUSP2 (Dual Specificity Phosphatase 2); LPCAT1 (Lysophosphatidylcholine Acyltransferase 1); RASAL3 (Ras Protein Activator Like 3); TRIM65 (Tripartite Motif Containing 65); FAM50A (Family With Sequence Similarity 50 Member A); PIM3 (Pim-3 Proto-Oncogene, Serine/Threonine Kinase); SIPA1 (Signal-Induced Proliferation-Associated 1); FAM89B (Family With Sequence Similarity 89 Member B); ZBTB7A (Zinc Finger And BTB (for BR-C, ttk and bab) Domain Containing 7A, Factor That Binds To Inducer Of Short Transcripts Protein 1); NIN (Ninein); NR1D2 (Nuclear Receptor Subfamily 1 Group D Member 2); SIK3 (Salt-Inducible Kinase 3); ARHGAP26 (Rho GTPase Activating Protein 26); IL18RAP (Interleukin 18 Receptor Accessory Protein); CNR2 (Cannabinoid Receptor 2); EOMES (Eomesodermin); KLRC1 (Killer Cell Lectin Like Receptor C1); SEL1L3 (Suppressor Of Lin-12-Like Protein 3); IL12RB2 (Interleukin 12 Receptor Subunit Beta 2); COTL1 (Coactosin Like F-Actin Binding Protein 1); PIK3AP1 (Phosphoinositide-3-Kinase Adaptor Protein 1); TBX21 (T-Box Transcription Factor 21); FAM43A (Family With Sequence Similarity 43 Member A); KLRD1 (Killer Cell Lectin Like Receptor D1); SLAMF7 (signaling lymphocytic activation molecule (SLAM) family member 7); S1PR5 (Sphingosine-1-Phosphate Receptor 5); LAG3 (Lymphocyte Activating 3); ABCG1 (ATP Binding Cassette Subfamily G Member 1); S100B (S100 Calcium-Binding Protein, Beta); CCL22 (C-C Motif Chemokine Ligand 22); CEBPD (CCAAT box Enhancer Binding Protein Delta); IL17F (Interleukin 17F); and CEACAM1 (CEA Cell Adhesion Molecule 1); (see e.g., FIG. 3B).

In some embodiments, the population of T cells derived using stromal-free methods and/or inhibition of an epigenetic regulator as described herein exhibits a more diverse TCR repertoire compared to T cells not derived using such stromal-free methods or without inhibition of an epigenetic regulator. In some embodiments, the population of T cells derived using stromal-free methods and/or inhibition of an epigenetic regulator as described herein exhibits a Productive Simpson Clonality value of about 0.000-0.025. A value closer to 0 represents a higher level of diversity compared to clonality. A value closer to 1 represents a higher level of clonality compared to diversity. In some embodiments, the population of T cells derived using stromal-free methods and/or inhibition of an epigenetic regulator as described herein exhibits a Productive Simpson Clonality value of at most 0.01, at most 0.015, at most 0.02, at most 0.025, at most 0.03, at most 0.035, at most 0.04, at most 0.045, at most 0.05, at most 0.055, at most 0.06, at most 0.065, at most 0.07, at most 0.075, at most 0.08, at most 0.085, at most 0.09, at most 0.095, or at most 0.1. In some embodiments, the population of T cells derived using stromal-free methods and/or inhibition of an epigenetic regulator as described herein exhibits a Productive Simpson Clonality value of about 0.025.

The variable domain of both the T-cell receptor (TCR) α-chain and β-chain each have three hypervariable or complementarity-determining regions (CDRs; e.g., CDR1, CDR2, CDR3). In some embodiments, the population of T cells derived using stromal-free methods and/or inhibition of an epigenetic regulator as described herein exhibits an increased CDR (e.g., CDR1, CDR2, CDR3) length compared to T cells derived using stromal methods or without inhibition of an epigenetic regulator. In some embodiments, the population of T cells derived using stromal-free methods and/or inhibition of an epigenetic regulator as described herein exhibits CDR (e.g., CDR1, CDR2, CDR3) length that is, on average, about 3 nucleotides (nt), 6 nt, 9 nt, or 12 nt or more longer than the CDRs of T cells derived using stromal methods or without inhibition of an epigenetic regulator. In some embodiments, the population of T cells derived using stromal-free methods and/or inhibition of an epigenetic regulator as described herein exhibits CDR (e.g., CDR1, CDR2, CDR3) length that is, on average, about 27 nt, 30 nt, 33 nt, 36 nt, 39 nt, 42 nt, 45 nt, 48 nt, 51 nt, 54 nt, 57 nt, or 60 nt or longer. In some embodiments, the population of T cells derived using stromal-free methods and/or inhibition of an epigenetic regulator as described herein exhibits a CDR3 length that is, on average, about 42 nt long, compared to 39 nt on average for control iPSC-derived T cells, or 45 on average for peripheral blood mononuclear cell (PBMC)-derived T cells.

### Genetic Modifications of T Cells

In some embodiments, the resultant population of CD34+ hemogenic endothelium or another population as described herein (e.g., ESCs; iPSCs; HSCs; CD5+CD7+ T cell progenitor cells; CD3+ T cells; CD4+CD8+ T cells; CD4+ T cells; CD8+ T cells) are genetically modified. In some embodiments, the native T cell receptor locus can be removed and/or replaced to enhance targeted specificity. In some embodiments, an endogenous HLA (e.g., class I and/or class II major histocompatibility complexes) can be edited or removed. In some embodiments, the genetic modification can comprise introduction and expression of non-canonical HLA-G and HLA-E to prevent NK cell-mediated lysis (see e.g., Riolobos L et al. 2013), which can provide a source of universal T cells for immunotherapy, e.g., cancer immune therapy.

In some embodiments, the genetic modification comprises expressing a chimeric antigen receptor (CAR). Chimeric antigen receptors (CARs, also known as chimeric immunoreceptors, chimeric T cell receptors or artificial T cell receptors) are receptor proteins that have been engineered to give T cells the new ability to target a specific protein. The receptors are chimeric because they combine both antigen-binding and T-cell activating functions into a single receptor. Methods of engineering chimeric antigen receptor T cells (also known as CAR T cells) are known in the art. See e.g., U.S. Pat. Nos. 7,446,190, 8,399,645, 8,822,647, 9,212,229, 9,273,283, 9,447,194, 9,587,020, 9,932,405, U.S. Ser. No. 10/125,193, U.S. Ser. No. 10/221,245, U.S. Ser. No. 10/273,300, U.S. Ser. No. 10/287,354; US patent publication US20160152723; PCT publication WO2009091826, WO2012079000, WO2014165707, WO2015164740, WO2016168595A1, WO2017040945, WO2017100428, WO2017117112, WO2017149515, WO2018067992, WO2018102787, WO2018102786, WO2018165228, WO2019084288; the contents of each of which are incorporated herein by reference in their entireties.

In some embodiments, methods of genetically modifying a cell to express a CAR can comprise but are not limited to: transfection or electroporation of a cell with a vector encoding a CAR; transduction with a viral vector (e.g., retrovirus, lentivirus) encoding a CAR; gene editing using zin finger nucleases (ZFNs), transcription activator-like effector nucleases (TALENs), meganuclease-TALENs, or CRISPR-Cas; or any other methods known in the art of genetically modifying a cell to express a CAR.

Preferably, a population of cells at an early stage of differentiation (e.g., ESCs; PSCs; iPSCs; hemogenic endothelium; HSCs) is genetically modified with the CAR.

In some embodiments, the antigen-binding region of the CAR is directed against an antigen involved in a disease or disorder, such as but not limited to cancer, autoimmune disease, or heart disease (e.g., cardiac fibrosis). As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments, the cancer is a primary cancer. In some embodiments, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, in the case of CAR T therapy, the cancer is a blood cancer such as a leukemia or lymphoma.

Immunotherapy with chimeric antigen receptor (CAR) T cells offers a promising method to improve cure rates and decrease morbidities for patients with cancer. In this regard, CD19-specific CAR T cell therapies have achieved dramatic objective responses for a high percent of patients with CD19-positive leukemia or lymphoma. Accordingly, in some embodiments, the antigen-binding region of the CAR is directed against CD19; see e.g., US patents U.S. Ser. No. 10/221,245, U.S. Ser. No. 10/357,514; US patent publication US20160152723; PCT publication WO2016033570; the contents of each of which are incorporated herein by reference in their entireties.

Tumor antigens are proteins that are produced by tumor cells that elicit an immune response, particularly T-cell mediated immune responses. The selection of the antigen binding domain of the invention will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, B7H3, Kit, CA-IX, CS-1, MUC1, BCMA, bcr-abl, HER2, β-human chorionic gonadotropin, alphafetoprotein (AFP), ALK, CD19, CD123, cyclin B 1, lectin-reactive AFP, Fos-related antigen 1, ADRB3, thyroglobulin, EphA2, RAGE-1, RU1, RU2, SSX2, AKAP-4, LCK, OY-TES1, PAX5, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYP1B1, BORIS, prostase, prostate-specific antigen (PSA), PAX3, PAP, NY-ESO-1, LAGE-1a, LMP2, NCAM, p53, p53 mutant, Ras mutant, gp100, prostein, OR51E2, PANX3, PSMA, PSCA, Her2/neu, hTERT, HMWMAA, HAVCR1, VEGFR2, PDGFR-beta, legumain, HPV E6,E7, survivin and telomerase, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART1, XAGE1, ELF2M, ERG (TMPRSS2 ETS fusion gene), NA17, neutrophil elastase, sarcoma translocation breakpoints, NY-BR-1, ephrinB2, CD20, CD22, CD24, CD30, CD33, CD38, CD44v6, CD97, CD171, CD179a, androgen receptor, insulin growth factor (IGF)-I, IGF-II, IGF-I receptor, GD2, o-acetyl-GD2, GD3, GM3, GPRC5D, GPR20, CXORF61, folate receptor (FRa), folate receptor beta, RORI, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, CD33, BCMA, GD2, CLL-1, CA-IX, MUC1, HER2, and any combination thereof; see e.g., US Patent publications 20170209492 and 20180022795, the contents of each of which are incorporated herein by reference in their entireties.

### Cellular Replacement Therapy

In one embodiment, provided herein a population of engineered immune cells produced by a method described herein, where in the T cell population is produced using a stroma-free differentiation method as described herein. In some embodiments, the population of engineered immune cells comprises an immune cell differentiated using methods described herein, including but not limited to: PSCs; iPSCs; hemogenic endothelium; HSCs; CD5+CD7+ T cell progenitor cells; CD3+ T cells; CD4+CD8+ T cells; CD4+ T cells; CD8+ T cells. In some embodiments, the immune cell exhibits a gene expression profile that is most similar to alpha beta T cells.

In one embodiment, the population of cells further comprises a pharmaceutically acceptable carrier. These engineered immune cells can be culture expanded to increase the number of cells for use.

The engineered immune cells described herein are useful in the laboratory for biological studies. For examples, these cells can be derived from an individual having a genetic disease or defect, and used in the laboratory to study the biological aspects of the disease or defect, and to screen and test for potential remedy for that disease or defect.

Alternatively, the engineered immune cells described herein are useful in cellular replacement therapy and other medical treatment in subjects having the need. For example, patients who have undergone chemotherapy or irradiation or both, and manifest deficiencies in immune function and/or lymphocyte reconstitution, or in cancer immune therapy.

In various embodiments, the engineered immune cells described herein are administered (i.e., implanted or transplanted) to a subject in need of cellular replacement therapy.

In one embodiment, provided herein is a method of cellular replacement therapy, or for the treatment of cancer, autoimmune disorders, hematological diseases, or other genetic diseases and disorders in a subject, comprising (a) providing a somatic cell from a donor subject, (b) generating multilineage hematopoietic progenitor cells (e.g., hemogenic endothelium, HSPCs) from pluripotent stem cells derived from the somatic cell as described in any of the preceding paragraphs; (c) optionally inhibiting a histone methyltransferase in the resultant population of multilineage hematopoietic progenitor cells as described in any of the preceding paragraphs; (d) differentiating the resultant population of multilineage hematopoietic progenitor cells in the presence of a notch ligand to promote differentiation into the lymphoid lineage (e.g., T cells) as described in any of the preceding paragraphs, and (e) implanting or administering the resultant differentiated lymphoid cells into a recipient subject.

In one embodiment, the host subject and the recipient subject are the same individual. Alternatively, the host subject and the recipient subject are not the same individual, but are at least HLA compatible.

Hematological diseases are disorders which primarily affect the blood. Non-limiting such diseases or disorders include myeloid derived disorders such as hemoglobinopathies (congenital abnormality of the hemoglobin molecule or of the rate of hemoglobin synthesis), examples, sickle-cell disease, thalassemia, and methemoglobinemia; Anemias (lack of red blood cells or hemoglobin), Pernicious anemia; disorders resulting in decreased numbers of cells, such as myelodysplastic syndrome, neutropenia (decrease in the number of neutrophils), and thrombotic thrombocytopenic purpura (TTP), thrombocytosis, hematological malignancies such as lymphomas, myelomas, and leukemia. Lymphomas such as Hodgkin's disease, Non-Hodgkin's lymphoma, Burkitt's lymphoma, Anaplastic large cell lymphoma, Splenic marginal zone lymphoma, Hepatosplenic T-cell lymphoma, and Angioimmunoblastic T-cell lymphoma (AILT); myelomas such as Multiple myeloma, Waldenström macroglobulinemia, Plasmacytoma; leukemias that increases defect WBC such as Acute lymphocytic leukemia (ALL), Chronic lymphocytic leukemia (CLL), Acute myelogenous leukemia (AML), Chronic Idiopathic Myelofibrosis (MF), Chronic myelogenous leukemia (CML), T-cell prolymphocytic leukemia (T-PLL), B-cell prolymphocytic leukemia (B-PLL), Chronic neutrophilic leukemia (CNL), Hairy cell leukemia (HCL), T-cell large granular lymphocyte leukemia (T-LGL), and Aggressive NK-cell leukemia.

Provided herein is a method of treating an autoimmune disease, which comprises administering an effective amount of an immune cell or population thereof, or a composition, or a pharmaceutical composition as described herein to a patient in need thereof. “Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Non-limiting examples of autoimmune diseases that can be treated include pemphigus (pemphigus vulgaris, pemphigus foliaceus or paraneoplastic pemphigus), Crohn's disease, idiopathic thrombocytopenic purpura (ITP), heparin induced thrombocytopenia (HIT), thrombotic thrombocytopenic purpura (TTP), Myasthenia Gravis (MG), and Chronic Inflammatory Demyelinating Polyneuropathy (CIDP). Additional non-limiting autoimmune diseases include autoimmune thrombocytopenia, immune neutropenia, antihemophilic FVIII inhibitor, antiphospholipid syndrome, Kawasaki Syndrome, ANCA-associated disease, polymyositis, bullous pemphigoid, multiple sclerosis (MS), Guillain-Barre Syndrome, chronic polyneuropathy, ulcerative colitis, diabetes mellitus, autoimmune thyroiditis, Graves' opthalmopathy, rheumatoid arthritis, ulcerative colitis, primary sclerosing cholangitis, systemic lupus erythematosus (SLE), autoimmune encephalomyelitis, Hashimoto's thyroiditis, Goodpasture's syndrome, autoimmune hemolytic anemia, scleroderma with anticollagen antibodies, mixed connective tissue disease, pernicious anemia, idiopathic Addison's disease, autoimmune-associated infertility, glomerulonephritis (e.g., crescentic glomerulonephritis, proliferative glomerulonephritis), insulin resistance, and autoimmune diabetes mellitus (type 1 diabetes mellitus; insulin dependent diabetes mellitus). Autoimmune disease has been recognized also to encompass atherosclerosis and Alzheimer's disease. In another embodiment, the autoimmune diseases include hepatitis, autoimmune hemophilia, autoimmune lymphoproliferative syndrome (ALPS), autoimmune uveoretinitis, glomerulonephritis, agammaglobulinemia, alopecia areata, amyloidosis, ankylosing spondylitis, autoimmune angioedema, autoimmune aplastic anemia, autoimmune dysautonomia, autoimmune hyperlipidemia, autoimmune immunodeficiency, autoimmune inner ear disease (AIED), autoimmune myocarditis, autoimmune pancreatitis, autoimmune retinopathy, autoimmune urticaria, autoimmune urticarial neuropathy, autoimmune axonal neuropathy, Balo disease, Behget's disease, Castleman disease, celiac disease, Chagas disease, chronic recurrent multifocal osteomyelitis (CRMO), Churg-Strauss syndrome, cicatricial pemphigoid, benign mucosal pemphigoid, Cogan's syndrome, cold agglutinin disease, coxsackie myocarditis, CREST disease, essential mixed cryoglobulinemia, dermatitis herpetiformis, dermatomyositis, Devic's disease (neuromyelitis optica), dilated cardiomyopathy, discoid lupus, Dressler's syndrome, endometriosis, eosinophilic angiocentric fibrosis, Eosinophilic fasciitis, Erythema nodosum, Evans syndrome, Fibrosing alveolitis, Giant cell arteritis (temporal arteritis), Hashimoto's encephalitis, Henoch-Schonlein purpura, Herpes gestationis, Idiopathic hypocomplementemic tubulointestitial nephritis, multiple myeloma, multifocal motor neuropathy, NMDA receptor antibody encephalitis, IgG4-related disease, IgG4-related sclerosing disease, inflammatory aortic aneurysm, inflammatory pseudotumour, inclusion body myositis, interstitial cystitis, juvenile arthritis, Kuttner's tumour, Lambert-Eaton syndrome, leukocytoclastic vasculitis, lichen planus, lichen sclerosus, Ligneous conjunctivitis, Linear IgA disease (LAD), Lyme disease, chronic, mediastinal fibrosis, Meniere's disease, Microscopic polyangiitis, Mikulicz's syndrome, Mooren's ulcer, Mucha-Habermann disease, multifocal fibrosclerosis, narcolepsy, optic neuritis, Ormond's disease (retroperitoneal fibrosis), palindromic rheumatism, PANDAS (pediatric autoimmune neuropsychiatric disorders associated with Streptococcus), paraneoplastic cerebellar degeneration, paraproteinemic polyneuropathies, paroxysmal nocturnal hemoglobinuria (PNH), Parry Romberg syndrome, Parsonnage-Turner syndrome, periaortitis, periarteritis, peripheral neuropathy, perivenous encephalomyelitis, POEMS syndrome, polyarteritis nodosa, Type I, II, & III autoimmune polyglandular syndromes, polymyalgia rheumatic, postpericardiotomy syndrome, progesterone dermatitis, primary biliary cirrhosis, psoriasis, psoriatic arthritis, idiopathic pulmonary fibrosis, pyoderma gangrenosum, pure red cell aplasia, Raynaud's phenomenon, reflex sympathetic dystrophy, Reiter's syndrome, relapsing polychondritis, restless legs syndrome, rheumatic fever, Riede's thyroiditis, sarcoidosis, Schmidt syndrome, scleritis, Sjogren's syndrome, sperm and testicular autoimmunity, stiff person syndrome, subacute bacterial endocarditis (SBE), Susac's syndrome, sympathetic ophthalmia, Takayasu's arteritis, Tolosa-Hunt syndrome, transverse myelitis, undifferentiated connective tissue disease (UCTD), vesiculobullous dermatosis, vitiligo, Rasmussen's encephalitis, Waldenstrom's macroglobulinaemia.

As used herein, the terms “administering,” “introducing” and “transplanting” are used interchangeably in the context of the placement of described cells, e.g. hematopoietic progenitor cells, into a subject, by a method or route which results in at least partial localization of the introduced cells at a desired site, such as a site of injury or repair, such that a desired effect(s) is produced. The cells e.g. hematopoietic progenitor cells, or their differentiated progeny (e.g., T cells) can be administered by any appropriate route which results in delivery to a desired location in the subject where at least a portion of the implanted cells or components of the cells remain viable.

In various embodiments, the engineered immune cells described herein are optionally expanded ex vivo prior to administration to a subject. In other embodiments, the engineered immune cells are optionally cryopreserved for a period, then thawed prior to administration to a subject.

The engineered immune cells used for cellular replacement therapy can be autologous/autogenic (“self”) or non-autologous (“non-self,” e.g., allogeneic, syngeneic or xenogeneic) in relation to the recipient of the cells. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells of the invention are allogeneic.

In various embodiments, the engineered immune cell described herein that is to be implanted into a subject in need thereof is autologous or allogeneic to the subject.

In various embodiments, the engineered immune cell described herein can be derived from one or more donors, or can be obtained from an autologous source. In some embodiments, the engineered immune cells are expanded in culture prior to administration to a subject in need thereof.

In various embodiments, the engineered immune cell described herein can be derived from one or more donors, or can be obtained from an autologous source.

In various embodiments, prior to implantation, the recipient subject is treated with chemotherapy and/or radiation.

In one embodiment, the chemotherapy and/or radiation is to reduce endogenous stem cells to facilitate engrafiment of the implanted cells.

In various embodiments, prior to implantation, the engineered immune cells or the histone methyltransferase inhibited, multilineage hematopoietic progenitor cells or T cells differentiated using a stroma-free method as described herein are treated ex vivo with prostaglandin E2 and/or antioxidant N-acetyl-L-cysteine (NAC) to promote subsequent engrafiment in a recipient subject.

In various embodiments, the recipient subject is a human.

In various embodiments, the subject has been previously diagnosed with HIV or other viral disease, a hematological disease, or undergoing a cancer treatment.

In one embodiment, a subject is selected to donate a somatic cell which would be used to produce iPSCs and an engineered immune cell described herein. In one embodiment, the selected subject has a genetic disease or defect.

In various embodiments, the donor subject is a human, non-human animal, rodent or non-rodent. For example, the subject can be any mammal, e.g., a human, other primate, pig, rodent such as mouse or rat, rabbit, guinea pig, hamster, cow, horse, cat, dog, sheep or goat, or a non-mammal such as a bird.

In various embodiments, the donor has been previously diagnosed with HIV, a hematological disease or cancer.

In one embodiment, a biological sample, a population of embryonic stem cells, somatic stem cells, progenitor cells, bone marrow cells, hematopoietic stem cells, or hematopoietic progenitor cells is obtained from the donor subject.

In various embodiments, the biological sample, a population of embryonic stem cells, somatic stem cells, progenitor cells, bone marrow cells, hematopoietic stem cells, or hematopoietic progenitor cells described herein can be derived from one or more donors, or can be obtained from an autologous source.

In one embodiment, the embryonic stem cells, somatic stem cells, progenitor cells, bone marrow cells, hematopoietic stem cells, hematopoietic progenitor cells are isolated from the donor subject, transfected, cultured (optional), and transplanted back into the same subject, i.e. an autologous cell transplant. Here, the donor and the recipient subject is the same individual. In another embodiment, the embryonic stem cells, somatic stem cells, progenitor cells, bone marrow cells, hematopoietic stem cells, or hematopoietic progenitor cells are isolated from a donor who is an HLA-type match with a subject (recipient). Donor-recipient antigen type-matching is well known in the art. The HLA-types include HLA-A, HLA-B, HLA-C, and HLA-D. These represent the minimum number of cell surface antigen matching required for transplantation. That is the transfected cells are transplanted into a different subject, i.e., allogeneic to the recipient host subject. The donor's or subject's embryonic stem cells, somatic stem cells, progenitor cells, bone marrow cells, hematopoietic stem cells, or hematopoietic progenitor cells can be transfected with a vector or nucleic acid comprising the nucleic acid molecule(s) described herein, the transfected cells are cultured, inhibited, and differentiated as disclosed, optionally expanded, and then transplanted into the recipient subject. In one embodiment, the transplanted engineered immune cells engraft in the recipient subject. In one embodiment, the transplanted engineered immune cells reconstitute the immune system in the recipient subject. The transfected cells can also be cryopreserved after transfected and stored, or cryopreserved after cell expansion and stored.

The engineered immune cells or the histone methyltransferase inhibited, multilineage hematopoietic progenitor cells or T cells differentiated using a stroma-free method as described herein may be administered as part of a bone marrow or cord blood transplant in an individual that has or has not undergone bone marrow ablative therapy. In one embodiment, genetically modified cells contemplated herein are administered in a bone marrow transplant to an individual that has undergone chemoablative or radioablative bone marrow therapy.

In one embodiment, a dose of cells is delivered to a subject intravenously. In one embodiment, the cells are intravenously administered to a subject.

In particular embodiments, patients receive a dose of the modified cells described herein, e.g., engineered immune cells or the histone methyltransferase inhibited, multilineage hematopoietic progenitor cells or T cells differentiated using a stroma-free method as described herein, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose.

In certain embodiments, patients receive a dose of the modified cells described herein, e.g., engineered immune cells or the histone methyltransferase inhibited, multilineage hematopoietic progenitor cells or T cells differentiated using a stroma-free method as described herein, of at least 1×10¹ cells/kg, at least 5×10¹ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In an additional embodiment, patients receive a dose of the modified cells described herein, e.g., engineered immune cells or the histone methyltransferase inhibited, multilineage hematopoietic progenitor cells or T cells differentiated using a stroma-free method as described herein, of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In general, the engineered immune cells or the histone methyltransferase inhibited, multilineage hematopoietic progenitor cell described herein or T cells differentiated using a stroma-free method as described herein are administered as a suspension with a pharmaceutically acceptable carrier. For example, as therapeutic compositions. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engrafiment upon administration. Such formulations and suspensions are known to those of skill in the art and/or can be adapted for use with the cells as described herein using routine experimentation.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

In one embodiment, the “pharmaceutically acceptable” carrier does not include in vitro cell culture media.

In some embodiments, the composition of engineered immune cells described further comprises a pharmaceutically acceptable carrier.

In various embodiments, at least a second or subsequent dose of cells is administered to the recipient subject. For example, a second administration can be given between about one day to 30 weeks from the previous administration. Two, three, four or more total subsequent administrations can be delivered to the individual, as needed, e.g., determined by a skilled clinician.

A cell composition can be administered by any appropriate route which results in effective cellular replacement treatment in the subject, i.e. administration results in delivery to a desired location in the subject where at least a portion of the composition delivered, i.e. at least 1×10⁴ cells are delivered to the desired site for a period of time. Modes of administration include injection, infusion, or instillation, “Injection” includes, without limitation, intravenous, intra-arterial, intraventricular, intracardiac injection and infusion. For the delivery of cells, administration by injection or infusion is generally preferred.

Efficacy testing can be performed during the course of treatment using the methods described herein. Measurements of the degree of severity of a number of symptoms associated with a particular ailment are noted prior to the start of a treatment and then at later specific time period after the start of the treatment. In some embodiments, a pharmaceutical composition comprising an immune as described herein or a population thereof can be used for cellular replacement therapy in a subject.

Accordingly, it is also the objective of this the present disclosure to provide compositions of modified (also referred to as engineered) cells for use in in vivo cellular replacement therapy, medical therapy such as cancer immune therapy, and for the in vitro studies of disease modeling, drug screening, and hematological diseases.

The advantage of the disclosure protocols is the methods permit semi-permanent bulk production of desired immune cells or other types of hematopoietic cells (i.e. cells differentiated from multipotent HSCs) from a variety of types of cell source, from stem cells, hematopoietic progenitor cells, and mature and differentiated somatic cells, all of which can be readily collected from the patient's body.

The produced engineered immune cells or engineered histone methyltransferase-inhibited, CD34⁺/CD38^{lo/-} hematopoietic progenitor cells (e.g., hemogenic endothelium) or T cells differentiated using a stroma-free method as described herein can be transplanted into a patient for various medical treatments such as immune system reconstruction therapy (e.g., after bone marrow ablation) or immunotherapy (e.g., in cancer therapy or autoimmune diseases). One added advantage is that if the donor of the source cells and recipient of the engineered immune cells are the same person, the produced engineered immune cells have HLA that are identical to the recipient and this avoids host-graft immune rejection after the transplantation. For recipient patients that are HLA allogeneic to the donor person of the source cells, host-graft immune rejection is greatly reduced.

The produced engineered immune cells or engineered histone methyltransferase-inhibited, CD34+/CD 38− hematopoietic progenitor cells or T cells differentiated using a stroma-free method as described herein can also be cryopreserved till needed in the future.

Currently, bone marrow transplantation is the most established cellular replacement therapy for a variety of hematological disorders. The functional unit of a bone marrow transplant is the hematopoietic stem cell (HSC), which resides at the apex of a complex cellular hierarchy and replenishes blood development throughout life. The scarcity of HLA-matched HSCs severely limits the ability to carry out transplantation, disease modeling and drug screening. As such, many studies have aimed to generate HSCs from alternative sources. Advances in reprogramming to induced pluripotent stem cells (iPSCs) has provided access to a wide array of patient-specific pluripotent cells, a promising source for disease modeling, drug screens and cellular therapies. However, the inability to derive engraftable hematopoietic stem and progenitor cells from human pluripotent stem cells (hPSCs) has limited the characterization of hematological diseases to in vitro assays. Generation of HSCs by directed differentiation has remained elusive, and there is a need for novel approaches to this problem.

Accordingly, in one aspect described herein is a method of cellular replacement therapy, the method comprising administering an immune cell as described herein or population thereof, or a composition comprising said immune cell or population thereof, or a pharmaceutical composition comprising said immune cell or population thereof to a recipient subject in need thereof.

In some embodiments, the recipient subject has undergone chemotherapy and/or irradiation. In some embodiments, the recipient subject has deficiencies in immune function and/or lymphocyte reconstitution. In some embodiments, prior to transplanting, the immune cell or population thereof is treated ex vivo with prostaglandin E2 and/or antioxidant N-acetyl-L-cysteine (NAC) to promote subsequent engraftment in a recipient subject.

### Kits

Another aspect of the technology described herein relates to kits for differentiating T cells using a stroma-free method as described herein, among others. Described herein are kit components that can be included in one or more of the kits described herein.

In some embodiments, the kit comprises an effective amount of CD3+ T-cell differentiation factors (e.g., IL-7, SCF, FLT3, and/or TPO); or an effective amount of iPSC differentiation factors (e.g., OCT4, SOX2, KLF4, c-MYC, nanog, and/or LIN28); or an effective amount of hemogenic endothelium differentiation factors (e.g., BMP4, SB-431542, CHIR99021, bFGF, VEGF, IL-6, IL-11, IGF-1, SCF, and EPO); or an effective amount of single-positive T-cell differentiation factors (e.g., IL-15 and/or a T cell activator such as a CD3/CD28 T cell activator); or an effective amount of an inhibitor of an epigenetic regulator (e.g., MC1568; CAY10591; UNC0224; UNC0638; A366; BRD4770; BIX01294; UNC0642; UNC0631; UNC0646; UNC0321; E72; BIX-01338; BRD9539; Chaetocin; or DCG066; e.g., an EZH1 RNA interference agent). As will be appreciated by one of skill in the art, such cell differentiation factors can be supplied in a lyophilized form or a concentrated form that can diluted prior to use with cultured cells. Preferred formulations include those that are non-toxic to the cells and/or does not affect growth rate or viability etc. T-cell differentiation factors can be supplied in aliquots or in unit doses.

In some embodiments, the kit comprises a cell culture vessel comprising an immobilized Notch ligand. In some embodiments, the kit comprises a cell culture vessel and a Notch ligand that can be immobilized to the cell culture vessel using reagents and/or instructions provided therein. In some embodiments, the kit does not comprise stromal cells as described herein.

In some embodiments, the kit further comprises a vector comprising a nucleic acid encoding a CAR.

In some embodiments, the components described herein can be provided singularly or in any combination as a kit. The kit includes the components described herein, e.g., a composition comprising Notch ligand that does not comprise stromal cells, a composition(s) comprising differentiation factor(s), a composition(s) that includes a vector comprising e.g., CAR as described throughout the specification. Such kits can optionally include one or more agents that permit the detection of markers for T cell maturation (e.g., CD5, CD7, CD3, CD4, CD8, TCRgd, TCR alpha or beta, etc.) or a set thereof. Such kits can optionally include one or more agents that permit the detection of markers for T cell activation (e.g., CD107a, CD69, CD25, HLA-DR, IFNg, TNFa, etc.) or a set thereof. Such kits can optionally include one or more agents that permit the detection of markers for hemogenic endothelium (e.g., CD34, CD38, CD45, KDR, CD235, CD43, etc.). In addition, the kit optionally comprises informational material. The kit can also contain a substrate for coating culture dishes, such as laminin, fibronectin, Poly-L-Lysine, or methylcellulose.

In some embodiments, the compositions in the kit can be provided in a watertight or gas tight container which in some embodiments is substantially free of other components of the kit. For example, a cell differentiation reagent can be supplied in more than one container, e.g., it can be supplied in a container having sufficient reagent for a predetermined number of differentiation assays, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of a cell culture vessel comprising immobilized Notch ligand; or the production of T cells differentiated using a stroma-free method as described herein; or the concentration, date of expiration, batch or production site information, and so forth of reagents used herein such as cell differentiation factors. In one embodiment, the informational material relates to methods for using or administering the components of the kit.

The kit can include a component for the detection of a marker for cell differentiation. In addition, the kit can include one or more antibodies that bind a cell marker, or primers for an RT-PCR or PCR reaction, e.g., a semi-quantitative or quantitative RT-PCR or PCR reaction. Such components can be used to assess the activation of cell maturation markers or the loss of undifferentiated or immature cell markers. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “cell” refers to a single cell as well as to a population of (i.e., more than one) cells. The population may be a pure population comprising one cell type, such as a population of pluripotent stem cells or a population of differentiated T cells. As used herein, the term “population” refers to a pure population or to a population comprising a majority (e.g., at least 50%, at least 60%, at least 70%, at least 80%, at least 90%, at least 95%, at least 99%) of one cell type. Alternatively, the population may comprise more than one cell type, for example a mixed cell population. It is not meant to limit the number of cells in a population; for example, a mixed population of cells may comprise at least one differentiated cell. In the present invention, there is no limit on the number of cell types that a mixed cell population may comprise.

As used herein, in one embodiment, the term “hematopoietic stem cell” or “HSC” refers to a stem cell that has self-renewal capacity and also give rise to all the blood cell types of the three hematopoietic lineages, erythroid, lymphoid, and myeloid. These cell types include the myeloid lineages (monocytes and macrophages, neutrophils, basophils, eosinophils, erythrocytes, megakaryocytes/platelets, dendritic cells), and the lymphoid lineages (T-cells, B-cells, NK-cells). Human HSCs are determined as CD34⁺, CD59⁺, CD90/Thy1⁺, CD38^{low/-}, c-kit/CD117^{-/low}, and Lin⁻. Mouse HSC- are considered CD34^{low/-}, SCA-1⁺, CD90/Thy1^{+/low}, CD38⁺, c-Kit/CD117⁺, and Lin⁻. Detecting the expression of these marker panels allows separation of specific cell populations via techniques like fluorescence-activated cell sorting (FACS). In one embodiment, the term “hematopoietic stem cell” or “HSC” refers to a stem cell that has self-renewal capacity and that have the following cell surface markers: CD34+, CD59+, Thy1/CD90⁺, CD38^{lo/-}, CD133+, c-Kit/CD117^{-/lo}, and Lin⁻. In one embodiment, the term “hematopoietic stem cell” or “HSC” refers to a stem cell that is at least CD34+. In one embodiment, the term “hematopoietic stem cell” or “HSC” refers to a stem cell that has self-renewal capacity and that is at least CD34⁺ and c-kit/CD117^{lo/-}. In one embodiment, the term “hematopoietic stem cell” or “HSC” refers to a stem cell that has self-renewal capacity and that is at least CD38^{low/-}, c-kit/CD117^{-/low}. The term HSC can be used interchangeably with the term “hematopoietic stem and progenitor cell” (HSPC).

As used herein, the terms “iPS cell”, “iPSC”, and “induced pluripotent stem cell” are used interchangeably and refers to a pluripotent cell artificially derived by the transfection of the following reprogramming factors OCT4, SOX2, KLF4, and optionally c-MYC or nanog and LIN28, from a differentiated cell, e.g., a somatic cell. Alternative combinations of reprogramming factors include OCT4, SOX2, NANOG, and LIN28. The term hPSC refers to a human pluripotent stem cell.

As used herein, the term “lineage” when used in the context of stem and progenitor cell differentiation and development refers to the cell differentiation and development pathway, which the cell can take to becoming a fully differentiated cell. For example, a HSC has three hematopoietic lineages, erythroid, lymphoid, and myeloid; the HSC has the potential, i.e., the ability, to differentiate and develop into those terminally differentiated cell types known for all these three lineages. When the term “multilineage” used, it means the cell is able to, in the future, differentiate and develop into those terminally differentiated cell types known for more than one lineage. For example, the HSC has multilineage potential. When the term “limited lineage” used, it means the cell can differentiate and develop into those terminally differentiated cell types known for one lineage. For example, a common myeloid progenitor cell (CMP) or a megakaryocyte-erythroid progenitor (MEP) has a limited lineage because the cell can only differentiate and develop into those terminally differentiated cell types of the myeloid lineage and not that of the lymphoid lineage. Terminally differentiated cells of the myeloid lineage include erythrocytes, monocytes, macrophages, megakaryocytes, myeloblasts, dendritic cells, and granulocytes (basophils, neutrophils, eosinophils, and mast cells); and terminally differentiated cells of the lymphoid lineage include T lymphocytes/T cells, B lymphocytes/B cells, dendritic cells, and natural killer cells.

As used herein, the term “a progenitor cell” refers to an immature or undifferentiated cell that has the potential later on to mature (differentiate) into a specific cell type (a fully differentiated or terminally differentiated cell), for example, a blood cell, a skin cell, a bone cell, or hair cells. Progenitor cells have a cellular phenotype that is more primitive (e.g., is at an earlier step along a developmental pathway or progression than is a fully differentiated cell) relative to a cell, which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. A progenitor cell also can proliferate to make more progenitor cells that are similarly immature or undifferentiated.

The term “differentiated cell” is meant any primary cell that is not, in its native form, pluripotent as that term is defined herein. The term a “differentiated cell” also encompasses cells that are partially differentiated, such as multipotent cells (e.g. adult somatic stem cells). In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell as this term is defined herein, can differentiate to lineage-restricted precursor cells (such as a mesodermal stem cell or a endodermal stem cell), which in turn can differentiate into other types of precursor cells further down the pathway (such as an tissue specific precursor, for example, a cardiomyocyte precursor, or a pancreatic precursor), and then to an end-stage differentiated cell, which plays a characteristic role in a certain tissue type, and may or may not retain the capacity to proliferate further.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult somatic stem cells, such as for example, hematopoietic stem cells and neural stem cells, hair follicle stem cells, liver stem cells etc. Multipotent means a stem cell may form many types of cells in a given lineage, but not cells of other lineages. For example, a multipotent blood stem cell can form the many different types of blood cells (red, white, platelets, etc.), but it cannot form neurons; cardiovascular progenitor cell (MICP) differentiation into specific mature cardiac, pacemaker, smooth muscle, and endothelial cell types; pancreas-derived multipotent progenitor (PMP) colonies produce cell types of pancreatic lineage (cells that produces insulin, glucagon, amylase or somatostatin) and neural lineage (cells that are morphologically neuron-like, astrocytes-like or oligodendrocyte-like).

The term a “reprogramming gene”, as used herein, refers to a gene whose expression, contributes to the reprogramming of a differentiated cell, e.g. a somatic cell to an undifferentiated cell (e.g. a cell of a pluripotent state or partially pluripotent state, multipotent state). A reprogramming gene can be, for example, genes encoding master transcription factors Sox2, Oct3/4, Klf4, Nanog, Lin-28, c-myc and the like. The term “reprogramming factor” refers to the protein encoded by the reprogramming gene.

The term “exogenous” refers to a substance present in a cell other than its native source. The terms “exogenous” when used herein refers to a nucleic acid (e.g. a nucleic acid encoding a reprogramming transcription factor, e.g. Sox2, Oct3/4, Klf4, Nanog, Lin-28, c-myc and the like) or a protein (e.g., a transcription factor polypeptide) that has been introduced by a process involving the hand of man into a biological system such as a cell or organism in which it is not normally found or in which it is found in lower amounts. A substance (e.g. a nucleic acid encoding a sox2 transcription factor, or a protein, e.g., a SOX2 polypeptide) will be considered exogenous if it is introduced into a cell or an ancestor of the cell that inherits the substance.

The term “isolated” as used herein signifies that the cells are placed into conditions other than their natural environment. The term “isolated” does not preclude the later use of these cells thereafter in combinations or mixtures with other cells.

As used herein, the term “expanding” refers to increasing the number of like cells through cell division (mitosis). The term “proliferating” and “expanding” are used interchangeably.

As used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markers often provide antigenic determinants to which antibodies can bind to. A cell-surface marker of particular relevance to the methods described herein is CD34. The useful hematopoietic progenitor cells (e.g., hemogenic endothelium) according to the present disclosure preferably express CD34 or in other words, they are CD34 positive.

A cell can be designated “positive” or “negative” for any cell-surface marker, and both such designations are useful for the practice of the methods described herein. A cell is considered “positive” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell may express messenger RNA for a cell-surface marker, in order to be considered positive for the methods described herein, the cell must express it on its surface. Similarly, a cell is considered “negative” or “negative/low” (abbreviated as “-/lo” or “lo/-”) for a cell-surface marker if the cell does not express the marker on its cell surface in amounts sufficient to be detected using methods known to those of skill in the art, such as contacting a cell with an antibody that binds specifically to that marker and subsequently performing flow cytometric analysis of such a contacted cell to determine whether the antibody is bound the cell. In some embodiments, where agents specific for cell-surface lineage markers used, the agents can all comprise the same label or tag, such as fluorescent tag, and thus all cells positive for that label or tag can be excluded or removed, to leave uncontacted hematopoietic stem or progenitor cells for use in the methods described herein.

As used herein, the term “a histone methyltransferase inhibitor” or “inhibitor” is any molecule that inhibits of expression of a histone methyltransferase (e.g., G9a, GLP, EZH1), or inhibits the catalytic activity of the enzyme to methylate lysine resides on the substrate histone protein. For example, a histone methyltransferase inhibitor can be an siRNA or dsRNA that inhibits of expression of G9a, GLP, or EZH1 in the inhibited cell, or a gRNA that promotes the degradation of the mRNA of G9a, GLP, or EZH1 in the inhibited cell. For example, a histone methyltransferase inhibitor is a small molecule that antagonizes the enzyme activity. Examples include but are not limited to small molecules AMI-1, A-366, BIX-01294, BIX01338, BRD4770, chaetocin, UNC0224, UNC0631, UNC0638, UNC0642, UNC0646, EPZ5676, EPZ005687, GSK343, EPZ-6438, 3-deazaneplanocin A (DZNeP) HCl, UNC1999, MM-102, SGC 0946, Entacapone, EPZ015666, UNC0379, Eli, MI-2 (Menin-MLL Inhibitor), MI-3 (Menin-MLL Inhibitor), PFI-2, GSK126, EPZ004777, BRD4770, and EPZ-6438 as described herein.

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. In some embodiments, the small molecule is a heterorganic compound or an organometallic compound.

The term “inhibitory RNA” is meant to include a nucleic acid molecule that contains a sequence that is complementary to a target nucleic acid (e.g., a target microRNA) that mediates a decrease in the level or activity of the target nucleic acid. Non-limiting examples of inhibitory RNAs include interfering RNA, shRNA, siRNA, ribozymes, antagomirs, and antisense oligonucleotides. Methods of making inhibitory RNAs are described herein. Additional methods of making inhibitory RNAs are known in the art. In one embodiment, the G9a/GLP or EZH1 microRNA described herein is an inhibitory RNA that causes a decrease in the activity of G9a/GLP or EZH1 mRNA.

As used herein, “an interfering RNA” refers to any double stranded or single stranded RNA sequence, capable—either directly or indirectly (i.e., upon conversion) of inhibiting or down-regulating gene expression by mediating RNA interference. Interfering RNA includes, but is not limited to, small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family. As used herein, the phrase “post-transcriptional processing” refers to mRNA processing that occurs after transcription and is mediated, for example, by the enzymes Dicer and/or Drosha.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long. Each siRNA duplex is formed by a guide strand and a passenger strand. The endonuclease Argonaute 2 (Ago 2) catalyzes the unwinding of the siRNA duplex. Once unwound, the guide strand is incorporated into the RNA Interference Specificity Complex (RISC), while the passenger strand is released. RISC uses the guide strand to find the mRNA that has a complementary sequence leading to the endonucleolytic cleavage of the target mRNA.

Retroviruses are RNA viruses that utilize reverse transcriptase during their replication cycle. The term “retrovirus” refers to any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus.

The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules, which encode the structural proteins and enzymes needed to produce new viral particles.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R, and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R, and U5 regions, appears at both the both the 5′ and 3′ ends of the viral genome. In one embodiment of the invention, the promoter within the LTR, including the 5′ LTR, is replaced with a heterologous promoter. Examples of heterologous promoters that can be used include, for example, a spleen focus-forming virus (SFFV) promoter, a tetracycline-inducible (TET) promoter, a pi-globin locus control region and a pi-globin promoter (LCR), and a cytomegalovirus (CMV) promoter.

The term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes, i.e., T-cells.

The term “R region” refers to the region within retroviral LTRs beginning at the start of the capping group (i.e., the start of transcription) and ending immediately prior to the start of the poly A tract. The R region is also defined as being flanked by the U3 and U5 regions. The R region plays an important role during reverse transcription in permitting the transfer of nascent DNA from one end of the genome to the other.

The term “promoter/enhancer” refers to a segment of DNA which contains sequences capable of providing both promoter and enhancer functions. For example, the long terminal repeats of retroviruses contain both promoter and enhancer functions. The enhancer/promoter may be “endogenous,” “exogenous,” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA, iRNA, miRNA, siRNA, etc.

The nucleic acid can be selected, for example, from a group including: nucleic acid encoding a protein of interest, oligonucleotides, nucleic acid analogues, for example peptide-nucleic acid (PNA), pseudo-complementary PNA (pc-PNA), and locked nucleic acid (LNA). Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, microRNAi (miRNA), and antisense oligonucleotides.

As used herein, the term “engraftment” in reference to a recipient host is when the new blood-forming cells start to grow and which are derived from the implanted cells and make healthy blood stem cells that show up in recipient's blood after a minimum period of 10 days after implantation. Engraftment can occur as early as 10 days after transplant but is more common around 14-20 days.

As used herein, the term “reconstitution” with respect to the immune system or the blood system in a recipient host refers to the rebuilding the innate reservoir or working system, or part thereof within the body of recipient host to a natural or a functionally state. For example, such as bone marrow after chemotherapy had obliterated the bone marrow stem cells.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cellular replacement therapy. A subject can be male or female.

A subject can be one who has been previously diagnosed with or identified as suffering from or having a condition in need of treatment (e.g. hematologic disease, cancer, etc.) or one or more complications related to such a condition, and optionally, have already undergone treatment for a hematologic disease or the one or more complications related to a hematologic disease. Alternatively, a subject can also be one who has not been previously diagnosed as having a hematologic disease or one or more complications related to a hematologic disease. For example, a subject can be one who exhibits one or more risk factors for a hematologic disease or one or more complications related to a hematologic disease or a subject who does not exhibit risk factors.

A “subject in need” of treatment for a particular condition can be a subject having that condition, diagnosed as having that condition, or at risk of developing that condition.

A variant amino acid or DNA sequence can be at least 85%, at least 87%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, the differentiated and/or engineered T cell described herein is exogenous. In some embodiments, the differentiated and/or engineered T cell described herein is ectopic. In some embodiments, the differentiated and/or engineered T cell described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

Nucleic acids encoding a polypeptide as described herein (e.g. a CAR polypeptide) can be comprised by a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

The vector can be recombinant, e.g., it comprises sequences originating from at least two different sources. In some embodiments, the vector comprises sequences originating from at least two different species. In some embodiments, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector of this invention include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. For example, the use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a hematological disease or cancer. The term “treating” includes reducing or alleviating at least one adverse effect or symptom of a condition, disease or disorder associated with a hematological disease or cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A method for generating CD3+ T cells, the method comprising:
   (a) contacting a CD34+ hemogenic endothelial (HE) cells with a first differentiation medium comprising interleukin-3 (IL-3) under conditions and for a sufficient time to generate CD5+CD7+ T cell progenitor cells,
   (b) contacting the CD5+CD7+ proT cells with a second differentiation medium under conditions and for a sufficient time to generate CD3+ T cells.
2. The method of paragraph 1, wherein the concentration of IL-3 is 1-10 ng/mL.
3. The method of any preceding paragraph, wherein the concentration of IL-3 is 5 ng/mL.
4. The method of any preceding paragraph, wherein the CD34+ HE cells are contacted with IL-3 for about one week.
5. The method of any preceding paragraph, wherein the CD5+CD7+ differentiation medium further comprises at least one of: stem cell factor (SCF), FLT-3, IL-7 and thrombopoietin (TPO).
6. The method of any preceding paragraph, wherein the CD5+CD7+ differentiation medium further comprises each of stem cell factor (SCF), FLT-3, IL-7 and thrombopoietin (TPO)
7. The method of any preceding paragraph, wherein the concentration of SCF is 5-50 ng/mL, and/or the concentration of FLT-3 is 5-30 ng/mL, and/or the concentration of IL-7 is 10-50 ng/mL, and/or the concentration of TPO is 1-10 ng/mL.
8. The method of any preceding paragraph, wherein the CD3+ T cell differentiation medium comprises Fms related receptor tyrosine kinase-3 (FLT3), and interleukin-7 (IL-7).
9. The method of claim 8, wherein the concentration of IL-7 is 1-30 ng/mL, and/or the concentration of FLT-3 is 5-30 ng/mL.
10. The method of any preceding paragraph, wherein the yield of CD3+ T cells is higher than a substantially similar method lacking IL-3 in step (a).
11. The method of any preceding paragraph, wherein the CD34+ HE cells undergo endothelial-to-hematopoietic transition (EHT).
12. The method of any preceding paragraph, wherein step (a) is performed for at least 1 week.
13. The method of any preceding paragraph, wherein step (a) is performed for 2 weeks.
14. The method of any preceding paragraph, wherein step (b) is performed for at least 1 week.
15. The method of any preceding paragraph, wherein the CD34+ HE cells are cultured in the presence of a Notch ligand.
16. The method of any preceding paragraph, wherein the Notch ligand is attached to a solid surface.
17. The method of any preceding paragraph, wherein the Notch ligand is attached to a cell culture dish.
18. The method of any preceding paragraph, wherein the Notch ligand is not expressed by a stromal cell.
19. The method of any preceding paragraph, wherein the method does not comprise co-culturing with a stromal cell expressing a Notch ligand.
20. The method of any preceding paragraph, wherein differentiating the hemogenic endothelium in the presence of a Notch ligand does not comprise co-culturing with OP9-DL1 cells or OP9-DL4 cells.
21. The method of any preceding paragraph, wherein the Notch ligand is selected from the group consisting of Delta-like-1 (DLL1), Delta-like-4 (DLL4), immobilized Delta1^{ext-IgG}, and immobilized Delta4^{ext-IgG}.
22. The method of any preceding paragraph, wherein immobilized Delta1^{ext-IgG} consists of an extracellular domain of human Delta-like-1 fused to the Fc domain of human IgG1.
23. The method of any preceding paragraph, wherein the concentration of DLL4 is in the range of 1-30 □g/mL.
24. The method of any preceding paragraph, wherein the concentration of DLL4 is in the range of 5-25 □g/mL.
25. The method of any preceding paragraph, wherein the concentration of DLL4 is 10□g/mL or 20 □g/mL.
26. The method of any preceding paragraph, further comprising culturing the CD34+ HE cells in the presence of vitronectin.
27. The method of any preceding paragraph, wherein the concentration of vitronectin is in the range of 1-20 □g/mL.
28. The method of any preceding paragraph, wherein the concentration of vitronectin is 10 □g/mL.
29. The method of any preceding paragraph, wherein the CD5+CD7+ T cell differentiation medium and/or the CD3′-T-cell-differentiation media are serum-free.
30. A method for generating CD3+ T cells comprising:
   (a) contacting CD34+ hemogenic endothelial (HE) cells with a first differentiation medium comprising interleukin-3 (IL-3), stem cell factor (SCF), FLT-3, IL-7 and thrombopoietin (TPO) under conditions and for a sufficient time to generate CD5+CD7+ T cell progenitors,
   (b) contacting the CD5+CD7+ T cell progenitors with a second differentiation medium comprising FLT-3 and IL-7 under conditions and for a sufficient time to generate CD3+ T cells.
31. The method of any preceding paragraph, wherein the sufficient time in step (a) is about 1 week.
32. The method of any preceding paragraph, wherein the sufficient time in step (b) is at least 1 week, at least 2 weeks, or at least 3 weeks.
33. The method of any preceding paragraph, further comprising a step of generating CD34+ hemogenic endothelium from a population of pluripotent stem cells, optionally induced pluripotent stem cells (iPSCs).
34. The method of any preceding paragraph, wherein the population of pluripotent stem cells is contacted with an aggregation medium for a sufficient time to generate the CD34+ hemogenic endothelium.
35. The method of any preceding paragraph, wherein the population of pluripotent stem cells is differentiated into a population of CD34⁺ hemogenic endothelium by way of embryoid bodies or 2D adherent cultures.
36. The method of any preceding paragraph, wherein the sufficient time to promote differentiation into a population of CD34+ hemogenic endothelium is at least 8 days.
37. The method of any preceding paragraph, wherein the aggregation media comprises BMP4, SB-431542, CHIR99021, bFGF, VEGF, IL-6, IL-11, IGF-1, SCF, and EPO.
38. The method of any preceding paragraph, wherein the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/mL IL-11, 25 ng/mL IGF-1, 50 ng/mL SCF, and 2 U/ml EPO.
39. The method of any preceding paragraph further comprising selecting or isolating the resultant population of CD34⁺ hemogenic endothelium using expression of surface markers on the population of CD34⁺ hemogenic endothelium.
40. The method of any preceding paragraph, wherein the population of CD34⁺ hemogenic endothelium is CD45 negative/low and/or CD38 negative/low.
41. The method of any preceding paragraph, further comprising the step of genetically modifying the resultant CD34+ hemogenic endothelial cells or the resultant CD3+ T cells.
42. The method of any preceding paragraph, wherein the genetic modification is editing an endogenous HLA, removing an endogenous TCR, and/or expressing a chimeric antigen receptor (CAR).
43. The method of any preceding paragraph, wherein the CD3+ T cells are CD3⁺TCRαβ⁺ T cells.
44. The method of any preceding paragraph, wherein the CD3+ T cells comprise a diverse T cell receptor (TCR) repertoire.
45. The method of any preceding paragraph, wherein the method further comprises inhibition of EZH1 activity and/or expression in the CD34+ HE cells.
46. The method of any preceding paragraph, wherein the EZH1 activity and expression are inhibited by an RNA-guided nuclease system.
47. The method of any preceding paragraph, wherein EZH1 activity and expression are inhibited using a doxycycline-inducible CRISPR interference (CRISPRi) system.
48. The method of any preceding paragraph, wherein the inhibition of EZH1 activity and/or expression comprises contacting the cells with an inhibitor of EZH1 expression.
49. The method of any preceding paragraph, wherein the inhibitor of EZH1 expression comprises an RNA interference molecule.
50. A cell or population of cells made by the method of any preceding paragraph.
51. A method of treating cancer, the method comprising administering a cell or population of cells of any preceding paragraph.
52. A method for generating mature □□T cells, the method comprising:
   (a) contacting a CD34+ hemogenic endothelium (HE) with a CD5+CD7+ differentiation medium comprising interleukin-3 (IL-3) under conditions and for a sufficient time to generate CD5+CD7+ T cell progenitor cells,
   (b) contacting the CD5+CD7+ T cell progenitor cells with a CD3+ T cell differentiation medium under conditions and for a sufficient time to generate mature □□T cells.
53. A method for generating CD3+ T cells, the method comprising:
   (a) contacting a CD34+ hemogenic endothelium (HE) lacking EZH1 activity and/or expression with a CD5+CD7+ differentiation medium comprising interleukin-3 (IL-3) under conditions and for a sufficient time to generate CD5+CD7+ T cell progenitor cells,
   (b) contacting the CD5+CD7+ T cell progenitor cells with a CD3+ T cell differentiation medium under conditions and for a sufficient time to generate CD3+ T cells.
54. The method of any preceding paragraph, wherein the CD34+ hemogenic endothelium lacking EZH1 activity and/or expression is generated using an RNA guided nuclease system.
55. The method of any preceding paragraph, wherein the EZH1 activity and/or expression is inhibited using an EZH1-specific RNA interference agent or a vector encoding an EZH1-specific RNA interference agent.
56. A method for generating mature □□T cells, the method comprising:
   (a) contacting a CD34+ hemogenic endothelium (HE) lacking EZH1 activity and/or expression with a CD5+CD7+ differentiation medium comprising interleukin-3 (IL-3) under conditions and for a sufficient time to generate CD5+CD7+ T cell progenitor cells,
   (b) contacting the CD5+CD7+ T cell progenitor cells with a CD3+ T cell differentiation medium under conditions and for a sufficient time to generate mature □□T cells.
57. The method of any preceding paragraph, wherein the CD34+ hemogenic endothelium lacking EZH1 activity and/or expression is generated using an RNA guided nuclease system.
58. The method of any preceding paragraph, wherein the EZH1 activity and/or expression is inhibited using an EZH1-specific RNA interference agent or a vector encoding an EZH1-specific RNA interference agent.

### EXAMPLES

### Example 1

Chimeric antigen receptor (CAR) T cell-based cancer immunotherapy has proven remarkably effective against lymphoid malignancies (June et al., 2018). In this approach, T cells collected from a patient's blood are expanded in vitro and engineered to express tumor antigen-specific CARs so that the resulting CAR T cells are capable of recognizing and attacking tumor cells. CAR T cell therapy has demonstrated durable therapeutic efficacy and holds great promise for the cure of lymphoid malignancies, but the broader application of this breakthrough anti-cancer strategy has been impeded by several factors. To date, patients have been their own donors, and those who have been heavily pre-treated sometimes lack adequate numbers of functional T cells available for autologous harvest (Fesnak et al., 2016a; Fesnak et al., 2016b). Additionally, the expansion and engineering of autologous T cells requires time that some patients cannot afford. Consequently, this highly effective yet cumbersome, labor-intensive and expensive therapy is still not widely available. An alternative and more readily accessible source for CAR T cells is needed.

Human iPSCs represent an ideal source for scalable manufacture of off-the-shelf products for cell therapy. As immortal cell lines with normal karyotype, iPSCs can be expanded indefinitely and differentiated into multiple cell types through step-wise modulation of select regulatory signals. An early study explored the possibility of using human iPSCs to generate T cells for adoptive T-cell therapy, and showed that iPSC-derived T cells engineered with a CAR against the CD19 antigen were capable of inhibiting tumor growth in tissue culture and murine models (Themeli et al., 2013). However, these iPSC-CAR T cells displayed the transcriptional signature of innate-like γδ T cells, and were not as functionally robust as mature αβ T cells, suggesting that knowledge of the mechanisms underlying in vitro differentiation and developmental maturation of iPSC-derived T cells is incomplete. More recent efforts have demonstrated enhanced T cell maturation when employing iPSCs generated from T cell sources that carry productively rearranged T cell receptors (TCRs) (Iriguchi et al., 2021) or incorporate organoid or thymic culture systems (Vizcardo et al., 2018) (Montel-Hagen et al., 2019). Therefore, methods that enable efficient stroma-free production of fully functional, developmentally mature iPSC-T cells are needed to realize a broad range of iPSC-based adoptive T cell therapies.

Recent studies have revealed key roles for epigenetic regulators during definitive hematopoiesis and lymphoid development. The inventors' lab identified EZH1, a component of polycomb repressive complex 2 (PRC2), as a critical negative regulator of definitive lymphoid commitment during embryonic hematopoietic development (Vo et al., 2018). In this study, the hypothesis that EZH1 repression might lead to more faithful in vitro recapitulation of T cell differentiation and developmental maturation from iPSCs was tested. Below it is shown that iPSC-T cells derived in a stroma-free, serum-free system following EZH1 knockdown (EZ-T Cells) display a molecular signature that more closely approximates peripheral blood αβ T cells rather than innate-like γδ T cells, and that upon activation, are capable of giving rise to not only effector cytotoxic T cells, but also T cell subsets that exhibit a memory-like phenotype. EZ-T cells expressing anti-CD19 CARs showed more robust tumor-killing activity and cytokine secretion compared to control CAR-loaded iPSC-T cells generated without EZH1 repression. Injection of CAR-loaded EZ-T cells into B cell lymphoma-bearing mice also led to superior persistence and more efficient tumor clearance than control iPSC-derived CAR T cells. Thus, a combination of EZH1 repression and stroma-free T cell differentiation allows efficient production of mature iPSC-T cells with enhanced functionality. Such an approach is compatible with commercial-scale production for CAR T cell-based immunotherapy.

a Serum-Free, Stroma-Free System Allows Efficient Differentiation of iPSCs into T Cells Expressing Diverse TCRs

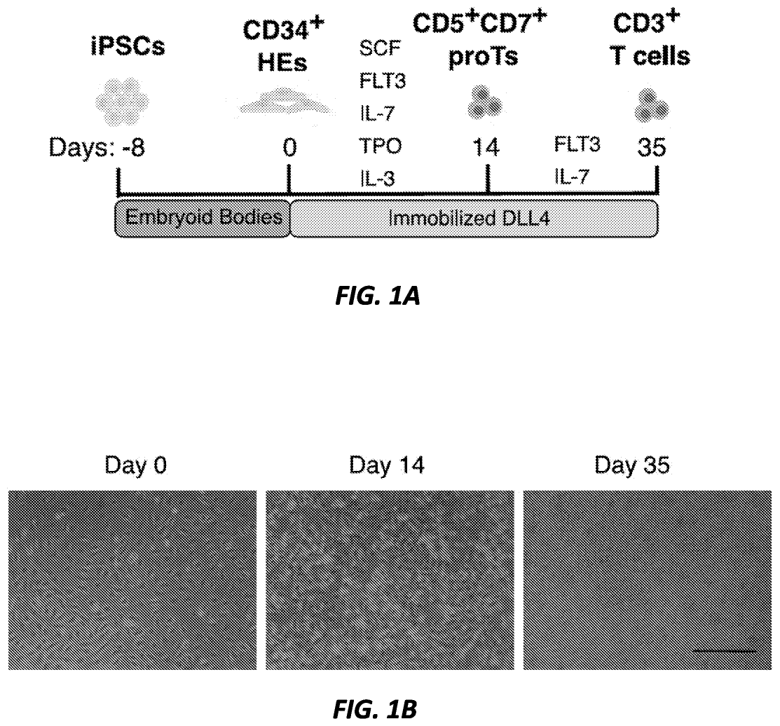

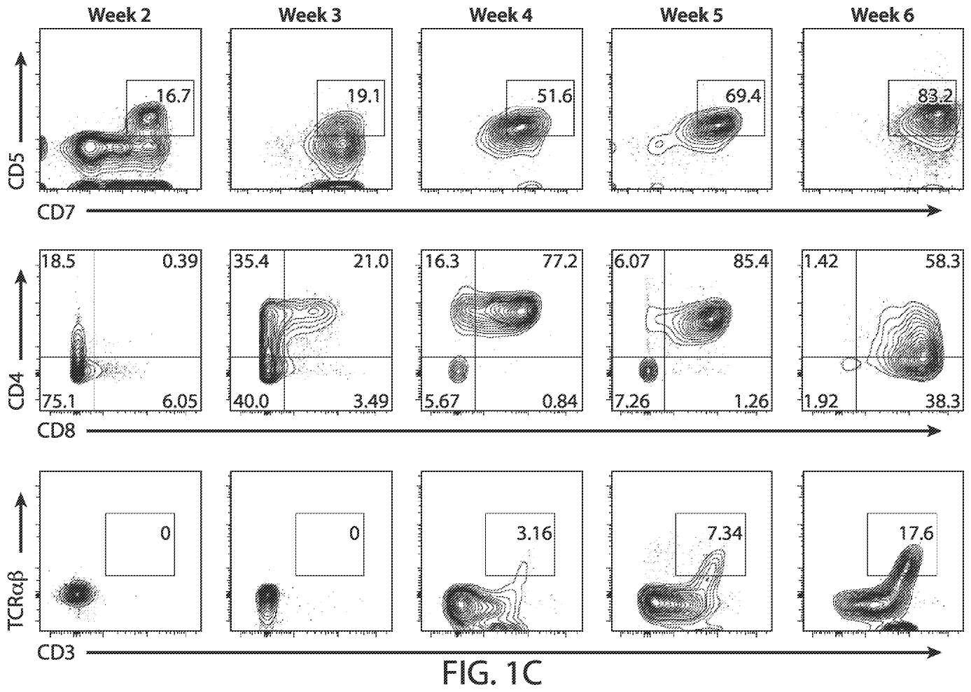

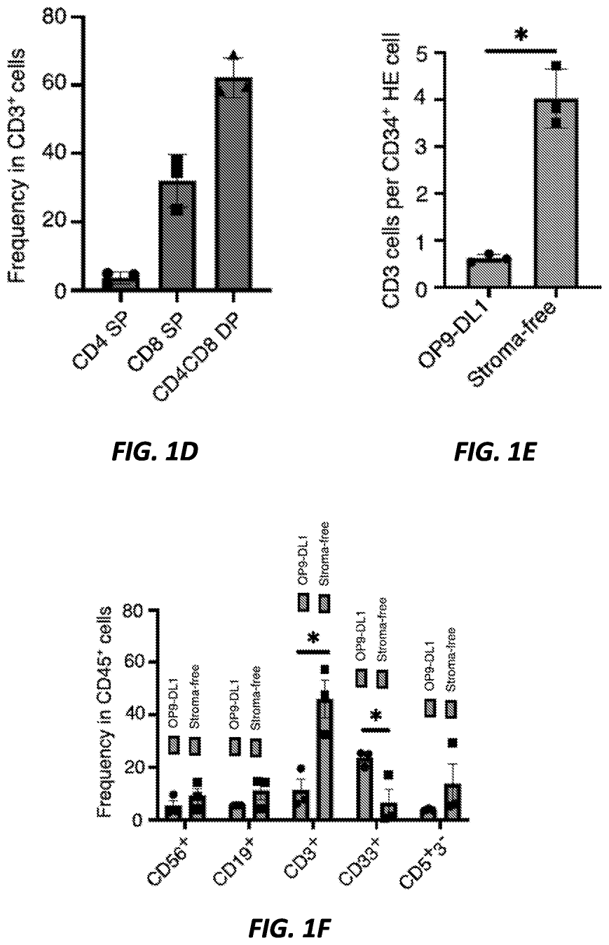

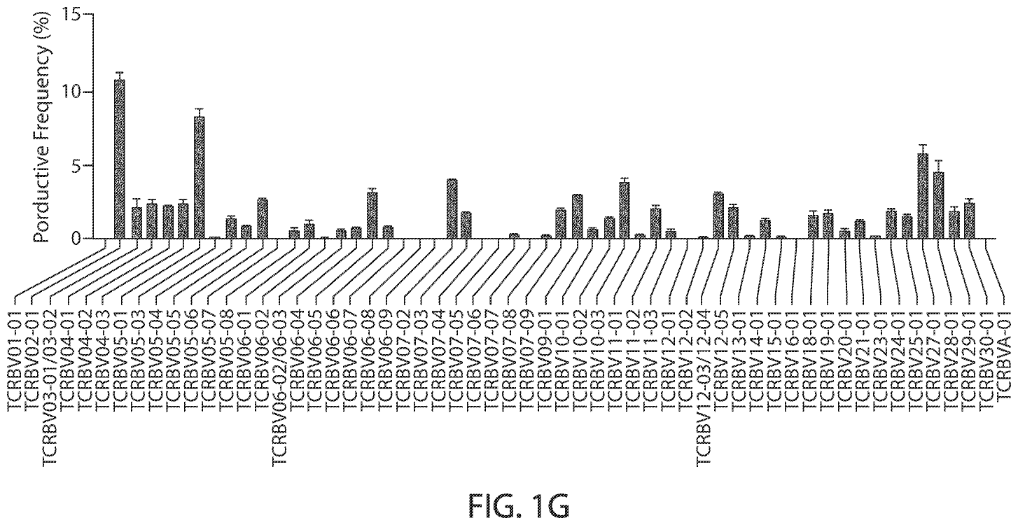

Existing in vitro T cell differentiation protocols largely rely on engineered mouse stromal cells expressing Notch ligands, such as OP9-DL1/DL4 or MS5-DL1 cells, to provide continuous Notch signaling needed for T cell development. (Holmes and Zúñiga-Pfücker, 2009) (Timmermans et al., 2009; Seet et al., 2017). Previous studies have shown that immobilized notch ligands support the induction of primary CD34+ HSPC into T cell lineages (Huijskens et al., 2014) (Shukla et al., 2017). Recently, a stroma-free differentiation protocol was reported to generate T cells from TCR-transduced iPSCs or antigen-specific cytotoxic T cell (CTL)-derived iPSCs (Iriguchi et al., 2021). These iPSCs carry pre-rearranged TCRs and display different T cell differentiation kinetics than their wild-type counterparts (Themeli et al., 2013). Additionally, premature expression of endogenous or transgenic TCR has been shown to produce T cells with innate-like features (Terrence et al., 2000) (Baldwin et al., 2005) (Egawa et al., 2008) (Zhao et al., 2007). Though it has been shown that co-culture with OP9-DL4 cells yields iPSC-T cells with a broad TCR repertoire (Chang et al., 2014), whether a stroma-free system can fully recapitulate normal T cell development and support developmental maturation and random TCR rearrangement in vitro remains unclear. To answer this question, the inventors sought to produce highly differentiated T cells from non-T cell-derived iPSCs while avoiding animal stromal cells and serum. The differentiation procedure is illustrated in FIG. 1A. In the first step, human erythroblast-derived 1157 iPSCs were induced to form embryoid bodies to generate HE cells with hematopoietic potential using a previously reported protocol (Ditadi et al., 2015). The CD34⁺ HE cells were collected and seeded as a monolayer on tissue culture dishes coated with delta-like notch ligand 4 (DL4) and VCAM-1 (Shukla et al., 2017). To initiate T cell specification, HE cells were cultured in SFEMII media supplemented with a cocktail of cytokines (SCF, FLT3, IL-7, IL-3, TPO). During this time period, HE cells underwent endothelial-to-hematopoietic transition (EHT), giving rise to floating cells that contain CD5⁺CD7⁺ T cell progenitors (proTs). On day 14, the floating hematopoietic cells were collected and replated on new DL4 coated plates followed by three weeks of culture with FLT3 and IL-7. The proT cells continued to expand and passed through a transient CD3⁻CD4⁺ immature CD4 single positive (ISP) stage before activating expression of CD8 to form CD4⁺CD8⁺ double positive (DP) cells (FIG. 1B, 1C). After five weeks of differentiation CD3⁺TCRαβ⁺ T cells were detected. Further culture of these cells in the presence of anti-CD3/CD28 antibodies and IL-15 facilitated the induction of single positive (SP) cells. At week 6, a majority of cells expressed CD3 with the population consisting of both DP and CD4/CD8 SP T cells (FIG. 1D).

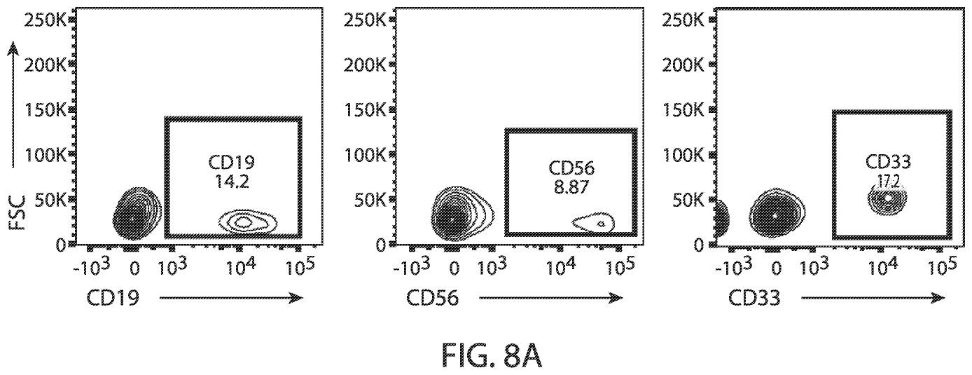

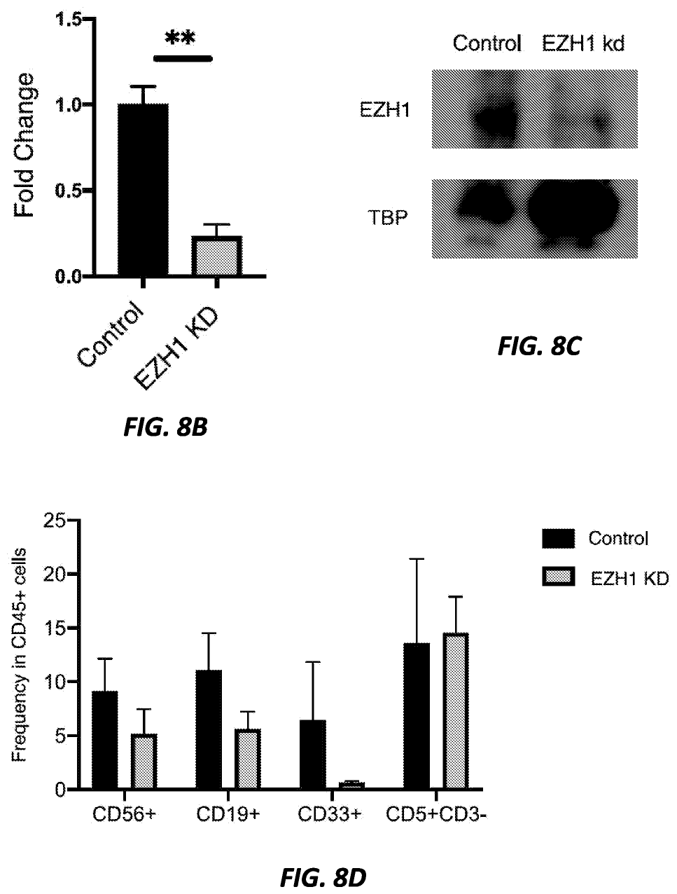

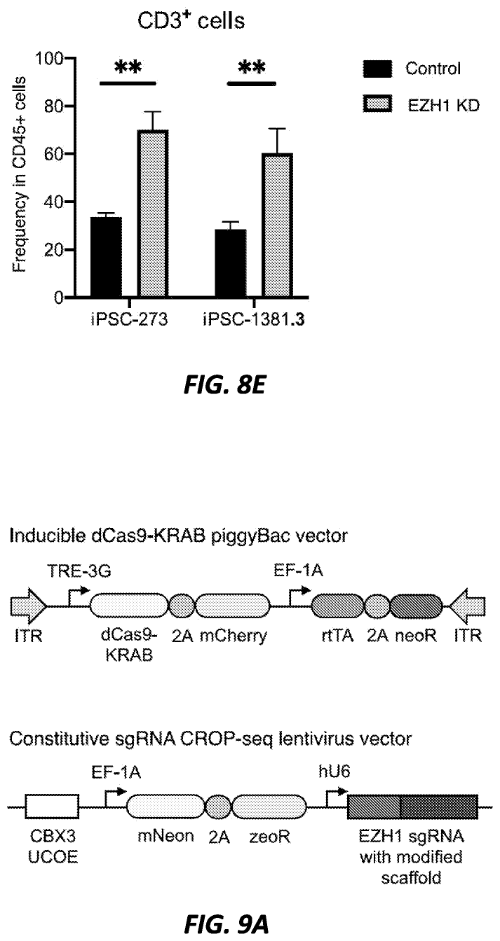

To further evaluate the yield and efficiency of this stroma-free protocol, iPSCs were differentiated into T cells using both the stroma-free method and a previously published OP9-DL1 co-culture system (Themeli et al., 2013). The stroma-free protocol resulted in a significant increase of CD3⁺ T cell production (FIG. 1E). Compared to OP9-DL1 co-culture, the stroma-free system also led to more efficient T cell-specific differentiation, indicated by an increased proportion of T cells and concomitant reductions of non-lymphoid cell frequencies (FIGS. 1F, 8A). To determine whether the stroma-free differentiation was accompanied by normal TCR rearrangement, genomic DNA was extracted from stroma-free iPSC-T cells and the complementarity-determining region 3 (CDR3) of TCRβ locus was sequenced to profile the TCR repertoire. ImmunoSEQ™ analysis identified tens of thousands of unique rearrangements with a high degree of diversity in the usage of Vβ family genes, demonstrating random recombination of CDR3 in the iPSC-T cells (FIG. 1G). Taken together, these data establish a stroma-free system that faithfully recapitulates normal T cell development to produce iPSC-T cells with a highly diverse TCR repertoire.

EZH1 Repression Facilitates In Vitro T Cell Differentiation from iPSCs.

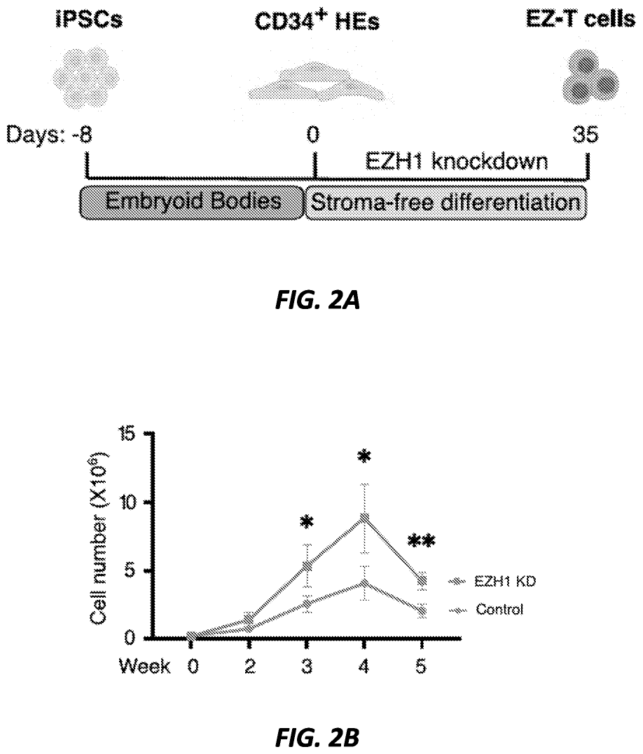

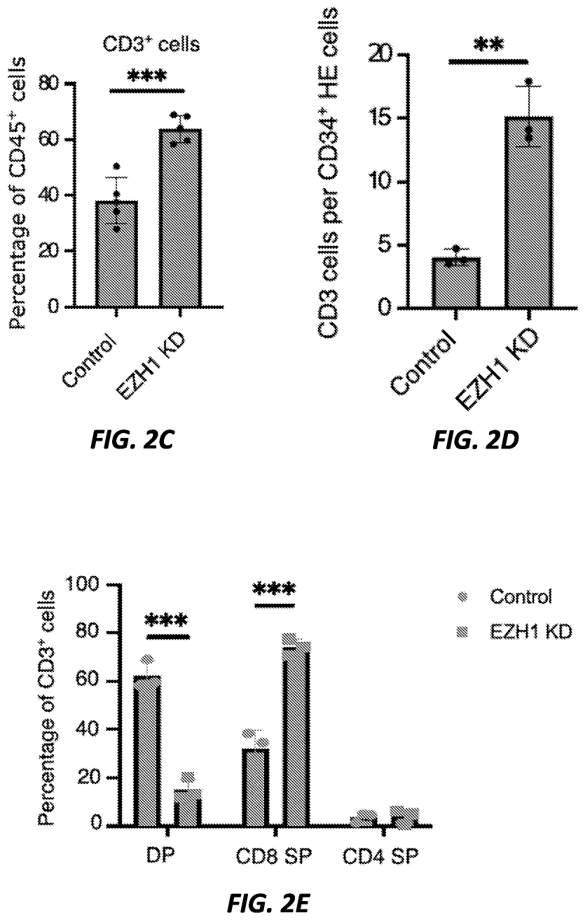

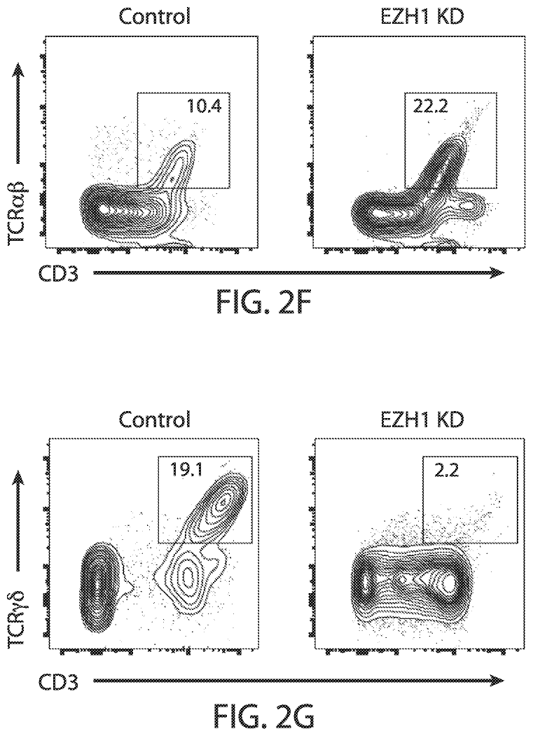

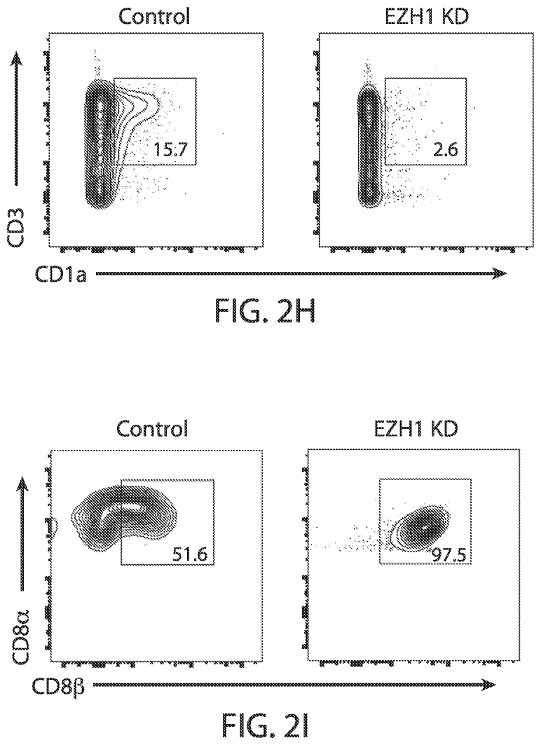

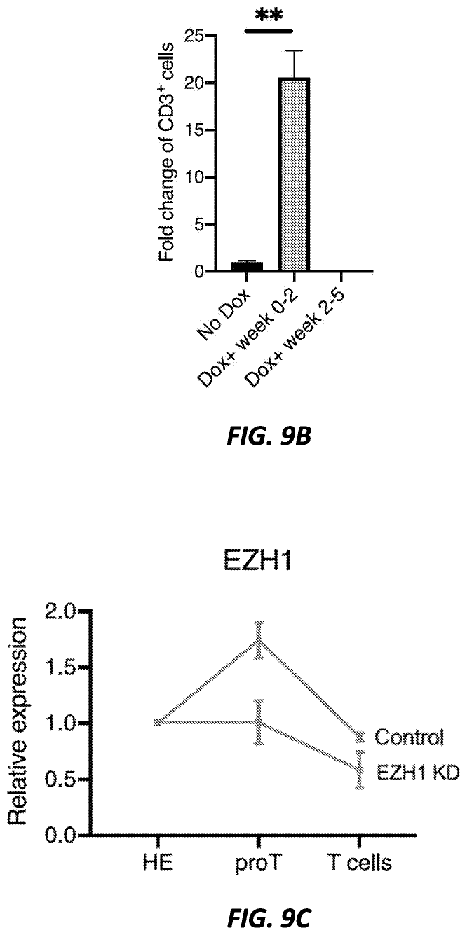

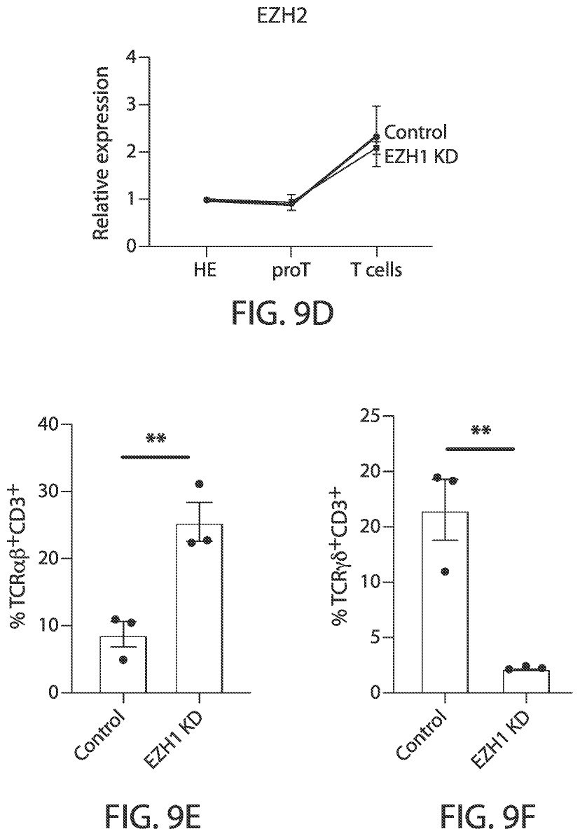

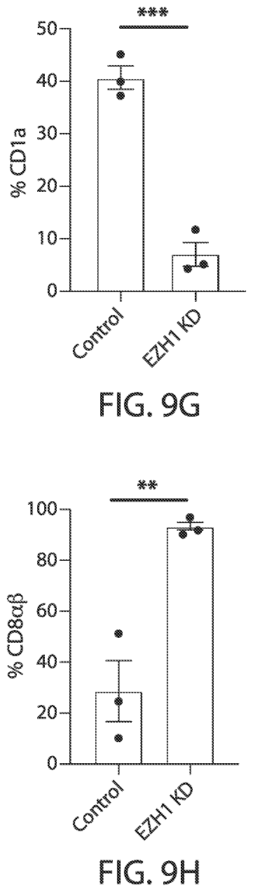

Previous studies have shown that EZH1 acts as a key regulator of hematopoietic multipotency, and repression of EZH1 function promotes lymphoid potential during mouse and zebrafish embryonic development. (Vo et al., 2018) (Soto et al., 2021). To determine whether inhibition of EZH1 would facilitate in vitro T cell differentiation from iPSCs, shRNA-mediated EZH1 knockdown was performed during T cell specification from iPSC-derived CD34⁺ HE cells (FIGS. 8B, 8C). EZH1-knockdown HE cells were then induced to differentiate into EZ-T cells via the stroma-free system, and compared with control iPSC-SF-T cells derived as above (FIG. 2A). EZH1 knockdown produced a ˜2-fold increase in live cells after 2 weeks of differentiation (FIG. 2B), and after 6 weeks resulted in both a higher proportion of T cells amongst CD45⁺ hematopoietic cells and an increased absolute number of T cells generated from each starting CD34⁺ cell, indicating enhanced T cell differentiation specificity and efficiency (FIGS. 2C, 2D, 8D). Similar results were obtained in multiple iPSC lines that were derived from distinct donors using different reprogramming strategies, indicating that the EHZ1-knockdown mediated enhancement of T cell differentiation was not cell line-restricted (FIG. 8E). As an alternative to the shRNA-mediated EZH1 knockdown, the inventors engineered a doxycycline-inducible CRISPR interference (CRISPRi) construct into iPSC-derived CD34⁺ HE cells to transcriptionally repress EZH1 expression (FIG. 9A). Interestingly, CRISPRi mediated EZH1 knockdown during T cell specification (week 0-2) led to a significant increase in the production of CD3⁺ T cells, while induction of EZH1-CRISPRi after the formation of proT cells (week 2-5) failed to promote T cell differentiation (FIG. 9B). In control cells, the expression of EHZ1 significantly increased during specification of HE into proT cells and was down-regulated after the proT stage (FIG. 9C). EZH2, a homolog of EZH1 that likewise acts as a catalytic subunit of the PRC2 complex, was highly expressed at the later stages of T cell differentiation in both control and EZH1 shRNA-treated cells (FIG. 9D). Such an observation is in agreement with previous findings that EZH1 but not EZH2 functions in HE cells to inhibit lymphoid lineage commitment (Vo et al., 2018) and explains why inhibition of EZH1 had no impact on later stages of T cell differentiation. After 6 weeks of differentiation, while the control iPSC-SF-T cells contained a substantial proportion of DP T cells, the EZ-T cells consisted predominantly of CD8 or CD4 SP cells (FIG. 2E). In contrast to the limited production of SP T cells when differentiated on OP9-DL1 stroma, (La Motte-Mohs et al., 2005; Montel-Hagen and Crooks, 2019), these data demonstrate that a combination of stroma-free differentiation with EZH1-knockdown supports more efficient induction of SP cells.

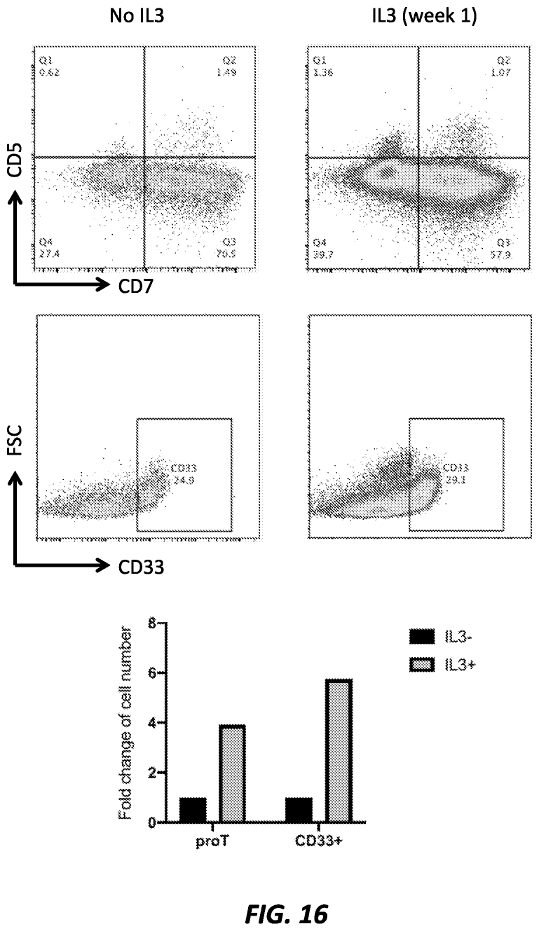

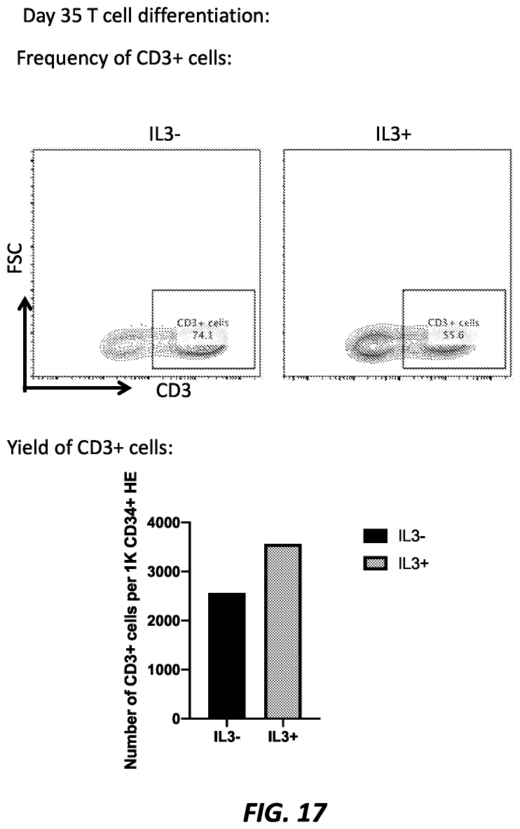

Moreover, data provided herein show that the inclusion of IL-3 in the first differentiation medium from CD34+ HE cells to CD5+CD7+ T cell progenitors for 1 week increased the yield of CD3+ cells per 1,000 CD34+ HE cells (FIGS. 16 and 17).

As previous studies have shown that iPSC-derived T cells tend to resemble innate-like γδ T cells (Nishimura et al., 2013) (Vizcardo et al., 2013) (Themeli et al., 2013), immunophenotypic and molecular analyses were performed to characterize the nature of T cells produced following EZH1 knockdown. It has been suggested that T cell-derived iPSCs (T-iPSCs) bearing rearranged endogenous TCR genes result in premature expression of TCR receptors during in vitro differentiation (Themeli et al., 2013), whereas T cell differentiation from non-T cell derived-PSCs displays similar kinetics to cord blood (CB) CD34⁺ cells, with both gradually upregulating surface TCR/CD3 expression only after the DP stage (Seet et al., 2017) (FIG. 1C). After differentiation, EZ-T cells displayed a significant increase in CD3⁺TCRαβ⁺ and decrease in CD3⁺TCRγδ T cells (FIGS. 2F-2G, 9E-9F), indicating that EZH1 knockdown promotes differentiation towards αβ T cell fate rather than γδ T cells. EZH1 knockdown also resulted in a decrease of CD1a, which is expressed in thymocytes but not mature peripheral blood T cells, further demonstrating that EZ-T cells exhibit a more mature T cell phenotype (FIGS. 2H, 9G). Unlike mature, conventional cytotoxic T cells that express the CD8αβ heterodimer, innate-like T cells such as γδ⁺ T cells or intestinal intraepithelial lymphocytes (IELs) express CD8 as a CD8aa homodimer, which has been considered a less robust co-receptor and may even suppress TCR activation (Bosselut et al., 2000; Holler and Kranz, 2003; Cheroutre and Lambolez, 2008). Studies using OP9-DL1 stroma have yielded CD8αα T cells (Themeli et al., 2013) (Maeda et al., 2016), while recent protocols using 3D artificial thymic organoids or a Notch ligand-based feeder-free system have yielded iPSC-T cells that express CD8αβ (Montel-Hagen et al., 2019) (Iriguchi et al., 2021). Similarly, the inventors' stroma-free differentiation protocol supports the production of CD8αβ T cells, with EZH1 knockdown promoting higher yields of CD8αβ T cells, and barely detectable quantities of innate-like CD8aa T cells (FIGS. 2I, 9H). Collectively, these analyses revealed that repression of EZH1 promotes efficient in vitro differentiation of iPSC cells into mature SP T cells.

### EZ-T Cells Display a Molecular Signature Similar to Peripheral Blood TCRαβ T Cells

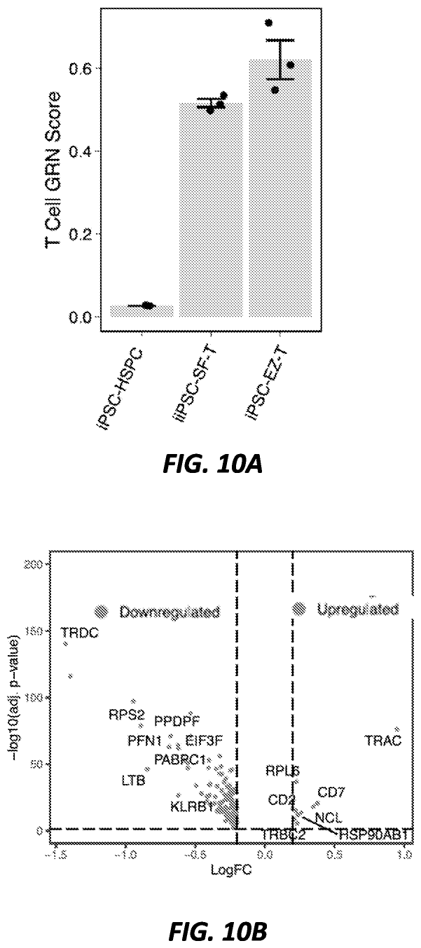

To assess the extent to which these iPSC-T cells resemble their in vivo counterparts, the fidelity of stroma-free in vitro T cell differentiation was evaluated via CellNet™, a network biology platform used to analyze the faithfulness of cell fate conversions and the similarity between in vitro derived cell types and the gold-standard native tissue equivalents (Cahan et al., 2014) (Radley et al., 2017). Analysis of RNA-seq gene expression profiles by CellNet™ revealed a high degree of similarity between iPSC-derived T cells and donor-derived T cells isolated from peripheral blood mononuclear cells (PBMC), and clear discrimination from less differentiated multipotent hematopoietic stem or progenitor cells (HSPCs) (FIGS. 3A, 10A). The inventors next sought a more refined analysis and therefore performed RNA-seq to compare the gene expression profile of EZ-T cells with PBMC-derived TCRαβ T cells, PBMC-TCRγδ T cells, and PBMC-NK cells, as well as T cells generated from CB CD34+ HSPCs or iPSCs in the absence of EZH1 knockdown via in vitro-differentiation on OP9-DL1 stroma or our stroma-free system. Examining the expression of T cell signature genes, it was found that iPSC-T cells differentiated via the stroma-free protocol without EZH1 knockdown (PSC-SF-T) were more similar to CB-HSPC-derived T cells than were iPSC-T cells differentiated via OP9-DL1 stroma; however, these iPSC-SF-T cell types were all closely related to innate-like TCRγδ T cells (FIG. 3B). In contrast, the EZ-T cells were most similar to PBMC TCRαβ T cells. Furthermore, the inventors compared the transcriptional signature of PBMC-derived TCRαβ T with that of TCRγδ T cells and identified a list of genes that can be used to distinguish αβ T from γδ T cells. Expression levels of these genes were determined across all cell types, and the result again showed that EZ-T cells exhibited a gene expression profile that was most similar to αβ T cells (FIG. 3C). To investigate the molecular mechanism underlying the mature phenotype of EZ-T cells, a gene set enrichment analysis (GSEA) was performed on the most significantly upregulated genes in EZ-T cells compared to control iPSC-SF-T cells (lacking EZH1 knockdown), and observed that these genes were highly enriched in biological processes directly associated with T cell differentiation or function (FIGS. 3D, 3E). To access whether the differences identified by bulk RNA-seq were due to changes in cell type compositions, scRNA-seq analysis was performed on control iPSC-SF-T cells and EZ-T cells and compared gene expression profiles for the sub-populations of TCRαβ T cells (TRAC⁺TRDC⁻). A relatively small number of genes were differentially expressed between control αβ T cells and those with EZH1 knockdown (Table 3).

TABLE 3

| gene | mean.np | mean.p | fc | pv | p.adj |
|---|---|---|---|---|---|
| TRAC | 1.51219841 | 2.45955177 | 0.94735335 | 5.59E−79 | 7.99E−77 |
| CD7 | 2.05349497 | 2.43175759 | 0.37826262 | 1.08E−23 | 1.55E−21 |
| NCL | 0.90248797 | 1.2507048 | 0.34821683 | 4.17E−21 | 5.96E−19 |
| HNRNPDL | 0.94196501 | 1.20256509 | 0.26060008 | 1.33E−16 | 1.90E−14 |
| HSP90AB1 | 1.27502213 | 1.51035074 | 0.2353286 | 6.45E−15 | 9.22E−13 |
| TRBC2 | 2.03617391 | 2.26619358 | 0.23001968 | 1.83E−08 | 2.62E−06 |
| RPL6 | 3.3475785 | 3.57311468 | 0.22553618 | 9.73E−40 | 1.39E−37 |
| CD2 | 0.09811966 | 0.31856884 | 0.22044917 | 5.93E−18 | 8.47E−16 |
| HSP90AA1 | 1.12572375 | 1.33591444 | 0.21019069 | 7.95E−12 | 1.14E−09 |
| DDX18 | 0.34979324 | 0.55898583 | 0.20919259 | 1.02E−11 | 1.46E−09 |
| FTL | 3.57849812 | 3.78186808 | 0.20336995 | 2.85E−25 | 4.07E−23 |
| LDHB | 2.18499007 | 2.38500281 | 0.20001274 | 4.05E−19 | 5.79E−17 |
| GZMM | 0.67501409 | 0.4749995 | −0.2000146 | 3.86E−09 | 5.52E−07 |
| LPAR6 | 0.57374031 | 0.37318457 | −0.2005557 | 1.88E−12 | 2.69E−10 |
| UBE2F | 0.38745867 | 0.18584008 | −0.2016186 | 4.88E−20 | 6.98E−18 |
| PIN1 | 0.71610849 | 0.51359339 | −0.2025151 | 4.75E−11 | 6.79E−09 |
| EGLN3 | 0.34723554 | 0.14450261 | −0.2027329 | 6.06E−23 | 8.67E−21 |
| COX5A | 0.2972553 | 0.09443279 | −0.2028225 | 1.09E−31 | 1.56E−29 |
| RBCK1 | 0.3762505 | 0.17335751 | −0.202893 | 2.08E−24 | 2.98E−22 |
| RALY | 0.29361149 | 0.08989931 | −0.2037122 | 1.17E−31 | 1.67E−29 |
| CD1E | 0.27972447 | 0.07559481 | −0.2041297 | 5.99E−17 | 8.56E−15 |
| CDK2AP2 | 0.51036695 | 0.30490729 | −0.2054597 | 1.96E−15 | 2.80E−13 |
| ID3 | 0.7286147 | 0.52147493 | −0.2071398 | 5.53E−09 | 7.91E−07 |
| MRFAP1 | 0.72106961 | 0.51383577 | −0.2072338 | 1.91E−10 | 2.73E−08 |
| MYO1F | 1.02468097 | 0.81678446 | −0.2078965 | 9.28E−09 | 1.33E−06 |
| DSTN | 0.70131786 | 0.48795944 | −0.2133584 | 5.65E−11 | 8.09E−09 |
| RHOF | 0.80429482 | 0.59059104 | −0.2137038 | 3.28E−10 | 4.69E−08 |
| LRCH4 | 0.30193116 | 0.08805861 | −0.2138725 | 3.38E−34 | 4.83E−32 |
| PLD3 | 0.46168313 | 0.24660331 | −0.2150798 | 1.29E−20 | 1.85E−18 |
| VAMP2 | 0.65150767 | 0.43480771 | −0.2167 | 9.13E−14 | 1.31E−11 |
| HSPA8 | 1.95693511 | 1.74012897 | −0.2168061 | 3.23E−08 | 4.62E−06 |
| CHST12 | 0.32727421 | 0.1089029 | −0.2183713 | 3.56E−33 | 5.09E−31 |
| RHOC | 1.25033404 | 1.03070145 | −0.2196326 | 5.57E−09 | 7.97E−07 |
| ATP5ME | 1.3762897 | 1.15573402 | −0.2205557 | 1.17E−09 | 1.68E−07 |
| VKORC1 | 0.34601717 | 0.12280969 | −0.2232075 | 1.94E−31 | 2.77E−29 |
| LSM2 | 0.53328612 | 0.30940377 | −0.2238823 | 8.53E−18 | 1.22E−15 |
| PKM | 1.6025405 | 1.37821983 | −0.2243207 | 1.04E−09 | 1.49E−07 |
| BST2 | 0.86722277 | 0.6421247 | −0.2250981 | 1.55E−11 | 2.22E−09 |
| ANXA2 | 1.04397276 | 0.81840621 | −0.2255665 | 2.26E−08 | 3.23E−06 |
| P4HB | 0.41203228 | 0.18508467 | −0.2269476 | 9.98E−24 | 1.43E−21 |
| STMN1 | 1.39017171 | 1.16255891 | −0.2276128 | 1.85E−05 | 0.00264614 |
| ID2 | 1.27346779 | 1.04460245 | −0.2288653 | 1.40E−06 | 0.00019952 |
| HIST1H4C | 1.03554302 | 0.80274859 | −0.2327944 | 1.93E−09 | 2.75E−07 |
| HM13 | 0.67444482 | 0.44133168 | −0.2331131 | 8.54E−15 | 1.22E−12 |
| MRPL12 | 0.36049672 | 0.12721714 | −0.2332796 | 8.80E−34 | 1.26E−31 |
| HNRNPA0 | 0.35478548 | 0.12068396 | −0.2341015 | 8.89E−33 | 1.27E−30 |
| RCBTB2 | 0.68105474 | 0.44443627 | −0.2366185 | 1.02E−12 | 1.46E−10 |
| ZAP70 | 0.45979154 | 0.2229802 | −0.2368113 | 2.51E−23 | 3.58E−21 |
| DNAJC4 | 0.31176977 | 0.07483069 | −0.2369391 | 1.47E−48 | 2.10E−46 |
| IFITM2 | 3.07802222 | 2.84094503 | −0.2370772 | 3.05E−26 | 4.36E−24 |
| MTRNR2L8 | 1.17561903 | 0.93798064 | −0.2376384 | 3.99E−11 | 5.70E−09 |
| MTRNR2L10 | 1.13679961 | 0.8985783 | −0.2382213 | 3.28E−11 | 4.69E−09 |
| MYO1G | 0.47538862 | 0.2370768 | −0.2383118 | 4.99E−22 | 7.14E−20 |
| GZMA | 0.71576303 | 0.47631188 | −0.2394511 | 3.86E−09 | 5.52E−07 |
| WAS | 0.77070375 | 0.5312258 | −0.2394779 | 1.02E−13 | 1.46E−11 |
| FKBP8 | 1.21538464 | 0.97447099 | −0.2409137 | 1.24E−11 | 1.78E−09 |
| DNPH1 | 0.45184395 | 0.21047021 | −0.2413737 | 4.93E−25 | 7.05E−23 |
| ARGLU1 | 0.47956697 | 0.23714185 | −0.2424251 | 5.35E−23 | 7.65E−21 |
| TUBB | 1.50787486 | 1.26480279 | −0.2430721 | 1.54E−09 | 2.20E−07 |
| LSM4 | 0.46215659 | 0.21846266 | −0.2436939 | 2.61E−25 | 3.74E−23 |
| ILK | 0.75888995 | 0.51480316 | −0.2440868 | 2.35E−15 | 3.36E−13 |
| ZBTB16 | 1.06440113 | 0.81986332 | −0.2445378 | 2.40E−10 | 3.44E−08 |
| EEF2 | 2.80601041 | 2.56079894 | −0.2452115 | 4.95E−29 | 7.08E−27 |
| RPS29 | 3.44791412 | 3.20044408 | −0.24747 | 4.40E−48 | 6.30E−46 |
| MT-ND3 | 2.31335996 | 2.06486939 | −0.2484906 | 4.80E−20 | 6.86E−18 |
| TKT | 1.54285611 | 1.2915926 | −0.2512635 | 1.52E−12 | 2.18E−10 |
| DDIT4 | 0.40622835 | 0.15469651 | −0.2515318 | 6.63E−32 | 9.48E−30 |
| ANXA1 | 0.36531777 | 0.1131372 | −0.2521806 | 7.36E−37 | 1.05E−34 |
| ITM2A | 1.04162653 | 0.78791912 | −0.2537074 | 2.94E−11 | 4.20E−09 |
| RAP1B | 0.9651904 | 0.7100421 | −0.2551483 | 3.07E−13 | 4.39E−11 |
| MT-ND2 | 3.01033333 | 2.75293563 | −0.2573977 | 1.36E−34 | 1.95E−32 |
| CCDC107 | 0.40438059 | 0.14456514 | −0.2598154 | 3.64E−35 | 5.21E−33 |
| TAGLN2 | 1.23775769 | 0.97747891 | −0.2602788 | 1.57E−11 | 2.24E−09 |
| BIN1 | 1.08341029 | 0.82301201 | −0.2603983 | 2.51E−13 | 3.59E−11 |
| MT-ND5 | 1.82149772 | 1.5602184 | −0.2612793 | 1.12E−13 | 1.60E−11 |
| YWHAQ | 0.45269958 | 0.18985964 | −0.2628399 | 4.61E−29 | 6.60E−27 |
| HERPUD1 | 0.50175239 | 0.23846709 | −0.2632853 | 6.53E−27 | 9.33E−25 |
| TIMP1 | 1.13619234 | 0.87103531 | −0.265157 | 1.90E−12 | 2.71E−10 |
| CALR | 0.77443412 | 0.50660204 | −0.2678321 | 3.70E−16 | 5.30E−14 |
| JAML | 1.02640106 | 0.7584826 | −0.2679185 | 2.43E−12 | 3.47E−10 |
| ATP6V0C | 0.43032079 | 0.15954796 | −0.2707728 | 7.65E−36 | 1.09E−33 |
| GLRX | 0.95035167 | 0.67912533 | −0.2712263 | 1.37E−14 | 1.95E−12 |
| PHB2 | 0.94513037 | 0.6735746 | −0.2715558 | 6.26E−16 | 8.96E−14 |
| SHKBP1 | 0.53397987 | 0.25881997 | −0.2751599 | 1.56E−27 | 2.24E−25 |
| UCP2 | 1.47892967 | 1.20358439 | −0.2753453 | 1.72E−13 | 2.46E−11 |
| PYCARD | 0.48181104 | 0.20497103 | −0.27684 | 1.43E−29 | 2.04E−27 |
| C12orf75 | 0.88927923 | 0.61222796 | −0.2770513 | 7.54E−16 | 1.08E−13 |
| GNG5 | 1.12159057 | 0.84409999 | −0.2774906 | 2.26E−15 | 3.23E−13 |
| TMED2 | 0.57866813 | 0.30117621 | −0.2774919 | 4.34E−26 | 6.21E−24 |
| JTB | 0.41147356 | 0.13123834 | −0.2802352 | 3.50E−42 | 5.00E−40 |
| AURKAIP1 | 0.76002431 | 0.47975239 | −0.2802719 | 3.03E−19 | 4.34E−17 |
| ADA | 0.46396348 | 0.18244262 | −0.2815209 | 5.00E−29 | 7.15E−27 |
| TPST2 | 0.63762339 | 0.35564352 | −0.2819799 | 5.39E−21 | 7.71E−19 |
| S100A6 | 2.05346181 | 1.76939196 | −0.2840699 | 3.80E−10 | 5.43E−08 |
| MAPKAPK3 | 0.55010732 | 0.26118843 | −0.2889189 | 2.17E−30 | 3.11E−28 |
| MPG | 0.44843907 | 0.15716918 | −0.2912699 | 1.72E−39 | 2.46E−37 |
| FKBP1A | 1.03135601 | 0.72947082 | −0.3018852 | 3.08E−17 | 4.41E−15 |
| TBCB | 0.70215042 | 0.39899846 | −0.303152 | 7.37E−25 | 1.05E−22 |
| SEPTIN1 | 0.43112999 | 0.12281992 | −0.3083101 | 2.70E−45 | 3.87E−43 |
| PDCD6 | 0.55998814 | 0.24880033 | −0.3111878 | 3.01E−33 | 4.31E−31 |
| CD44 | 1.78285722 | 1.46770408 | −0.3151531 | 2.30E−19 | 3.29E−17 |
| TNFRSF18 | 0.40824853 | 0.09199417 | −0.3162544 | 5.57E−51 | 7.96E−49 |
| NDUFB10 | 1.02911646 | 0.71032961 | −0.3187869 | 1.98E−20 | 2.83E−18 |
| GUK1 | 0.83932707 | 0.51843371 | −0.3208934 | 3.33E−23 | 4.76E−21 |
| SLC25A3 | 1.78833687 | 1.46617935 | −0.3221575 | 1.89E−22 | 2.71E−20 |
| TRIR | 0.4112468 | 0.08893452 | −0.3223123 | 5.70E−59 | 8.15E−57 |
| TXNIP | 1.11892346 | 0.79563051 | −0.3232929 | 3.12E−17 | 4.45E−15 |
| S100A11 | 2.185397 | 1.86190167 | −0.3234953 | 1.32E−22 | 1.89E−20 |
| CISH | 0.63885526 | 0.3153338 | −0.3235215 | 8.10E−24 | 1.16E−21 |
| ALKBH7 | 0.49538179 | 0.16937328 | −0.3260085 | 7.44E−46 | 1.06E−43 |
| PRF1 | 1.05152258 | 0.71413872 | −0.3373839 | 5.82E−19 | 8.33E−17 |
| FCER1G | 2.07827332 | 1.73569132 | −0.342582 | 1.60E−17 | 2.29E−15 |
| PPP4C | 0.62379382 | 0.28092326 | −0.3428706 | 2.24E−36 | 3.20E−34 |
| ACTB | 4.63510324 | 4.28536729 | −0.3497359 | 1.97E−49 | 2.82E−47 |
| TSC22D4 | 0.83466903 | 0.47772322 | −0.3569458 | 2.71E−28 | 3.88E−26 |
| CD247 | 1.83030705 | 1.46943609 | −0.360871 | 1.35E−23 | 1.93E−21 |
| CALM1 | 1.43464033 | 1.04870197 | −0.3859384 | 4.01E−22 | 5.73E−20 |
| SARAF | 0.78204795 | 0.38890046 | −0.3931475 | 6.42E−38 | 9.18E−36 |
| C9orf16 | 1.42667201 | 1.02897036 | −0.3977017 | 1.34E−29 | 1.91E−27 |
| VSIR | 0.74762317 | 0.34983305 | −0.3977901 | 2.21E−37 | 3.16E−35 |
| FTH1 | 0.57774986 | 0.17545968 | −0.4022902 | 7.02E−56 | 1.00E−53 |
| LIME1 | 0.58576663 | 0.18024036 | −0.4055263 | 5.73E−50 | 8.20E−48 |
| ITM2C | 1.00656029 | 0.59860172 | −0.4079586 | 8.89E−29 | 1.27E−26 |
| CD99 | 1.7924677 | 1.37622746 | −0.4162402 | 3.37E−25 | 4.82E−23 |
| CTSD | 1.22159877 | 0.80466334 | −0.4169354 | 2.32E−28 | 3.31E−26 |
| S100A10 | 1.18001085 | 0.72550212 | −0.4545087 | 6.41E−31 | 9.16E−29 |
| VIM | 1.18311841 | 0.68989248 | −0.4932259 | 8.14E−37 | 1.16E−34 |
| CYBA | 0.66354315 | 0.12852253 | −0.5350206 | 6.34E−91 | 9.07E−89 |
| TBC1D10C | 0.77105091 | 0.23570737 | −0.5353435 | 7.30E−74 | 1.04E−71 |
| LY6E | 1.30346169 | 0.75032282 | −0.5531389 | 5.15E−50 | 7.37E−48 |
| ARL6IP4 | 1.14259449 | 0.58891911 | −0.5536754 | 6.37E−54 | 9.11E−52 |
| GYPC | 1.58382311 | 1.00787172 | −0.5759514 | 7.91E−56 | 1.13E−53 |
| TSPO | 1.1622586 | 0.57990367 | −0.5823549 | 2.38E−55 | 3.41E−53 |
| PABPC1 | 1.86255527 | 1.24254091 | −0.6200144 | 8.70E−65 | 1.24E−62 |
| KLRB1 | 2.07077878 | 1.44953222 | −0.6212466 | 1.56E−29 | 2.24E−27 |
| EIF3F | 1.02366418 | 0.40000124 | −0.6236629 | 3.21E−67 | 4.59E−65 |
| PPDPF | 1.30639371 | 0.62910217 | −0.6772915 | 5.72E−74 | 8.18E−72 |
| PFN1 | 1.87388437 | 1.18660476 | −0.6872796 | 7.34E−66 | 1.05E−63 |
| LTB | 1.57184477 | 0.7271201 | −0.8447247 | 3.82E−49 | 5.46E−47 |
| RPS2 | 2.04452455 | 1.1518362 | −0.8926884 | 1.02E−81 | 1.46E−79 |
| MIF | 2.06540836 | 1.12105865 | −0.9443497 | 5.36E−100 | 7.66E−98 |
| RPL13 | 3.11250484 | 1.71627326 | −1.3962316 | 6.12E−119 | 8.76E−117 |
| TRDC | 1.85493222 | 0.42477924 | −1.430153 | 3.35E−143 | 4.79E−141 |

Interestingly, TCRαβ T cells with EZH1 knockdown more abundantly expressed TRAC, TRBC2, CD2, and CD7, and showed a greater down-regulation of residue “innate” genes (TRDC, KLRB1) (FIG. 10B). Collectively, EZH1 repression during in vitro iPSC differentiation promotes the production of αβ T cells that display a more mature phenotype.

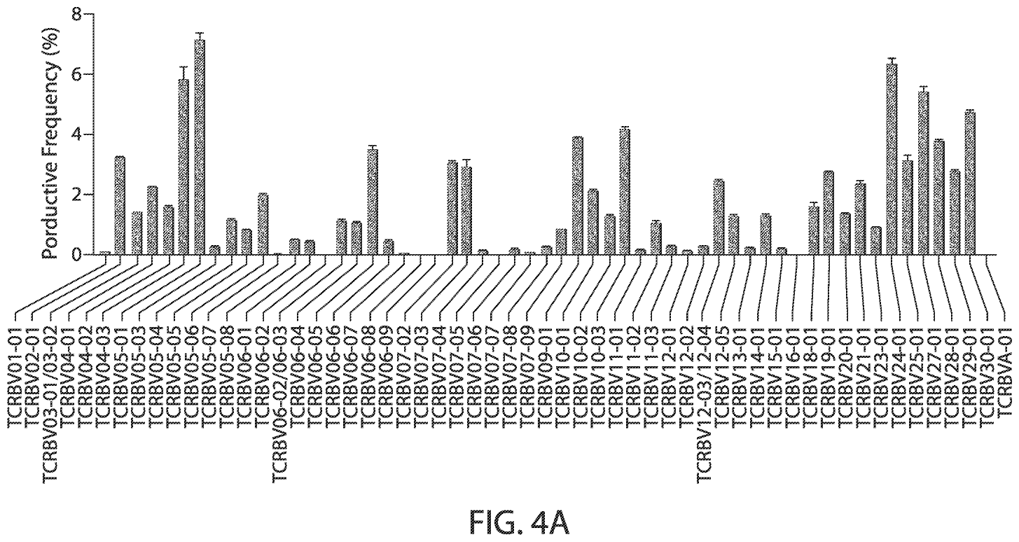

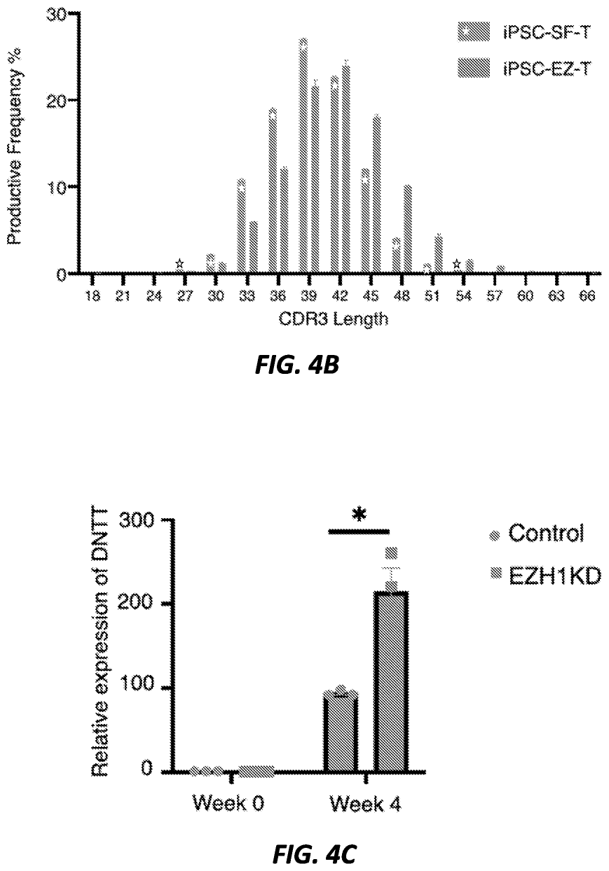

The inventors next performed immunosequencing to determine the TCR repertoire of EZ-T cells, and observed a high degree of TCRβ diversity with no preferential Vβ gene usage (FIG. 4A), indicating that EZH1-knockdown did not cause significant clonal expansion of individual T cells with specific TCR rearrangements. Notably, EZ-T cells displayed longer CDR3 regions than iPSC-SF-T cells without EZH1 knockdown (FIG. 4B). iPSC-derived T cells tend to have shorter CDR3 regions than mature peripheral blood T cells, likely due to lower expression levels of Terminal Deoxynucleotide Transferase (TdT), the enzyme responsible for random nucleotide insertion during VDJ recombination, which is encoded by the DNTT gene (Montel-Hagen et al., 2019). To test this, the expression levels of TdT/DNTT were determined in both iPSC-derived HE cells and week 4 DP T cells harboring control or EZH1 shRNA. As expected, TdT/DNTT was only expressed in DP T cells and more abundantly expressed in cells with EZH1-knockdown (FIG. 4C). These data indicate that EZH1-knockdown during iPSC differentiation promotes T cell maturation, with the resulting EZ-T cells exhibiting molecular signatures that most closely resemble peripheral blood TCRαβ T cells.

### EZ-T Cell Subsets Display Effector and Memory-Like Phenotypes.

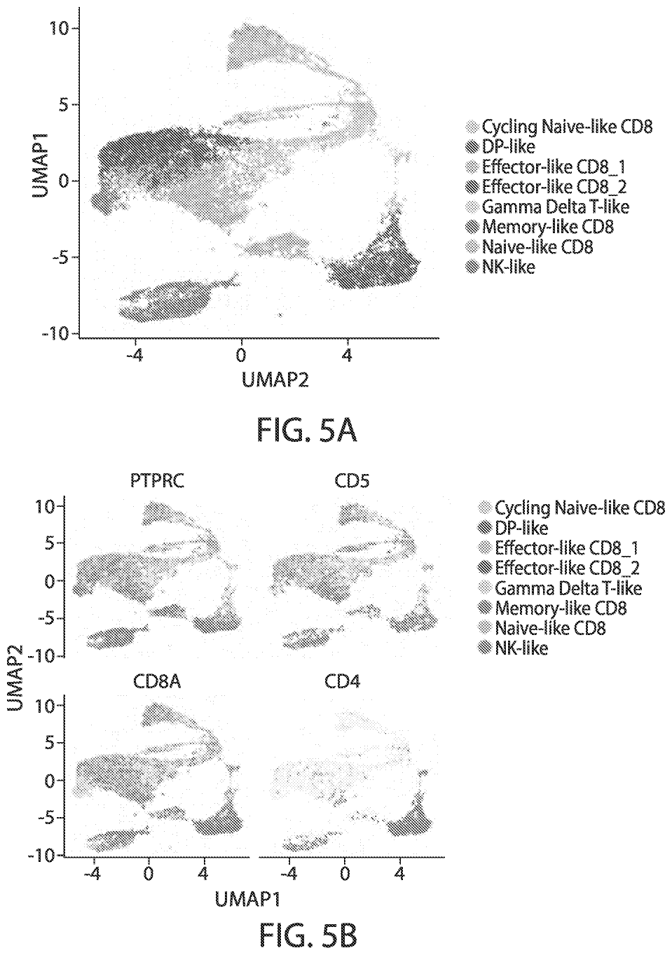

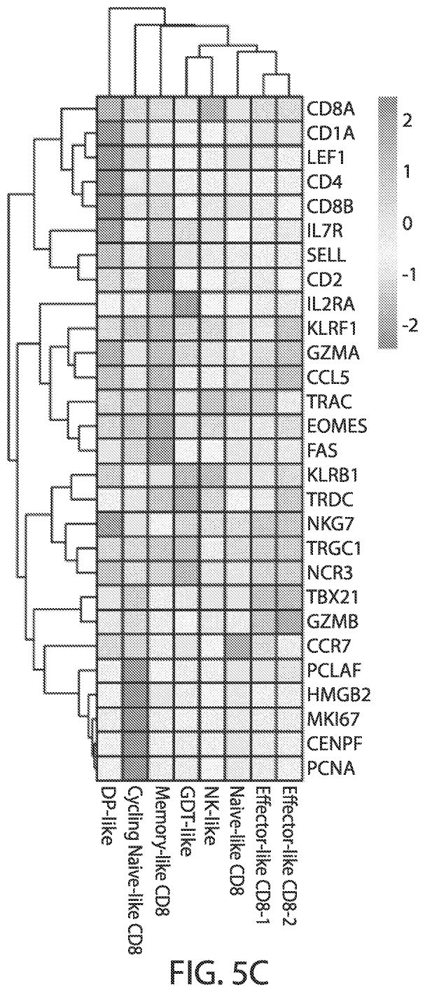

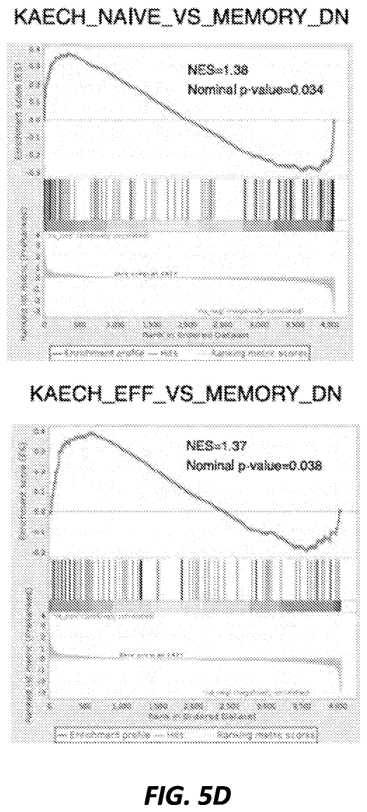

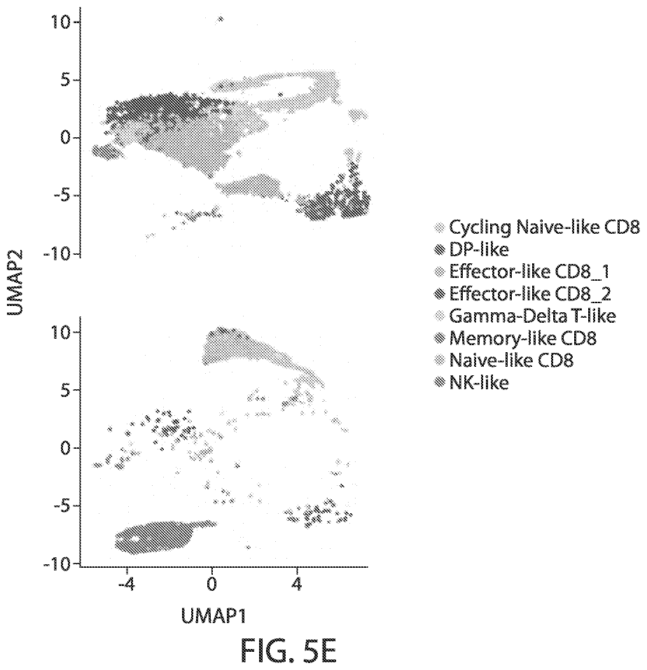

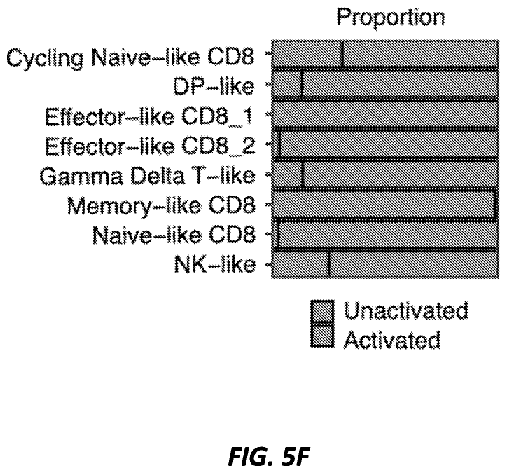

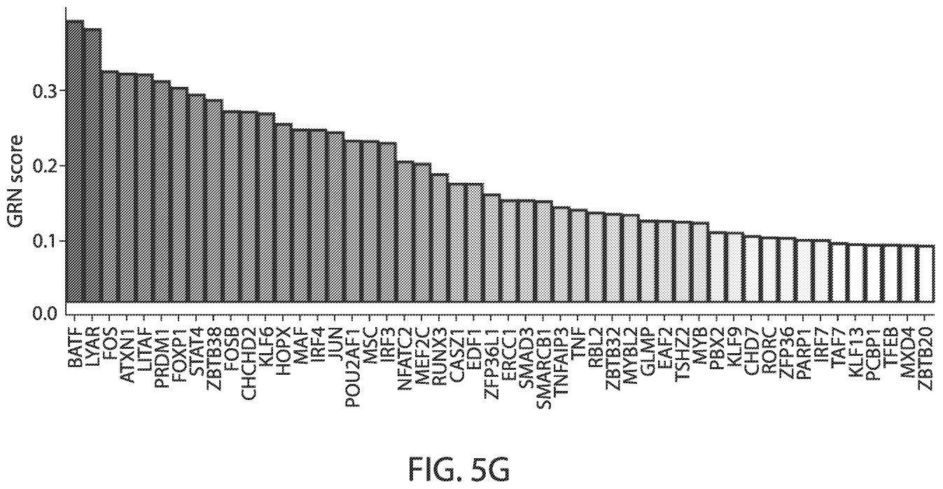

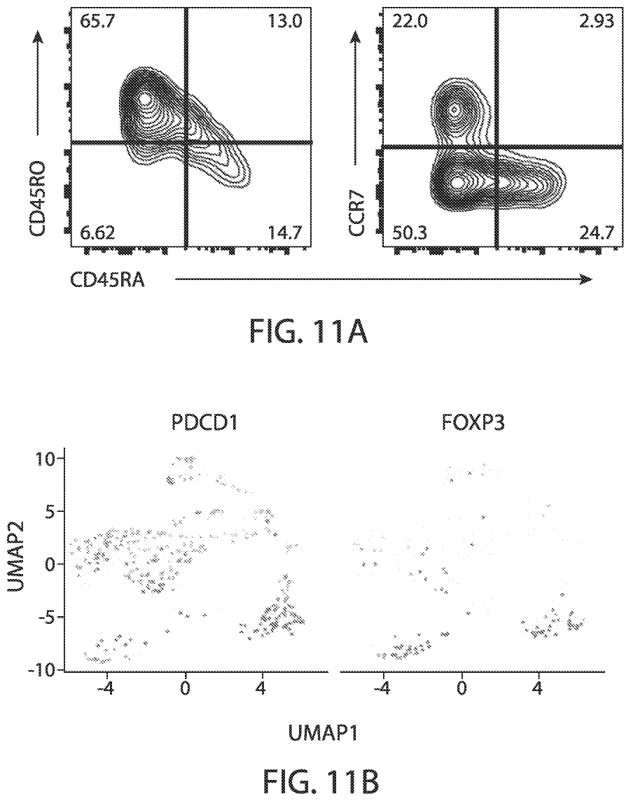

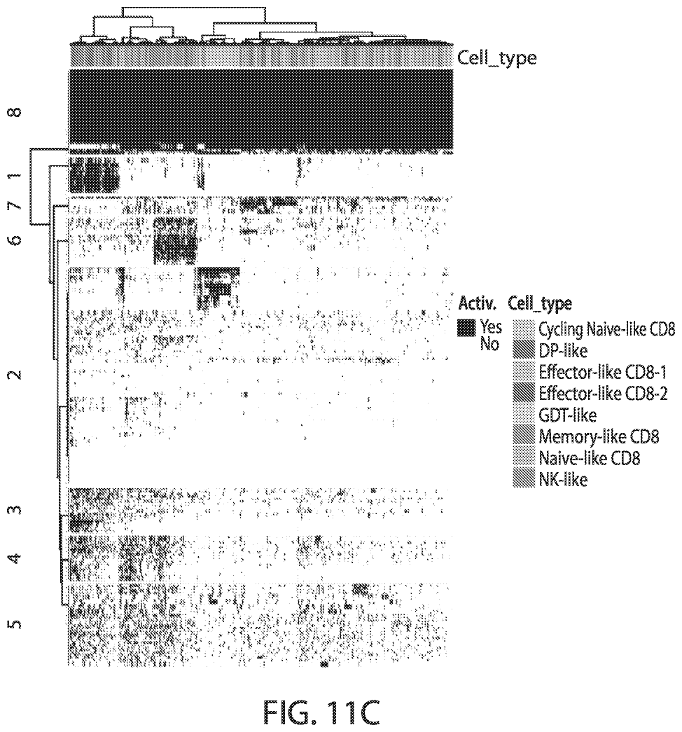

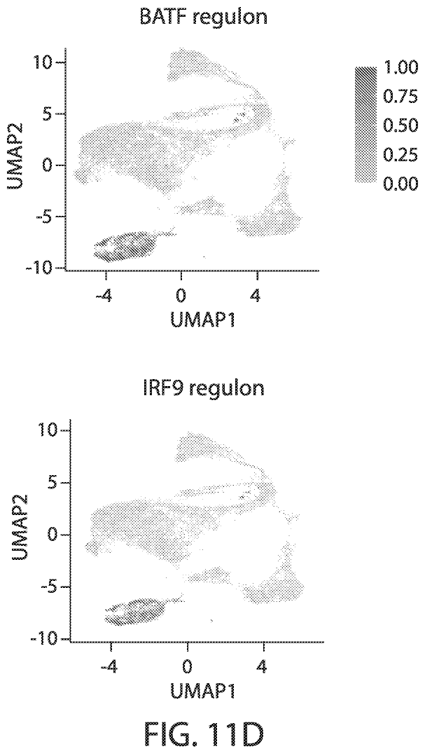

After exiting the thymus, newly produced naive T cells give rise to effector and memory subsets which are characterized by distinct phenotypic and functional features and cumulatively shape T-cell immunity (Kumar et al., 2018). T cells generated from iPSCs have previously been shown to display a naive phenotype (Seet et al., 2017) (Iriguchi et al., 2021), but which T cell subsets can be derived from naive iPSC-T cells is still largely unknown. To answer this question, single-cell RNA sequencing (scRNA-seq) was used to profile the distinct changes and characterize the T cell subsets that arise during T cell differentiation and activation. Upon the completion of EZ-T cell differentiation, the inventors sorted CD45⁺ hematopoietic cells before and after T cell activation and generated 11,131 single-cell transcriptomes. Eight clusters were identified across all samples, with the majority of cells manifesting a T cell fate (CD5+) (FIGS. 5A, 5B). Consistent with previous immunophenotypic analyses, cells were predominantly CD8 SP cells with lower amounts of CD4 SP and DP T cells (FIG. 5B). Further analyses on the signature gene expression profiles revealed only small proportions of innate NK-like (CD56⁺CD5⁻KLRB1⁺) or γδ T cells (TRDC⁺TRGC1⁺). For the mature T cell compartment, the inventors identified two naive-like T cell clusters (CCR7⁺SELL⁺IL2RA^{low}LEF1^{high}) distinguished by their cycling status (Willinger et al., 2006), two effector-like clusters (CCR7⁻SELL⁻GZMB^{high}GZMA^{high}NKG7⁺), and notably, a memory-like T cell cluster that express low levels of cytotoxicity-related genes (GZMB, NKG7) and highly expressed genes that have been linked to memory T cell identities (CCR7⁺SELL⁺IL7R⁺CD2^{high}CCL5^{high}FAS^{high}EOMES^{high}) (Sanders et al., 1988) (Huster et al., 2004; Intlekofer et al., 2005; Margais et al., 2006). The presence of a memory-like T cell subset was further validated by detecting the expression of CD45RA, CD45RA, and CCR7 (FIG. 11A). Moreover, it is known that cytotoxic T cells can express NK cell genes, including inhibitory NK cell receptors that raise the threshold of TCR stimulation and dampen T cell responses (Vivier and Anfossi, 2004). Among the inhibitory NK cell receptors, KLRB1 is expressed by tumor infiltrating effector T cells for several types of human cancer, negatively regulating their antitumor activity (Mathewson et al., 2021). A more recent study further identified KLRB1, together with other NK cell receptors, as a hallmark of exhausted, dysfunctional CAR T cells (Good et al., 2021). In these cell populations, the memory-like T cell cluster expressed lower levels of NK cell genes including KLRB1, which further distinguishes it from the terminally differentiated effector-like T cell clusters (FIG. 5C). In addition to the expression of major marker genes, GSEA analysis indicated a gene expression profile similar to that of memory T cells rather than naive or effector T cells (FIG. 5D). None of the clusters showed substantial expression of inhibitory receptors or regulatory T cell markers (FIG. 11B).

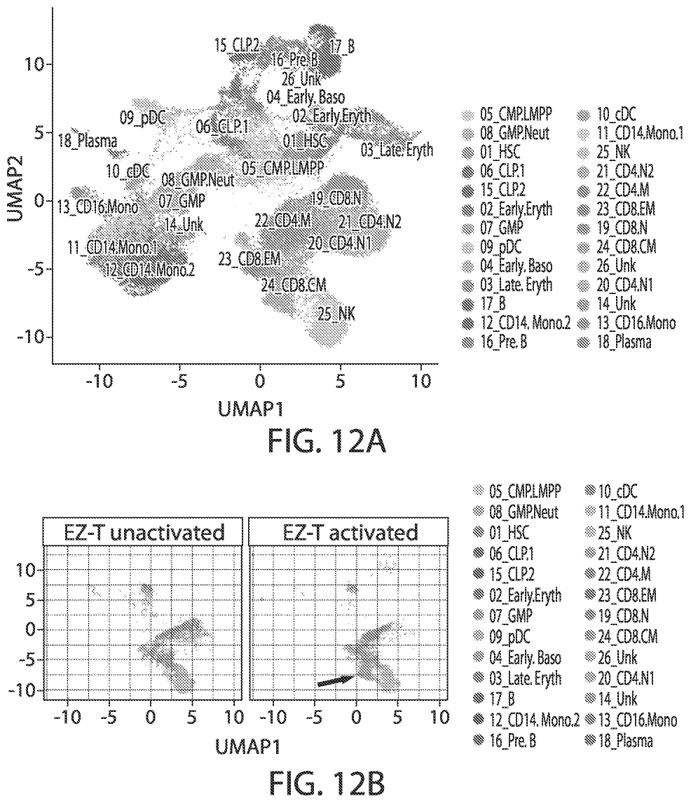

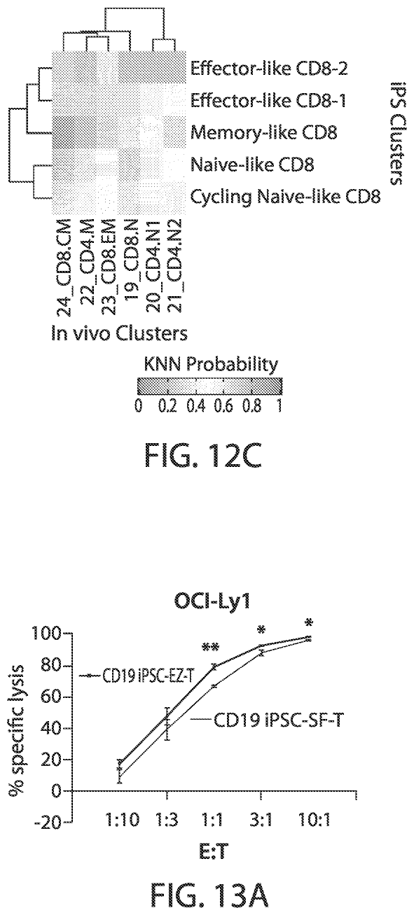

It was next sought to capture the compositional changes in immune cell clusters during T cell expansion. Compared to unactivated cells, T cell expansion resulted in enrichment of mature T cell populations and a concomitant reduction of immature DP T cells and innate-like cells (NK-like cells, γδ T-like cells). Similarly, naive-like T cells were predominantly present in unactivated samples and significantly reduced upon activation. Importantly, T cells displaying a memory-like phenotype were exclusively detected in activated cells after extended expansion, indicating the occurrence of cell fate conversions from naive-like cells into more differentiated subsets (FIGS. 5E, 5F). To examine this, gene regulatory network (GRN) analysis was performed using CellRouter™, aiming to reconstruct the trajectories of cell state transitions from naive to memory-like T cells (Lummertz da Rocha et al., 2018). A series of key transcriptional networks governing the changes in T cell composition was identified, and multiple top-ranked transcriptional regulators, including BATF, LYAR, LITAF, IRF4, and RUNX3 were found, which were previously known to drive the development of long-lived memory T cells (FIG. 5G) (Seo et al., 2021; Chen et al., 2020; Mackay et al., 2013; Harberts et al., 2021) (Wang et al., 2018b). Additionally, Single-Cell rEgulatory Network Inference and Clustering (SCENIC) analysis was performed to map regulons enriched in each annotated cell type (Aibar et al., 2017) (FIG. 11C). Consistent with the CellRouter analysis, SCENIC identified a similar set of regulons associated with the memory-like subset. In particular, regulatory networks that have been linked to the generation and homeostasis of memory-T cells, such as BATF and IRF9 regulons, were exclusively enriched in the memory-like cluster (Kurachi et al., 2014) (Martinet et al., 2015; Seo et al., 2021) (FIG. 11D). Finally, the inventors compared EZ-T cells with a range of hematopoietic cells by mapping the scRNA-seq data on a publicly available reference dataset that includes hematopoietic stem cells, lineage-restricted blood progenitors, and terminally differentiated lymphoid/erythroid/myeloid cells collected from healthy bone marrow and peripheral blood samples (Granja et al., 2019) (FIG. 12A). Almost all the CD45+ cells derived via EZ-T cell differentiation overlap with peripheral blood T cells; cells that represent early HSPCs or other non-lymphoid lineages were barely detectable (FIG. 12B). Consistent with previous analysis, following activation a subset of EZ-T cells emerged as CD8 central memory T cells (FIG. 12B). A cross comparison between iPSC-derived T cells with their in vivo counterparts also indicates that the memory-like CD8 cluster in EZ-T cells closely correlates with peripheral blood central memory CD8 T cells (FIG. 12C). Taken together, iPSC-derived EZ-T cells recapitulate the differentiation of naive T cells that give rise to effector cells and T cell subsets that exhibit a memory-like phenotype.

CAR T Cells Generated from EZ-T Cells Exhibit Enhanced Antitumor Activity

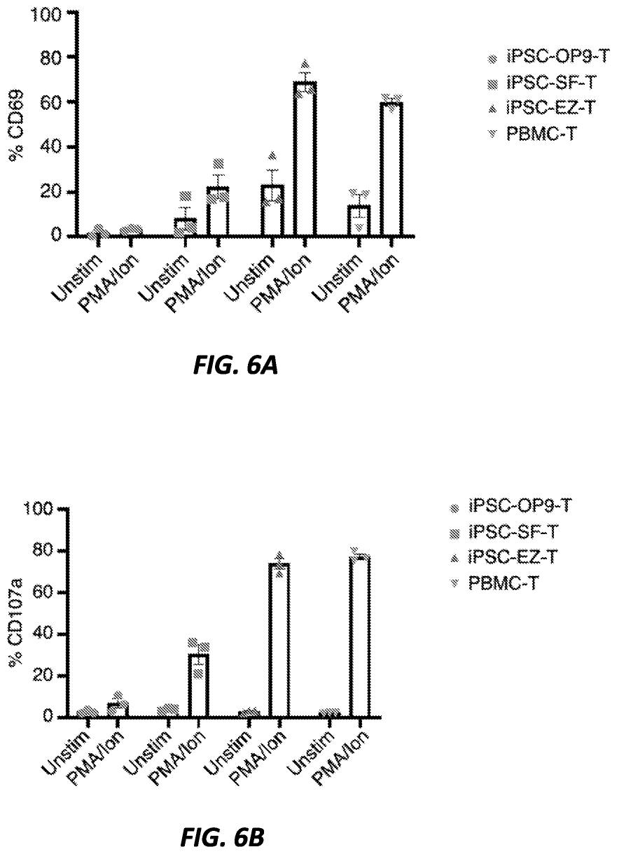

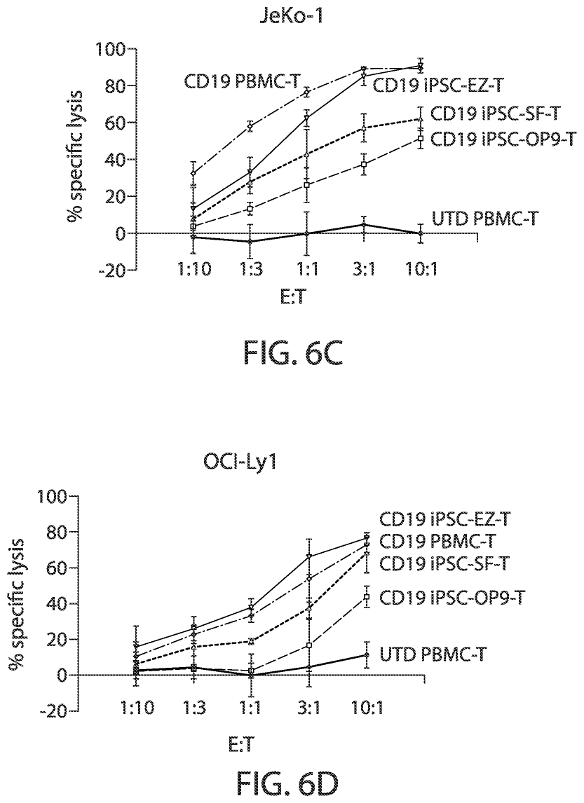

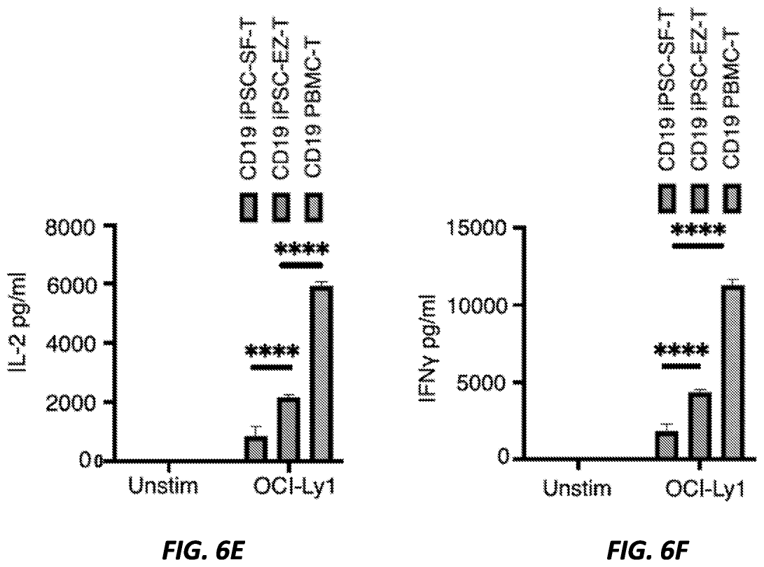

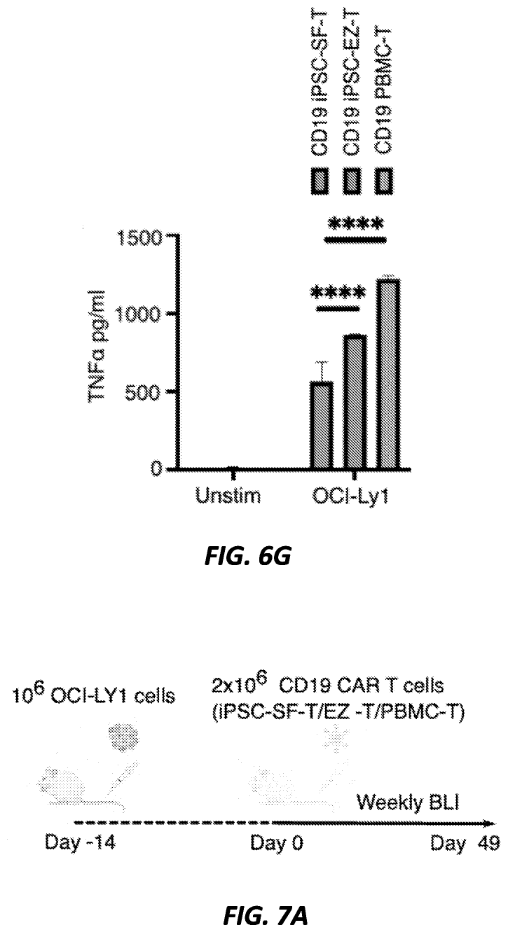

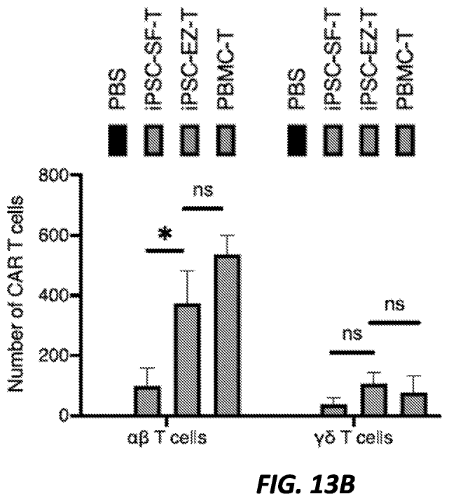

Having shown that EZ-T cells display molecular features similar to those of mature peripheral blood TCRαβ T cells, the inventors next performed functional characterizations of effector cell properties. Compared to iPSC-OP9-T cells or iPSC-SF-T cells, EZ-T cells showed more robust upregulation of CD69 in response to PMA/ionomycin treatment (FIG. 6A). Consistently, EZ-T cell expressed higher levels of CD107a than control iPSC-SF-T cells upon PMA/ionomycin stimulation; the degranulation efficiency was comparable to peripheral blood-derived T cells (FIG. 6B). In light of the EZ-T cells' superior activation/degranulation efficiency, it was next explored whether EZ-T cells could be used to generate CAR T cells with enhanced cytotoxic effector functions. The inventors transduced control iPSC-OP9-T cells, iPSC-SF-T cells EZ-T cells, and donor-derived peripheral blood T cells with anti-CD19 CARs containing a 4-1BB costimulatory domain, and co-cultured with two different types of CD19′lymphoma cells to compare their cytotoxicity profiles. CD19 CAR EZ-T cells caused more efficient specific target cytolysis against both Jeko-1 and OCI-Ly1 cells than CAR T cells derived from control iPSC-OP-9 and iPSC-SF-T cells, and displayed a specific killing capacity as robust as PBMC-T cells (FIGS. 6C, 6D). Cytotoxic assays were also performed using presorted TCRαβ T cells with or without EZH1 knockdown. Similarly, TCRαβ T cells with EZH1 knockdown elicited more efficient killing of target tumor cells (FIG. 13A). Moreover, co-culture with tumor cells triggered EZ-T cells to secrete higher levels of cytokines that are essential for T cell antitumor responses, including IL-2, interferon-γ (IFN-γ), and tumor necrosis factor α (TNFα) (FIGS. 6E, 6F, 6G). Notably, both control SF-T and EZ-T cells produced substantially lower levels of IL-2 and IFN-γ than PBMC T cells, whereas TNFα production was comparable. Since iPSC-derived T cells are predominantly CD8⁺ cytotoxic cells whereas the PBMC T cells include a large proportion of CD4⁺ cells, this observation is consistent with the distinct cytokine production capacity/profile between CD8 and CD4 T cells (Pfizenmaier et al., 1984) (Ngai et al., 2007). Collectively, these data indicate that EZ-T cells exhibit enhanced cytotoxic and cytokine-producing effector functions against tumor cells in vitro.

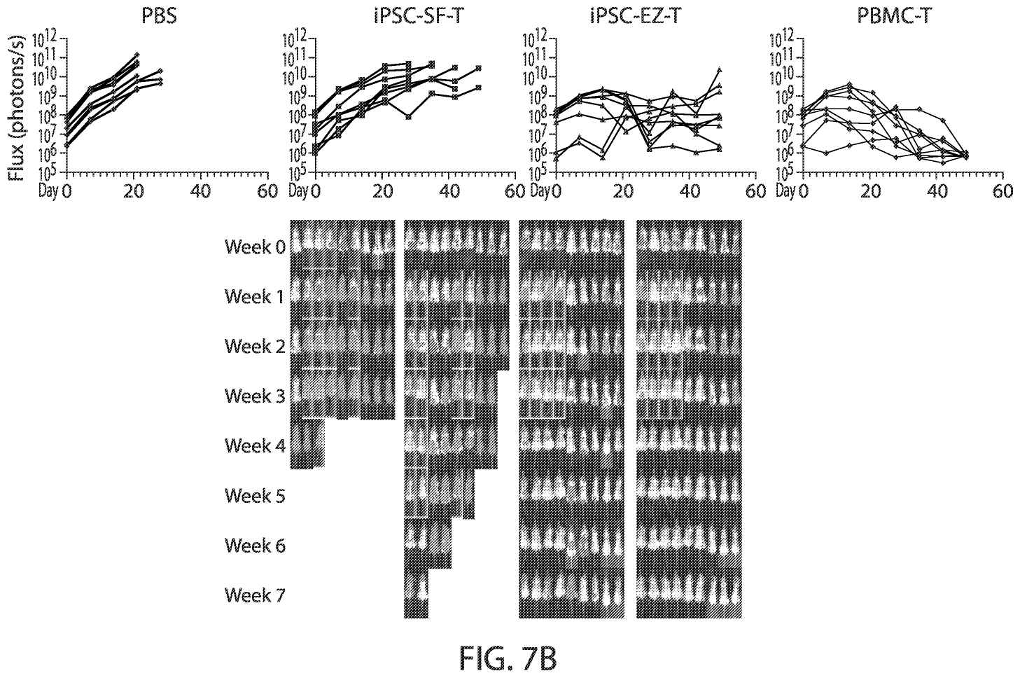

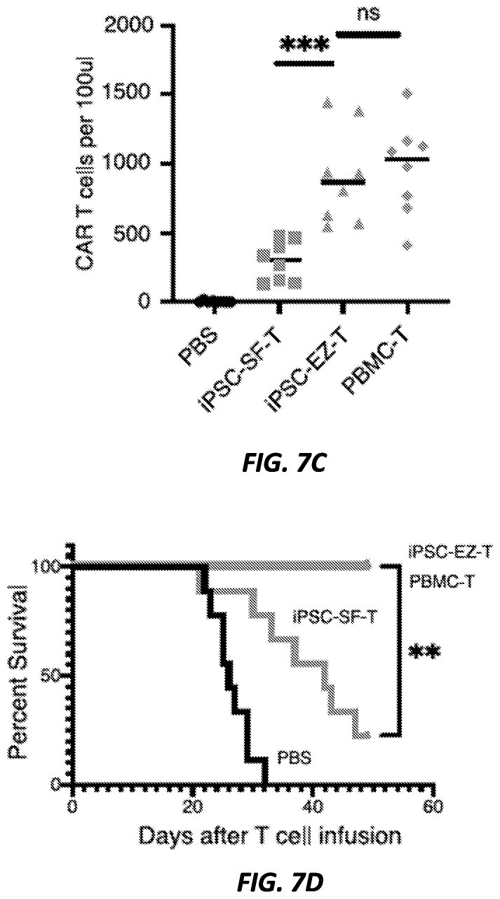

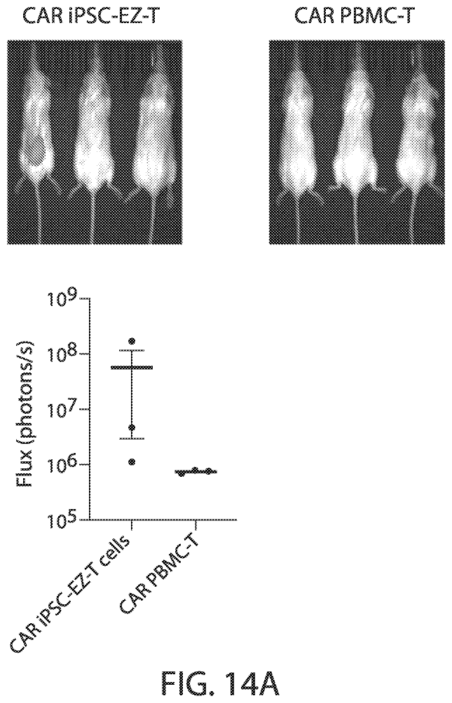

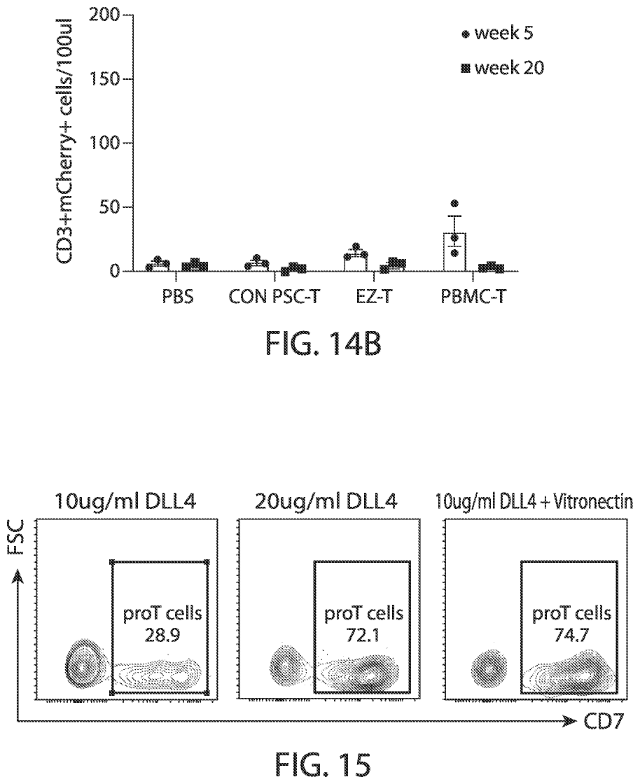

To further evaluate the efficacy of EZ-T cell-derived CAR T cells, a xenograft mouse model was established by intravenously injecting luciferase-expressing diffuse large B-cell lymphoma (DLBCL) cells (OCI-Ly1) into immunodeficient Non-obese diabetic-SCID IL2Rgamma^{null} (NSG) mice. These animals were then treated with PBS, CD19 CAR iPSC-SF-T cells, CD19 CAR EZ-T cells, or PBMC-derived CD19 CAR T cells and subjected to weekly bioluminescence imaging (BLI) to assess tumor burden (FIG. 7A). Although CAR T cells generated from iPSC-SF-T cells suppressed tumor growth, they failed to eradicate tumor cells in any animal after 7 weeks. Notably, CAR EZ-T cells displayed significant improvement of efficacy and were capable of eradicating tumor cells and caused complete remissions in some animals (FIG. 7B). Consistent with improved tumor clearance, more CAR EZ-T cells were detected in peripheral blood than control CAR iPSC-SF-T cells 3 weeks after injection (FIG. 7C), and most circulating EZ-T cells expressed αβTCR and not γδ TCR (FIG. 13B), indicating that the enhanced persistence of EZ-T cells is largely due to the enrichment of TCRαβ T cells. As a result, though some mice showed persistent BLI signal intensity, injection of CAR EZ-T cells produced comparable survival rates to PBMC CAR T cell treatment for the 7 week duration of the experiment (FIGS. 7D, 14A). To exclude that EZH1 repression may cause abnormal expansion of EZ-T cells, the inventors administrated both control and EZ-T cells into healthy mice and monitored the presence of CAR T cells over a long time period. Without simultaneous tumor engraftment, CAR T cells were barely detectable in peripheral blood after 5 weeks, and these animals remained healthy and were free of human T cells after 20 weeks (FIG. 14B). In summary, when engineered with anti-CD19 CARs, EZ-T cells elicit superior antitumor effects than control iPSC-SF-T cells lacking EZH1 knockdown both in vitro and in vivo.

The identification of signaling pathways that are essential for T cell development has led to the design of protocols that allow the generation of T cells in vitro from human pluripotent stem cells (Holmes and Zúñiga-Pfücker, 2009) (Timmermans et al., 2009) (Seet et al., 2017). However, past in vitro differentiation approaches have been plagued by dependency on mouse stromal cells as well as a failure to recapitulate the terminal stages of T effector cell maturation, thereby limiting their clinical application in adoptive immunotherapy. The generation of definitive (adult-type) hematopoietic stem cells (HSCs) with lymphoid potential and differentiation of functional T cells from iPSCs has proven difficult, as in vitro differentiation of iPSCs tends to default into the production of embryonic cell types (Doulatov et al., 2013; Sugimura et al., 2017). Past studies have demonstrated that iPSC-derived T cells resemble innate-like γδ T cells and are not as robustly functional as primary αβ T cells, again indicating that the ability to recapitulate the mechanisms underlying commitment and progression of T cell development is incomplete. Screening of critical epigenetic modifying enzymes during the generation of HSPCs discovered that EZH1 plays a central role in regulating multipotency and lymphoid potential in embryonic blood progenitors (Vo et al., 2018). As a component of PRC2, EZH1 modulates chromatin accessibility by mediating histone H3 lys27 trimethylation (H3K27me3) (Shen et al., 2008). During embryonic hematopoiesis, EZH1 represses the transcription of genes associated with definitive hematopoietic fates, and EZH1 deficiency in genetically engineered mice promotes the precocious emergence of bona fide HSC and lymphoid progenitors (Vo et al., 2018). A recent study in the zebrafish model further showed that EZH1 suppresses HSPC formation by regulating HE commitment. Specifically, EZH1 enhances Notch signaling to facilitate arterial gene expression at the expense of HE specification and HSPC development. As a result, knockdown of EZH1 unlocks definitive hematopoiesis and leads to enhanced production of multipotent HSPCs with lymphoid potential (Soto et al., 2021).

In light of the repressive function of EZH1 in lymphopoiesis, the inventors investigated the impact of EZH1 knockdown during in vitro T cell differentiation. Although 3D thymic-like culture systems have been established to mimic mouse or human T cell development and yield iPSC-derived T cells that display a mature phenotype, these methods rely on engineered stromal cells or fetal thymic cells derived from mouse (Vizcardo et al., 2018; Montel-Hagen et al., 2019) (Wang et al., 2022). Therefore, the inventors developed a stroma-free system that supports efficient T cell differentiation without using mouse-derived feeder cells. Such a strategy based on immobilized Notch ligands has recently been employed to induce the iPSC-derived CD34⁺ HSPCs to differentiate into CD3⁺TCRαβ⁺CD8αβ T cells that are immunophenotypically similar to prior iPSC-SF-T cells (Iriguchi et al., 2021) (Trotman-Grant et al., 2021). However, whether such stroma-free differentiation could support normal TCR rearrangement remained unclear. Here it is shown that the stroma-free system can faithfully recapitulate T cell development by differentiating non-T cell-derived iPSCs (without pre-rearranged TCRs) into T cells with a high degree of TCR diversity. Compared to iPSC-OP9-T cells, iPSC-SF-T cells exhibit a marginally more mature phenotype, similar to T cells differentiated from CB CD34⁺ HSPCs. Moreover, by avoiding the cumbersome co-culture with mouse feeder cells, the stroma-free protocol minimizes batch-to-batch variation that can confound phenotypic characterization. Leveraging this new stroma-free differentiation platform, the inventors further demonstrate that repression of EZH1 expression during lymphoid specification facilitates T cell differentiation from human iPSCs and leads to robust generation of developmentally mature iPSC-derived T cells. Importantly, while iPSC-SF-T cells exhibit some innate-like phenotypes that have been previously reported in OP9-stroma dependent iPSC differentiation systems (Themeli et al., 2013), EZ-T cells display molecular signatures resembling peripheral blood TCRαβ T cells. These results are in agreement with the hypothesis that EZH1 knockdown produces definitive progenitors that preferentially support adult-like lymphopoiesis rather than the generation of immature primitive embryonic lymphoid cells. Such a mechanism has also been supported by recent findings that hPSC-derived CD34⁺ progenitors with a primary phenotype, defined by restricted hematopoietic potential, produce fetal-like NK cells via in vitro differentiation (Dege et al., 2020). In contrast, the multipotent CD34+ progenitors with full lymphoid potential are capable of giving rise to adult-like NK cells that are functionally distinct from their fetal counterparts (Dege et al., 2020).

Another focus of this study was to test the impact of EZH1 repression on the functional properties of iPSC-derived T cells and to evaluate the potential of using EZ-T cells for adoptive T cell immunotherapy. iPSC-derived T cells were engineered with anti-CD19 CARs and assessed their antitumor capacities. Compared to CAR T cells generated from iPSC-SF-T cells, CD19-CAR EZ-T cells exhibit superior antitumor activity measured by tumor cell killing and cytokine production against different types of CD19⁺ tumor cells in vitro, and elicit more efficient tumor clearance in a xenograft mouse model. Notably, scRNA-seq analysis revealed that EZ-T cells, after activation, give rise to a subset of T cells that express relatively low levels of cytotoxicity genes and high levels of memory T cell signature genes. Such an observation is consistent with previous findings that in vitro T cell stimulation allows faster effector/memory commitment than physiological T cell differentiation/expansion (Li and Kurlander, 2010) (McLellan and Ali Hosseini Rad, 2019).

Trajectory analyses based on the activity of GRNs also identified transcriptional regulators that drive the conversion of naive-like EZ-T cells into memory-like cells. Among these networks are BATF, IRF4/9, and RUNX3, all of which are known to promote T cell longevity or prevent exhaustion (Martinet et al., 2015) (Huber et al., 2017; Wang et al., 2018b) (Istaces et al., 2019; Seo et al., 2021). This is of particular interest because a growing body of evidence has shown that enrichment of memory T cells rather than terminally differentiated effector cells correlates with superior T cell persistence and improved clinical outcomes in adoptive T cell therapies (Gattinoni et al., 2005) (Klebanoff et al., 2005) (Stark et al., 2009) (Fraietta et al., 2018b) (Fraietta et al., 2018a). Therefore, the presence of a memory-like T cell subset may contribute to the enhanced persistence and antitumor activity of EZ-T cells in the DLBCL model.

New cell engineering strategies have profoundly changed the paradigm of CAR T cell therapy. Multiple studies have generated CAR T cells with disrupted endogenous TCR to avoid the risk of graft-versus-host disease (GVHD) inherent in allogeneic T cell therapy (MacLeod et al., 2017) (Eyquem et al., 2017) (Georgiadis et al., 2018). Recent success in producing hypoimmunogenic iPSCs that can evade immune rejection has further enhanced the prospects for off-the-shelf, universal iPSC-derived CAR T cells (Han et al., 2019) (Deuse et al., 2019) (Wang et al., 2021). Given the fact that the stroma-free system produces iPSC-derived T cells expressing a highly diverse TCR repertoire, genetic ablation of the endogenous TCR will be required before allogeneic transplantation. Compared to primary T cells, iPSCs are more amenable to genetic manipulations and could facilitate the engineering of “armored” CAR T cells that can secrete specific cytokines or checkpoint inhibitor antibodies to overcome the suppressive tumor microenvironment (Pegram et al., 2015) (Adachi et al., 2018) (Rafiq et al., 2018). In addition to T cell-based immunotherapies, NK cells have also shown great promise in the treatment of both blood and solid tumors, and are currently being tested in multiple clinical trials (Basar et al., 2020) (Liu et al., 2021). Compared to T cells, NK cell cytotoxicity is not constrained by MHC recognition and could target tumor cells that are resistant to T cell killing. Moreover, NK cells are less prone to GVHD and CAR-associated toxicity (Ruggeri et al., 2002; Liu et al., 2020). Multiple studies have successfully generated iPSC-derived NK cells that elicit antitumor activities (Knorr et al., 2013) (Zeng et al., 2017) (Li et al., 2018) (Woan et al., 2021). How EZH1-mediated regulation of lymphoid commitment might affect NK cell differentiation from iPSCs remains an open question. Here it is shown that iPSC-derived EZ-T cells, which are molecularly most similar to T cells from adult peripheral blood display more mature phenotypes and enhanced antitumor activity and consequently might serve as an ideal source for the production of CAR-T cells.

### Methods

### Cell Line

Human 1157.2 iPSCs were maintained on Matrigel matrix (Corning, 354277) using Stemflex media (Gibco, A3349401) in 5% CO² at 37° C.

### In Vivo Tumor Xenograft Model

NOD-scid IL2Rgamma^{null} (NSG) mice (Jackson Laboratories, 005557) were housed at the Boston Children's Hospital animal care facility following institutional guidelines. 8 to 12-week-old male and female mice were intravenously injected with 1×10⁶ OCI-Ly1 DLBCL tumor cells expressing green firefly luciferase. The inoculated animals were subjected to bioluminescence imaging (BLI) using the IVIS 200 system (PerkinElmer) twice per week following intraperitoneal injections of Vivoglo™ luciferin (Promega, P1043) at 150 mg/kg body weight. After two weeks, animals with substantial tumor cell engraftment (Mean total flux>5×10⁵ photons/sec) were randomly assigned into four groups and intravenously injected with PBS (untreated) or 2×10⁶ CAR T cells generated from control iPSC-SF-T, iPSC-EZ-T, or PBMC-T cells. Tumor burden was measured by BLI weekly, and images were processed and analyzed using Aura imaging software (Spectral Instruments Imaging). To monitor CAR T cell persistence, peripheral blood cells were collected via retro-orbital bleeding after 3 weeks of T cell injections, and absolute numbers of CAR T cells were determined by flow cytometry analysis. All animal experiments were performed under protocols approved by the Institutional Animal Care and Use Committee of Boston Children's Hospital.

Culture of iPSCs and Generation of CD34⁺ HE

Human 1157.2 iPSCs were maintained on Matrigel-coated plates using Stemflex™ media (Gibco, A3349401) and transferred to mouse embryonic fibroblast (MEF) feeder cells (Gibico, A34181) prior to differentiation. After one week of culture of on MEFs, iPSCs were collected for the generation CD34⁺ HE cells using a previous described protocol (Ditadi et al., 2015). Briefly, iPSC colonies were scraped and transferred to ultra-low attachment plates and cultured with aggregation media contains BMP4 (10 ng/ml, day 0-2), bFGF (5 ng/ml, day 1-2), CHIR99021 (3 μM, day 2), and SB431542 (6 μM, day2) to allow EB formation. From day 4, EBs were cultured with StemPro-34 media (Gibco, 10639011) supplemented with VEGF (15 ng/ml, day3-8), bFGF (5 ng/ml, day3-8), SCF (50 ng/ml, day 6-8), EPO (day6-8), IL-6 (day6-8), IL-1 (day6-8), and IGF-1 (day6-8). After 8 days of culture, EBs were dissociated using EB Dissociation Kit (Miltenyi, 130096348), and HE cells were isolated by magnetic-activated cell sorting (MSCS) using the human CD34 Microbead Kit (Miltenyi, 130046702).

Differentiation of iPSCs into T Cells

24 well non-tissue culture treated plates were coated with 10 μg/ml recombinant human DL4-Fc protein (Life Technologies, A42510) and 2 μg/ml VCAM-1 (Shukla et al., 2017) (R&D, 862VC100) in PBS for 2 hours at 4° C. CD34+ HE cells (1×10⁵ cells/well) were then seeded on DL4-coated plates and cultured in SFEMII (StemCell Tech, 09605) media supplemented with 10% BIT serum substitute (StemCell Tech, 09500), 2 mM L-Glutamine (Gibco, 35050061), 1 mM sodium pyruvate (Gibco, 11360070), 50 μg/ml 2-phospho-L-ascorbic acid (Huijskens et al., 2014) (sigma, 49752), 55 μM 2-Mercaptoethanol (Gibco, 21985023), 1 mM None-essential amino acids (Gibco, 11140050), 1% penicillin-streptomycin (Corning, 30002CI), 30 ng/ml SCF, 20 ng/ml FLT3, 30 ng/ml IL-7, 5 ng/ml IL-3 (first week only), and 5 ng/ml TPO for 2 weeks to induced the differentiation into proT cells. For the production of EZ-T cells, EZH1 knockdown was performed via lentiviral transduction (MOI=5) after 24 hours of HE seeding. After 2 weeks of differentiation, SCF and TPO were withdrawn from the media, and the proT cells were replated into new DL4-coated plates and cultured in the presence of 20 ng/ml FLT3 and 15 ng/ml IL-7 till week 5, followed by 1 week of treatment with CD3/CD28 T activator (StemCell Tech, 10971) and 5 ng/ml IL-15 to induced SP T cells. During T cell differentiation, half media changes were conducted every 2-3 days. T cell differentiation using the OP9-DL1 co-culture system was conducted following previous reports (Holmes and Zúñiga-Pfücker, 2009) (Themeli et al., 2013). Briefly, day 8 CD34⁺ HE cells were seeded on OP-DL1 stromal cells and co-cultured with OP9 media (α-MEME, 20% FBS, 1% penicillin-streptomycin, 1 mM None-essential amino acids, 2 mM L-Glutamine, 10 μM 2-Mercaptoethanol, 50 μg/ml 2-phospho-L-ascorbic acid) supplemented with 10 ng/ml SCF, 5 ng/ml FLT3, and 10 ng/ml IL-7. Cells were mechanically dissociated and filtered through 40 μm strainers to be seeded on new stromal cells every five days.

CRISPR Interference in iPSs

gRNA targeting the TSS of EZH1 was pulled from the Broad Institute's genome-wide Dolcetto library (Addgene #92385, Library Set A) (Sanson et al., 2018) and ordered as oligos from IDT. gRNA oligos (GGTGAGTGAGTAAACAAGCC (SEQ ID NO: 57)) were annealed, phosphorylated and cloned by Golden Gate Assembly with BsmBI into a modified CROPseq-Zeo vector constitutively expressing mNeon™ and a modified gRNA scaffold sequence as previously described (Dang et al., 2015). Lentiviruses for each gRNA vector was generated by transfection of pMD2.G (Addgene #12259), psPAX2 (Addgene #12260), and the successfully cloned CROPseq transfer plasmid (2:3:4 ratio by mass and 3 ug total plasmid) into HEK293FT cells using Lipofectamine™ 3000 (Thermo Fisher L3000015). Viral supernatant was harvested 48 hours after transfection and filtered through 0.45 μm PVDF filters (Millipore SLHVRO4NL). Human 1157.2 iPSC line expressing a doxycycline-inducible dCas9-KRAB-2A-mCherry cassette (from Boston Children's Hospital Stem Cell Core) were singularized with TrypLE, plated in a matrigel-coated 6-well plate at 165,000 cells per well, and then infected with individual CROPseq lentiviruses at MOI=0.25 with 8 ug/mL polybrene. The following day the media was replenished and the cells were selected with 200 μg/mL zeocin (ThermoFisher R25001) for 24 hours. Selected hiPSCs were scaled up, banked, and used for downstream differentiation.

### Immunoblot

Whole cell lysates were collected using NP-40 lysis buffer (Invitrogen, FNN0021) with protease and phosphatase inhibitor cocktail (Thermo Scientific, 78443). 50 μg total protein was separated by SDS-PAGE using Any kD™ Mini-protean TGX precast polyacrylamide gels (BioRad, 4569033), and transferred to PVDF membranes using the Trans-Blot Cell Transfer System (BioRad). Membranes were incubated overnight with antibodies against TBP (Cell Signaling, 85155, 1:1000), EZH1 (Abcam, ab64850, 1:1000) at 4° C., followed by incubation with horseradish peroxidase-conjugated secondary antibodies (1:2000) for 1 hour at room temperature. Chemiluminescence was detected using SuperSignal™ West Pico Plus Chemiluminescent substrate (Thermo Scientific, 34579).

### Quantitative Real-Time PCR Analysis

RNA extraction and removal of genomic DNA was performed using the RNeasy Mini Kit (Qiagen, 74104). First strand cDNA was synthesized using Maxiam™ First Strand cDNA Synthesis Kit (Thermo Scientific, K1641). Quantitative real-time PCR was performed on a QuantStudio™ 7 Flex Real-Time PCR machine (Applied Biosystems, 4485701) using Power SYBR Green PCR Master Mix (Applied Biosystems, 4367659 following the manufacturer's directions. The following oligonucleotides were used:

DNTT forward primer:
(SEQ ID NO: 49)
5′-CAGAGCGTTCCTCATGGAGCTG-3′;
DNTT reverse primer:
(SEQ ID NO: 50)
5′-GTGCTTGAAGCCACTCCAGAAC-3′;
EZH1 forward primer:
(SEQ ID NO: 51)
5′-CACCACATAGTCAGTGCTTCCTG-3′;
EZH1 reverse primer:
(SEQ ID NO: 52)
5′-AGTCTGACAGCGAGAGTTAGCC-3′;
GAPDH forward primer:
(SEQ ID NO: 53)
5′-ACCCAGAAGACTGTGGATGG-3′;
GAPDH reverse primer:
(SEQ ID NO: 54)
5′-TTCAGCTCAGGGATGACCTT-3′;
EZH2 forward primer:
(SEQ ID NO: 55)
5′-GACCTCTGTCTTACTTGTGGAGC-3′;
EZH2 reverse primer:
(SEQ ID NO: 56)
5′-CGTCAGATGGTGCCAGCAATAG-3′.

### Flow Cytometry

Cells were stained with PI/DAPI (BD Biosciences, RUO) and antibodies at 1:100 dilution in PBS with 2% FBS for 30 min at room temperature in the dark. BD LSRII and BD FACSAria™ JJ were used for flow cytometry analysis and cells sorting. Compensation was performed by automated compensation with anti-mouse Igk and negative beads (BD Biosciences) using the BD FACSDiva™ software. The following human antibodies were used: CD45RA-BV510 (BD Biosciences, H100), CD45RO-PE (BD Biosciences, UCHL1), CCR7-APC (BD Biosciences, 2-L1-A), CD3-PE/Cy7 (Biolegend, SK7), CD8α-BV421 (BD Biosciences, RPA-T8), TCRαβ-APC (Biolegend, IP26), TCRγ6-PE (Biolegend, B1), CD7-PE (BD Biosciences, M-T701), CD5-BV510 (BD Biosciences, UCHT2), CD4-PE/Cy5 (Biolegend, RPA-T4), CD8β-APC (Miltenyi, REA715), CD45-APC/Cy7 (BD Biosciences, 2D1), CD279-BV421 (BD Biosciences, EH12.1), CD366-APC (BD Biosciences, F38-2E2), CD223-PE (BD Biosciences, T47-530), CD107a-PE (Biolegend, LAMP-1), CD69 APC (Biolegend, FN50), CD33-APC (Biolegend, WM53).

### Bulk RNA-Seq and Data Analysis

Total RNA samples were isolated from iPSC-derived CD3⁺ cells or PBMC NK/T cells using a column assay with the DNase treatment (Direct-zol MicroPrep, ZYMO). Quantity and quality of the RNA were evaluated using the nanodrop machine and RNA screen tape. High quality RNA (Both 280/260 and 230/260 over 1.7 with RNA integrity number >7) underwent ribosomal RNA depletion and then library construction. For regular gene expression analysis, adaptor trimmed reads from the sequencer were mapped to the human genome, quantified, and analyzed using seq data analysis tools (Cutadapt, Bowtie, TopHat, HTSeq, R, and edgeR). The RNA-seq data is available in GEO database (GSE195667). Portions of this research were conducted on the 02 High Performance Compute Cluster, supported by the Research Computing Group, at Harvard Medical School.

### TCR Repertoire Analysis

CD3+ T cells were FACS-isolated from control PBMC T cells or iPSC-derived T cells, followed by gDNA extraction using the Qiagen DNeasy Blood & Tissue Kit (Qiagen, 69504). DNA samples were then subjected TCRB sequencing via immunoSEQ assay. (Adaptive Biotechnologies). TCR rearrangements, Vβgene usage, and CDR3 length were analyzed using the immunoSEQ Analyzer 3.0 software (Adaptive Biotechnologies).

### Single-Cell RNA-Sequencing and Data Analysis

T cells were FACS-isolated for DAPI-CD45+ expression following in vitro differentiation and/or activation. Single-cell suspensions were loaded onto a Chromium Single Cell Chip (10× Genomics) according to the manufacturer's instructions for a target recovery of ˜6000 cells per lane. Each sample was loaded into two lanes to serve as technical replicates for scRNA-Seq library preparation. Libraries were then prepared per the 10× scRNA-Seq v2 protocol in parallel for all conditions. Final 10× scRNA-Seq libraries were assayed via an Agilent High Sensitivity dsDNA Bioanalyzer, normalized, pooled and shallow sequenced on a MiniSeq™ to quantify the number of high confidence cell barcodes. The libraries were then renormalized per the distribution of reads/library from the MiniSeq™ run and deep sequenced on a NextSeq™ to a depth of 50,000 reads per cell barcode.

Sequencing libraries were computationally demultiplexed and the data were aligned to the GRCh38 reference genome using cellranger v2.1 (10× Genomics). CellRouter™ was used for quality control and downstream analysis (Lummertz da Rocha et al., 2018). Cell by gene count matrices of all samples were combined to a single gene expression matrix. Cells with 500-3000 detected genes and expressing <10% mitochondrial genes as well as genes expressed in >25 cells were retained for downstream analysis. The final dataset after quality control was composed of 10907 cells, with a median of 1,456 genes detected per cell. Variation caused by mitochondrial gene expression and sample replicates were regressed out. All genes passing QC metrics were used for principal component analysis. Clustering analysis was performed using parameters k=150 and 30 principal components. UMAP analysis was performed using 10 principal components. Cluster annotation was performed using differential expression and marker genes. Gene signatures, as well as ranked gene lists ordered by log fold change for GSEA analysis, were generated by testing for differential expression of a cluster/cell type against all other cells using a Wilcox test, as implemented in CellRouter™. Trajectory analysis and calculation of GRN scores was performed with CellRouter™. SCENIC analysis was used to identify regulons enriched in each annotated cell type (Aibar et al., 2017). Data generated by Granja et al. (Granja et al., 2019) were downloaded from https://github.com/GreenleaffLab/MPAL-Single-Cell-2019 and converted into a FUSCA object (Lummertz da Rocha et al., 2022). FUSCA was used to perform UMAP analysis and prepare input parameters for reference mapping using Symphony (Kang et al., 2021).

### T Cell Activation and Degranulation

Control T cells generated from primary PBMC T cells of heathy donors and iPSC-T cells were treated with T cell stimulation cocktail (Invitrogen, 00497093) containing phorbol 12-myristate 13-acetate (PMA) and ionomycin for 6 hours. Percentage of iPSC cells that express CD3 and CD69 was measured by flow cytometry to determine T cell activation. Similarly, percentage of CD8⁺CD3⁺ iPSC cells that express CD107a was measured by flow cytometry to detect T cell degranulation.

### CAR T Cell Functional Assays

Control PBMC T cells and iPSC-derived T cells were activated (day 0) using anti-CD3/CD28 Dynabeads (Gibco, 11131D) or CD3/CD28 T activator (StemCell Tech, 10971), followed by transduction with a lentiviral vector encoding the CD19-CAR 24-hours later (Scarfo et al., 2018). T cells were cultured in RPMI media containing 10% fetal bovine serum, penicillin, streptomycin and supplemented with 20 IU/ml rhIL-2 beginning on day 0 of culture and were maintained at a constant cell concentration of 0.5×10⁶/mL by counting every 2-3 days. On day 10 cells were de-beaded (when using Dynabeads) and used for assays. For cytotoxicity assays, control PBMC or iPSC-T cells were co-cultured with luciferase-expressing Jeko-1 or OCI-Ly1 tumor cells at the indicated ratios for 18 hours. Luciferase activity was measured with a Synergy Neo2 luminescence microplate reader (Biotek). Percentage of specific lysis was calculated as (total RLU/target cells only RLU)×100. Cell-free supernatants were collected for cytokine release assay. Levels of cytokines were measured using a LEGENDplex Multiplex Assay Kit (Biolegend, 741030) following manufacturer's instructions.

### QUANTIFICATION AND STATISTICAL ANALYSIS

The significance of the difference between control and experimental results generated from in vitro assays was determined by two tailed student's T test, and P<0.05 was considered statistically significant. For in vivo experiments, survival results were presented in a Kaplan-Meier survival plot and compared by log-rank (Mantel-Cox) test. Statistic parameters in each experiment (value of n, SEM, or SD) are described in the figure legends.

1. A method for generating CD3+ T cells, the method comprising:
(a) contacting a CD34+ hemogenic endothelial (HE) cells with a CD5+CD7+ differentiation medium comprising interleukin-3 (IL-3) under conditions and for a sufficient time to generate CD5+CD7+ T cell progenitor cells,
(b) contacting the CD5+CD7+ proT cells with a CD3+ T cell differentiation medium under conditions and for a sufficient time to generate CD3+ T cells.

2. The method of claim 1, wherein the concentration of IL-3 is 1-10 ng/mL;
wherein the concentration of IL-3 is 5 ng/mL; and/or
wherein the CD34+ HE cells are contacted with IL-3 for about one week.

3-4. (canceled)

5. The method of claim 1, wherein the CD5+CD7+ differentiation medium further comprises at least one of: stem cell factor (SCF), FLT-3, IL-7 and thrombopoietin (TPO);
wherein the CD5+CD7+ differentiation medium further comprises each of stem cell factor (SCF), FLT-3, IL-7 and thrombopoietin (TPO); and/or
wherein the concentration of SCF is 5-50 ng/mL, and/or the concentration of FLT-3 is 5-30 ng/mL, and/or the concentration of IL-7 is 10-50 ng/mL, and/or the concentration of TPO is 1-10 ng/mL.

6-7. (canceled)

8. The method of claim 1, wherein the CD3+ T cell differentiation medium comprises Fins related receptor tyrosine kinase-3 (FLT3), and interleukin-7 (IL-7); and/or
wherein the concentration of IL-7 is 1-30 ng/mL, and/or the concentration of FLT-3 is 5-30 ng/mL.

9. (canceled)

10. The method of claim 1, wherein the yield of CD3+ T cells is higher than a substantially similar method lacking IL-3 in step (a).

11. The method of claim 1, wherein the CD34+ HE cells undergo endothelial-to-hematopoietic transition (EHT).

12. The method of claim 1, wherein step (a) is performed for at least 1 week;
wherein step (a) is performed for 2 weeks; and/or
wherein step (b) is performed for at least 1 week.

13-14. (canceled)

15. The method of claim 1, wherein the CD34+ HE cells are cultured in the presence of a Notch ligand;
wherein the Notch ligand is attached to a solid surface; and/or
the Notch ligand is attached to a cell culture dish.

16-17. (canceled)

18. The method of claim 15, wherein the Notch ligand is not expressed by a stromal cell;
wherein the method does not comprise co-culturing with a stromal cell expressing a Notch ligand; and/or
wherein differentiating the hemogenic endothelium in the presence of a Notch ligand does not comprise co-culturing with OP9-DL1 cells or OP9-DL4 cells.

19-20. (canceled)

21. The method of claim 15, wherein the Notch ligand is selected from the group consisting of Delta-like-1 (DLL1), Delta-like-4 (DLL4), immobilized Delta1^{ext-IgG}, and immobilized Delta4^{ext-IgG};
wherein immobilized Delta1^{ext-IgG} consists of an extracellular domain of human Delta-like-1 fused to the Fc domain of human IgG1;
wherein the concentration of DLL4 is in the range of 1-30 μg/mL;
wherein the concentration of DLL4 is in the range of 5-25 μg/mL; and/or
wherein the concentration of DLL4 is 10 μg/mL or 20 μg/mL.

22-25. (canceled)

26. The method of claim 1, further comprising culturing the CD34+ HE cells in the presence of vitronectin;
wherein the concentration of vitronectin is in the range of 1-20 μg/mL; and/or
wherein the concentration of vitronectin is 10 μg/mL.

27-28. (canceled)

29. The method of claim 1, wherein the CD5+CD7+ T cell differentiation medium and/or the CD3⁺-T-cell-differentiation media are serum-free.

30. A method for generating CD3+ T cells comprising:
(a) contacting CD34+ hemogenic endothelial (HE) cells with a first differentiation medium comprising interleukin-3 (IL-3), stem cell factor (SCF), FLT-3, IL-7 and thrombopoietin (TPO) under conditions and for a sufficient time to generate CD5+CD7+ T cell progenitors,
(b) contacting the CD5+CD7+ T cell progenitors with a second differentiation medium comprising FLT-3 and IL-7 under conditions and for a sufficient time to generate CD3+ T cells.

31-32. (canceled)

33. The method of claim 1, further comprising a step of generating CD34+ hemogenic endothelium from a population of pluripotent stem cells, optionally induced pluripotent stem cells (iPSCs);
wherein the population of pluripotent stem cells is contacted with an aggregation medium for a sufficient time to generate the CD34+ hemogenic endothelium;
wherein the population of pluripotent stem cells is differentiated into a population of CD34⁺ hemogenic endothelium by way of embryoid bodies or 2D adherent cultures;
wherein the sufficient time to promote differentiation into a population of CD34+ hemogenic endothelium is at least 8 days;
wherein the aggregation media comprises BMP4, SB-431542, CHIR99021, bFGF, VEGF, IL-6, IL-11, IGF-1, SCF, and EPO;
wherein the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/mL IL-11, 25 ng/mL IGF-1, 50 ng/mL SCF, and 2 U/ml EPO;
further comprising selecting or isolating the resultant population of CD34hemogenic endothelium using expression of surface markers on the population of CD34+ hemogenic endothelium; and/or
wherein the population of CD34+ hemogenic endothelium is CD45 negative/low and/or CD38 negative/low.

34-40. (canceled)

41. The method of claim 1, further comprising the step of genetically modifying the resultant CD34+ hemogenic endothelial cells or the resultant CD3+ T cells; and/or
wherein the genetic modification is editing an endogenous HLA, removing an endogenous TCR, and/or expressing a chimeric antigen receptor (CAR).

42. (canceled)

43. The method of claim 1, wherein the CD3+ T cells are CD3⁺TCRαβ⁺ T cells; and/or
wherein the CD3+ T cells comprise a diverse T cell receptor (TCR) repertoire.

44. (canceled)

45. The method of claim 1, wherein the method further comprises inhibition of EZH1 activity and/or expression in the CD34+ HE cells;
wherein the EZH1 activity and expression are inhibited by an RNA-guided nuclease system;
wherein EZH1 activity and expression are inhibited using a doxycycline-inducible CRISPR interference (CRISPRi) system;
wherein the inhibition of EZH1 activity and/or expression comprises contacting the cells with an inhibitor of EZH1 expression; and/or
wherein the inhibitor of EZH1 expression comprises an RNA interference molecule.

46-49. (canceled)

50. A cell or population of cells made by the method of claim 1.

51. A method of treating cancer, the method comprising administering a cell or population of cells of claim 50.

52. A method for generating mature αβ T cells, the method comprising:
(a) contacting a CD34+ hemogenic endothelium (HE) with a CD5+CD7+ differentiation medium comprising interleukin-3 (IL-3) under conditions and for a sufficient time to generate CD5+CD7+ T cell progenitor cells,
(b) contacting the CD5+CD7+ T cell progenitor cells with a CD3+ T cell differentiation medium under conditions and for a sufficient time to generate mature αβ T cells.

53-58. (canceled)


SEQUENCE LISTING (58 sequences)

SEQ ID NO: 1 - DNA, 8873 bp, Homo sapiens
  source 1..8873: mol_type = genomic DNA
actgaccatttggcgatccattgagaggagggtttggaaaagtggctcctttgtgacagctctcgccagattggggggctgctgatttgcatctcattagccatgcgggcggccggctgaatataagggcggcaggcgccggcgagagccagatcctctgcgcgcacccgcggagacccgacccggccgagggcagagcgcaggggaacccgggcagccgcggcgcagagcctcctcccacggcccggcccctccggtcctgcgcgtgtgtactggatggcattggctggattcatcggaaagacgcggatctttgctgtgacaccggagatcggagcccggagtgctcccggaacgaccgccgccgccgagtgacaccgggccgcgatccgcaggggccgccgcgcacacccgccgccgccgaccgtcccctcagcgcgcgccgctggccccggattatcgccttgcccgtgggatttccagaccgcggctttctaatcggctcgggaggaagctctgcagctctcttgggaattaagctcaatctctggactctctctctttctctttctccccctccctctcctgcgaagaagctcaagacaaaaccaggaagccggcgaccctcacctcctcgggggctgggaggaaggaggaaaacgaaagtcgccgccgccgcgctgtcccccgagagctgcctttcctcgggcatccctggggctgccgcgggacctcgcagggcggatataaagaaccgcggccttgggaagaggcggagaccggcttttaaagaaagaagtcctgggtcctgcggtctggggcgaggcaagggcgcttttctgcccacgctccccgtggcccatcgatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtaggcgggcaggtgggggcgccgcggccccgcggggtctcacgggtagccggggcgcggggcaggagcgcgcggggaggggcggacagcggcacgggccgcgccagccacggcccggaagatgaatcccgggggcgacgaccccagcgccggccgtgcagcgagcgcgctcggcccctgagcccttccaggctctccgcacaccccccacccaggcctcacgccccctagctcgggcgggacccgcgtcctcacgcccccgccctcccccgtgcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccggtgagtgccgcacctgcgcgcgccgggccggccctgaagctgggcgggctgcaggacgcgctgggatcccgccttgggcgctcggtggcgggacctcggggaccccgcgaggcgcaggtgggcgctgcgatctgcctagcggcggccccaggactccagcccagcagcgcggacacctcgccccggggccccgcggcctgcaggaggggaccgcgctggggcgaggaggagaggccgagcgcgcccgggagatttccgtatccggcctctgtgccaggtctccagtcagaggcgccccttcacgtgggaaggttctggtttcccgactcctagacgcgttggtggcgcgattacccgcgcagcgcgaccgctaccacccggagcgtgcccatcccccaagaaaaatgacaagggccctcgggcctcttccaccccatcctgcctgcattctctctctctctctaattaaaaaaacaacgtaatatcctgtagtacaggctgaaaaaacacgtcaggaaaccactctttaaaaagttcttccatttccttagggaaggtgagagcaggcaggaggtgcgtggagaccctctccagacacgctgccccagacctgcagccttcaggcctctgttgctgacctggctgttaggaatgactgctttttgccgttttcttttcgttacctttctgggttgtctaacgtcttctcccctctctcccagggcaccttctctctgattattgaagctctccacacagattctcctgatgacctcgcaacaggtaaaaacaaaacccaaaccccaaaactgctttccccagttaatagcattggactttgcccacccatcccccagccaaacccggacagctttcattctgcacgtgccccagaaagttcagggtggagcagcttgggcctccttcccgtgctgaatgtctcggcccacccccgctctgtcccgagtcacagggttctcgttcagaaccaaccaggagcatcttctccccgtagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagcgtgagtctctgggaaggcaccgctggctcactcgtccacgaacacggaccgcgcgcagggacggggcttcctgagccacggggggcttgggactgtagagatgttctggtggggaaactgaggcccagaggacagaagtggattgctataagtcacagctcgtcagtgggggggttggggtcaacgcagacattttaacatcccaggctgtgtttatccactatcggaactgcctttcttaatcagggaggattttagagacagggccaggggtcaggaagtaaagccagtgctacccccagggtgtgtgtattagagagggagaggaggaaggaagggaggaacacagagagagcttgtgtgtcaggggcaccatttcaacccgagttcccagtgctggaacagcatcacactgggaaacgttccattttctctctggagctggtgtgcttgacctctctggagcaaacgcctttccggatactccctgtgacacgcactgtctatgctggccagagagcaggctttcactcctgtgggctgctgaggccaggtctccaaggcctgtgtgggcgaggggtgcacagccccgtctggcttgaatgctcaggcagcaccttgtctggagaagcaatgtcttcccaatagtgacagaggctctacctgcctcttattaggtattgatgtgtcaatgtcatggcaggcaggtgactagggcagggttggggccgtgctggctcctggttctggctcatggggacctcaggagccctctctccagctgactgaggcctcgcctgcacgcctggccgtcccagcccattggtaccggatttctctacagctggggattgggtaggtcctggagctgcccagaaactccagggaactgtcattctccttccttggaactggacaaccttggagaggggctctgggaggcccagaacctctggcaggagctgggtagtgcctggggttgagggtgggtcttcccattcactgagtgccttgatgtccttgctccttagcttcccaaattccctccggaacttactgagctccttctaagctttgccttggcctgaactggttctggggaaaaacaaaaaaacaaaaaacaacttgtggagctgcttgttaatgagtttcataaccaggcagcaagagccagctccaagcctcaagcccactgtctactccctgccctgcgggagcctctggccagtctgctgcctcccacccttcctccctgcctctcttcaccacagggtagccagaaacttaaacttttttcttcaaacactgaagtctctccccgcccccagctcgcgcgtgccatagattagatctctccggggataggcgcagggacacccgccggctcccattggcggaaggggtgcgtgtgcgtgtgtgtgtgtgtgtgtgtgtgtacacgcgaggggtgtgtgtgaggaggtggggccgggggcgcgggggaggccggcattgttgcgctggggcagctgccgtggaggacagacaatggagcagctgtcctgccctggcaccctgcataccagctgtccactcttatctgcacacacactttctgggatattaagaggtggagctttgtgcacagaattgggaagtgggggaggaggagggggaagacttctgaccctctcttagaagaaaaggggatagggtgggggtgggggcttccgagagcccttttgtccttgagcccctgtgttaagaagaatgctcatccccagggctgagtcaagtcccaggctactaggcaggggggtcagtcctccacaacctgggaagattaactcagctgggatttgctgactgaagccggcgagttgtgtcctggccccaagggcggcagccctgttgggacgtacttggcgtggggcttgaccctgtttttcctttgcttgtagcgatctgcctgcctggatgtgatgagcagcatggattttgtgacaaaccaggggaatgcaagtaagtctgcacaaggtggtgttttgttttgttgccttttcttgttatcttttcacagctggtgtatttgtaaaaacagccctaggtgatcattcgaaaaactccagtaagattgattgaacagggggccgttttctcatgtttctacttaatcaatgtttggcagcatgtaaggtcatggagttgtcattcgtctaagccccttaacggctatgagaatttacagatagtagtttaaaaagagttggcacaggaaatgatagtatagttcaatggttctcaaatgttgcctcatcctagaatcactcagggagtgatttttgagatgctgacactggtgctgccctaacacccaagaagccagaacctctggtggggcccaggcccaggctgcagctcccaaggtgacccagtgttctgctaatctggagaaccagaggctcactggtgctgcgggaagatggtttctagggtgagaatgtccactgcaaagccagcaacagtcaacgtccatctgagtcttctgcttttctccaaggtgcagagtgggctggcagggccggtactgtgacgagtgtatccgctatccaggctgtctccatggcacctgccagcagccctggcagtgcaactgccaggaaggctgggggggccttttctgcaaccagggtaagccttctctccctgaggcagcctgctccctccagagcagccctggacttccctggctgtttgatcactggaaaaataaagtcttcctgcatttgatgtcgagcttcctatctcctacttttcctgtccccacccttcacagacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggtgagtcggaggctccatggcatctcacccggaagctggggtgccctggtgttgaatggagtgtgtgggctccttggagcaactttggaaagccttttctgacctctccatcgtgtaggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtaagggggcagctggtgattgctcagagactcgggcgagcggtcaatactgaggtggcattaaaaacaagcatttgtgagtgacctcgagtttatgaatcacttttatccagaccgccaggaattctcgatggaaactctatctttgagtctggaaaggcctggggaatgagagaggccagggcatttgttatgaagttctctgtggaaacctagaccaagcagtgaatgacttgctcagggccacaaggtgcttcgggcacctgcggccgcctgaggttcagtaagtgatgcccacaggtgccggccactccagcttgggaggatggcccagctgtgtggccacccagcacagtagttgggggtgtccctgagtgaggacagagagcctcctgctagcagcgaggggctggctgcccaaaggagacacacagcaaggagagctgggccccagatgtgccggagcattccggaatggtcatccttcccctccctccctcccctgttgtcagtgcctgctcctctcacttgctgtgtaactgtgggcaaggacaccctcgttaagcctcagtttccccatctgaaacctgggtcgagtggcacatgctcttgcccggctgttgtggcgactaatgcagccaccagagtgttctgcacagcgcctgtccagatgctggccgtgtggtttctgacttgtagagctagacctggacacctctcgtatttgaggtcctaaaccatgtcaccttgcgctgtggactcattcaggccacagactgtctttggtttgtctggtttctacagtgtcagacagatagatgcttcagagtgactttttggtgaacaaacctacgaggagacacgtgatgttcatgtccctgtgttccaggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcagggggtgcgtgctgcgggccgggcatcaggagggggtacctggggggtgtcttcctggaaccactgctccgtttctcttcccaaatgttctcatgcattcattgtggattttctctattttccttttagtggagaagcatctgaaagaaaaaggccggactcgggctgttcaacttcaaaagacaccaagtaccagtcggtgtacgtcatatccgaggagaaggatgagtgcgtcatagcaactgaggtcagtgcaggcagcagccgctccctcctcctcggcatgggagcacctgaagctggagcacgggaatcggtctcaggctaacttcccatttgtcttgtggccccccaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaatgacaa

SEQ ID NO: 2 - DNA, 3779 bp, Homo sapiens
  source 1..3779: mol_type = genomic DNA
actgaccatttggcgatccattgagaggagggtttggaaaagtggctcctttgtgacagctctcgccagattggggggctgctgatttgcatctcattagccatgcgggcggccggctgaatataagggcggcaggcgccggcgagagccagatcctctgcgcgcacccgcggagacccgacccggccgagggcagagcgcaggggaacccgggcagccgcggcgcagagcctcctcccacggcccggcccctccggtcctgcgcgtgtgtactggatggcattggctggattcatcggaaagacgcggatctttgctgtgacaccggagatcggagcccggagtgctcccggaacgaccgccgccgccgagtgacaccgggccgcgatccgcaggggccgccgcgcacacccgccgccgccgaccgtcccctcagcgcgcgccgctggccccggattatcgccttgcccgtgggatttccagaccgcggctttctaatcggctcgggaggaagctctgcagctctcttgggaattaagctcaatctctggactctctctctttctctttctccccctccctctcctgcgaagaagctcaagacaaaaccaggaagccggcgaccctcacctcctcgggggctgggaggaaggaggaaaacgaaagtcgccgccgccgcgctgtcccccgagagctgcctttcctcgggcatccctggggctgccgcgggacctcgcagggcggatataaagaaccgcggccttgggaagaggcggagaccggcttttaaagaaagaagtcctgggtcctgcggtctggggcgaggcaagggcgcttttctgcccacgctccccgtggcccatcgatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaatgacaa

SEQ ID NO: 3 - DNA, 2172 bp, Homo sapiens
  source 1..2172: mol_type = genomic DNA
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaa

SEQ ID NO: 4 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 5 - protein, 536 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKL

SEQ ID NO: 6 - DNA, 9734 bp, Homo sapiens
  source 1..9734: mol_type = genomic DNA
agtagcggcgctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcaggtaacacgtcccgcgccctctccgtcccctctgccgcgctctgggcctcagccccgggcaccagctgagctgaccggtcccctccctccttccctcggtccctgtgcaatagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccggtgagcacagcctgggcgcactgggaggtcgcagaagccgagagaggaggcgccctgggaccaaagccccctccccagatttccttgtacacacacccccacccccaaaaagcccaggatgcattctttcctggctcttcccgactctctcctgagactgatcccagaaaaggctctcaccagtctccgtcttcccagtttatgtcctcccgtccccagctcttgggacacgattttcattacctaccactctggggcggtaccctaccaccccctcctccagtggctctcccttacactctcccgtctctcaaccctccctctaccgggggttctcctctcgccttccctgctcaagcgctacactgtgcacagccccgttatgttgacccgggcgcagtaactgaatcctgcaattagattaattaaacaggctgccgcaaggcacccccacctctccccgcttgctcatctcgccatctctccgtccccccaccccctttcccagggtaccttctcgctcatcatcgaagcttggcacgcgccaggagacgacctgcggccaggtgagtagctcgctccgccaccacaggggggcgacacggcgcagcgccgaaagagttaatctgttctaggcgggggaagtgcgggcttgggggtgggaggcaggacgcttagcttggcctggagctgcgccccgcgctggacgctcggattccgctcgctgcctggactcagagcacaattgcgtttcctgcgggttatttttggcgtgggaacgcggggagtacggcggtgagaaaggctgaagctgccagcgccgctgacgggccccttcctgtattttacacctttcgcgaattccgctcctttggaaagggaataatggctttgggatgttgttctgacacagaggaaaaggatatttcagcagcacaacaattctcactttgaaaaggaaaaaagaaaaccattacccacctctggaggcagaacccctgaatgggcaccaaaggaccccctgctcccagggtcctctctagcctggggagcttttctttctttttctcttttttccattttgacctcttttcctctttcccctccctatctgcctccaagaccctgggatatcttaacatccttctattgtcccctttttgaatactatcaggccccctgcacatgcacacacgtagggcagctacgtagcggggctttgggtccctctggcctgttcttgctggcaggcgggggtcatctggataactgggctgattggttggctgatcaccatcatcacagccaagaaggacattggccagccgtcactggcacccttggggactggcgacccttccctgacccgaccctctgccccctcagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcgtaagcagtcaagctcccacctgtgtggaaggggagggtcccctgaggaaacacagtggagcttcttggtcacagcttgcctcccttgaagagtgggtctgggcctcctactagctgggcctcagggatgctgagggtgggcttgacctcagacctcctgtctcttcccagtgctcctcccatcatgccaaagcccacaagaaccccatcatgacattccatccagtttggcttctccttccctgtgccattatttcactttaagacactcggggctcctctgggaggccaggagtaggaagagggcccaggagagctaggggatccccagggccagcaggtgagaatggggcttaagagtccttggtatcccagcctcacccagctctgtgttcttcccttagctatctgtctttcgggctgtcatgaacagaatggctactgcagcaagccagcagagtgcctgtgagtaggggacaggaagtggtgagtgggagccctcccttggccaaggcctctcacctcactctgcctctctcttgttccccagctgccgcccaggctggcagggccggctgtgtaacgaatgcatcccccacaatggctgtcgccacggcacctgcagcactccctggcaatgtacttgtgatgagggctggggaggcctgttttgtgaccaaggtgagtcagggtgaagagagggtgcagagggtgcaagagatatggggctggggggtggaaatccgattcgtcacctggatccttcttacttggtgactgcagacttggctttcccatgatcttccaaggatcttgggtcttttaaggatctttacaactggcccagaatgaggcggtgggtccttctccaggtgcggcggcagggggtggtggagccagggtggctgaaaaacccaggggggtgacaaggtcggcagcctggaggttgcactcataaatcctagcaaagccaaagagagagggatggcaggctcagttcctctttcaaccccgtagttacctattaaccccctgagtgtttgcttaccttccagggctgtttgagcagctctcccctaaacagctgtccggtggggtgtgcccaccggccacctgaggctgtgggtgagctgggcctctgggcggagtggcatctaaccgacttttcggtgtgggcacaaacggcctcccctgctcttacctagttaccacctgcctgaacccatgcggtctctacctggtgtttaggggtagtcactctctggctatacaggggcctttcagccccaaccttgggggaggaggaagccttttttcttgcatcctgctagccagctgcagccagctgcagctcccattttcaggatcaaatgggtgcacctgctgcccagagacaccggcgcaggcctgggtagggtgggcagagagcttgccagggtggaaagaaattgcctaggccctgacttgctgtcaacaaggggcttgggattcagtccctgtgttgtgtgtgtgtgtgtgtgtgtgtgtgtgtgtctgtccctttactaccatccccaccccaacactcacacacctggttcctgctcattctcttccctctccaccatatttgctcccaggtgacacagtcatatactcatcatatgcaaacacagcacttgcaggccatatatttactctgtctggttctccctccctgtccttcccaaataaaaaaacaaatacttatatttcaaaatacccttgtaacacctcttcctttaaaaaatgcccgattactgcctatggtggctctcatctctcctctaccatttctacctgttgaaattttatccctccttccaggcttatctcagctgcccctcctccatgaagccttttctgacttcctccccgacatgtggccttgccctctgctcttcttccttatcttcatcctacttgggttggcagtttgtgagtttccctggcaggacgtcttccagttccagttgtgttgtttcacttttggttgactgcactggtcatatgtgattcaaggtgctttaagaaacatgattttcatcctggctaacacagtgaaaccctgtctgtattaaaaatacaaaagttagccaggtgtggtggcaggcacctgtagccccagctgctgggaaggctgaggcaggagaatggcgaagtagagcttgcagtgagccgaggtcgtgccactgcactccagcctgagtgacagagcaagactccgtctcaaaaaaaaaaaaaaaaaaaaaaaaaagaaacatgattttaggctgggtgcgatggcctgtaatcccagcactttgggaggccgaggtaggtggatcacttgaagtcaggagttcgagaccatcctggccatcctggtgaaacccctgtaaaaatacaaatattaatcgggcacagtggcgcatgcctgtaatcccagctacttagaaggttgaggtatgagaatcgcttgaacccggaaggcgaaggttgtagtgagcctatatcacatcactgcactccagcctgggcgacagagtgagactctgttaaaaaaaaaaaaaaaagaaggaaagaaagagaaagagagagaaagaaagaaagaaagagaaagaaaaaagattttattggtggtggaggaaggatgtttgggcctgggagactttgagttgaggtgtctttgagccaaacatgggggcaaacatggactgcaaggagcctggaggtgagtgcattccctggccctgctcagctgcttggttcctgtttctgcagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggtgaggcccagaccagcgcaggaagacagaggtgtcaggtggtgtctgggcatccctaacctaggcagttagtggatgtacagccatggacaggcattgtgggcaggtggagcccagccttcagtcacacatccctgccccccagggtctgactttggcccctttatggtctctctccaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacggtgcgtgctgctgccctgctaacctggtggactggccctggggctgagagagacttctggtgagggagggtcaggagaggagcgaggcattgtctgccactctggccccccatctgctctggagggcgaagagcttgcttgatcagctggggggctgtggaagcggagctggttagttgcacgcaggccttaggagcaggggtggtatgcaccctgcatagcttccattcctattcccatgtcagaaccccgtcctggctggggtggcctctgaccctccccaggaagtcctgagctggagagagggatgttggaggcttcatgtttctcctcaaaggaggcagtgattcagtcagagccctgctcctggaggcctcatcttgccccgtgcccaggtagagcatgaggtagcatgaggcatcttgaatgtttgcaccttttgaggcacaaagcctgttggtaatccttgtctatctggctcccaggtgaccctctgtgaggcaggcaggcaggcagcgctcaggagctggagaggggtgggaagggctgagagggagtctgctctctcactgaagcctctggcactgccatttcttcatcactgaatgggaaactataatacctgtcctctgtccttcatgtggttgtgaagatgaagtaaaacagtcatgattgtacttatccgagcattaactatataccaaacatgggctcttgccttcatgtaccttcccggctatcctatgaaggggctagcattctactccagtctaacaaatggggaaactgaggcttagagacacggttaagcagcaagtgccagatctcaggccacagagtgacagctgaggtcccaactcaagcctatctgtctgattctacgttaaagttctgtaagatgctagtcatttttatacatgagcccactgaggccgagagaatcaaggtcatgctaaactccaggtctcctgactctgtgcagttctctttgtagtgggctctgcaggtggaggtagaagggcccgaacgtgttcctggaatggggctcccaccccctgccccagggagctcccaggctatcactgacttgtgtctcatgcgtcctcacagggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacaggtgagtggcacccagaagcccagggcctggccaccggccccgacatggttctgcctaggctcctcttaggccaggcgggaagcagttaagcagctgaggttttgttactgacaggaagatcctccagtaggatttctgtcaggggtcctttgtccttccctcccattcattcatttgttcattcacacatgtcaagtgtccctagggtgtctcttgtgacttccgtctttccacagtgtggcttgcctctagtggcagcactggctttatgcagggctcagacccttctggtgaggttgggaggcctgtgactctcttaggggccttttcctaagtgcccccctgcagcagcccagcactgggcacgtccagcccctgtgtcttccccaagaaccaccctgcagatgccctttggctctccagggtcctccctccccccaagcctctccccgtccctcccttacacgcctgtcttgtgttccctcagtgaaaagccagagtgtcggatatcagcgatatgctcccccagggactccatgtaccagtctgtgtgtttgatatcagaggagaggaatgaatgtgtcattgccacggaggtgagtgctgggctcgcctttccttctgccttttgtgggagggaaagtggcctggtcactcttgacccatgggccattcctgaagggtaggtcagaaccctgccttggcaggccaagttcagtggactcttgggtccctgctggcctcattgccactaagggtgtgaaacaggaaccatggcggcaagcctggtctggtcctttcctgctgtattggtgctgggttgggcagccacggcactgctggccagcctctgatgggtgagggggcccctcaccccttgtgcccttcctgccccttcccactggcttcctccattgacctcatgagcgcaagctcccaggcccgtgtgtgtgttgggccgaagactggggaggactgccccacctgcccttagcccctgcctgccccatcgccttctcccagggaggcccagggagggcctggagggagtgcgcatgcccagggtaacctgtttccctgccttccgcttgctcccaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataatgtgtatttataatatgaaacagatgtgtacagga

SEQ ID NO: 7 - DNA, 3426 bp, Homo sapiens
  source 1..3426: mol_type = genomic DNA
agtagcggcgctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataatgtgtatttataatatgaaacagatgtgtacagga

SEQ ID NO: 8 - DNA, 2058 bp, Homo sapiens
  source 1..2058: mol_type = genomic DNA
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 9 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 10 - protein, 526 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPS

SEQ ID NO: 11 - DNA, 21 bp, synthetic construct
  source 1..21: mol_type = other DNA
ctatctggcagtgcgagaatg

SEQ ID NO: 12 - DNA, 21 bp, synthetic construct
  source 1..21: mol_type = other DNA
agacgtgcaagcaggtctttc

SEQ ID NO: 13 - DNA, 21 bp, synthetic construct
  source 1..21: mol_type = other DNA
tggatgacttatgcgtgattt

SEQ ID NO: 14 - DNA, 21 bp, synthetic construct
  source 1..21: mol_type = other DNA
caacagaactttatggtagaa

SEQ ID NO: 15 - DNA, 21 bp, synthetic construct
  source 1..21: mol_type = other DNA
ccgccgtggtttgtattcatt

SEQ ID NO: 16 - DNA, 21 bp, synthetic construct
  source 1..21: mol_type = other DNA
gcttcctcttcaacctcaata

SEQ ID NO: 17 - DNA, 21 bp, synthetic construct
  source 1..21: mol_type = other DNA
ccgccgtggtttgtattcatt

SEQ ID NO: 18 - DNA, 21 bp, synthetic construct
  source 1..21: mol_type = other DNA
gctcttctttgattacaggta

SEQ ID NO: 19 - DNA, 21 bp, synthetic construct
  source 1..21: mol_type = other DNA
gctactcggaaaggaaacaaa

SEQ ID NO: 20 - DNA, 128 bp, Homo sapiens
  source 1..128: mol_type = genomic DNA
attacagcaagatggaaataccaaatccccctacctccaaatgtatcacttactggaaaagaaaagtgaaatctgaatacatgcgacttcgacaacttaaacggcttcaggcaaatatgggtgcaaag

SEQ ID NO: 21 - DNA, 20 bp, synthetic construct
  source 1..20: mol_type = other DNA
tcgacaacttaaacggcttc

SEQ ID NO: 22 - DNA, 20 bp, synthetic construct
  source 1..20: mol_type = other DNA
tgcgacttcgacaacttaaa

SEQ ID NO: 23 - DNA, 20 bp, synthetic construct
  source 1..20: mol_type = other DNA
cctccaaatgtatcacttac

SEQ ID NO: 24 - DNA, 20 bp, synthetic construct
  source 1..20: mol_type = other DNA
taaacggcttcaggcaaata

SEQ ID NO: 25 - DNA, 20 bp, synthetic construct
  source 1..20: mol_type = other DNA
aaacggcttcaggcaaatat

SEQ ID NO: 26 - DNA, 20 bp, synthetic construct
  source 1..20: mol_type = other DNA
catttggaggtagggggatt

SEQ ID NO: 27 - DNA, 20 bp, synthetic construct
  source 1..20: mol_type = other DNA
ccagtaagtgatacatttgg

SEQ ID NO: 28 - DNA, 20 bp, synthetic construct
  source 1..20: mol_type = other DNA
gtgatacatttggaggtagg

SEQ ID NO: 29 - DNA, 20 bp, synthetic construct
  source 1..20: mol_type = other DNA
aagtgatacatttggaggta

SEQ ID NO: 30 - DNA, 20 bp, synthetic construct
  source 1..20: mol_type = other DNA
agtgatacatttggaggtag

SEQ ID NO: 31 - DNA, 20 bp, synthetic construct
  source 1..20: mol_type = other DNA
tttccagtaagtgatacatt

SEQ ID NO: 32 - DNA, 20 bp, synthetic construct
  source 1..20: mol_type = other DNA
taagtgatacatttggaggt

SEQ ID NO: 33 - DNA, 129 bp, Homo sapiens
  source 1..129: mol_type = genomic DNA
gctttgtatgtggcaaattttgcaaaggttcaagaaaaaacccagatcctcaatgaagaatggaagaagcttcgtgtccaacctgttcagtcaatgaagcctgtgagtggacacccttttctcaaaaag

SEQ ID NO: 34 - DNA, 20 bp, synthetic construct
  source 1..20: mol_type = other DNA
gcttcattgactgaacaggt

SEQ ID NO: 35 - DNA, 20 bp, synthetic construct
  source 1..20: mol_type = other DNA
acaggcttcattgactgaac

SEQ ID NO: 36 - DNA, 20 bp, synthetic construct
  source 1..20: mol_type = other DNA
agaaaagggtgtccactcac

SEQ ID NO: 37 - DNA, 20 bp, synthetic construct
  source 1..20: mol_type = other DNA
tccattcttcattgaggatc

SEQ ID NO: 38 - DNA, 20 bp, synthetic construct
  source 1..20: mol_type = other DNA
ccattcttcattgaggatct

SEQ ID NO: 39 - DNA, 20 bp, synthetic construct
  source 1..20: mol_type = other DNA
cccagatcctcaatgaagaa

SEQ ID NO: 40 - DNA, 20 bp, synthetic construct
  source 1..20: mol_type = other DNA
gtatgtggcaaattttgcaa

SEQ ID NO: 41 - DNA, 20 bp, synthetic construct
  source 1..20: mol_type = other DNA
cagtcaatgaagcctgtgag

SEQ ID NO: 42 - protein, 754 aa, synthetic construct
SGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEIEGRMDPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 729 aa, synthetic construct
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 3702 bp, Homo sapiens
  source 1..3702: mol_type = genomic DNA
atgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatga

SEQ ID NO: 46 - protein, 1233 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 47 - DNA, 2427 bp, Homo sapiens
  source 1..2427: mol_type = genomic DNA
atggccgccgccgatgccgaggcagttccggcgaggggggagcctcagcaggattgctgtgtgaaaaccgagctgctgggagaagagacacctatggctgccgatgaaggctcagcagagaaacaggcaggagaggcccacatggctgcggacggtgagaccaatgggtcttgtgaaaacagcgatgccagcagtcatgcaaatgctgcaaagcacactcaggacagcgcaagggtcaacccccaggatggcaccaacacactaactcggatagcggaaaatggggtttcagaaagagactcagaagcggcgaagcaaaaccacgtcactgccgacgactttgtgcagacttctgtcatcggcagcaacggatacatcttaaataagccggccctacaggcacagcccttgaggactaccagcactctggcctcttcgctgcctggccatgctgcaaaaacccttcctggaggggctggcaaaggcaggactccaagcgcttttccccagacgccagccgccccaccagccacccttggggaggggagtgctgacacagaggacaggaagctcccggcccctggcgccgacgtcaaggtccacagggcacgcaagaccatgccgaagtccgtcgtgggcctgcatgcagccagtaaagatcccagagaagttcgagaagctagagatcataaggaaccaaaagaggagatcaacaaaaacatttctgactttggacgacagcagcttttaccccccttcccatcccttcatcagtcgctacctcagaaccagtgctacatggccaccacaaaatcacagacagcttgcttgccttttgttttagcagctgcagtatctcggaagaaaaaacgaagaatgggaacctatagcctggttcctaagaaaaagaccaaagtattaaaacagaggacggtgattgagatgtttaagagcataactcattccactgtgggttccaagggggagaaggacctgggcgccagcagcctgcacgtgaatggggagagcctggagatggactcggatgaggacgactcagaggagctcgaggaggacgacggccatggtgcagagcaggcggccgcgttccccacagaggacagcaggacttccaaggagagcatgtcggaggctgatcgcgcccagaagatggacggggagtccgaggaggagcaggagtccgtggacaccggggaggaggaggaaggcggtgacgagtctgacctgagttcggaatccagcattaagaagaaatttctcaagaggaaaggaaagaccgacagtccctggatcaagccagccaggaaaaggaggcggagaagtagaaagaagcccagcggtgccctcggttctgagtcgtataagtcatctgcaggaagcgctgagcagacggcaccaggagacagcacagggtacatggaagtttctctggactccctggatctccgagtcaaaggaattctgtcttcacaagcagaagggttggccaacggtccagatgtgctggagacagacggcctccaggaagtgcctctctgcagctgccggatggaaacaccgaagagtcgagagatcaccacactggccaacaaccagtgcatggctacagagagcgtggaccatgaattgggccggtgcacaaacagcgtggtcaagtatgagctgatgcgcccctccaacaaggccccgctcctcgtgctgtgtgaagaccaccggggccgcatggtgaagcaccagtgctgtcctggctgtggctacttctgcacagcgggtaattttatggagtgtcagcccgagagcagcatctctcaccgtttccacaaagactgtgcctctcgagtcaataacgccagctattgtccccactgtggggaggagagctccaaggccaaagaggtgacgatagctaaagcagacaccacctcgaccgtgacaccagtccccgggcaggagaagggctcggccctggagggcagggccgacaccacaacgggcagtgctgccgggccaccactctcggaggacgacaagctgcagggtgcagcctcccacgtgcccgagggctttgatccaacgggacctgctgggcttgggaggccaactcccggcctttcccagggaccagggaaggaaaccttggagagcgctctcatcgccctcgactcggaaaaacccaagaagcttcgcttccacccaaagcagctgtacttctccgccaggcaaggggagcttcagaaggtgctcctcatgctggtggacggaattgaccccaacttcaaaatggagcaccagaataagcgctctccactgcacgccgcggcagaggctggacacgtggacatctgccacatgctggttcagttctgcaggctgggaagcccaaggtcgaggggctgcctttggtga

SEQ ID NO: 48 - protein, 808 aa, Homo sapiens
MAAADAEAVPARGEPQQDCCVKTELLGEETPMAADEGSAEKQAGEAHMAADGETNGSCENSDASSHANAAKHTQDSARVNPQDGTNTLTRIAENGVSERDSEAAKQNHVTADDFVQTSVIGSNGYILNKPALQAQPLRTTSTLASSLPGHAAKTLPGGAGKGRTPSAFPQTPAAPPATLGEGSADTEDRKLPAPGADVKVHRARKTMPKSVVGLHAASKDPREVREARDHKEPKEEINKNISDFGRQQLLPPFPSLHQSLPQNQCYMATTKSQTACLPFVLAAAVSRKKKRRMGTYSLVPKKKTKVLKQRTVIEMFKSITHSTVGSKGEKDLGASSLHVNGESLEMDSDEDDSEELEEDDGHGAEQAAAFPTEDSRTSKESMSEADRAQKMDGESEEEQESVDTGEEEEGGDESDLSSESSIKKKFLKRKGKTDSPWIKPARKRRRRSRKKPSGALGSESYKSSAGSAEQTAPGDSTGYMEVSLDSLDLRVKGILSSQAEGLANGPDVLETDGLQEVPLCSCRMETPKSREITTLANNQCMATESVDHELGRCTNSVVKYELMRPSNKAPLLVLCEDHRGRMVKHQCCPGCGYFCTAGNFMECQPESSISHRFHKDCASRVNNASYCPHCGEESSKAKEVTIAKADTTSTVTPVPGQEKGSALEGRADTTTGSAAGPPLSEDDKLQGAASHVPEGFDPTGPAGLGRPTPGLSQGPGKETLESALIALDSEKPKKLRFHPKQLYFSARQGELQKVLLMLVDGIDPNFKMEHQNKRSPLHAAAEAGHVDICHMLVQFCRLGSPRSRGCLW

SEQ ID NO: 49 - DNA, 22 bp, synthetic construct
  source 1..22: mol_type = other DNA
cagagcgttcctcatggagctg

SEQ ID NO: 50 - DNA, 22 bp, synthetic construct
  source 1..22: mol_type = other DNA
gtgcttgaagccactccagaac

SEQ ID NO: 51 - DNA, 23 bp, synthetic construct
  source 1..23: mol_type = other DNA
caccacatagtcagtgcttcctg

SEQ ID NO: 52 - DNA, 22 bp, synthetic construct
  source 1..22: mol_type = other DNA
agtctgacagcgagagttagcc

SEQ ID NO: 53 - DNA, 20 bp, synthetic construct
  source 1..20: mol_type = other DNA
acccagaagactgtggatgg

SEQ ID NO: 54 - DNA, 20 bp, synthetic construct
  source 1..20: mol_type = other DNA
ttcagctcagggatgacctt

SEQ ID NO: 55 - DNA, 23 bp, synthetic construct
  source 1..23: mol_type = other DNA
gacctctgtcttacttgtggagc

SEQ ID NO: 56 - DNA, 22 bp, synthetic construct
  source 1..22: mol_type = other DNA
cgtcagatggtgccagcaatag

SEQ ID NO: 57 - DNA, 20 bp, synthetic construct
  source 1..20: mol_type = other DNA
ggtgagtgagtaaacaagcc

SEQ ID NO: 58 - protein, 7 aa, synthetic construct
IEGRMDP